(19) United States
(12) Patent Application Publication
(10) Pub. No.: US 2010/0048570 A1
(43) Pub. Date: Feb. 25, 2010

(54) THIAZOLIDINE DERIVATIVES AND METHODS FOR THE PREPARATION THEREOF

(75) Inventors: Sung Soo Kim, Daejeon (KR); Jin Hee Ahn, Daejeon (KR); Hyae Gyeong Cheon, Daejeon (KR); Sang Dal Rhee, Daejeon (KR); Nam Sook Kang, Daejeon (KR); Ki Young Kim, Daejeon (KR); Seung Kyu Kang, Daejeon (KR); Won Hoon Jung, Daejeon (KR); Sung Gyu Kim, Seoul (KR); Sun Young Kim, Seoul (KR); Jae Hong Kweon, Seoul (KR); Sang Kwon Sohn, Seoul (KR); Min Ki Shin, Seoul (KR); Ni Na Ha, Seoul (KR)

Correspondence Address: HOXIE & ASSOCIATES LLC, 75 MAIN STREET , SUITE 301, MILLBURN, NJ, 07041, US

(73) Assignees: Kainos Medicine, Inc, Gangnam-gu, Seoul (KR); Korea Research Institute of Chemical Technology, Daejeon (KR); Yungjin Pharmaceutical Company, Ltd, Gangdong-gu, Seoul (KR)

(21) Appl. No.: 12/523,285

(22) PCT Filed: Jan. 16, 2008
(86) PCT No.: PCT/IB08/00773

§ 371 Date: Jul. 15, 2009

(30) Foreign Application Priority Data
Jan. 16, 2007 (KR) 10-2007-0004577

(51) Int. Cl. A61K 31/5377; C07D 277/00; C07D 415/00; A61K 31/426

(52) U.S. Cl. 514/236.8; 548/200; 544/133; 514/365

(57) ABSTRACT

The present invention relates to novel 2-carbonyl-3-acyl-1,3-thiazolidines having a β-amino group on the acyl chain, in free, prodrug form or pharmaceutically acceptable salt thereof, including their enantiomers, diastereomers and racemates, as efficient inhibitors against DPP-IV. The invention further relates to the pharmaceutical compositions comprising the disclosed compounds. The present invention also relates to methods for preparing the disclosed compounds and for treating DPP-IV-mediated diseases.

This application claims priority from Korean Patent Application 10-2007-0004577, filed Jan. 16, 2007, the contents of which are incorporated by reference herein.

### FIELD OF THE INVENTION

The present invention relates to novel 2-carbonyl-3-acyl-1,3-thiazolidine derivatives having a β-amino group on the acyl chain, in free or pharmaceutically acceptable salts thereof and methods for preparing same.

Dipeptidyl peptidase IV (DPP-IV) is an enzyme that inactivates a hormone such as glucagon-like peptide 1 (GLP-1) and gastric inhibitory peptide (GIP) associated with the regulation of postprandial glucose levels. GLP-1 and GIP are incretins and are produced when food is consumed. GLP-1 acts to increase insulin secretion, inhibit glucagon secretion, delay gastric emptying, maintain satiety and increase beta-cell proliferation and differenctiation. However, active GLP-1 (7-36) is degraded to inactive GLP-1 (9-36) by DPP-IV.

Inhibition of DPP-IV increases the level of circulating GLP-1 and thus increase insulin secretion, which can ameliorate hyperglycemia in type 2 diabetes.

DPP-IV inhibitors also have other therapeutic utilities. DPP-IV inhibitors have not been studied extensively to date, especially for utilities other than diabetes. New compounds are needed so that improved DPP-IV inhibitors can be found for the treatment of diabetes and potentially other diseases and conditions.

Although a variety of DPP-IV inhibitors have been disclosed, so far only one has been approved for use in the United States, and there is still a need for DPP-IV inhibitors with improved efficacy and/or safety.

### SUMMARY OF THE INVENTION

The present inventors have endeavored to develop novel DPP-IV inhibitors and surprisingly found that novel 2-carbonyl-3-acyl-1,3-thiazolidines having a β-amino group on the acyl chain, e.g., compounds of formula Q below, are efficient inhibitors against DPP-IV. Accordingly, it is a primary object of the present invention to provide novel compounds which are 2-carbonyl-3-acyl-1,3-thiazolidines having a β-amino group on the acyl chain, in free, prodrug form or pharmaceutically acceptable salt form, including enantiomers, diastereomers and racemates thereof.

It is another object of the present invention to provide methods for preparing the disclosed compound.

It is further object of the present invention to provide pharmaceutical compositions comprising the disclosed compounds in free, prodrug form or pharmaceutically acceptable salt thereof, including their enantiomers, diastereomers and racemates.

In accordance with one aspect of the present invention, there is provided a compound of formula (Q):

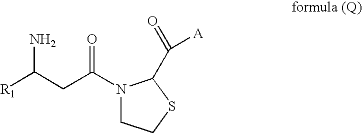

in free, salt or prodrug form, including its enantiomers, diastereoisomers and racemates, wherein:

A is

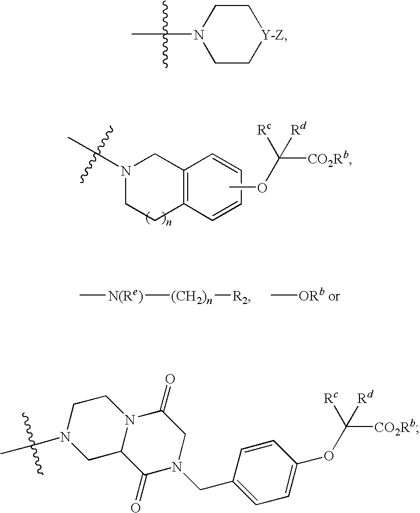

R₁ is

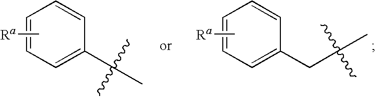

R₂ is C₁₋₆alkyl (e.g., methyl),

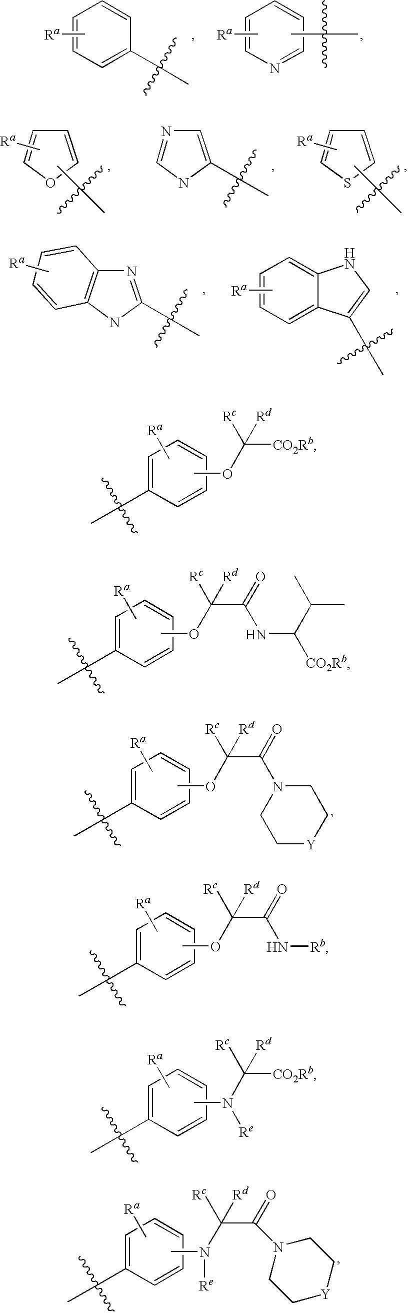

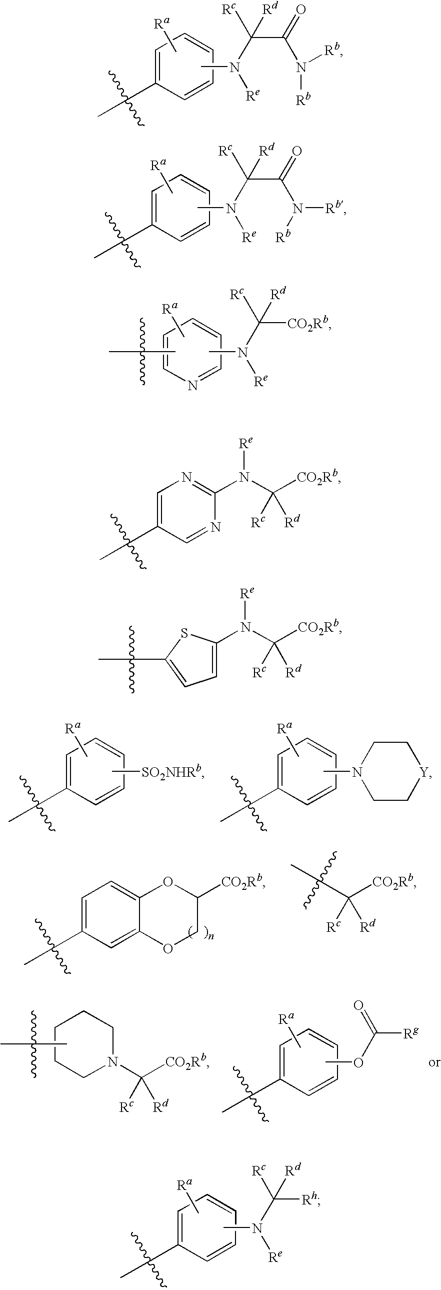

R^{a} is one or more substitutents selected from the group consisting of hydrogen, C₁₋₆ alkyl, C₃₋₆ cycloalkyl, C₁₋₆ alkoxy, —OCF₃, hydroxy, halogen (e.g., fluoro or bromo), —CN, —CF₃, —COOR^{b}, —CH₂COOR^{b}, and —NR^{d}R^{e};

R^{b} and R^{b′} are independently selected from a group consisting of hydrogen, C₁₋₆ alkyl (e.g., methyl, ethyl or isopropyl), C₃₋₆ cycloalkyl or —C₁₋₆alkylC₃₋₆cycloalkyl wherein said cycloalkyl optionally contains one or more heteroatom selected from a group consisting of N, O, or S (e.g., piperazinyl, morpholinyl, morpholin-4-ylethyl, piperidinyl (e.g., piperidin-4-yl or piperidin-1-yl), piperidinylmethyl or piperazinylmethyl), —CH₂CH₂OH, —CH₂CH₂NH₂, —CH₂CH₂N(CH₂CH₂)₂O, —CH₂CH₂N(CH₂CH₃)₂ or —CH₂CH₂NHCOCH₃; CH₂CH₂NHCOCF₃; CH(CH₂OH)₂; CH₂CH₂OCH₃; CH₂CH₂NHCH₃; CH(CH₂CH₂)₂NH and CH₂OCOC(CH₃)₃;

R^{c} is hydrogen, C₁₋₆ alkyl (e.g., methyl, isopropyl, sec-butyl, t-butyl), C₃₋₆ cycloalkyl, or arylC₁₋₆alkyl- (e.g., benzyl);

R^{d} and R^{e} are each independently hydrogen, C₁₋₆ alkyl (e.g., methyl, isopropyl, sec-butyl, t-butyl) or C₃₋₆ cycloalkyl;

R^{g} is C₁₋₆ alkyl (e.g., methyl);

R^{h} is a substituent selected from the group consisting of hydrogen, C₁₋₆ alkyl (e.g., methyl), hydroxyC₁₋₆alkyl (e.g., —CH₂OH);

Y is C, O, S or N;

Z is hydrogen, C₁₋₆ alkyl (e.g., methyl), C₃₋₆ cycloalkyl or —CO₂R^{b} with the proviso that when Y is O or S, Z is absent; and

n is an integer of 0, 1 or 2.

In yet another aspect of the present invention, there is provided a compound of formula (Q) as follows:
1.1. formula (Q), wherein R₁ is

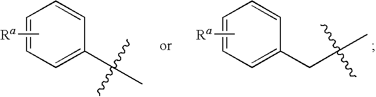

1.2. formula (Q) or 1.1, wherein R₁ is

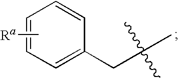

1.3. formula (Q), 1.1 or 1.2, wherein R^{a} is one or more substitutents selected from the group consisting of hydrogen, C₁₋₆ alkyl, C₃₋₆ cycloalkyl, C₁₋₆ alkoxy, —OCF₃, hydroxy, halogen (e.g., fluoro or bromo), —CN, —CF₃, —COOR^{b}, —CH₂COOR^{b}, and —NR^{d}R^{e};
1.4. formula (Q) or any of formulae 1.1-1.3, wherein R^{a} is halo (e.g., fluoro or bromo);
1.5. formula (Q) or any of formulae 1.1-1.4 wherein R₁ is

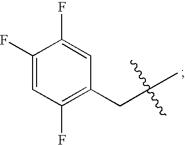

1.6. formula (Q) or any of formulae 1.1-1.5, wherein A is

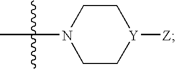

1.7. formula 1.6 wherein Y is C, O, S or N with the proviso that when Y is O or S, Z is absent;
1.8. formula (Q) or any of formulae 1.6-1.7 wherein Y is C;
1.9. formula 1.8 wherein Z is —CO₂R^{b};
1.10. formula 1.9 wherein R^{b} is hydrogen or C₁₋₆ alkyl (e.g., methyl, ethyl);
1.11. formula (Q) or any of formulae 1.6-1.7 wherein Y is N;
1.12. formula 1.11 wherein Z is hydrogen or alkyl (e.g., methyl);
1.13. formula (Q) or any of formulae 1.6-1.7 wherein Y is O and Z is absent;
1.14. formula (Q) or any of formulae 1.6-1.7 wherein Y is S and Z is absent;
1.15. formula (Q) or any of formulae 1.1-1.5, wherein A is

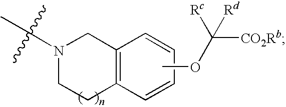

1.16. formula (Q) or any of formulae 1.1-1.5, wherein A is —N(R^{e})—(CH₂)ₙR₂;
1.17. formula (Q) or any of formulae 1.1-1.16, wherein R₂ is selected from the following:

C₁₋₆alkyl (e.g., methyl),

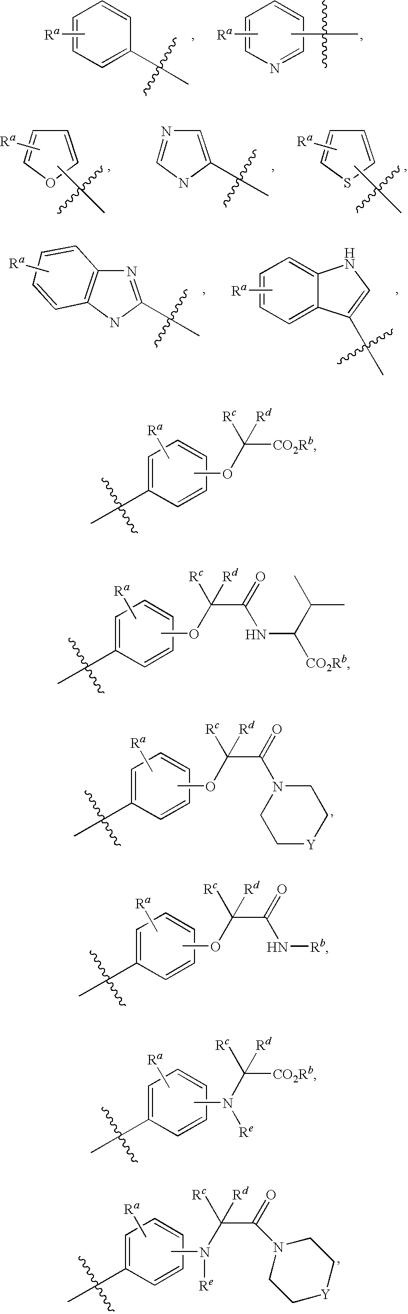

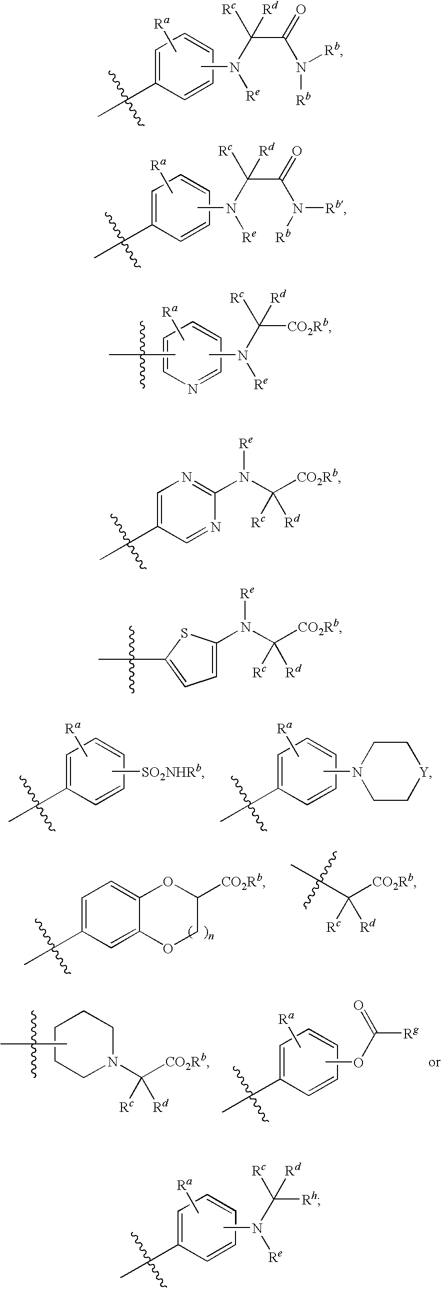

1.18. formula (Q) or any of formulae 1.1-1.17, wherein R₂ is

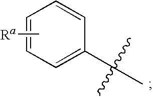

1.19. formula (Q) or any of formulae 1.1-1.17, wherein R₂ is

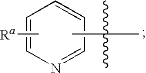

1.20. formula (Q) or any of formulae 1.1-1.17, wherein R₂ is

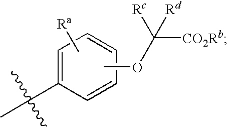

1.21. formula (Q) or any of formulae 1.1-1.17, wherein R₂ is

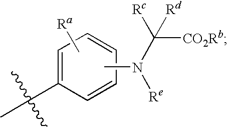

1.22. formula (Q) or any of formulae 1.1-1.17, wherein R₂ is

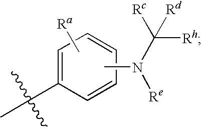

1.23. formula 1.22, wherein R^{h} is hydrogen, C₁₋₆ alkyl (e.g., methyl) or hydroxyC₁₋₆alkyl (e.g., —CH₂OH);
1.24. formula 1.22 or 1.23, wherein R^{h} is CH₂OH;
1.25. formula (Q) or any of formulae 1.1-1.17, wherein R₂ is

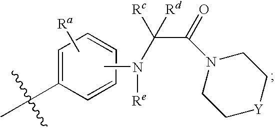

1.26. formula 1.25, wherein Y is O;
1.27. formula 1.25, wherein Y is NH;
1.28. formula 1.25, wherein Y is S;
1.29. formula (Q) or any of formulae 1.1-1.17, wherein R₂ is

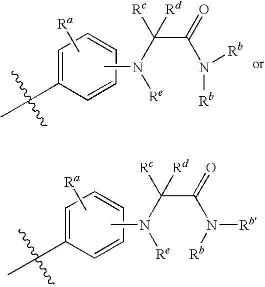

1.30. formula 1.29 wherein R^{b} or R^{b′} is hydrogen or C₁₋₆ alkyl (e.g., methyl);
1.31. any of formulae 1.29-1.30 wherein R^{b} or R^{b′} is methyl;
1.32. formula 1.29-1.30 wherein R^{b} or R^{b′} is hydrogen;
1.33. formula (Q) or any of formulae 1.1-1.17, wherein R₂ is C₁₋₆alkyl (e.g., methyl);
1.34. formula (Q) or any of formulae 1.1-1.17, wherein R₂ is methyl;
1.35. formula (Q) or any of formulae 1.1-1.17, wherein R₂ is

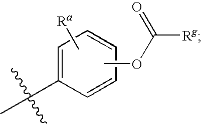

1.36. formula 1.35, wherein R^{g} is C₁₋₆ alkyl (e.g., CH₃);
1.37. formula 1.35 or 1.36 or, wherein R^{g} is —CH₃;
1.38. formula (Q) or any of formulae 1.1-1.37, wherein R^{c} is hydrogen, C₁₋₆ alkyl (e.g., methyl, isopropyl, sec-butyl, t-butyl), C₃₋₆ cycloalkyl or arylC₁₋₆alkyl-(e.g., benzyl);
1.39. formula (Q) or any of formulae 1.1-1.38, R^{c} is hydrogen;
1.40. formula (Q) or any of formulae 1.1-1.38, R^{c} is methyl, isopropyl, sec-butyl, or tert-butyl;
1.41. formula (Q) or any of formulae 1.1-1.38, R^{c} is benzyl;
1.42. formula (Q) or any of formulae 1.1-1.41, wherein R^{d} and R^{e} are each independently hydrogen, C₁₋₆ alkyl (e.g., methyl, isopropyl, sec-butyl, t-butyl) or C₃₋₆ cycloalkyl;
1.43. formula (Q) or any of formulae 1.1-1.42, wherein R^{c} is hydrogen and R^{d} is isopropyl;
1.44. formula (Q) or any of formulae 1.1-1.42, wherein R^{c} is hydrogen and R^{d} is methyl;
1.45. formula (Q) or any of formulae 1.1-1.42, wherein R^{c} is hydrogen R^{d} is benzyl;
1.46. formula (Q) or any of formulae 1.1-1.42, wherein R^{c} is hydrogen R^{d} is sec-butyl;
1.47. formula (Q) or any of formulae 1.1-1.46, wherein R^{c} is hydrogen and the carbon bearing R^{c} and R^{d} has an absolute configuration of (S);
1.48. formula (Q) or any of formulae 1.1-1.46, wherein R^{c} is hydrogen and the carbon bearing R^{c} and R^{d} has an absolute configuration of (R);
1.49. formula (Q) or any of formulae 1.1-1.48, wherein R^{e} is hydrogen, C₁₋₆ alkyl (e.g., methyl);
1.50. formula (Q) or any of formulae 1.1-1.49, wherein R^{e} is hydrogen;
1.51. formula (Q) or any of formulae 1.1-1.49, wherein R^{e} is methyl;
1.52. formula (Q) or any of formulae 1.1-1.51, wherein R^{b} and R^{b′} are independently selected from a group consisting of hydrogen, C₁₋₆ alkyl (e.g., methyl, ethyl, isopropyl), C₃₋₆ cycloalkyl or —C₁₋₆alkyl-C₃₋₆cycloalkyl wherein said cycloalkyl optionally contains one or more heteroatom selected from a group consisting of N, O, or S (e.g., piperazinyl, morpholinyl, morpholin-4-ylethyl, piperidinyl (e.g., piperidin-1-yl or piperidin-4-yl), piperidinylmethyl or piperazinylmethyl), —CH₂CH₂OH, —CH₂CH₂NH₂, —CH₂CH₂N(CH₂CH₂)₂O, —CH₂CH₂N(CH₂CH₃)₂ or —CH₂CH₂NHCOCH₃; CH₂CH₂NHCOCF₃; CH(CH₂OH)₂; CH₂CH₂OCH₃; CH₂CH₂NHCH₃; CH(CH₂CH₂)₂NH; and CH₂OCOC(CH₃)₃;
1.53. formula (Q) or any of formulae 1.1-1.52, wherein R^{b} or R^{b′} is hydrogen;
1.54. formula (Q) or any of formulae 1.1-1.52, wherein R^{b} or R^{b′} is C₁₋₆ alkyl;
1.55. formula (Q) or any of formulae 1.1-1.52, wherein R^{b} is ethyl;
1.56. formula (Q) or any of formulae 1.1-1.52, wherein R^{b} is —C₁₋₆alkyl-C₃₋₆cycloalkyl wherein said cycloalkyl optionally contains one or more heteroatom selected from a group consisting of N, O, or S (e.g., piperazinyl, morpholinyl, morpholin-4-ylethyl, piperidinyl (e.g., piperidin-1-yl or piperidin-4-yl), piperidinylmethyl or piperazinylmethyl);
1.57. formula 1.56 wherein R^{b} is morpholin-4-yl-ethyl;
1.58. formula (Q) or any of formulae 1.1-1.52, wherein R^{b} is isopropyl, —CH₂CH₂OCH₃, —CH₂CH₂OH, —CH₂CH₂NHCH₃, —CH₂CH₂NH₂, CH(CH₂OH)₂, CH₂CH₂NHCOCF₃, or CH₂OCOC(CH₃)₃;
1.59. formula (Q), or any of formulae 1.1-1.58, wherein R^{a} is one or more substitutents selected from the group consisting of hydrogen, C₁₋₆ alkyl, C₃₋₆ cycloalkyl, C₁₋₆ alkoxy, —OCF₃, hydroxy, halogen (e.g., fluoro or bromo), —CN, —CF₃, —COOR^{b}, —CH₂COOR^{b}, and —NR^{d}R^{e};
1.60. formula 1.29 wherein R^{a} is hydroxy;
1.61. formula 1.30 wherein R^{a} is halogen (e.g., fluoro);
1.62. formula 1.30 wherein R^{a} is fluoro or bromo;
1.63. formula 1.30 wherein R^{a} is —CF₃;
1.64. any of the preceding formulae wherein n is 0, 1 or 2;
1.65. any of the preceding formulae wherein n is 1;
1.66. any of the preceding formulae wherein the carbon bearing the amine and the R₁ group of formula (Q) has an absolute configuration of (R);
1.67. any of the preceding formulae wherein the carbon bearing the amine and the R₁ group of formula (Q) has an absolute configuration of (S);
1.68. any of the preceding formulae wherein the carbon bearing —C(O)-A of formula (Q) has an absolute configuration of (R);
1.69. any of the preceding formulae wherein the carbon bearing —C(O)-A of formula (Q) has an absolute configuration of (S);
1.70.
1.71. any of the preceding formulae, selected from the following:
(1) methyl 3-((R)-3-amino-4-(2,4,5-trifluorophenyl)butanoyl)thiazolidine-2-carboxylate,
(2) 3-((R)-3-amino-4-(2,4,5-trifluorophenyl)butanoyl)thiazolidine-2-carboxylic acid,
(3) 3-((R)-3-amino-4-(2,4,5-trifluorophenyl)butanoyl)-N-benzylthiazolidine-2-carboxamide,
(4) ethyl 2-(4-((3-((R)-3-amino-4-(2,4,5-trifluorophenyl)butanoyl)thiazolidine-2-carboxamido)methyl)phenoxy)acetate,
(5) 2-4-((3-((R)-3-amino-4-(2,4,5-trifluorophenyl)butanoyl)thiazolidine-2-carboxamido)methyl)phenoxy)acetic acid,
(6) ethyl 2-(4-(3-((R)-3-amino-4-(2,4,5-trifluorophenyl)butanoyl)thiazolidine-2-carboxamido)phenoxy)acetate,
(7) 2-(4-(3-((R)-3-amino-4-(2,4,5-trifluorophenyl)butanoyl)thiazolidine-2-carboxamido)phenoxy)acetic acid,
(8) ethyl 2-(4-((3-((R)-3-amino-4-(2,4,5-trifluorophenyl)butanoyl)thiazolidine-2-carboxamido)methyl)phenoxy)-3-methylbutanoate,
(9) 2-(4-((3-((R)-3-amino-4-(2,4,5-trifluorophenyl)butanoyl)thiazolidine-2-carboxamido)methyl)phenoxy)-3-methylbutanoic acid,
(10) pivaloyloxymethyl 2-(4-((3-((R)-3-amino-4-(2,4,5-trifluorophenyl) butanoyl)thiazolidine-2-carboxamido)methyl)phenoxy)-3-methylbutanoate,
(11) ethyl 1-(3-((R)-3-amino-4-(2,4,5-trifluorophenyl)butanoyl)thiazolidine-2-carbonyl)piperidine-4-carboxylate,
(12) 1-(3-((R)-3-amino-4-(2,4,5-trifluorophenyl)butanoyl)thiazolidine-2-carbonyl)piperidine-4-carboxylic acid,
(13) 2-(4-((3-((R)-3-amino-4-(2,4,5-trifluorophenyl)butanoyl)thiazolidine-2-carboxamido)methyl)phenyl)acetic acid,
(14) ethyl 2-(2-(3-((R)-3-amino-4-(2,4,5-trifluorophenyl)butanoyl)thiazolidine-2-carbonyl)-1,2,3,4-tetrahydroisoquinolin-7-yloxy)-3-methylbutanoate,
(15) 2-(2-(3-((R)-3-amino-4-(2,4,5-trifluorophenyl)butanoyl)thiazolidine-2-carbonyl)-1,2,3,4-tetrahydroisoquinolin-7-yloxy)-3-methylbutanoic acid,
(16) ethyl 6-((3-((R)-3-amino-4-(2,4,5-trifluorophenyl)butanoyl)thiazolidine-2-carboxamido)methyl)-2,3-dihydrobenzo[b][1,4]dioxin-2-carboxylate,
(17) 6-((3-((R)-3-amino-4-(2,4,5-trifluorophenyl)butanoyl)thiazolidine-2-carboxamido)methyl)-2,3-dihydrobenzo[b][1,4]dioxin-2-carboxylic acid,
(18) (S)-3-((R)-3-amino-4-(2,4,5-trifluorophenyl)butanoyl)thiazolidine-2-carboxylic acid,
(19) ethyl 2-(4-((3-((R)-3-((1-acetoxyethoxy)carbonylamino)-4-(2,4,5-trifluorophenyl)butanoyl)thiazolidine-2-carboxamido)methyl)phenoxy)-3-methylbutanoate,
(20) (3R)-3-amino-1-(2-(morpolin-4-carbonyl)thiazolidin-3-yl)-4-(2,4,5-trifluorophenyl)butan-1-one,
(21) N-(2-(1H-imidazol-5-yl)ethyl)-3-((R)-3-amino-4-(2,4,5-trifluorophenyl) butanoyl)thiazolidine-2-carboxamide,
(22) (S)-2-(4-(((S)-3-((R)-3-amino-4-(2,4,5-trifluorophenyl)butanoyl) thiazolidine-2-carboxamido)methyl)phenoxy)-3-methylbutanoic acid,
(23) (R)-2-(4-(((S)-3-((R)-3-amino-4-(2,4,5-trifluorophenyl)butanoyl) thiazolidine-2-carboxamido)methyl)phenoxy)-3-methylbutanoic acid,
(24) (S)-2-(4-(((R)-3-((R)-3-amino-4-(2,4,5-trifluorophenyl)butanoyl) thiazolidine-2-carboxamido)methyl)phenoxy)-3-methylbutanoic acid,
(25) (R)-2-(4-(((R)-3-((R)-3-amino-4-(2,4,5-trifluorophenyl)butanoyl) thiazolidine-2-carboxamido)methyl)phenoxy)-3-methylbutanoic acid,
(26) (S)-2-(4-(((S)-3-((R)-3-amino-4-(2,4,5-trifluorophenyl)butanoyl) thiazolidine-2-carboxamido)methyl)phenylamino)-3-methylbutanoic acid,
(27) (R)-2-(4-(((S)-3-((R)-3-amino-4-(2,4,5-trifluorophenyl)butanoyl) thiazolidine-2-carboxamido)methyl)phenylamino)-3-methylbutanoic acid,
(28) (S)-2-(4-(((R)-3-((R)-3-amino-4-(2,4,5-trifluorophenyl)butanoyl) thiazolidine-2-carboxamido)methyl)phenylamino)-3-methylbutanoic acid,
(29) (R)-2-(4-(((R)-3-((R)-3-amino-4-(2,4,5-trifluorophenyl)butanoyl) thiazolidine-2-carboxamido)methyl)phenylamino)-3-methylbutanoic acid,
(30) ethyl 2-(3-((3-((R)-3-amino-4-(2,4,5-trifluorophenyl)butanoyl) thiazolidine-2-carboxamido)methyl)phenoxy)acetate,
(31) 2-(3-((3-((R)-3-amino-4-(2,4,5-trifluorophenyl)butanoyl)thiazolidine-2-carboxamido)methyl)phenoxy)acetic acid,
(32) ethyl 2-(3-(3-((R)-3-amino-4-(2,4,5-trifluorophenyl)butanoyl) thiazolidine-2-carboxamido)phenoxy)acetate,
(33) 2-(3-(3-((R)-3-amino-4-(2,4,5-trifluorophenyl)butanoyl)thiazolidine-2-carboxamido)phenoxy)acetic acid,
(34) 2-(4-(3-((R)-3-amino-4-(2,4,5-trifluorophenyl)butanoyl)thiazolidine-2-carboxamido)piperidine-1-yl)-3-methylbutanoic acid,
(35) (S)-ethyl 2-(4-(((S)-3-((R)-3-amino-4-(2,4,5-trifluorophenyl)butanoyl) thiazolidine-2-carboxamido)methyl)phenylamino)-3-methylbutanoate,
(36) (R)-ethyl 2-(4-(((S)-3-((R)-3-amino-4-(2,4,5-trifluorophenyl)butanoyl) thiazolidine-2-carboxamido)methyl)phenylamino)-3-methylbutanoate,
(37) (S)-ethyl 2-(4-(((R)-3-((R)-3-amino-4-(2,4,5-trifluorophenyl)butanoyl) thiazolidine-2-carboxamido)methyl)phenylamino)-3-methylbutanoate,
(38) (R)-ethyl 2-(4-(((R)-3-((R)-3-amino-4-(2,4,5-trifluorophenyl)butanoyl) thiazolidine-2-carboxamido)methyl)phenylamino)-3-methylbutanoate,
(39) (3R)-3-amino-1-(2-(thiomorpolin-4-carbonyl)thiazolidin-3-yl)-4-(2,4,5-trifluorophenyl)butan-1-one,
(40) (3R)-3-amino-1-(2-(piperazine-1-carbonyl)thiazolidin-3-yl)-4-(2,4,5-trifluorophenyl)butan-1-one,
(41) (3R)-3-amino-1-(2-(4-methylpiperazine-1-carbonyl)thiazolidin-3-yl)-4-(2,4,5-trifluorophenyl)butan-1-one,
(42) 3-((R)-3-amino-4-(2,4,5-trifluorophenyl)butanoyl)-N,N-dimethyl thiazolidine-2-carboxamide,
(43) 3-((R)-3-amino-4-(2,4,5-trifluorophenyl)butanoyl)-N-(furan-3-yl)methyl) thiazolidine-2-carboxamide,
(44) ethyl 2-(3-((R)-3-amino-4-(2,4,5-trifluorophenyl)butanoyl)thiazolidine-2-carboxamido)acetate,
(45) 2-(3-((R)-3-amino-4-(2,4,5-trifluorophenyl)butanoyl)thiazolidine-2-carboxamido)acetic acid,
(46) N-(2-(1H-indol-3-yl)ethyl)-3-((R)-3-amino-4-(2,4,5-trifluorophenyl) butanoyl)thiazolidine-2-carboxamide,
(47) 3-((R)-3-amino-4-(2,4,5-trifluorophenyl)butanoyl)-N-(4-morpholinophenyl) thiazolidine-2-carboxamide,
(48) 3-((R)-3-amino-4-(2,4,5-trifluorophenyl)butanoyl)-N-(4-sulfamoylphenyl) thiazolidine-2-carboxamide,
(49) 3-((R)-3-amino-4-(2,4,5-trifluorophenyl)butanoyl)-N-(4-sulfamoylbenzyl) thiazolidine-2-carboxamide,
(50) N-((1H-benzo[d]imidazol-2-yl)methyl)-3-((R)-3-amino-4-(2,4,5-trifluorophenyl)butanoyl)thiazolidine-2-carboxamide,
(51) ethyl 2-(4-((3-((R)-3-amino-4-(2,4,5-trifluorophenyl)butanoyl) thiazolidine-2-carboxamido)methyl)-3-fluorophenoxy)butanoate,
(52) 2-(4-((3-((R)-3-amino-4-(2,4,5-trifluorophenyl)butanoyl)thiazolidine-2-carboxamido)methyl)-3-fluorophenoxy)butanoic acid,
(53) ethyl 2-(4-((3-((R)-3-amino-4-(2,4,5-trifluorophenyl)butanoyl)thiazolidine-2-carboxamido)methyl)-3-fluorophenoxy)-2-methylpropanoate,
(54) 2-(4-((3-((R)-3-amino-4-(2,4,5-trifluorophenyl)butanoyl)thiazolidine-2-carboxamido)methyl)-3-fluorophenoxy)-2-methylpropanoic acid,
(55) ethyl 2-(4-((3-((R)-3-amino-4-(2,4,5-trifluorophenyl)butanoyl)thiazolidine-2-carboxamido)methyl)-2-fluorophenoxy)-2-methylpropanoate,
(56) 3-((R)-3-amino-4-(2,4,5-trifluorophenyl)butanoyl)-N-(pyridin-4-yl methyl)thiazolidine-2-carboxamide,
(57) (S)-2-(2-(4-((3-((R)-3-amino-4-(2,4,5-trifluorophenyl)butanoyl)thiazolidine-2-carboxamido)methyl)phenoxy)-3-methylbutanamido)-3-methylbutanoic acid,
(58) (R)-ethyl 2-(4-((8-(3-((R)-3-amino-4-(2,4,5-trifluorophenyl)butanoyl) thiazolidine-2-carbonyl)-1,4-dioxo-hexahydro-1H-pyrazino[1,2-a]pyrazin-2(6H)-yl)methyl)phenoxy)-3-methylbutanoate,
(59) (R)-2-(4-((8-(3-((R)-3-amino-4-(2,4,5-trifluorophenyl)butanoyl)thiazolidine-2-carbonyl)-1,4-dioxo-hexahydro-1H-pyrazino[1,2-a]pyrazin-2(6H)-yl)methyl)phenoxy)-3-methylbutanoic acid,
(60) ethyl 2-(2-(3-((R)-3-amino-4-(2,4,5-trifluorophenyl)butanoyl)thiazolidine-2-carbonyl)-1,2,3,4-tetrahydroisoquinoline-6-yloxy)-3-methylbutanoate,
(61) 2-(2-(3-((R)-3-amino-4-(2,4,5-trifluorophenyl)butanoyl)thiazolidine-2-carbonyl)-1,2,3,4-tetrahydroisoquinoline-6-yloxy)-3-methylbutanoic acid,
(62) ethyl 5-((3-((R)-3-amino-4-(2,4,5-trifluorophenyl)butanoyl)thiazolidine-2-carboxamido)methyl)benzo[d][1,3]dioxol-2-carboxylate,
(63) 5-((3-((R)-3-amino-4-(2,4,5-trifluorophenyl)butanoyl)thiazolidine-2-carboxamido)methyl)benzo[d][1,3]dioxol-2-carboxylic acid,
(64) 2-(4-((3-((R)-3-amino-4-(2,4,5-trifluorophenyl)butanoyl)thiazolidine-2-carboxamido)methyl)phenoxy)-2-methylpropanoic acid,
(65) (R)-2-(4-((3-((R)-3-amino-4-(2,4,5-trifluorophenyl)butanoyl)thiazolidine-2-carboxamido)methyl)phenoxy)-3-phenylpropanoic acid,
(66) 2-(4-((3-((R)-3-amino-4-(2,4,5-trifluorophenyl)butanoyl)-N-methyl thiazolidine-2-carboxamido)methyl)phenoxy)-3-methylbutanoic acid,
(67) ethyl 2-(4-((3-((R)-3-amino-4-(2,4,5-trifluorophenyl)butanoyl)thiazolidine-2-carboxamido)methyl)phenylamino)-3-methylbutanoate,
(68) 2-(4-((3-((R)-3-amino-4-(2,4,5-trifluorophenyl)butanoyl)thiazolidine-2-carboxamido)methyl)phenylamino)-3-methylbutanoic acid,
(69) ethyl 2-(4-((3-((R)-3-amino-4-(2,4,5-trifluorophenyl)butanoyl)thiazolidine-2-carboxamido)methyl)-3-fluorophenoxy)-3-methylbutanoate,
(70) 2-(4-((3-((R)-3-amino-4-(2,4,5-trifluorophenyl)butanoyl)thiazolidine-2-carboxamido)methyl)-3-fluorophenoxy)-3-methylbutanoic acid,
(71) 2-(4-((3-((R)-3-amino-4-(2,4,5-trifluorophenyl)butanoyl)thiazolidine-2-carboxamido)methyl)-2-fluorophenoxy)-2-methylpropanoic acid,
(72) ethyl 2-(4-(((S)-3-((R)-3-amino-4-(2,4,5-trifluorophenyl)butanoyl) thiazolidine-2-carboxamido)methyl)phenylamino)-2-methylpropanoate,
(73) 2-(4-(((S)-3-((R)-3-amino-4-(2,4,5-trifluorophenyl)butanoyl)thiazolidine-2-carboxamido)methyl)phenylamino)-2-methylpropanoic acid,
(74) (S)-methyl 2-(2-(((S)-3-((R)-3-amino-4-(2,4,5-trifluorophenyl)butanoyl)thiazolidine-2-carboxamido)methyl)-5-bromophenylamino)-3-methylbutanoate,
(75) (S)-ethyl 2-(3-((R)-3-amino-4-(2,4,5-trifluorophenyl)butanoyl)thiazolidine-2-carboxamido)-3-methylbutanoate,
(76) (S)-2-(3-((R)-3-amino-4-(2,4,5-trifluorophenyl)butanoyl)thiazolidine-2-carboxamido)-3-methylbutanoic acid,
(77) (2S,3S)-ethyl 2-(3-((R)-3-amino-4-(2,4,5-trifluorophenyl)butanoyl) thiazolidine-2-carboxamido)-3-methylpentanoate,
(78) (2S,3S)-2-(3-((R)-3-amino-4-(2,4,5-trifluorophenyl)butanoyl)thiazolidine-2-carboxamido)-3-methylpentanoic acid,
(79) ethyl 2-(4-(3-((R)-3-amino-4-(2,4,5-trifluorophenyl)butanoyl) thiazolidine-2-carboxamido)piperidine-1-yl)-3-methylbutanoate,
(80) 4-((3-((R)-3-amino-4-(2,4,5-trifluorophenyl)butanoyl)thiazolidine-2-carboxamido)methyl)phenyl acetate,
(81) 3-((R)-3-amino-4-(2,4,5-trifluorophenyl)butanoyl)-N-(4-hydroxybenzyl) thiazolidine-2-carboxamide,
(82) ethyl 2-((4-((3-((R)-3-amino-4-(2,4,5-trifluorophenyl)butanoyl)thiazolidine-2-carboxamido)methyl)phenyl)(methyl)amino)-3-methylbutanoate,
(83) methyl 4-((3-((R)-3-amino-4-(2,4,5-trifluorophenyl)butanoyl)thiazolidine-2-carboxamido)methyl)-2-hydroxybenzoate,
(84) ethyl 2-(4-((3-((R)-3-amino-4-(2,4,5-trifluorophenyl)butanoyl)thiazolidine-2-carboxamido)methyl)phenoxy)propanoate,
(85) 2-((4-((3-((R)-3-amino-4-(2,4,5-trifluorophenyl)butanoyl)thiazolidine-2-carboxamido)methyl)phenyl)(methyl)amino)-3-methylbutanoic acid,
(86) 4-((3-((R)-3-amino-4-(2,4,5-trifluorophenyl)butanoyl)thiazolidine-2-carboxamido)methyl)-2-hydroxybenzoic acid,
(87) (S)-2-(2-(((S)-3-((R)-3-amino-4-(2,4,5-trifluorophenyl)butanoyl)thiazolidine-2-carboxamido)methyl)-5-bromophenylamino)-3-methylbutanoic acid,
(88) (S)-ethyl 2-(4-(((S)-3-((R)-3-amino-4-(2,4,5-trifluorophenyl)butanoyl) thiazolidine-2-carboxamido)methyl)phenoxy)-3-methylbutanoate,
(89) (S)-ethyl 2-(4-(((S)-3-((R)-3-amino-4-(2,4,5-trifluorophenyl)butanoyl) thiazolidine-2-carboxamido)methyl)-2-fluorophenylamino)-3-methylbutanoate,
(90) (S)-2-(4-(((S)-3-((R)-3-amino-4-(2,4,5-trifluorophenyl)butanoyl) thiazolidine-2-carboxamido)methyl)-2-fluorophenylamino)-3-methylbutanoic acid,
(91) (S)-ethyl 2-(6-(((S)-3-((R)-3-amino-4-(2,4,5-trifluorophenyl)butanoyl) thiazolidine-2-carboxamido)methyl)pyridin-3-ylamino)-3-methylbutanoate,
(92) (S)-2-(6-(((S)-3-((R)-3-amino-4-(2,4,5-trifluorophenyl)butanoyl) thiazolidine-2-carboxamido)methyl)pyridin-3-ylamino)-3-methylbutanoic acid,
(93) 2-(4-((3-((R)-3-amino-4-(2,4,5-trifluorophenyl)butanoyl)thiazolidine-2-carboxamido)methyl)phenoxy)propanoic acid,
(94) (S)-2-(4-(((S)-3-((R)-3-amino-4-(2,4,5-trifluorophenyl)butanoyl) thiazolidine-2-carboxamido)methyl)phenylamino)-3,3-dimethylbutanoic acid,
(95) (S)-2-(2-(((S)-3-((R)-3-amino-4-(2,4,5-trifluorophenyl)butanoyl) thiazolidine-2-carboxamido)methyl)phenylamino)-3-methylbutanoic acid,
(96) (S)-2-(3-(((S)-3-((R)-3-amino-4-(2,4,5-trifluorophenyl)butanoyl) thiazolidine-2-carboxamido)methyl)phenylamino)-3-methylbutanoic acid,
(97) 4-((3-((R)-3-amino-4-(2,4,5-trifluorophenyl)butanoyl)thiazolidine-2-carboxamido)methyl)benzoic acid,
(98) 3-((R)-3-amino-4-(2,4,5-trifluorophenyl)butanoyl)-N-(4-(2-oxo-2-(piperazine-1-yl)ethoxy)benzyl)thiazolidine-2-carboxamide,
(99) 3-((R)-3-amino-4-(2,4,5-trifluorophenyl)butanoyl)-N-(4-(2-oxo-2-thiomorpholinoethoxy)benzyl)thiazolidine-2-carboxamide,
(100) 3-((R)-3-amino-4-(2,4,5-trifluorophenyl)butanoyl)-N-(4-(2-morpholino-2-oxoethoxy)benzyl)thiazolidine-2-carboxamide,
(101) (S)-ethyl 2-(5-(((S)-3-((R)-3-amino-4-(2,4,5-trifluorophenyl)butanoyl) thiazolidine-2-carboxamido)methyl)pyridin-2-ylamino)-3-methylbutanoate,
(102) (S)-2-(5-(((S)-3-((R)-3-amino-4-(2,4,5-trifluorophenyl)butanoyl) thiazolidine-2-carboxamido)methyl)pyridin-2-ylamino)-3-methylbutanoic acid,
(103) (S)-3-((R)-3-amino-4-(2,4,5-trifluorophenyl)butanoyl)-N-(4-((R)-3-methyl-1-morpholino-1-oxobutan-2-ylamino)benzyl)thiazolidine-2-carboxamide,
(104) (R)-ethyl 2-(5-(((S)-3-((R)-3-amino-4-(2,4,5-trifluorophenyl)butanoyl) thiazolidine-2-carboxamido)methyl)pyridin-2-ylamino)-3-methylbutanoate,
(105) (R)-2-(5-(((S)-3-((R)-3-amino-4-(2,4,5-trifluorophenyl)butanoyl) thiazolidine-2-carboxamido)methyl)pyridin-2-ylamino)-3-methylbutanoic acid,
(106) (S)-3-((R)-3-amino-4-(2,4,5-trifluorophenyl)butanoyl)-N-(4-((S)-3-methyl-1-morpholino-1-oxobutan-2-ylamino)benzyl)thiazolidine-2-carboxamide,
(107) (R)-ethyl 2-(5-(((S)-3-((R)-3-amino-4-(2,4,5-trifluorophenyl)butanoyl) thiazolidine-2-carboxamido)methyl)pyrimidin-2-ylamino)-3-methylbutanoate,
(108) (R)-2-(5-(((S)-3-((R)-3-amino-4-(2,4,5-trifluorophenyl)butanoyl) thiazolidine-2-carboxamido)methyl)pyrimidin-2-ylamino)-3-methylbutanoic acid,
(109) (R)-ethyl 2-(5-(((S)-3-((R)-3-amino-4-(2,4,5-trifluorophenyl)butanoyl) thiazolidine-2-carboxamido)methyl)pyridin-2-yloxy)-3-methylbutanoate,
(110) (R)-2-(5-(((S)-3-((R)-3-amino-4-(2,4,5-trifluorophenyl)butanoyl) thiazolidine-2-carboxamido)methyl)pyridin-2-yloxy)-3-methylbutanoic acid,
(111) (R)-ethyl 2-(4-(((S)-3-((R)-3-amino-4-(2,4,5-trifluorophenyl)butanoyl) thiazolidine-2-carboxamido)methyl)-3-fluorophenylamino)-3-methylbutanoate,
(112) (R)-2-(4-(((S)-3-((R)-3-amino-4-(2,4,5-trifluorophenyl)butanoyl) thiazolidine-2-carboxamido)methyl)-3-fluorophenylamino)-3-methylbutanoic acid,
(113) (S)-3-((R)-3-amino-4-(2,4,5-trifluorophenyl)butanoyl)-N-(4-((R)-1-hydroxy-3-methylbutan-2-ylamino)benzyl)thiazolidine-2-carboxamide,
(114) (R)-2-methoxyethyl 2-(4-(((S)-3-((R)-3-amino-4-(2,4,5-trifluorophenyl) butanoyl)thiazolidine-2-carboxamido)methyl)phenylamino)-3-methylbutanoate,
(115) (S)-3-((R)-3-amino-4-(2,4,5-trifluorophenyl)butanoyl)-N-(4-((R)-3-methyl-1-(methylamino)-1-oxobutan-2-ylamino)benzyl)thiazolidine-2-carboxamide,
(116) (S)-3-((R)-3-amino-4-(2,4,5-trifluorophenyl)butanoyl)-N-(4-((R)-1-(dimethylamino)-3-methyl-1-oxobutan-2-ylamino)benzyl)thiazolidine-2-carboxamide,
(117) (R)-2-morpholinoethyl 2-(4-(((S)-3-((R)-3-amino-4-(2,4,5-trifluorophenyl) butanoyl)thiazolidine-2-carboxamido)methyl)phenylamino)-3-methylbutanoate,
(118) (R)-2-hydroxyethyl 2-(4-(((S)-3-((R)-3-amino-4-(2,4,5-trifluorophenyl) butanoyl)thiazolidine-2-carboxamido)methyl)phenylamino)-3-methylbutanoate,
(119) (R)-2-(methylamino)ethyl 2-(4-(((S)-3-((R)-3-amino-4-(2,4,5-trifluorophenyl)butanoyl)thiazolidine-2-carboxamido)methyl)phenylamino)-3-methylbutanoate,
(120) (S)—N-(4-((R)-1-amino-3-methyl-1-oxobutan-2-ylamino)benzyl)-3-((R)-3-amino-4-(2,4,5-trifluorophenyl)butanoyl)thiazolidine-2-carboxamide,
(121) (S)-3-((R)-3-amino-4-(2,4,5-trifluorophenyl)butanoyl)-N-(4-((R)-1-(ethylamino)-3-methyl-1-oxobutan-2-ylamino)benzyl)thiazolidine-2-carboxamide,
(122) (S)-3-((R)-3-amino-4-(2,4,5-trifluorophenyl)butanoyl)-N-(4-((R)-3-methyl-1-oxo-1-(piperazin-1-yl)butan-2-ylamino)benzyl)thiazolidine-2-carboxamide,
(123) (S)-3-((R)-3-amino-4-(2,4,5-trifluorophenyl)butanoyl)-N-(4-((R)-1-(2-hydroxyethylamino)-3-methyl-1-oxobutan-2-ylamino)benzyl)thiazolidine-2-carboxamide,
(124) (S)-3-((R)-3-amino-4-(2,4,5-trifluorophenyl)butanoyl)-N-(4-((R)-3-methyl-1-oxo-1-(piperidin-4-ylamino)butan-2-ylamino)benzyl)thiazolidine-2-carboxamide,
(125) (S)-3-((R)-3-amino-4-(2,4,5-trifluorophenyl)butanoyl)-N-(4-((R)-3-methyl-1-(2-(methylamino)ethylamino)-1-oxobutan-2-ylamino)benzyl)thiazolidine-2-carboxamide,
(126) (R)-2-aminoethyl 2-(4-(((S)-3-((R)-3-amino-4-(2,4,5-trifluorophenyl) butanoyl)thiazolidine-2-carboxamido)methyl)phenylamino)-3-methylbutanoate,
(127) (R)-isopropyl 2-(4-(((S)-3-((R)-3-amino-4-(2,4,5-trifluorophenyl) butanoyl)thiazolidine-2-carboxamido)methyl)phenylamino)-3-methylbutanoate,
(128) (R)-1,3-dihydroxypropan-2-yl 2-(4-(((S)-3-((R)-3-amino-4-(2,4,5-trifluorophenyl)butanoyl)thiazolidine-2-carboxamido)methyl)phenylamino)-3-methylbutanoate,
(129) (R)-2-(2,2,2-trifluoroacetamido)ethyl 2-(4-(((S)-3-((R)-3-amino-4-(2,4,5-trifluorophenyl)butanoyl)thiazolidine-2-carboxamido)methyl)phenylamino)-3-methylbutanoate,
(130) (R)-ethyl 2-(4-(((S)-3-((R)-3-amino-4-(2,4,5 trifluorophenyl)butanoyl)thiazolidine-2-carboxamido)methyl)-2-fluorophenylamino)-3-methylbutanoate,
(131) (R)-2-(4-(((S)-3-((R)-3-amino-4-(2,4,5-trifluorophenyl)butanoyl)thiazolidine-2-carboxamido)methyl)-2-fluorophenylamino)-3-methylbutanoic acid,
(132) (R)-ethyl 2-(4-(((S)-3-((R)-3-amino-4-(2,4,5-trifluorophenyl)butanoyl)thiazolidine-2-carboxamido)methyl)-2-(trifluoromethyl)phenylamino)-3-methylbutanoate,
(133) (R)-2-(4-(((S)-3-((R)-3-amino-4-(2,4,5-trifluorophenyl)butanoyl)thiazolidine-2-carboxamido)methyl)-2-(trifluoromethyl)phenylamino)-3-methylbutanoic acid,
(134) (R)-ethyl 2-(4-(((S)-3-((R)-3-amino-4-(2,4,5-trifluorophenyl)butanoyl)thiazolidine-2-carboxamido)methyl)-3-fluorophenylamino)-3-methylbutanoate, and
(135) (R)-2-(4-(((S)-3-((R)-3-amino-4-(2,4,5-trifluorophenyl)butanoyl)thiazolidine-2-carboxamido)methyl)-3-fluorophenylamino)-3-methylbutanoic acid,
1.72. any of the preceding formulae, selected from the following:

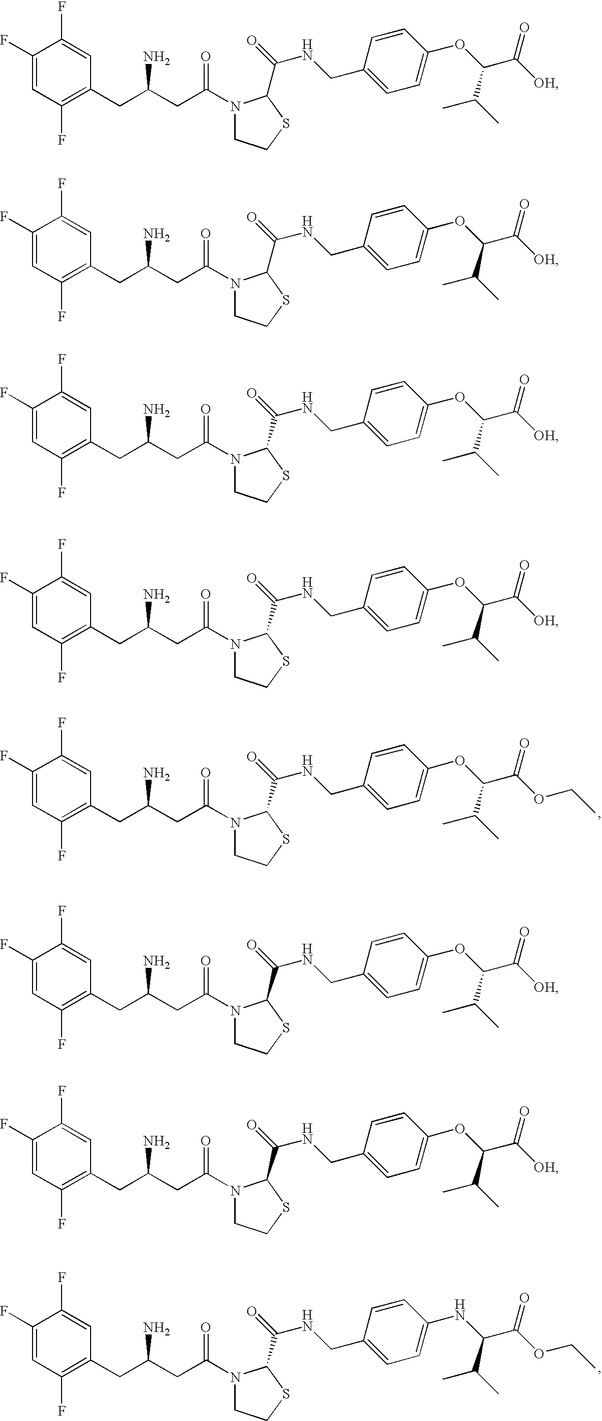

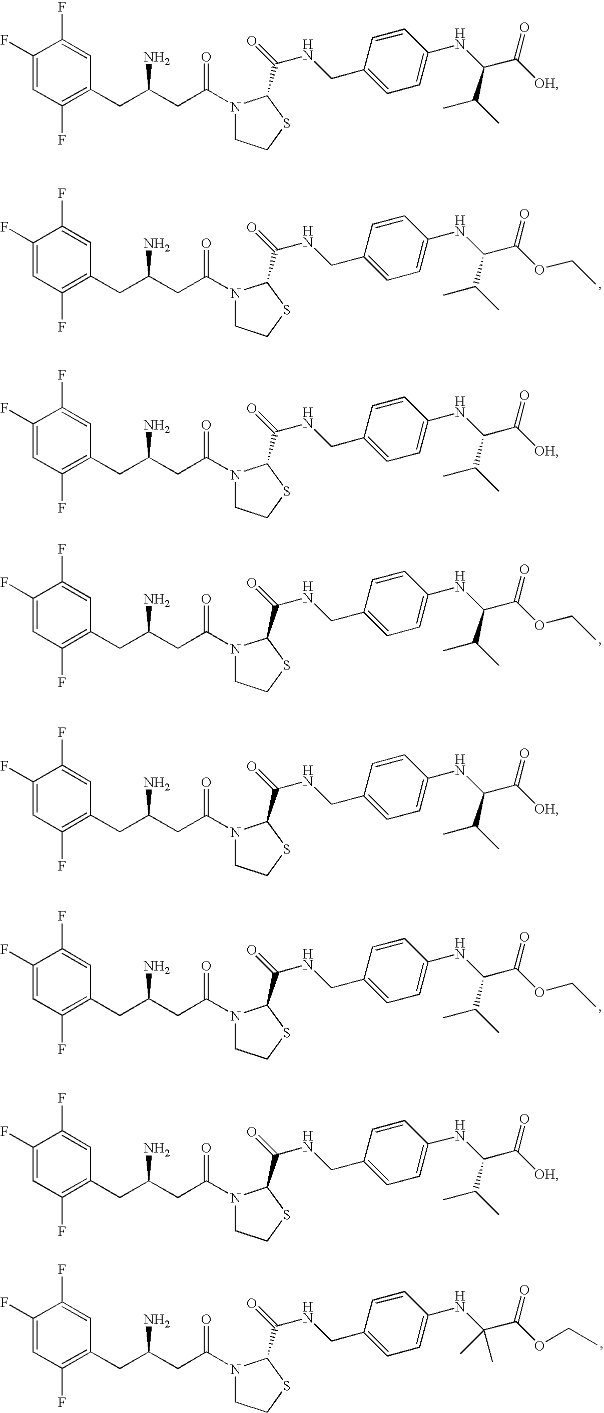

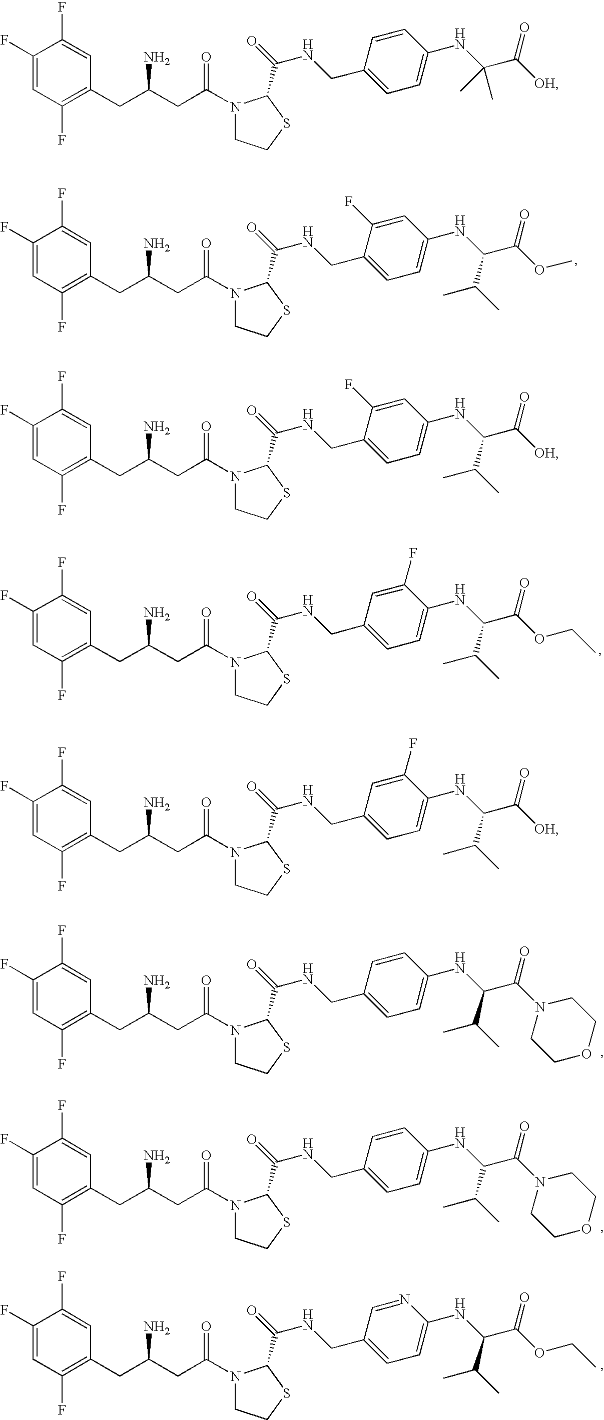

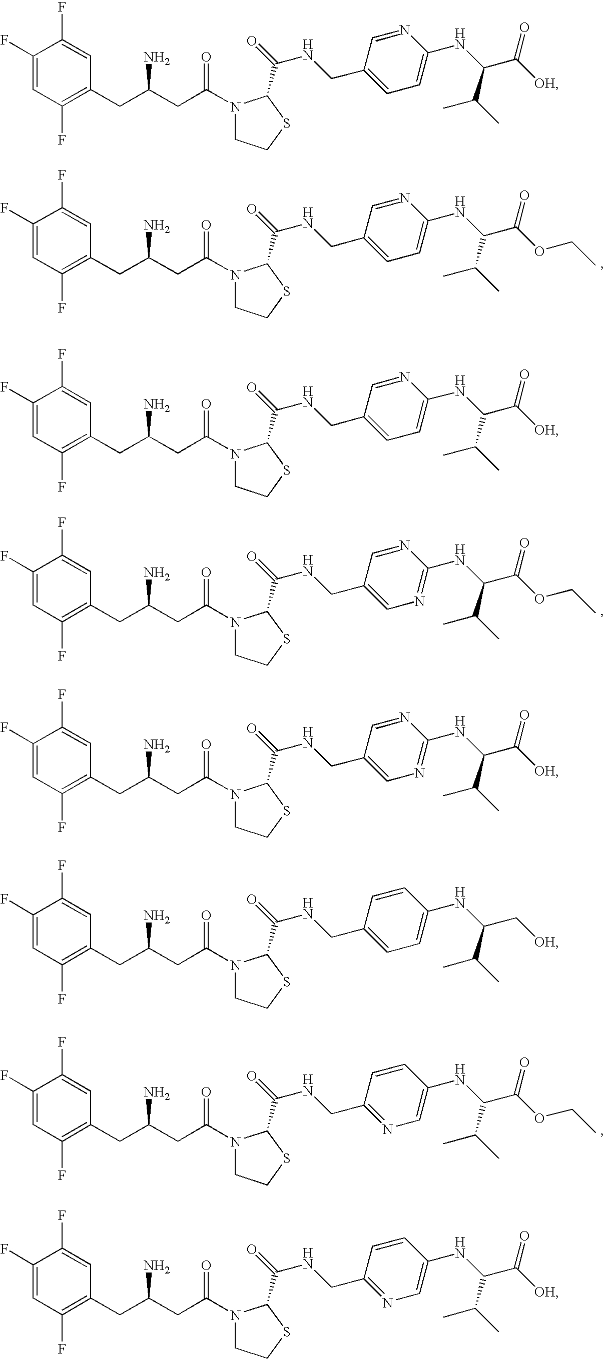

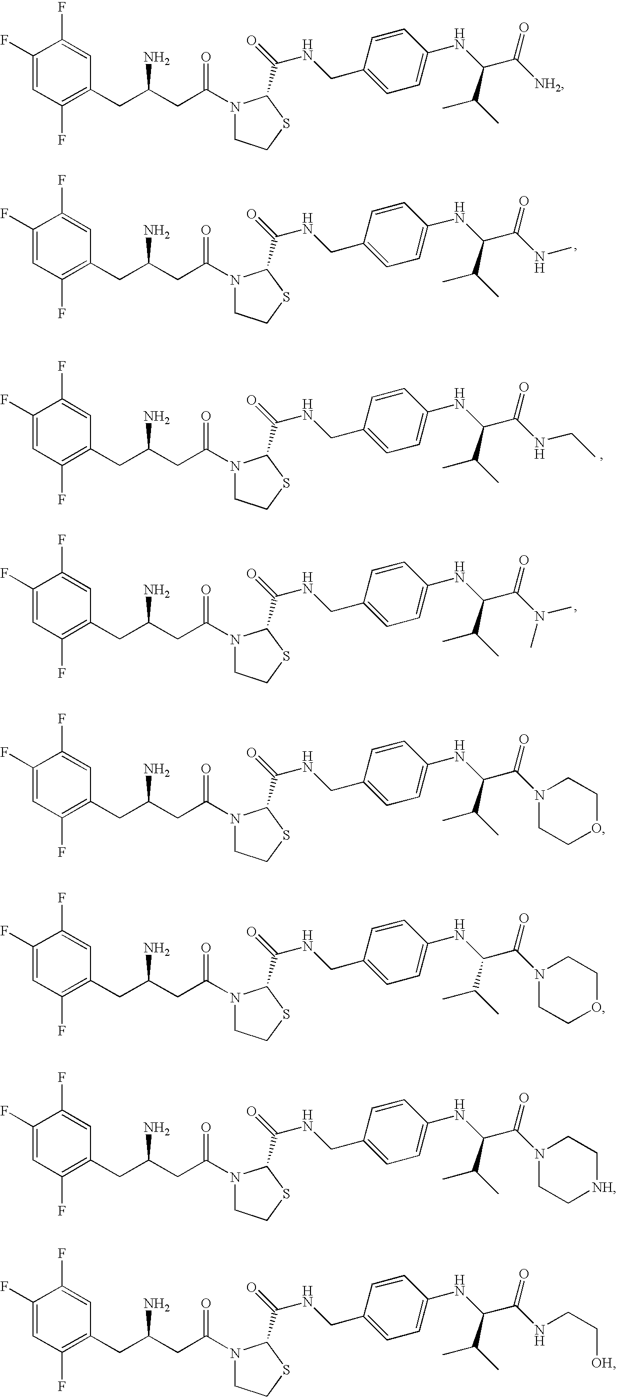

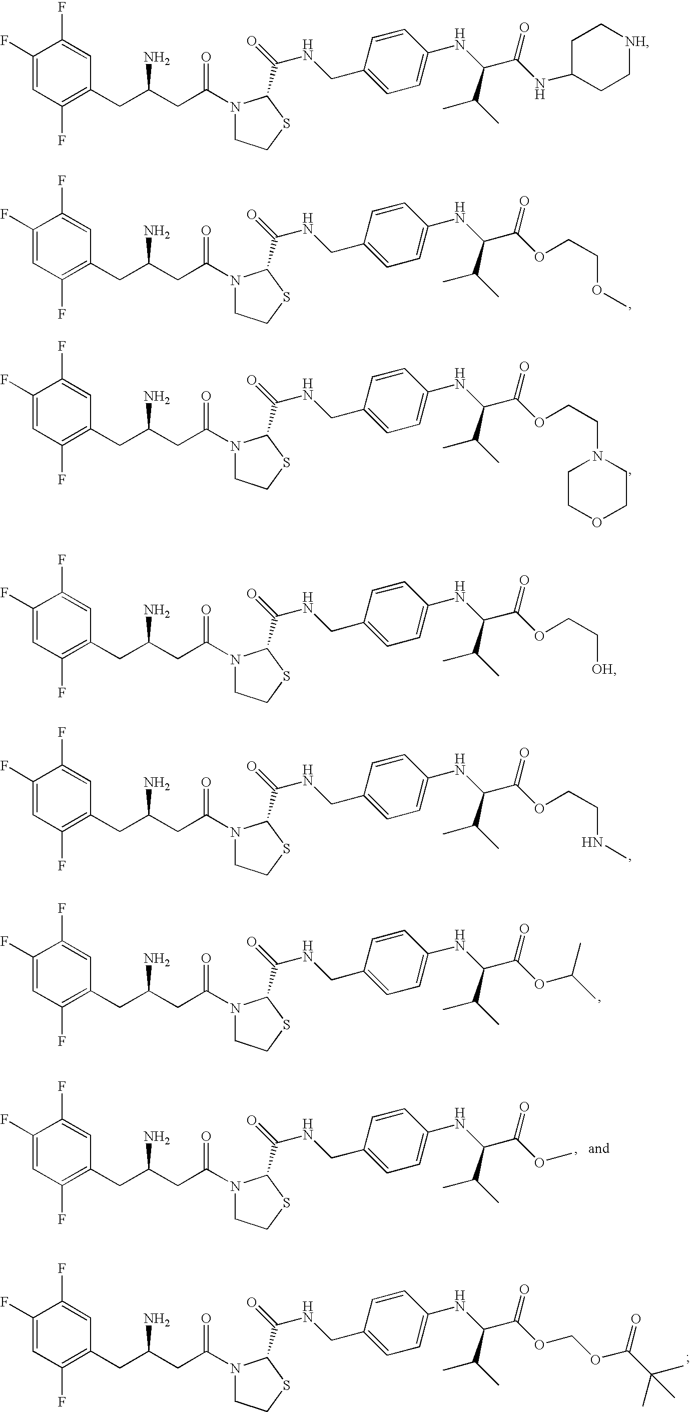

in free, salt or prodrug form, including its enantiomers, diastereoisomers and racemates;
1.73. formula (Q), or any of formulae 1.1-1.71, wherein said compound is selected from:

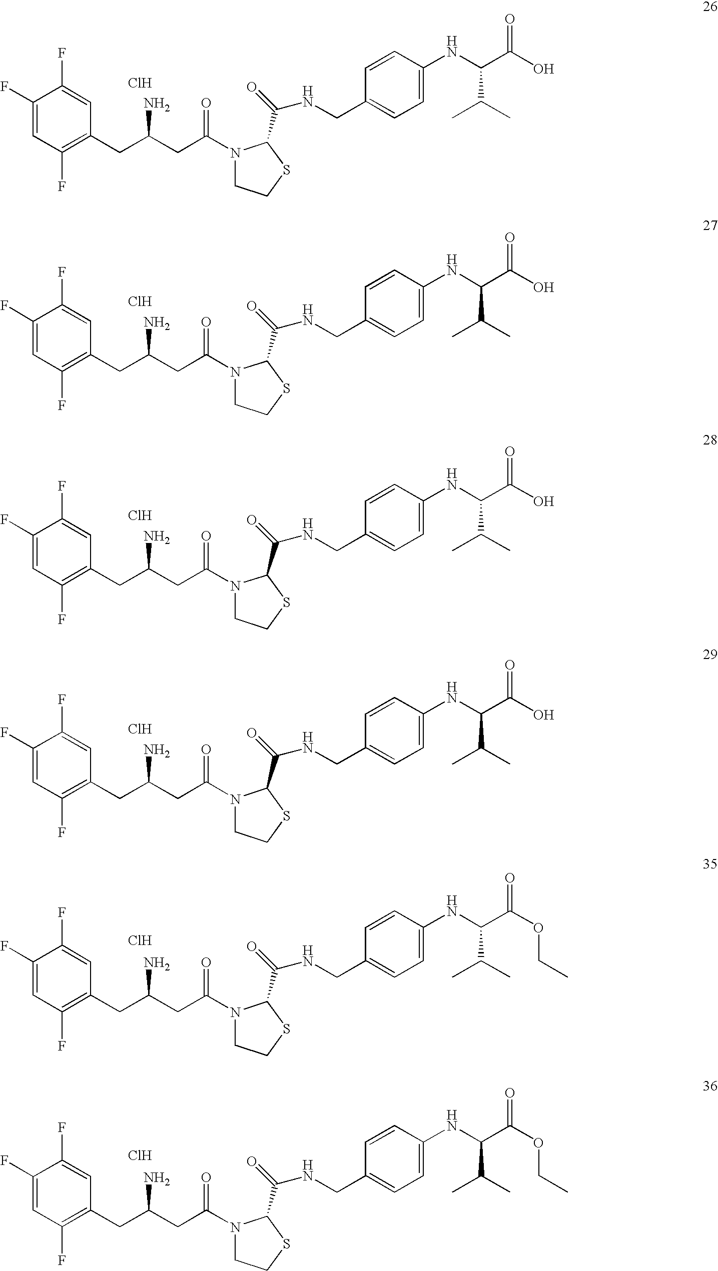

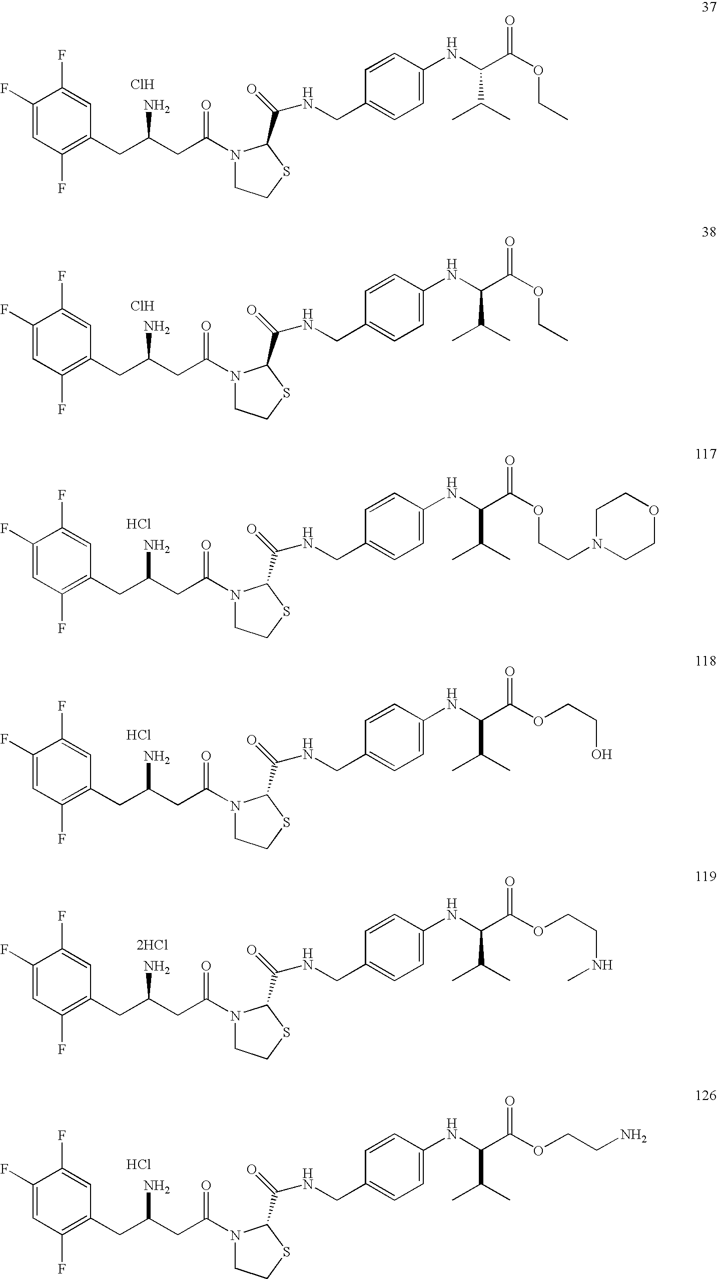

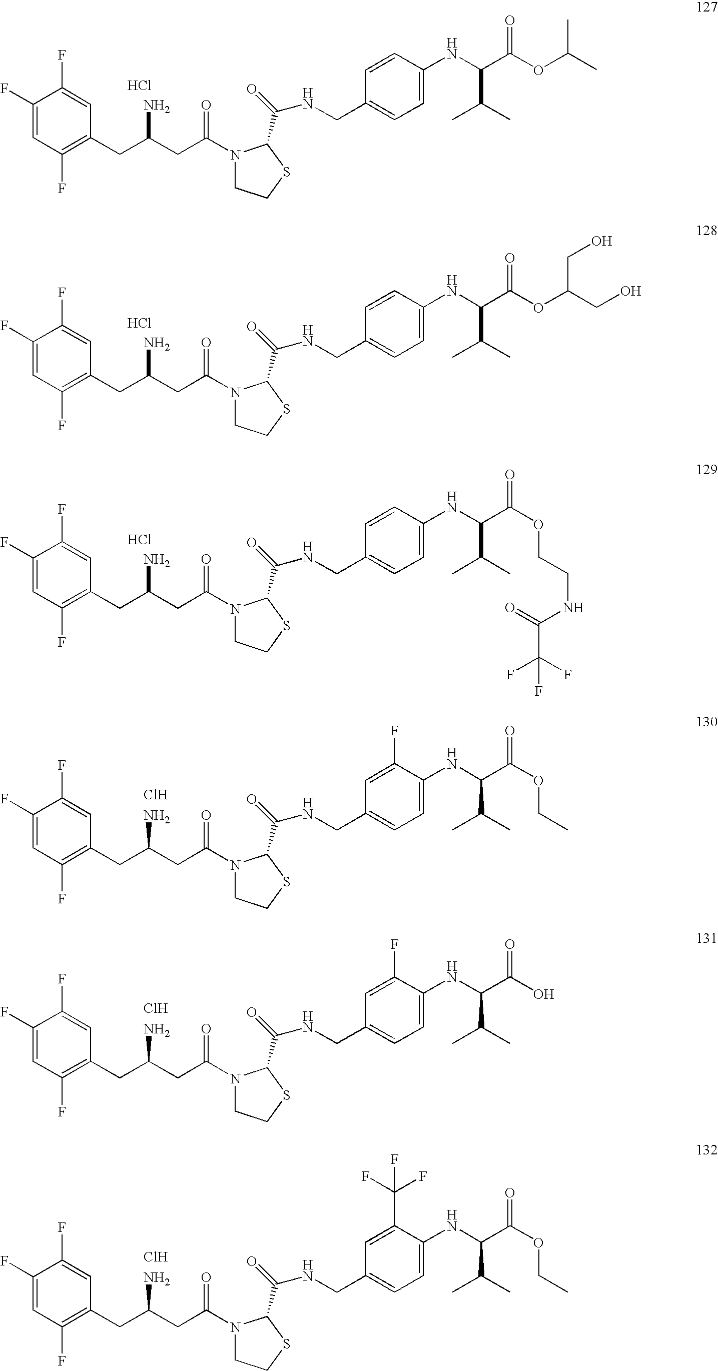

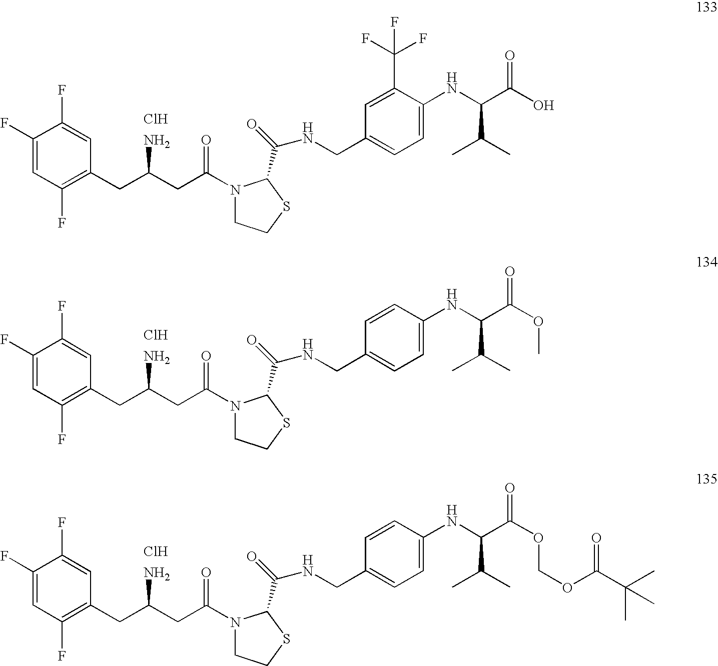

1.74. any of the preceding formulae wherein said compound is in a hydrochloride salt form;
1.75. any of the preceding formulae wherein said compounds inhibit DPP-IV, e.g., with an IC50 value of less than 10 μM, preferably less than 1 μM, most preferably less than 0.05 μM in an assay as shown in the Experimental Example for Table 5 below.

In accordance with another aspect of the present invention, there is provided a compound of 2-carbonyl-3-acyl-1,3-thiazolidines having a β-amino group on the acyl chain derivative having β-amino group on the acyl chain represented by formula 1 or a pharmaceutically acceptable salt thereof:

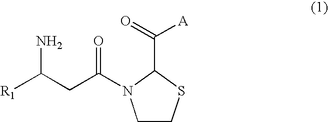

wherein,

A is

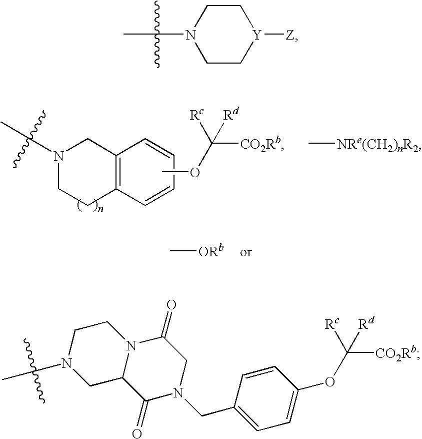

R₁ is

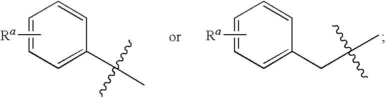

R₂ is

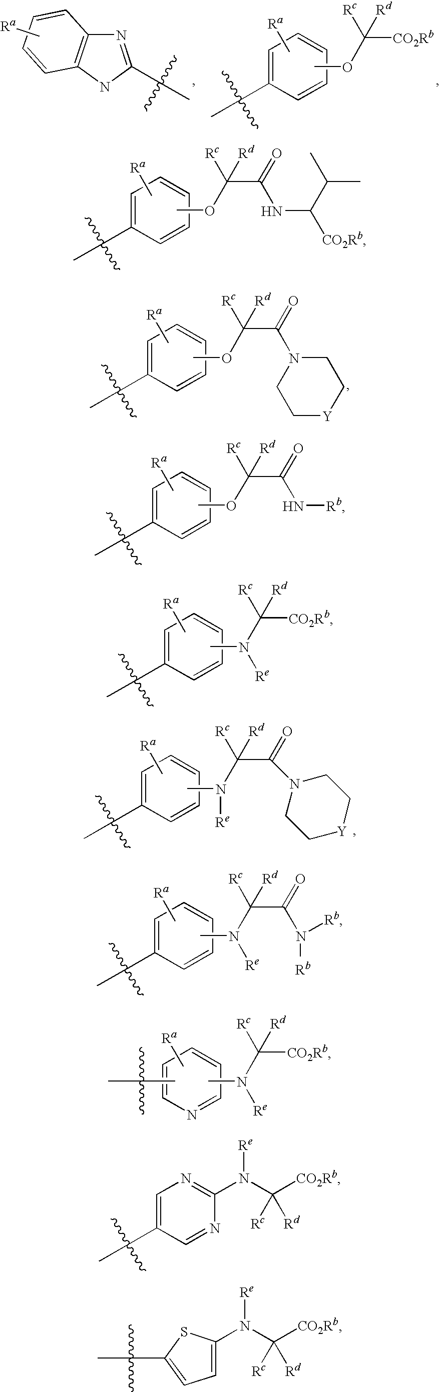

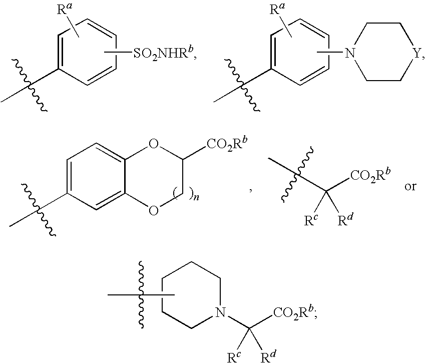

R^{a} is one or more substitutents selected from the group consisting of hydrogen, C₁₋₆ alkyl, C₃₋₆ cycloalkyl, C₁₋₆ alkoxy, —OCF₃, hydroxy, halogen, —CN, —CF₃, —COOR^{b}, —COOR^{b} and —NR^{d}R^{e};

R^{b} is hydrogen, C₁₋₆ alkyl, C₃₋₆ cycloalkyl, isopropyl, t-butyl, —CH₂CH₂OH, —CH₂CH₂NH₂, —CH₂CH₂N(CH₂CH₂)₂O, —CH₂CH₂N(CH₂CH₃)₂ or —CH₂CH₂NHCOCH₃;

R^{c} is hydrogen, C₁₋₆ alkyl, C₃₋₆ cycloalkyl, benzyl, isopropyl or t-butyl;

R^{d} and R^{e} are each independently hydrogen, C₁₋₆ alkyl or C₃₋₆ cycloalkyl;

Y is C, O, S or N;

Z is hydrogen, C₁₋₆ alkyl, C₃₋₆ cycloalkyl or —CO₂R^{b}; and

n is an integer of 0, 1 or 2.

In accordance with yet another aspect of the present invention, there is provided a method (Method (I)) for preparing a compound of 2-carbonyl-3-acyl-1,3-thiazolidine derivative of formula Q-1a, comprising the steps of:

(i) subjecting an amino acid of formula Q-2 to a condensation reaction with a 2-carbonyl-1,3-thiazolidine-based compound of formula Q-3 to form a compound of formula Q-4; and

(ii) deprotecting the compound of formula Q-4 to obtain the compound of 2-carbonyl-3-acyl-1,3-thiazolidine derivative of formula Q-1a:

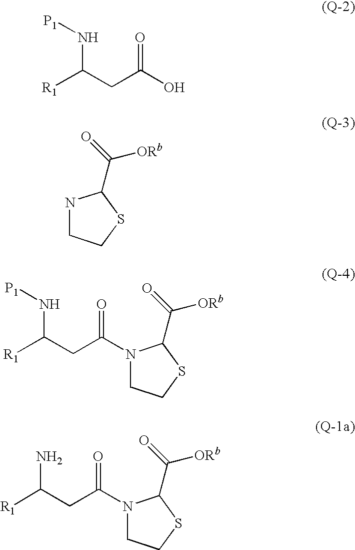

wherein, P₁ is an amine protecting group including, but are not limited to tert-butyloxycarbonyl (BOC), carbobenzyloxy (CBz), benzyl, Phthalimides (Pht), sulfonyl protecting groups (e.g., p-toluenesulfonyl) and other protecting groups well known in the art, including those found in “Protective Groups in Organic Synthesis” by Theodora Green (publisher: John Wiley & Sons), the disclosure of which is hereby incorporated by reference; and R₁ and R^{b} are the same as defined above in formula (Q).

In a further embodiment, step (i) of Method I comprises a condensing reagent (e.g., 1,1′-carbonyldiimidazole (CDI), 1,3-dicyclohexylcarbodiimide (DCC), 1-(3-dimethylaminopropyl)-3-ethylcarbodiimide hydrochloride (EDCI), DCC/HOBt (1-Hydroxybenzotriazole)) or EDCI/HOBt, and optionally a base (e.g., triethylamine, diisopropylethylamine (DIPEA), pyridine, piperidine, sodium bicarbonate, potassium bicarbonate, cesium carbonate, or potassium hydroxide);

In yet a further embodiment, step (ii) of Method I comprises the use of a deprotecting agent. Depending on the protecting group used, appropriate deprotecing agent may be employed. For example, to remove a BOC or CBz protecting group, an acid or combination of acids (e.g., trifluoroacetic acid, hydrobromic acid, acetic acid or hydrochloric acid) may be used. Benzyl protecting group may be removed by hydrogenation method (H₂ and palladium on carbon). Phthalimide protecting group may be removed by employing hydrazine. Sulfonyl protecting group may be removed by reduction method (e.g., using sodium or lithium in liquid ammonia). This list is not intended to be exhaustive and therefore does not exclue other deprotecting agents well known in the art such as those found in “Protective Groups in Organic Synthesis” by Theodora Green (publisher: John Wiley & Sons).

In yet another embodiment, the present invention provides a method (Method (II)) for preparing a compound of 2-carbonyl-3-acyl-1,3-thiazolidine derivative of formula Q-1b, comprising the steps of:

(i) subjecting an amino acid of formula Q-2 to a condensation reaction with a 2-carbonyl-1,3-thiazolidine-based compound of formula Q-3 (e.g., by using a condensing agent such as DCC, EDCI, CDI, EDCI/HOBt or CDI/HOBt optionally in the presence of a base such as triethylamine, diisopropylethylamine, pyridine, piperidine, sodium bicarbonate, potassium bicarbonate, cesium carbonate, or potassium hydroxide) to form a compound of formula Q-4;

(ii) forming a compound of formula Q-5 from the compound of formula Q-4 (e.g., by using a condensing agent such as such as DCC, EDCI, CDI, EDCI/HOBt or CDI/HOBt optionally in the presence of a base such as triethylamine, diisopropylethylamine, pyridine, piperidine, sodium bicarbonate, potassium bicarbonate, cesium carbonate, or potassium hydroxide); and

(iii) deprotecting the compound of formula Q-5 to obtain the compound of 2-carbonyl-3-acyl-1,3-thiazolidine derivative of formula Q-1b:

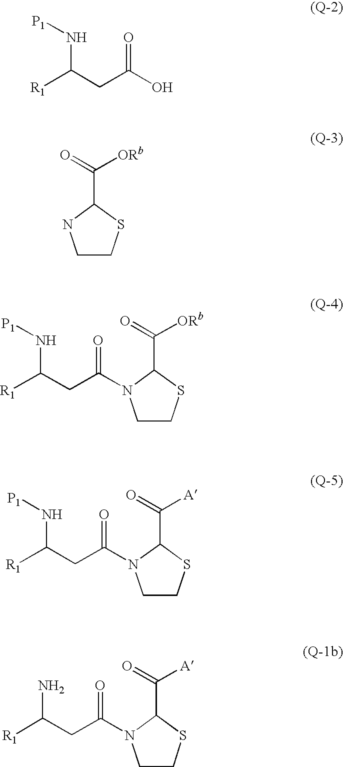

wherein, A′ is

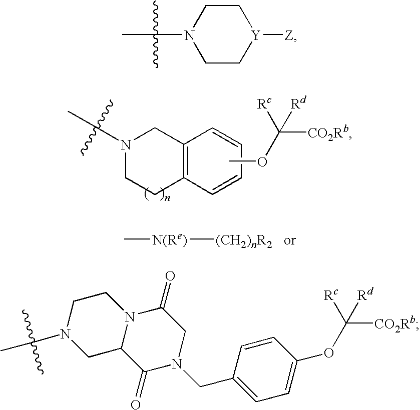

P₁, R₁, R₂, R^{b} to R^{e}, Y, Z and n are the same as defined above.

In addition, the present invention provides a method (Method (III)) for preparing a 2-carbonyl-3-acyl-1,3-thiazolidine derivative of formula Q-1b-1, comprising the steps of:

(i) hydrolyzing a compound of formula Q-6 (e.g., with a base such as sodium hydroxide, lithium hydroxide or potassium hydroxide) to form a compound of formula Q-7;

(ii) subjecting the compound of formula Q-7 to a condensation reaction (e.g., by reacting Q-7 with a condensing agent such as DCC, EDCI, CDI, EDCI/HOBt or CDI/HOBt optionally in the presence of a base such as triethylamine, diisopropylethylamine, pyridine, piperidine, sodium bicarbonate, potassium bicarbonate, cesium carbonate or potassium hydroxide) with a compound of formula Q-8 to form a compound of formula Q-9; and

(iii) deprotecting the compound of formula Q-9 to obtain a compound of 2-carbonyl-3-acyl-1,3-thiazolidine derivative of formula Q-1b-1:

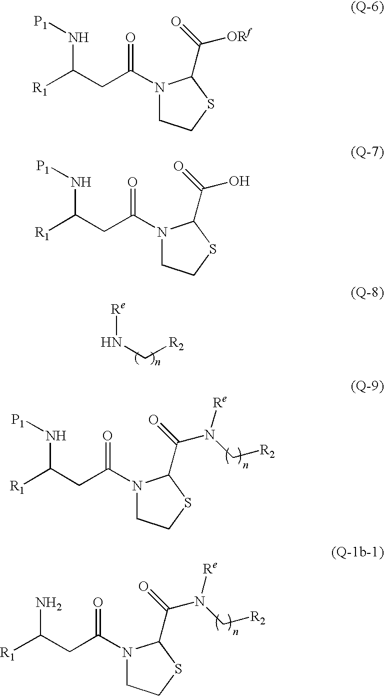

wherein, R^{f} is alkyl (e.g., methyl or ethyl), P₁ and R₁, R₂, R^{e} and n are the same as defined above.

The present invention also provides a method (Method (IV)) for preparing a 2-carbonyl-3-acyl-1,3-thiazolidine derivative of formula Q-1b-2, comprising the steps of:

(i) subjecting a compound of formula Q-7 to a condensation reaction (e.g., by reacting compound of formula Q-7 with a condensing agent such as DCC, EDCI, CDI, EDCI/HOBt or CDI/HOBt optionally in the presence of a base such as triethylamine, diisopropylethylamine, pyridine, piperidine, sodium bicarbonate, potassium bicarbonate, cesium carbonate or potassium hydroxide) with a compound of formula Q-10 to form a compound of formula Q-5a; and

(ii) deprotecting the compound of formula Q-5a as similarly described in Method (I) to obtain a compound of 2-carbonyl-3-acyl-1,3-thiazolidine derivative of formula Q-1b-2:

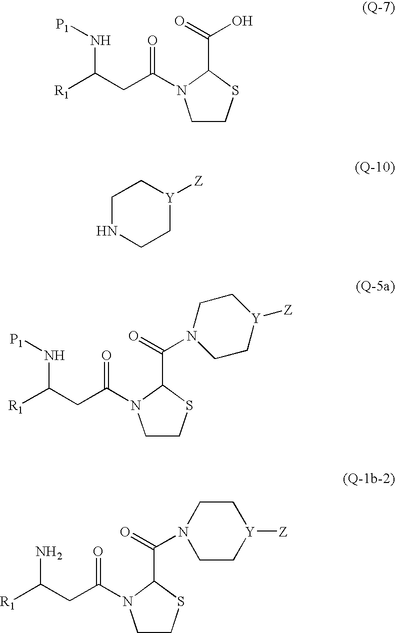

wherein, P₁, R₁, Y and Z are the same as defined above.

The present invention also provides a method (Method (V)) for preparing a compound of 2-carbonyl-3-acyl-1,3-thiazolidine derivative of formula Q-1b-3, comprising the steps of:

(i) hydrolyzing a compound of formula Q-11 (e.g., with a base such as potassium hydroxide, lithium hydroxide or sodium hydroxide) to form a compound of formula Q-12; and

(ii) deprotecting the compound of formula Q-12 as similarly described in Method (I) to obtain a compound of 2-carbonyl-3-acyl-1,3-thiazolidine derivative of formula Q-1b-3:

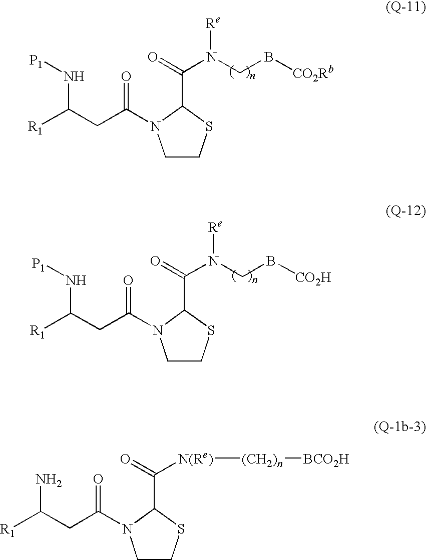

wherein, B is a substitutent selected from the group consisting of,

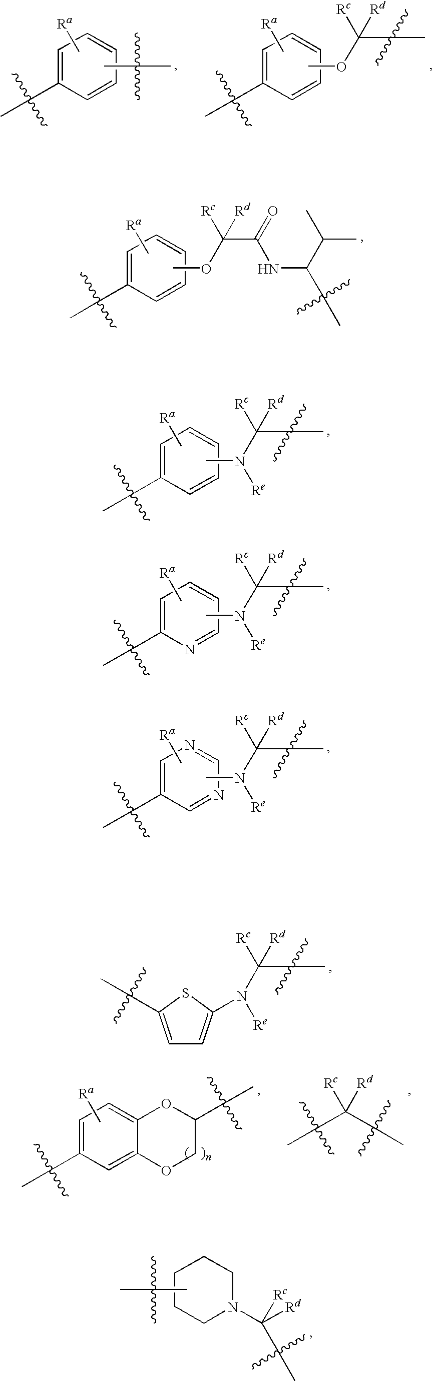

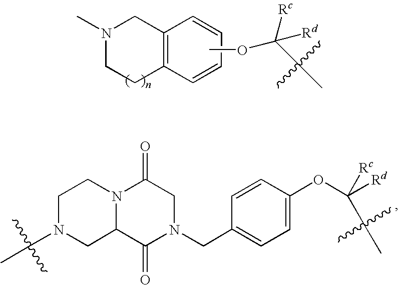

wherein N(R^{e})—(CH₂)ₙ— is attached to the left side of the B and —CO₂R^{b} or CO₂H is attached to the right side of B; and P₁, R₁, R^{a} to R^{g} and n are the same as defined above.

In accordance with another aspect of the present invention, there is provided a method (Method (VI)) for preparing a compound of 2-carbonyl-3-acyl-1,3-thiazolidine derivative of formula 1a, comprising the steps of:

(i) subjecting an amino acid of formula 2 to a condensation reaction with a 2-carbonyl-1,3-thiazolidine-based compound of formula 3 to form a compound of formula 4; and

(ii) deprotecting the compound of formula 4 to obtain the compound of 2-carbonyl-3-acyl-1,3-thiazolidine derivative of formula 1a:

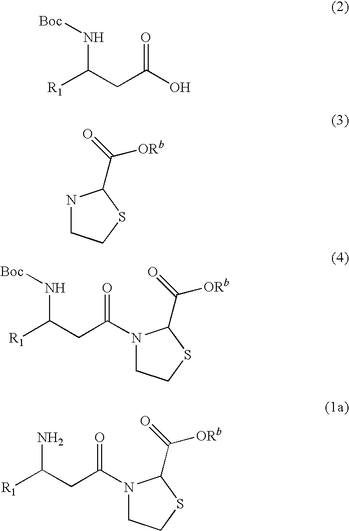

wherein, Boc is a protecting group; and R₁ and R^{b} are the same as defined above in formula (1).

The present invention also provides a method (Method (VII)) for preparing a compound of 2-carbonyl-3-acyl-1,3-thiazolidine derivative of formula 1b, comprising the steps of:

(i) subjecting an amino acid of formula 2 to a condensation reaction with a 2-thiazolidine-based compound of formula 3 to form a compound of formula 4;

(ii) forming a compound of formula 5 from the compound of formula 4; and

(iii) deprotecting the compound of formula 5 to obtain the compound of 2-carbonyl-3-acyl-1,3-thiazolidine derivative of formula 1b:

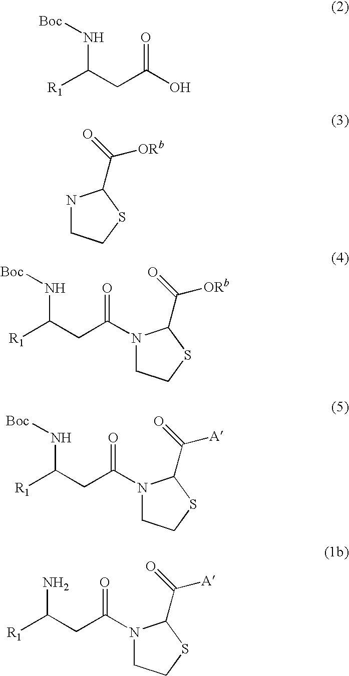

wherein, A′ is

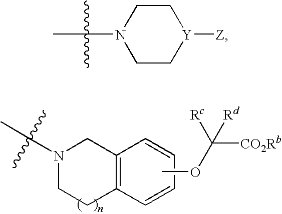

or —NR^{e} (CH₂)ₙR₂; Boc, R₁, R₂, R^{b} to R^{e}, Y, Z and n are the same as defined above in Method VI and in formula (1).

In addition, the present invention provides a method (Method (VIII)) for preparing a 2-carbonyl-3-acyl-1,3-thiazolidine derivative of formula 1b-1, comprising the steps of:

(i) hydrolyzing a compound of formula 6 to form a compound of formula 7;

(ii) subjecting the compound of formula 7 to a condensation reaction with a compound of formula 8 to form a compound of formula 9; and

(iii) deprotecting the compound of formula 9 to obtain a compound of 2-carbonyl-3-acyl-1,3-thiazolidine derivative of formula 1b-1:

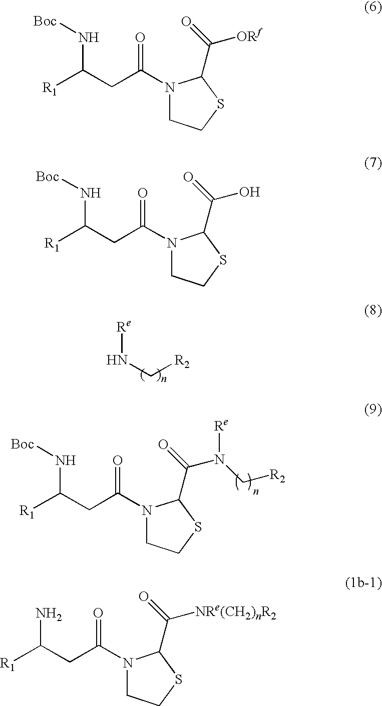

wherein, R^{f} is methyl or ethyl, and Boc, R₁, R₂, R^{e} and n are the same as defined above in Methods VI-VII.

The present invention also provides a method (Method (IX)) for preparing a 2-carbonyl-3-acyl-1,3-thiazolidine derivative of formula 1b-2, comprising the steps of:

(i) subjecting a compound of formula 7 to a condensation reaction with a compound of formula 10 to form a compound of formula 5a; and

(ii) deprotecting the compound of formula 5a to obtain a compound of 2-carbonyl-3-acyl-1,3-thiazolidine derivative of formula 1b-2:

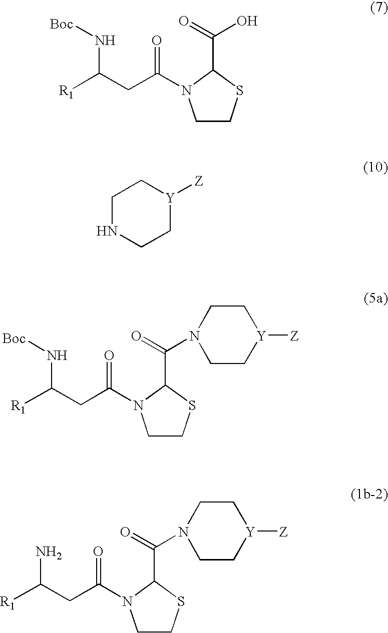

wherein, Boc, R₁, Y and Z are the same as defined above in Methods (VI)-(VIII) or in formula (1).

The present invention also provides a method (Method (X)) for preparing a compound of 2-carbonyl-3-acyl-1,3-thiazolidine derivative of formula 1b-3, comprising the steps of:

(i) hydrolyzing a compound of formula 11 to form a compound of formula 12; and

(ii) deprotecting the compound of formula 12 to obtain a compound of 2-carbonyl-3-acyl-1,3-thiazolidine derivative of formula 1b-3:

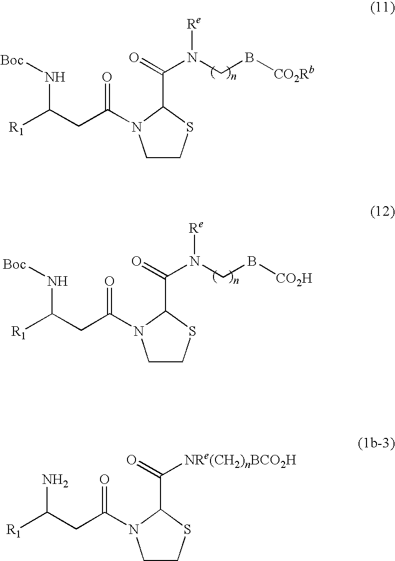

wherein, BCO₂H is a carboxylic acid-containing substituent selected from the group consisting of

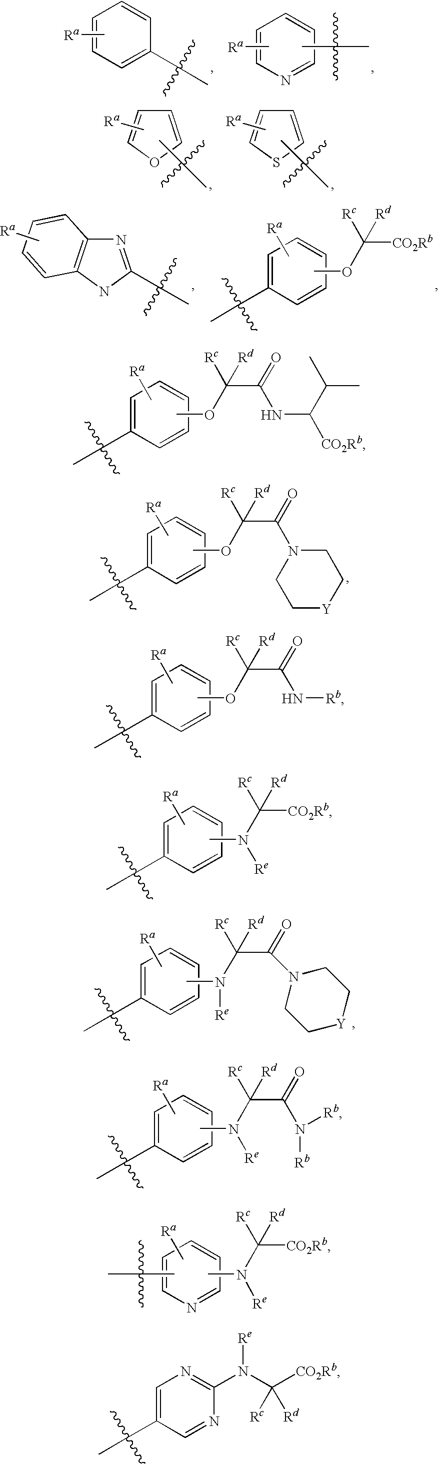

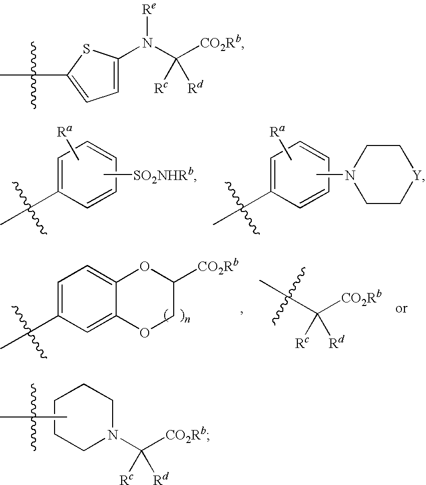

and

Boc, R₁, R^{a} to R^{e}, Y and n are the same as defined above in Methods (VI)-(IX) or in formula (1).

In accordance with further aspect of the present invention, there is provided a pharmaceutical composition comprising the disclosed compound or a pharmaceutically acceptable salt thereof and a pharmaceutically acceptable carrier. For example, a pharmaceutical composition comprising a compound of formula (Q), e.g., any of 1.1-1.75, or formula (1), in free, pharmaceutically acceptable salt, prodrug, enantiomeric, diastereoisomeric or racemate form, and a pharmaceutically acceptable diluents or carrier.

The present invention also provides a method for inhibiting DPP-IV in a mammal, comprising administering the disclosed compound or a pharmaceutically acceptable salt thereof to the mammal in an amount effective for the inhibition of DPP-IV. For example, a method for inhibiting DPP-IV in a mammal comprising administering a compound of formula (Q), e.g., any of 1.1-1.75, or formula (1), in free, pharmaceutically acceptable salt, prodrug, enantiomeric, diastereoisomeric or racemate form to the mammal in an amount effective for the inhibition of DPP-IV.

Further, the present invention provides a method for treating DPP-IV-mediated diseases in a mammal, comprising administering the disclosed compound or a pharmaceutically acceptable salt thereof to the mammal in a therapeutically effective amount. For example, a method for treating DPP-IV-mediated diseases in a mammal, comprising administering a compound of formula (Q), e.g., any of 1.1-1.75, or formula (1), in free, pharmaceutically acceptable salt, prodrug, enantiomeric, diastereoisomeric or racemate form to the mammal in a therapeutically effective amount. DPP-IV-mediated diseases may be selected from a group consisting of Type 1 diabetes (insulin-dependent diabetes mellitus), Type 2 diabetes (insulin-independent diabetes mellitus), arthritis, obesity, osteoporosis and impaired glucose tolerance.

In accordance with yet another aspect of the present invention, there is provided use of a compound of formula (Q), e.g., any of 1.1-1.75, or formula (1), in free, pharmaceutically acceptable salt, prodrug, enantiomeric, diastereoisomeric or racemate form, in the manufacture of a medicament for the treatment of DPP-IV-mediated diseases, e.g., selected from a group consisting of Type 1 diabetes (insulin-dependent diabetes mellitus), Type 2 diabetes (insulin-independent diabetes mellitus), arthritis, obesity, osteoporosis and impaired glucose tolerance.

In accordance to a further aspect of the invention, the invention provides compounds of formula (Q), e.g., any of 1.1-1.75, or formula (1), and their physiologically hydrolysable and acceptable esters thereof. The term “physiologically hydrolysable and acceptable ester” as used herein in relation to compounds of formula (Q) or formula (1) is meant esters of such compounds which are hydrolysable under physiological conditions to yield their respective acids and alcohols which are themselves physiologically tolerable at doses to be administered. For example, wherein A of formula (Q) is —N(R^{e})—(CH₂)ₙ—R₂ and R₂ is

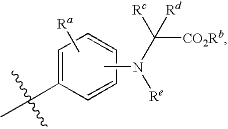

—OR^{b} may be a residue of a physiologically acceptable alcohol, HO—R^{b}, e.g. ethanol in the case where R^{b} is ethyl. As will be appreciated, the term thus embraces conventional pharmaceutical prodrug forms.

### DETAILED DESCRIPTION OF THE INVENTION

The present invention provides novel compounds of 2-carbonyl-3-acyl-1,3-thiazolidine derivatives having β-amino group represented by formula 1 or a pharmaceutically acceptable salt thereof, which show superior activity for the inhibition of DPP-IV.

Accordingly, the compounds of formula 1 or formula (Q) can be useful for preventing or treating DPP-IV-mediated diseases, for example, Type 1 diabetes (insulin-dependent diabetes mellitus), Type 2 diabetes (insulin-independent diabetes mellitus), arthritis, obesity, osteoporosis and impaired glucose tolerance.

Among the compounds of formula 1 and formula (Q) of the present invention, preferred are those wherein R₁ is

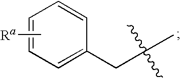

and R^{a} is one or more substitutents selected from the group consisting of hydrogen, C₁₋₆ alkyl, C₁₋₆ alkoxy, —OCF₃, halogen, —CN and —CF₃. More preferred are those wherein R₁ is

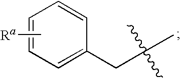

and R^{a} is one or more halogen substituents which can be same or different, and still more preferably those having A of —NH(CH₂)ₙR₂ together with R₁ and R^{a} as defined above.

The disclosed compound of formula 1 or formula (Q) may contain one or more asymmetric carbon atoms (e.g., carbon atom having the amino group and R₁ substituent) and may exist in the forms of enantiomers of R or S configuration, diastereomers or other stereoisomers. Preferably, the disclosed compound has the form of R-isomer in the carbon atom having the amino group and R₁ substituent, in terms of the inhibition activity against DPP-IV.

The compound of formula 1 may be used in the form of a pharmaceutically acceptable addition salt formed with an acid. Exemplary acids which may be used in the present invention include, but are not limited to, hydrochloric, sulfuric, acetic, trifluoroacetic, phosphoric, fumaric, maleic, citric, methanesulfonic and lactic acids. The compound of formula (Q) may also be used in the form of a pharmaceutically acceptable addition salt formed with an acid, including, but are not limited to, hydrochloric, sulfuric, acetic, trifluoroacetic, phosphoric, fumaric, maleic, citric, methanesulfonic and lactic acids.

In particular embodiments of the invention, compounds of formula 1 useful for inhibiting DPP-IV include the following:
(1) methyl 3-((R)-3-amino-4-(2,4,5-trifluorophenyl)butanoyl)thiazolidine-2-carboxylate.HCl,
(2) 3-((R)-3-amino-4-(2,4,5-trifluorophenyl)butanoyl)thiazolidine-2-carboxylic acid.HCl,
(3) 3-((R)-3-amino-4-(2,4,5-trifluorophenyl)butanoyl)-N-benzylthiazolidine-2-carboxamide.HCl,
(4) ethyl 2-(4-((3-((R)-3-amino-4-(2,4,5-trifluorophenyl)butanoyl)thiazolidine-2-carboxamido)methyl)phenoxy)acetate.HCl,
(5) 2-4-((3-((R)-3-amino-4-(2,4,5-trifluorophenyl)butanoyl)thiazolidine-2-carboxamido)methyl)phenoxy)acetic acid.HCl,
(6) ethyl 2-(4-(3-((R)-3-amino-4-(2,4,5-trifluorophenyl)butanoyl)thiazolidine-2-carboxamido)phenoxy)acetate.HCl,
(7) 2-(4-(3-((R)-3-amino-4-(2,4,5-trifluorophenyl)butanoyl)thiazolidine-2-carboxamido)phenoxy)acetic acid.HCl,
(8) ethyl 2-(4-((3-((R)-3-amino-4-(2,4,5-trifluorophenyl)butanoyl)thiazolidine-2-carboxamido)methyl)phenoxy)-3-methylbutanoate.HCl,
(9) 2-(4-((3-((R)-3-amino-4-(2,4,5-trifluorophenyl)butanoyl)thiazolidine-2-carboxamido)methyl)phenoxy)-3-methylbutanoic acid.HCl,
(10) pivaloyloxymethyl 2-(4-((3-((R)-3-amino-4-(2,4,5-trifluorophenyl) butanoyl)thiazolidine-2-carboxamido)methyl)phenoxy)-3-methylbutanoate.HCl,
(11) ethyl 1-(3-((R)-3-amino-4-(2,4,5-trifluorophenyl)butanoyl)thiazolidine-2-carbonyl)piperidine-4-carboxylate.HCl,
(12) 1-(3-((R)-3-amino-4-(2,4,5-trifluorophenyl)butanoyl)thiazolidine-2-carbonyl)piperidine-4-carboxylic acid.HCl,
(13) 2-(4-((3-((R)-3-amino-4-(2,4,5-trifluorophenyl)butanoyl)thiazolidine-2-carboxamido)methyl)phenyl)acetic acid.HCl,
(14) ethyl 2-(2-(3-((R)-3-amino-4-(2,4,5-trifluorophenyl)butanoyl)thiazolidine-2-carbonyl)-1,2,3,4-tetrahydroisoquinolin-7-yloxy)-3-methylbutanoate.HCl,
(15) 2-(2-(3-((R)-3-amino-4-(2,4,5-trifluorophenyl)butanoyl)thiazolidine-2-carbonyl)-1,2,3,4-tetrahydroisoquinolin-7-yloxy)-3-methylbutanoic acid.HCl,
(16) ethyl 6-((3-((R)-3-amino-4-(2,4,5-trifluorophenyl)butanoyl)thiazolidine-2-carboxamido)methyl)-2,3-dihydrobenzo[b][1,4]dioxin-2-carboxylate.HCl,
(17) 6-((3-((R)-3-amino-4-(2,4,5-trifluorophenyl)butanoyl)thiazolidine-2-carboxamido)methyl)-2,3-dihydrobenzo[b][1,4]dioxin-2-carboxylic acid.HCl,
(18) pivaloyloxymethyl 2-(4-((3-((R)-3-amino-4-(2,4,5-trifluorophenyl) butanoyl)thiazolidine-2-carboxamido)methyl)phenoxy)-3-methylbutanoate.HCl,
(19) ethyl 2-(4-((3-((R)-3-((1-acetoxyethoxy)carbonylamino)-4-(2,4,5-trifluorophenyl)butanoyl)thiazolidine-2-carboxamido)methyl)phenoxy)-3-methylbutanoate,
(20) (3R)-3-amino-1-(2-(morpolin-4-carbonyl)thiazolidin-3-yl)-4-(2,4,5-trifluorophenyl)butan-1-on.HCl,
(21) N-(2-(1H-imidazol-5-yl)ethyl)-3-((R)-3-amino-4-(2,4,5-trifluorophenyl) butanoyl)thiazolidine-2-carboxamide.2HCl,
(22) (S)-2-(4-(((S)-3-((R)-3-amino-4-(2,4,5-trifluorophenyl)butanoyl) thiazolidine-2-carboxamido)methyl)phenoxy)-3-methylbutanoic acid.HCl,
(23) (R)-2-(4-(((S)-3-((R)-3-amino-4-(2,4,5-trifluorophenyl)butanoyl) thiazolidine-2-carboxamido)methyl)phenoxy)-3-methylbutanoic acid.HCl,
(24) (S)-2-(4-(((R)-3-((R)-3-amino-4-(2,4,5-trifluorophenyl)butanoyl) thiazolidine-2-carboxamido)methyl)phenoxy)-3-methylbutanoic acid.HCl,
(25) (R)-2-(4-(((R)-3-((R)-3-amino-4-(2,4,5-trifluorophenyl)butanoyl) thiazolidine-2-carboxamido)methyl)phenoxy)-3-methylbutanoic acid.HCl,
(26) (S)-2-(4-(((S)-3-((R)-3-amino-4-(2,4,5-trifluorophenyl)butanoyl) thiazolidine-2-carboxamido)methyl)phenylamino)-3-methylbutanoic acid.HCl,
(27) (R)-2-(4-(((S)-3-((R)-3-amino-4-(2,4,5-trifluorophenyl)butanoyl) thiazolidine-2-carboxamido)methyl)phenylamino)-3-methylbutanoic acid.HCl,
(28) (S)-2-(4-(((R)-3-((R)-3-amino-4-(2,4,5-trifluorophenyl)butanoyl) thiazolidine-2-carboxamido)methyl)phenylamino)-3-methylbutanoic acid.HCl,
(29) (R)-2-(4-(((R)-3-((R)-3-amino-4-(2,4,5-trifluorophenyl)butanoyl) thiazolidine-2-carboxamido)methyl)phenylamino)-3-methylbutanoic acid.HCl,
(30) ethyl 2-(3-((3-((R)-3-amino-4-(2,4,5-trifluorophenyl)butanoyl) thiazolidine-2-carboxamido)methyl)phenoxy)acetate.HCl,
(31) 2-(3-((3-((R)-3-amino-4-(2,4,5-trifluorophenyl)butanoyl)thiazolidine-2-carboxamido)methyl)phenoxy)acetic acid.HCl,
(32) ethyl 2-(3-(3-((R)-3-amino-4-(2,4,5-trifluorophenyl)butanoyl) thiazolidine-2-carboxamido)phenoxy)acetate.HCl,
(33) 2-(3-(3-((R)-3-amino-4-(2,4,5-trifluorophenyl)butanoyl)thiazolidine-2-carboxamido)phenoxy)acetic acid.HCl,
(34) 2-(4-(3-((R)-3-amino-4-(2,4,5-trifluorophenyl)butanoyl)thiazolidine-2-carboxamido)piperidine-1-yl)-3-methylbutanoic acid.2HCl,
(35) (S)-ethyl 2-(4-(((S)-3-((R)-3-amino-4-(2,4,5-trifluorophenyl)butanoyl) thiazolidine-2-carboxamido)methyl)phenylamino)-3-methylbutanoate.HCl,
(36) (R)-ethyl 2-(4-(((S)-3-((R)-3-amino-4-(2,4,5-trifluorophenyl)butanoyl) thiazolidine-2-carboxamido)methyl)phenylamino)-3-methylbutanoate.HCl,
(37) (S)-ethyl 2-(4-(((R)-3-((R)-3-amino-4-(2,4,5-trifluorophenyl)butanoyl) thiazolidine-2-carboxamido)methyl)phenylamino)-3-methylbutanoate.HCl,
(38) (R)-ethyl 2-(4-(((R)-3-((R)-3-amino-4-(2,4,5-trifluorophenyl)butanoyl) thiazolidine-2-carboxamido)methyl)phenylamino)-3-methylbutanoate.HCl,
(39) (3R)-3-amino-1-(2-(thiomorpolin-4-carbonyl)thiazolidin-3-yl)-4-(2,4,5-trifluorophenyl)butan-1-on.HCl,
(40) (3R)-3-amino-1-(2-(piperazine-1-carbonyl)thiazolidin-3-yl)-4-(2,4,5-trifluorophenyl)butan-1-on.HCl,
(41) (3R)-3-amino-1-(2-(4-methylpiperazine-1-carbonyl)thiazolidin-3-yl)-4-(2,4,5-trifluorophenyl)butan-1-on.HCl,
(42) 3-((R)-3-amino-4-(2,4,5-trifluorophenyl)butanoyl)-N,N-dimethyl thiazolidine-2-carboxamide.HCl,
(43) 3-((R)-3-amino-4-(2,4,5-trifluorophenyl)butanoyl)-N-(furan-3-yl)methyl) thiazolidine-2-carboxamide.HCl,
(44) ethyl 2-(3-((R)-3-amino-4-(2,4,5-trifluorophenyl)butanoyl)thiazolidine-2-carboxamido)acetate.HCl,
(45) 2-(3-((R)-3-amino-4-(2,4,5-trifluorophenyl)butanoyl)thiazolidine-2-carboxamido)acetic acid.HCl,
(46) N-(2-(1H-indol-3-yl)ethyl)-3-((R)-3-amino-4-(2,4,5-trifluorophenyl) butanoyl)thiazolidine-2-carboxamide.2HCl,
(47) 3-((R)-3-amino-4-(2,4,5-trifluorophenyl)butanoyl)-N-(4-morpholinophenyl) thiazolidine-2-carboxamide.HCl,
(48) 3-((R)-3-amino-4-(2,4,5-trifluorophenyl)butanoyl)-N-(4-sulfamoylphenyl) thiazolidine-2-carboxamide.HCl,
(49) 3-((R)-3-amino-4-(2,4,5-trifluorophenyl)butanoyl)-N-(4-sulfamoylbenzyl) thiazolidine-2-carboxamide.HCl,
(50) N-((1H-benzo[d]imidazol-2-yl)methyl)-3-((R)-3-amino-4-(2,4,5-trifluorophenyl)butanoyl)thiazolidine-2-carboxamide.HCl,
(51) ethyl 2-(4-((3-((R)-3-amino-4-(2,4,5-trifluorophenyl)butanoyl) thiazolidine-2-carboxamido)methyl)-3-fluorophenoxy)butanoate.HCl,
(52) 2-(4-((3-((R)-3-amino-4-(2,4,5-trifluorophenyl)butanoyl)thiazolidine-2-carboxamido)methyl)-3-fluorophenoxy)butanoic acid.HCl,
(53) ethyl 2-(4-((3-((R)-3-amino-4-(2,4,5-trifluorophenyl)butanoyl)thiazolidine-2-carboxamido)methyl)-3-fluorophenoxy)-2-methylpropanoate.HCl,
(54) 2-(4-((3-((R)-3-amino-4-(2,4,5-trifluorophenyl)butanoyl)thiazolidine-2-carboxamido)methyl)-3-fluorophenoxy)-2-methylpropanoic acid.HCl,
(55) ethyl 2-(4-((3-((R)-3-amino-4-(2,4,5-trifluorophenyl)butanoyl)thiazolidine-2-carboxamido)methyl)-2-fluorophenoxy)-2-methylpropanoate.HCl,
(56) 3-((R)-3-amino-4-(2,4,5-trifluorophenyl)butanoyl)-N-(pyridin-4-yl methyl)thiazolidine-2-carboxamide.2HCl,
(57) (S)-2-(2-(4-((3-((R)-3-amino-4-(2,4,5-trifluorophenyl)butanoyl)thiazolidine-2-carboxamido)methyl)phenoxy)-3-methylbutanamido)-3-methylbutanoic acid.HCl,
(58) (R)-ethyl 2-(4-((8-(3-((R)-3-amino-4-(2,4,5-trifluorophenyl)butanoyl) thiazolidine-2-carbonyl)-1,4-dioxo-hexahydro-1H-pyrazino[1,2-a]pyrazin-2(6H)-yl)methyl)phenoxy)-3-methylbutanoate.HCl,
(59) (R)-2-(4-((8-(3-((R)-3-amino-4-(2,4,5-trifluorophenyl)butanoyl)thiazolidine-2-carbonyl)-1,4-dioxo-hexahydro-1H-pyrazino[1,2-a]pyrazin-2(6H)-yl)methyl)phenoxy)-3-methylbutanoic acid.HCl,
(60) ethyl 2-(2-(3-((R)-3-amino-4-(2,4,5-trifluorophenyl)butanoyl)thiazolidine-2-carbonyl)-1,2,3,4-tetrahydroisoquinoline-6-yloxy)-3-methylbutanoate.HCl,
(61) 2-(2-(3-((R)-3-amino-4-(2,4,5-trifluorophenyl)butanoyl)thiazolidine-2-carbonyl)-1,2,3,4-tetrahydroisoquinoline-6-yloxy)-3-methylbutanoic acid.HCl,
(62) ethyl 5-((3-((R)-3-amino-4-(2,4,5-trifluorophenyl)butanoyl)thiazolidine-2-carboxamido)methyl)benzo[d][1,3]dioxol-2-carboxylate.HCl,
(63) 5-((3-((R)-3-amino-4-(2,4,5-trifluorophenyl)butanoyl)thiazolidine-2-carboxamido)methyl)benzo[d][1,3]dioxol-2-carboxylic acid.HCl,
(64) 2-(4-((3-((R)-3-amino-4-(2,4,5-trifluorophenyl)butanoyl)thiazolidine-2-carboxamido)methyl)phenoxy)-2-methylpropanoic acid.HCl,
(65) (R)-2-(4-((3-((R)-3-amino-4-(2,4,5-trifluorophenyl)butanoyl)thiazolidine-2-carboxamido)methyl)phenoxy)-3-phenylpropanoic acid.HCl,
(66) 2-(4-((3-((R)-3-amino-4-(2,4,5-trifluorophenyl)butanoyl)-N-methyl thiazolidine-2-carboxamido)methyl)phenoxy)-3-methylbutanoic acid.HCl,
(67) ethyl 2-(4-((3-((R)-3-amino-4-(2,4,5-trifluorophenyl)butanoyl)thiazolidine-2-carboxamido)methyl)phenylamino)-3-methylbutanoate.2HCl,
(68) 2-(4-((3-((R)-3-amino-4-(2,4,5-trifluorophenyl)butanoyl)thiazolidine-2-carboxamido)methyl)phenylamino)-3-methylbutanoic acid.HCl,
(69) ethyl 2-(4-((3-((R)-3-amino-4-(2,4,5-trifluorophenyl)butanoyl)thiazolidine-2-carboxamido)methyl)-3-fluorophenoxy)-3-methylbutanoate.HCl,
(70) 2-(4-((3-((R)-3-amino-4-(2,4,5-trifluorophenyl)butanoyl)thiazolidine-2-carboxamido)methyl)-3-fluorophenoxy)-3-methylbutanoic acid.HCl,
(71) 2-(4-((3-((R)-3-amino-4-(2,4,5-trifluorophenyl)butanoyl)thiazolidine-2-carboxamido)methyl)-2-fluorophenoxy)-2-methylpropanoic acid.HCl,
(72) ethyl 2-(4-(((S)-3-((R)-3-amino-4-(2,4,5-trifluorophenyl)butanoyl) thiazolidine-2-carboxamido)methyl)phenylamino)-2-methylpropanoate.HCl,
(73) 2-(4-(((S)-3-((R)-3-amino-4-(2,4,5-trifluorophenyl)butanoyl)thiazolidine-2-carboxamido)methyl)phenylamino)-2-methylpropanoic acid.HCl,
(74) (S)-methyl 2-(4-(((S)-3-((R)-3-amino-4-(2,4,5-trifluorophenyl)butanoyl) thiazolidine-2-carboxamido)methyl)-3-fluorophenylamino)-3-methylbutanoate.HCl,
(75) (S)-ethyl 2-(3-((R)-3-amino-4-(2,4,5-trifluorophenyl)butanoyl)thiazolidine-2-carboxamido)-3-methylbutanoate.HCl,
(76) (S)-2-(3-((R)-3-amino-4-(2,4,5-trifluorophenyl)butanoyl)thiazolidine-2-carboxamido)-3-methylbutanoic acid.HCl,
(77) (2S,3S)-ethyl 2-(3-((R)-3-amino-4-(2,4,5-trifluorophenyl)butanoyl) thiazolidine-2-carboxamido)-3-methylpentanoate.HCl,
(78) (2S,3S)-2-(3-((R)-3-amino-4-(2,4,5-trifluorophenyl)butanoyl)thiazolidine-2-carboxamido)-3-methylpentanoic acid.HCl,
(79) ethyl 2-(4-(3-((R)-3-amino-4-(2,4,5-trifluorophenyl)butanoyl) thiazolidine-2-carboxamido)piperidine-1-yl)-3-methylbutanoate.HCl,
(80) 4-((3-((R)-3-amino-4-(2,4,5-trifluorophenyl)butanoyl)thiazolidine-2-carboxamido)methyl)phenyl acetate.HCl,
(81) 3-((R)-3-amino-4-(2,4,5-trifluorophenyl)butanoyl)-N-(4-hydroxybenzyl) thiazolidine-2-carboxamide.HCl,
(82) ethyl 2-((4-((3-((R)-3-amino-4-(2,4,5-trifluorophenyl)butanoyl)thiazolidine-2-carboxamido)methyl)phenyl)(methyl)amino)-3-methylbutanoate.HCl,
(83) methyl 4-((3-((R)-3-amino-4-(2,4,5-trifluorophenyl)butanoyl)thiazolidine-2-carboxamido)methyl)-2-hydroxybenzoate.HCl,
(84) ethyl 2-(4-((3-((R)-3-amino-4-(2,4,5-trifluorophenyl)butanoyl)thiazolidine-2-carboxamido)methyl)phenoxy)propanoate.HCl,
(85) 2-((4-((3-((R)-3-amino-4-(2,4,5-trifluorophenyl)butanoyl)thiazolidine-2-carboxamido)methyl)phenyl)(methyl)amino)-3-methylbutanoic acid.HCl,
(86) 4-((3-((R)-3-amino-4-(2,4,5-trifluorophenyl)butanoyl)thiazolidine-2-carboxamido)methyl)-2-hydroxybenzoic acid.HCl,
(87) (S)-2-(4-(((S)-3-((R)-3-amino-4-(2,4,5-trifluorophenyl)butanoyl) thiazolidine-2-carboxamido)methyl)-3-fluorophenylamino)-3-methylbutanoic acid.HCl,
(88) (S)-ethyl 2-(4-(((S)-3-((R)-3-amino-4-(2,4,5-trifluorophenyl)butanoyl) thiazolidine-2-carboxamido)methyl)phenoxy)-3-methylbutanoate.HCl,
(89) (S)-ethyl 2-(4-(((S)-3-((R)-3-amino-4-(2,4,5-trifluorophenyl)butanoyl) thiazolidine-2-carboxamido)methyl)-2-fluorophenylamino)-3-methylbutanoate.HCl,
(90) (S)-2-(4-(((S)-3-((R)-3-amino-4-(2,4,5-trifluorophenyl)butanoyl) thiazolidine-2-carboxamido)methyl)-2-fluorophenylamino)-3-methylbutanoic acid.HCl,
(91) (S)-ethyl 2-(6-(((S)-3-((R)-3-amino-4-(2,4,5-trifluorophenyl)butanoyl) thiazolidine-2-carboxamido)methyl)pyridin-3-ylamino)-3-methylbutanoate.HCl,
(92) (S)-2-(6-(((S)-3-((R)-3-amino-4-(2,4,5-trifluorophenyl)butanoyl) thiazolidine-2-carboxamido)methyl)pyridin-3-ylamino)-3-methylbutanoic acid.HCl,
(93) 2-(4-((3-((R)-3-amino-4-(2,4,5-trifluorophenyl)butanoyl)thiazolidine-2-carboxamido)methyl)phenoxy)propanoic acid.HCl,
(94) (S)-2-(4-(((S)-3-((R)-3-amino-4-(2,4,5-trifluorophenyl)butanoyl) thiazolidine-2-carboxamido)methyl)phenylamino)-3,3-dimethylbutanoic acid.HCl,
(95) (S)-2-(2-(((S)-3-((R)-3-amino-4-(2,4,5-trifluorophenyl)butanoyl) thiazolidine-2-carboxamido)methyl)phenylamino)-3-methylbutanoic acid.HCl,
(96) (S)-2-(3-(((S)-3-((R)-3-amino-4-(2,4,5-trifluorophenyl)butanoyl) thiazolidine-2-carboxamido)methyl)phenylamino)-3-methylbutanoic acid.HCl,
(97) 4-((3-((R)-3-amino-4-(2,4,5-trifluorophenyl)butanoyl)thiazolidine-2-carboxamido)methyl)benzoic acid.HCl,
(98) 3-((R)-3-amino-4-(2,4,5-trifluorophenyl)butanoyl)-N-(4-(2-oxo-2-(piperazine-1-yl)ethoxy)benzyl)thiazolidine-2-carboxamide.2HCl,
(99) 3-((R)-3-amino-4-(2,4,5-trifluorophenyl)butanoyl)-N-(4-(2-oxo-2-thiomorpholinoethoxy)benzyl)thiazolidine-2-carboxamide.HCl, and
(100) 3-((R)-3-amino-4-(2,4,5-trifluorophenyl)butanoyl)-N-(4-(2-morpholino-2-oxoethoxy)benzyl)thiazolidine-2-carboxamide.HCl.

In particular embodiments of the invention, compounds of formula (Q) useful for inhibiting DPP-IV include the following:
(1) methyl 3-((R)-3-amino-4-(2,4,5-trifluorophenyl)butanoyl)thiazolidine-2-carboxylate,
(2) 3-((R)-3-amino-4-(2,4,5-trifluorophenyl)butanoyl)thiazolidine-2-carboxylic acid,
(3) 3-((R)-3-amino-4-(2,4,5-trifluorophenyl)butanoyl)-N-benzylthiazolidine-2-carboxamide,
(4) ethyl 2-(4-((3-((R)-3-amino-4-(2,4,5-trifluorophenyl)butanoyl)thiazolidine-2-carboxamido)methyl)phenoxy)acetate,
(5) 2-4-((3-((R)-3-amino-4-(2,4,5-trifluorophenyl)butanoyl)thiazolidine-2-carboxamido)methyl)phenoxy)acetic acid,
(6) ethyl 2-(4-(3-((R)-3-amino-4-(2,4,5-trifluorophenyl)butanoyl)thiazolidine-2-carboxamido)phenoxy)acetate,
(7) 2-(4-(3-((R)-3-amino-4-(2,4,5-trifluorophenyl)butanoyl)thiazolidine-2-carboxamido)phenoxy)acetic acid,
(8) ethyl 2-(4-((3-((R)-3-amino-4-(2,4,5-trifluorophenyl)butanoyl)thiazolidine-2-carboxamido)methyl)phenoxy)-3-methylbutanoate,
(9) 2-(4-((3-((R)-3-amino-4-(2,4,5-trifluorophenyl)butanoyl)thiazolidine-2-carboxamido)methyl)phenoxy)-3-methylbutanoic acid,
(10) pivaloyloxymethyl 2-(4-((3-((R)-3-amino-4-(2,4,5-trifluorophenyl) butanoyl)thiazolidine-2-carboxamido)methyl)phenoxy)-3-methylbutanoate,
(11) ethyl 1-(3-((R)-3-amino-4-(2,4,5-trifluorophenyl)butanoyl)thiazolidine-2-carbonyl)piperidine-4-carboxylate,
(12) 1-(3-((R)-3-amino-4-(2,4,5-trifluorophenyl)butanoyl)thiazolidine-2-carbonyl)piperidine-4-carboxylic acid,
(13) 2-(4-((3-((R)-3-amino-4-(2,4,5-trifluorophenyl)butanoyl)thiazolidine-2-carboxamido)methyl)phenyl)acetic acid,
(14) ethyl 2-(2-(3-((R)-3-amino-4-(2,4,5-trifluorophenyl)butanoyl)thiazolidine-2-carbonyl)-1,2,3,4-tetrahydroisoquinolin-7-yloxy)-3-methylbutanoate,
(15) 2-(2-(3-((R)-3-amino-4-(2,4,5-trifluorophenyl)butanoyl)thiazolidine-2-carbonyl)-1,2,3,4-tetrahydroisoquinolin-7-yloxy)-3-methylbutanoic acid,
(16) ethyl 6-((3-((R)-3-amino-4-(2,4,5-trifluorophenyl)butanoyl)thiazolidine-2-carboxamido)methyl)-2,3-dihydrobenzo[b][1,4]dioxin-2-carboxylate,
(17) 6-((3-((R)-3-amino-4-(2,4,5-trifluorophenyl)butanoyl)thiazolidine-2-carboxamido)methyl)-2,3-dihydrobenzo[b][1,4]dioxin-2-carboxylic acid,
(18) (S)-3-((R)-3-amino-4-(2,4,5-trifluorophenyl)butanoyl)thiazolidine-2-carboxylic acid,
(19) ethyl 2-(4-((3-((R)-3-((1-acetoxyethoxy)carbonylamino)-4-(2,4,5-trifluorophenyl)butanoyl)thiazolidine-2-carboxamido)methyl)phenoxy)-3-methylbutanoate,
(20) (3R)-3-amino-1-(2-(morpolin-4-carbonyl)thiazolidin-3-yl)-4-(2,4,5-trifluorophenyl)butan-1-one,
(21) N-(2-(1H-imidazol-5-yl)ethyl)-3-((R)-3-amino-4-(2,4,5-trifluorophenyl) butanoyl)thiazolidine-2-carboxamide,
(22) (S)-2-(4-(((S)-3-((R)-3-amino-4-(2,4,5-trifluorophenyl)butanoyl) thiazolidine-2-carboxamido)methyl)phenoxy)-3-methylbutanoic acid,
(23) (R)-2-(4-(((S)-3-((R)-3-amino-4-(2,4,5-trifluorophenyl)butanoyl) thiazolidine-2-carboxamido)methyl)phenoxy)-3-methylbutanoic acid,
(24) (S)-2-(4-(((R)-3-((R)-3-amino-4-(2,4,5-trifluorophenyl)butanoyl) thiazolidine-2-carboxamido)methyl)phenoxy)-3-methylbutanoic acid,
(25) (R)-2-(4-(((R)-3-((R)-3-amino-4-(2,4,5-trifluorophenyl)butanoyl) thiazolidine-2-carboxamido)methyl)phenoxy)-3-methylbutanoic acid,
(26) (S)-2-(4-(((S)-3-((R)-3-amino-4-(2,4,5-trifluorophenyl)butanoyl) thiazolidine-2-carboxamido)methyl)phenylamino)-3-methylbutanoic acid,
(27) (R)-2-(4-(((S)-3-((R)-3-amino-4-(2,4,5-trifluorophenyl)butanoyl) thiazolidine-2-carboxamido)methyl)phenylamino)-3-methylbutanoic acid,
(28) (S)-2-(4-(((R)-3-((R)-3-amino-4-(2,4,5-trifluorophenyl)butanoyl) thiazolidine-2-carboxamido)methyl)phenylamino)-3-methylbutanoic acid,
(29) (R)-2-(4-(((R)-3-((R)-3-amino-4-(2,4,5-trifluorophenyl)butanoyl) thiazolidine-2-carboxamido)methyl)phenylamino)-3-methylbutanoic acid,
(30) ethyl 2-(3-((3-((R)-3-amino-4-(2,4,5-trifluorophenyl)butanoyl) thiazolidine-2-carboxamido)methyl)phenoxy)acetate,
(31) 2-(3-((3-((R)-3-amino-4-(2,4,5-trifluorophenyl)butanoyl)thiazolidine-2-carboxamido)methyl)phenoxy)acetic acid,
(32) ethyl 2-(3-(3-((R)-3-amino-4-(2,4,5-trifluorophenyl)butanoyl) thiazolidine-2-carboxamido)phenoxy)acetate,
(33) 2-(3-(3-((R)-3-amino-4-(2,4,5-trifluorophenyl)butanoyl)thiazolidine-2-carboxamido)phenoxy)acetic acid,
(34) 2-(4-(3-((R)-3-amino-4-(2,4,5-trifluorophenyl)butanoyl)thiazolidine-2-carboxamido)piperidine-1-yl)-3-methylbutanoic acid,
(35) (S)-ethyl 2-(4-(((S)-3-((R)-3-amino-4-(2,4,5-trifluorophenyl)butanoyl) thiazolidine-2-carboxamido)methyl)phenylamino)-3-methylbutanoate,
(36) (R)-ethyl 2-(4-(((S)-3-((R)-3-amino-4-(2,4,5-trifluorophenyl)butanoyl) thiazolidine-2-carboxamido)methyl)phenylamino)-3-methylbutanoate,
(37) (S)-ethyl 2-(4-(((R)-3-((R)-3-amino-4-(2,4,5-trifluorophenyl)butanoyl) thiazolidine-2-carboxamido)methyl)phenylamino)-3-methylbutanoate,
(38) (R)-ethyl 2-(4-(((R)-3-((R)-3-amino-4-(2,4,5-trifluorophenyl)butanoyl) thiazolidine-2-carboxamido)methyl)phenylamino)-3-methylbutanoate,
(39) (3R)-3-amino-1-(2-(thiomorpolin-4-carbonyl)thiazolidin-3-yl)-4-(2,4,5-trifluorophenyl)butan-1-one,
(40) (3R)-3-amino-1-(2-(piperazine-1-carbonyl)thiazolidin-3-yl)-4-(2,4,5-trifluorophenyl)butan-1-one,
(41) (3R)-3-amino-1-(2-(4-methylpiperazine-1-carbonyl)thiazolidin-3-yl)-4-(2,4,5-trifluorophenyl)butan-1-one,
(42) 3-((R)-3-amino-4-(2,4,5-trifluorophenyl)butanoyl)-N,N-dimethyl thiazolidine-2-carboxamide,
(43) 3-((R)-3-amino-4-(2,4,5-trifluorophenyl)butanoyl)-N-(furan-3-yl)methyl) thiazolidine-2-carboxamide,
(44) ethyl 2-(3-((R)-3-amino-4-(2,4,5-trifluorophenyl)butanoyl)thiazolidine-2-carboxamido)acetate,
(45) 2-(3-((R)-3-amino-4-(2,4,5-trifluorophenyl)butanoyl)thiazolidine-2-carboxamido)acetic acid,
(46) N-(2-(1H-indol-3-yl)ethyl)-3-((R)-3-amino-4-(2,4,5-trifluorophenyl) butanoyl)thiazolidine-2-carboxamide,
(47) 3-((R)-3-amino-4-(2,4,5-trifluorophenyl)butanoyl)-N-(4-morpholinophenyl) thiazolidine-2-carboxamide,
(48) 3-((R)-3-amino-4-(2,4,5-trifluorophenyl)butanoyl)-N-(4-sulfamoylphenyl) thiazolidine-2-carboxamide,
(49) 3-((R)-3-amino-4-(2,4,5-trifluorophenyl)butanoyl)-N-(4-sulfamoylbenzyl) thiazolidine-2-carboxamide,
(50) N-((1H-benzo[d]imidazol-2-yl)methyl)-3-((R)-3-amino-4-(2,4,5-trifluorophenyl)butanoyl)thiazolidine-2-carboxamide,
(51) ethyl 2-(4-((3-((R)-3-amino-4-(2,4,5-trifluorophenyl)butanoyl) thiazolidine-2-carboxamido)methyl)-3-fluorophenoxy)butanoate,
(52) 2-(4-((3-((R)-3-amino-4-(2,4,5-trifluorophenyl)butanoyl)thiazolidine-2-carboxamido)methyl)-3-fluorophenoxy)butanoic acid,
(53) ethyl 2-(4-((3-((R)-3-amino-4-(2,4,5-trifluorophenyl)butanoyl)thiazolidine-2-carboxamido)methyl)-3-fluorophenoxy)-2-methylpropanoate,
(54) 2-(4-((3-((R)-3-amino-4-(2,4,5-trifluorophenyl)butanoyl)thiazolidine-2-carboxamido)methyl)-3-fluorophenoxy)-2-methylpropanoic acid,
(55) ethyl 2-(4-((3-((R)-3-amino-4-(2,4,5-trifluorophenyl)butanoyl)thiazolidine-2-carboxamido)methyl)-2-fluorophenoxy)-2-methylpropanoate,
(56) 3-((R)-3-amino-4-(2,4,5-trifluorophenyl)butanoyl)-N-(pyridin-4-yl methyl)thiazolidine-2-carboxamide,
(57) (S)-2-(2-(4-((3-((R)-3-amino-4-(2,4,5-trifluorophenyl)butanoyl)thiazolidine-2-carboxamido)methyl)phenoxy)-3-methylbutanamido)-3-methylbutanoic acid,
(58) (R)-ethyl 2-(4-((8-(3-((R)-3-amino-4-(2,4,5-trifluorophenyl)butanoyl) thiazolidine-2-carbonyl)-1,4-dioxo-hexahydro-1H-pyrazino[1,2-a]pyrazin-2(6H)-yl)methyl)phenoxy)-3-methylbutanoate,
(59) (R)-2-(4-((8-(3-((R)-3-amino-4-(2,4,5-trifluorophenyl)butanoyl)thiazolidine-2-carbonyl)-1,4-dioxo-hexahydro-1H-pyrazino[1,2-a]pyrazin-2(6H)-yl)methyl)phenoxy)-3-methylbutanoic acid,
(60) ethyl 2-(2-(3-((R)-3-amino-4-(2,4,5-trifluorophenyl)butanoyl)thiazolidine-2-carbonyl)-1,2,3,4-tetrahydroisoquinoline-6-yloxy)-3-methylbutanoate,
(61) 2-(2-(3-((R)-3-amino-4-(2,4,5-trifluorophenyl)butanoyl)thiazolidine-2-carbonyl)-1,2,3,4-tetrahydroisoquinoline-6-yloxy)-3-methylbutanoic acid,
(62) ethyl 5-((3-((R)-3-amino-4-(2,4,5-trifluorophenyl)butanoyl)thiazolidine-2-carboxamido)methyl)benzo[d][1,3]dioxol-2-carboxylate,
(63) 5-((3-((R)-3-amino-4-(2,4,5-trifluorophenyl)butanoyl)thiazolidine-2-carboxamido)methyl)benzo[d][1,3]dioxol-2-carboxylic acid,
(64) 2-(4-((3-((R)-3-amino-4-(2,4,5-trifluorophenyl)butanoyl)thiazolidine-2-carboxamido)methyl)phenoxy)-2-methylpropanoic acid,
(65) (R)-2-(4-((3-((R)-3-amino-4-(2,4,5-trifluorophenyl)butanoyl)thiazolidine-2-carboxamido)methyl)phenoxy)-3-phenylpropanoic acid,
(66) 2-(4-((3-((R)-3-amino-4-(2,4,5-trifluorophenyl)butanoyl)-N-methyl thiazolidine-2-carboxamido)methyl)phenoxy)-3-methylbutanoic acid,
(67) ethyl 2-(4-((3-((R)-3-amino-4-(2,4,5-trifluorophenyl)butanoyl)thiazolidine-2-carboxamido)methyl)phenylamino)-3-methylbutanoate,
(68) 2-(4-((3-((R)-3-amino-4-(2,4,5-trifluorophenyl)butanoyl)thiazolidine-2-carboxamido)methyl)phenylamino)-3-methylbutanoic acid,
(69) ethyl 2-(4-((3-((R)-3-amino-4-(2,4,5-trifluorophenyl)butanoyl)thiazolidine-2-carboxamido)methyl)-3-fluorophenoxy)-3-methylbutanoate,
(70) 2-(4-((3-((R)-3-amino-4-(2,4,5-trifluorophenyl)butanoyl)thiazolidine-2-carboxamido)methyl)-3-fluorophenoxy)-3-methylbutanoic acid,
(71) 2-(4-((3-((R)-3-amino-4-(2,4,5-trifluorophenyl)butanoyl)thiazolidine-2-carboxamido)methyl)-2-fluorophenoxy)-2-methylpropanoic acid,
(72) ethyl 2-(4-(((S)-3-((R)-3-amino-4-(2,4,5-trifluorophenyl)butanoyl) thiazolidine-2-carboxamido)methyl)phenylamino)-2-methylpropanoate,
(73) 2-(4-(((S)-3-((R)-3-amino-4-(2,4,5-trifluorophenyl)butanoyl)thiazolidine-2-carboxamido)methyl)phenylamino)-2-methylpropanoic acid,
(74) (S)-methyl 2-(2-(((S)-3-((R)-3-amino-4-(2,4,5-trifluorophenyl)butanoyl)thiazolidine-2-carboxamido)methyl)-5-bromophenylamino)-3-methylbutanoate,
(75) (S)-ethyl 2-(3-((R)-3-amino-4-(2,4,5-trifluorophenyl)butanoyl)thiazolidine-2-carboxamido)-3-methylbutanoate,
(76) (S)-2-(3-((R)-3-amino-4-(2,4,5-trifluorophenyl)butanoyl)thiazolidine-2-carboxamido)-3-methylbutanoic acid,
(77) (2S,3S)-ethyl 2-(3-((R)-3-amino-4-(2,4,5-trifluorophenyl)butanoyl) thiazolidine-2-carboxamido)-3-methylpentanoate,
(78) (2S,3S)-2-(3-((R)-3-amino-4-(2,4,5-trifluorophenyl)butanoyl)thiazolidine-2-carboxamido)-3-methylpentanoic acid,
(79) ethyl 2-(4-(3-((R)-3-amino-4-(2,4,5-trifluorophenyl)butanoyl) thiazolidine-2-carboxamido)piperidine-1-yl)-3-methylbutanoate,
(80) 4-((3-((R)-3-amino-4-(2,4,5-trifluorophenyl)butanoyl)thiazolidine-2-carboxamido)methyl)phenyl acetate,
(81) 3-((R)-3-amino-4-(2,4,5-trifluorophenyl)butanoyl)-N-(4-hydroxybenzyl) thiazolidine-2-carboxamide,
(82) ethyl 2-((4-((3-((R)-3-amino-4-(2,4,5-trifluorophenyl)butanoyl)thiazolidine-2-carboxamido)methyl)phenyl)(methyl)amino)-3-methylbutanoate,
(83) methyl 4-((3-((R)-3-amino-4-(2,4,5-trifluorophenyl)butanoyl)thiazolidine-2-carboxamido)methyl)-2-hydroxybenzoate,
(84) ethyl 2-(4-((3-((R)-3-amino-4-(2,4,5-trifluorophenyl)butanoyl)thiazolidine-2-carboxamido)methyl)phenoxy)propanoate,
(85) 2-((4-((3-((R)-3-amino-4-(2,4,5-trifluorophenyl)butanoyl)thiazolidine-2-carboxamido)methyl)phenyl)(methyl)amino)-3-methylbutanoic acid,
(86) 4-((3-((R)-3-amino-4-(2,4,5-trifluorophenyl)butanoyl)thiazolidine-2-carboxamido)methyl)-2-hydroxybenzoic acid,
(87) (S)-2-(2-(((S)-3-((R)-3-amino-4-(2,4,5-trifluorophenyl)butanoyl)thiazolidine-2-carboxamido)methyl)-5-bromophenylamino)-3-methylbutanoic acid,
(88) (S)-ethyl 2-(4-(((S)-3-((R)-3-amino-4-(2,4,5-trifluorophenyl)butanoyl) thiazolidine-2-carboxamido)methyl)phenoxy)-3-methylbutanoate,
(89) (S)-ethyl 2-(4-(((S)-3-((R)-3-amino-4-(2,4,5-trifluorophenyl)butanoyl) thiazolidine-2-carboxamido)methyl)-2-fluorophenylamino)-3-methylbutanoate,
(90) (S)-2-(4-(((S)-3-((R)-3-amino-4-(2,4,5-trifluorophenyl)butanoyl) thiazolidine-2-carboxamido)methyl)-2-fluorophenylamino)-3-methylbutanoic acid,
(91) (S)-ethyl 2-(6-(((S)-3-((R)-3-amino-4-(2,4,5-trifluorophenyl)butanoyl) thiazolidine-2-carboxamido)methyl)pyridin-3-ylamino)-3-methylbutanoate,
(92) (S)-2-(6-(((S)-3-((R)-3-amino-4-(2,4,5-trifluorophenyl)butanoyl) thiazolidine-2-carboxamido)methyl)pyridin-3-ylamino)-3-methylbutanoic acid,
(93) 2-(4-((3-((R)-3-amino-4-(2,4,5-trifluorophenyl)butanoyl)thiazolidine-2-carboxamido)methyl)phenoxy)propanoic acid,
(94) (S)-2-(4-(((S)-3-((R)-3-amino-4-(2,4,5-trifluorophenyl)butanoyl) thiazolidine-2-carboxamido)methyl)phenylamino)-3,3-dimethylbutanoic acid,
(95) (S)-2-(2-(((S)-3-((R)-3-amino-4-(2,4,5-trifluorophenyl)butanoyl) thiazolidine-2-carboxamido)methyl)phenylamino)-3-methylbutanoic acid,
(96) (S)-2-(3-(((S)-3-((R)-3-amino-4-(2,4,5-trifluorophenyl)butanoyl) thiazolidine-2-carboxamido)methyl)phenylamino)-3-methylbutanoic acid,
(97) 4-((3-((R)-3-amino-4-(2,4,5-trifluorophenyl)butanoyl)thiazolidine-2-carboxamido)methyl)benzoic acid,
(98) 3-((R)-3-amino-4-(2,4,5-trifluorophenyl)butanoyl)-N-(4-(2-oxo-2-(piperazine-1-yl)ethoxy)benzyl)thiazolidine-2-carboxamide,
(99) 3-((R)-3-amino-4-(2,4,5-trifluorophenyl)butanoyl)-N-(4-(2-oxo-2-thiomorpholinoethoxy)benzyl)thiazolidine-2-carboxamide,
(100) 3-((R)-3-amino-4-(2,4,5-trifluorophenyl)butanoyl)-N-(4-(2-morpholino-2-oxoethoxy)benzyl)thiazolidine-2-carboxamide,
(101) (S)-ethyl 2-(5-(((S)-3-((R)-3-amino-4-(2,4,5-trifluorophenyl)butanoyl) thiazolidine-2-carboxamido)methyl)pyridin-2-ylamino)-3-methylbutanoate,
(102) (S)-2-(5-(((S)-3-((R)-3-amino-4-(2,4,5-trifluorophenyl)butanoyl) thiazolidine-2-carboxamido)methyl)pyridin-2-ylamino)-3-methylbutanoic acid,
(103) (S)-3-((R)-3-amino-4-(2,4,5-trifluorophenyl)butanoyl)-N-(4-((R)-3-methyl-1-morpholino-1-oxobutan-2-ylamino)benzyl)thiazolidine-2-carboxamide,
(104) (R)-ethyl 2-(5-(((S)-3-((R)-3-amino-4-(2,4,5-trifluorophenyl)butanoyl) thiazolidine-2-carboxamido)methyl)pyridin-2-ylamino)-3-methylbutanoate,
(105) (R)-2-(5-(((S)-3-((R)-3-amino-4-(2,4,5-trifluorophenyl)butanoyl) thiazolidine-2-carboxamido)methyl)pyridin-2-ylamino)-3-methylbutanoic acid,
(106) (S)-3-((R)-3-amino-4-(2,4,5-trifluorophenyl)butanoyl)-N-(4-((S)-3-methyl-1-morpholino-1-oxobutan-2-ylamino)benzyl)thiazolidine-2-carboxamide,
(107) (R)-ethyl 2-(5-(((S)-3-((R)-3-amino-4-(2,4,5-trifluorophenyl)butanoyl) thiazolidine-2-carboxamido)methyl)pyrimidin-2-ylamino)-3-methylbutanoate,
(108) (R)-2-(5-(((S)-3-((R)-3-amino-4-(2,4,5-trifluorophenyl)butanoyl) thiazolidine-2-carboxamido)methyl)pyrimidin-2-ylamino)-3-methylbutanoic acid,
(109) (R)-ethyl 2-(5-(((S)-3-((R)-3-amino-4-(2,4,5-trifluorophenyl)butanoyl) thiazolidine-2-carboxamido)methyl)pyridin-2-yloxy)-3-methylbutanoate,
(110) (R)-2-(5-(((S)-3-((R)-3-amino-4-(2,4,5-trifluorophenyl)butanoyl) thiazolidine-2-carboxamido)methyl)pyridin-2-yloxy)-3-methylbutanoic acid,
(111) (R)-ethyl 2-(4-(((S)-3-((R)-3-amino-4-(2,4,5-trifluorophenyl)butanoyl) thiazolidine-2-carboxamido)methyl)-3-fluorophenylamino)-3-methylbutanoate,
(112) (R)-2-(4-(((S)-3-((R)-3-amino-4-(2,4,5-trifluorophenyl)butanoyl) thiazolidine-2-carboxamido)methyl)-3-fluorophenylamino)-3-methylbutanoic acid,
(113) (S)-3-((R)-3-amino-4-(2,4,5-trifluorophenyl)butanoyl)-N-(4-((R)-1-hydroxy-3-methylbutan-2-ylamino)benzyl)thiazolidine-2-carboxamide,
(114) (R)-2-methoxyethyl 2-(4-(((S)-3-((R)-3-amino-4-(2,4,5-trifluorophenyl) butanoyl)thiazolidine-2-carboxamido)methyl)phenylamino)-3-methylbutanoate,
(115) (S)-3-((R)-3-amino-4-(2,4,5-trifluorophenyl)butanoyl)-N-(4-((R)-3-methyl-1-(methylamino)-1-oxobutan-2-ylamino)benzyl)thiazolidine-2-carboxamide,
(116) (S)-3-((R)-3-amino-4-(2,4,5-trifluorophenyl)butanoyl)-N-(4-((R)-1-(dimethylamino)-3-methyl-1-oxobutan-2-ylamino)benzyl)thiazolidine-2-carboxamide,
(117) (R)-2-morpholinoethyl 2-(4-(((S)-3-((R)-3-amino-4-(2,4,5-trifluorophenyl) butanoyl)thiazolidine-2-carboxamido)methyl)phenylamino)-3-methylbutanoate,
(118) (R)-2-hydroxyethyl 2-(4-(((S)-3-((R)-3-amino-4-(2,4,5-trifluorophenyl) butanoyl)thiazolidine-2-carboxamido)methyl)phenylamino)-3-methylbutanoate,
(119) (R)-2-(methylamino)ethyl 2-(4-(((S)-3-((R)-3-amino-4-(2,4,5-trifluorophenyl)butanoyl)thiazolidine-2-carboxamido)methyl)phenylamino)-3-methylbutanoate,
(120) (S)—N-(4-((R)-1-amino-3-methyl-1-oxobutan-2-ylamino)benzyl)-3-((R)-3-amino-4-(2,4,5-trifluorophenyl)butanoyl)thiazolidine-2-carboxamide,
(121) (S)-3-((R)-3-amino-4-(2,4,5-trifluorophenyl)butanoyl)-N-(4-((R)-1-(ethylamino)-3-methyl-1-oxobutan-2-ylamino)benzyl)thiazolidine-2-carboxamide,
(122) (S)-3-((R)-3-amino-4-(2,4,5-trifluorophenyl)butanoyl)-N-(4-((R)-3-methyl-1-oxo-1-(piperazin-1-yl)butan-2-ylamino)benzyl)thiazolidine-2-carboxamide,
(123) (S)-3-((R)-3-amino-4-(2,4,5-trifluorophenyl)butanoyl)-N-(4-((R)-1-(2-hydroxyethylamino)-3-methyl-1-oxobutan-2-ylamino)benzyl)thiazolidine-2-carboxamide,
(124) (S)-3-((R)-3-amino-4-(2,4,5-trifluorophenyl)butanoyl)-N-(4-((R)-3-methyl-1-oxo-1-(piperidin-4-ylamino)butan-2-ylamino)benzyl)thiazolidine-2-carboxamide,
(125) (S)-3-((R)-3-amino-4-(2,4,5-trifluorophenyl)butanoyl)-N-(4-((R)-3-methyl-1-(2-(methylamino)ethylamino)-1-oxobutan-2-ylamino)benzyl)thiazolidine-2-carboxamide,
(126) (R)-2-aminoethyl 2-(4-(((S)-3-((R)-3-amino-4-(2,4,5-trifluorophenyl) butanoyl)thiazolidine-2-carboxamido)methyl)phenylamino)-3-methylbutanoate,
(127) (R)-isopropyl 2-(4-(((S)-3-((R)-3-amino-4-(2,4,5-trifluorophenyl) butanoyl)thiazolidine-2-carboxamido)methyl)phenylamino)-3-methylbutanoate,
(128) (R)-1,3-dihydroxypropan-2-yl 2-(4-(((S)-3-((R)-3-amino-4-(2,4,5-trifluorophenyl)butanoyl)thiazolidine-2-carboxamido)methyl)phenylamino)-3-methylbutanoate,
(129) (R)-2-(2,2,2-trifluoroacetamido)ethyl 2-(4-(((S)-3-((R)-3-amino-4-(2,4,5-trifluorophenyl)butanoyl)thiazolidine-2-carboxamido)methyl)phenylamino)-3-methylbutanoate,
(130) (R)-ethyl 2-(4-(((S)-3-((R)-3-amino-4-(2,4,5 trifluorophenyl)butanoyl)thiazolidine-2-carboxamido)methyl)-2-fluorophenylamino)-3-methylbutanoate,
(131) (R)-2-(4-(((S)-3-((R)-3-amino-4-(2,4,5-trifluorophenyl)butanoyl)thiazolidine-2-carboxamido)methyl)-2-fluorophenylamino)-3-methylbutanoic acid,
(132) (R)-ethyl 2-(4-(((S)-3-((R)-3-amino-4-(2,4,5-trifluorophenyl)butanoyl)thiazolidine-2-carboxamido)methyl)-2-(trifluoromethyl)phenylamino)-3-methylbutanoate,
(133) (R)-2-(4-(((S)-3-((R)-3-amino-4-(2,4,5-trifluorophenyl)butanoyl)thiazolidine-2-carboxamido)methyl)-2-(trifluoromethyl)phenylamino)-3-methylbutanoic acid,
(134) (R)-ethyl 2-(4-(((S)-3-((R)-3-amino-4-(2,4,5-trifluorophenyl)butanoyl)thiazolidine-2-carboxamido)methyl)-3-fluorophenylamino)-3-methylbutanoate, and
(135) (R)-2-(4-(((S)-3-((R)-3-amino-4-(2,4,5-trifluorophenyl)butanoyl)thiazolidine-2-carboxamido)methyl)-3-fluorophenylamino)-3-methylbutanoic acid, in free, salt or prodrug form.

In a preferred embodiment, said compounds are in a hydrochloride salt form.

In an especially preferred embodiment, the compounds of formula (Q) useful for inhibiting DPP-IV are selected from:

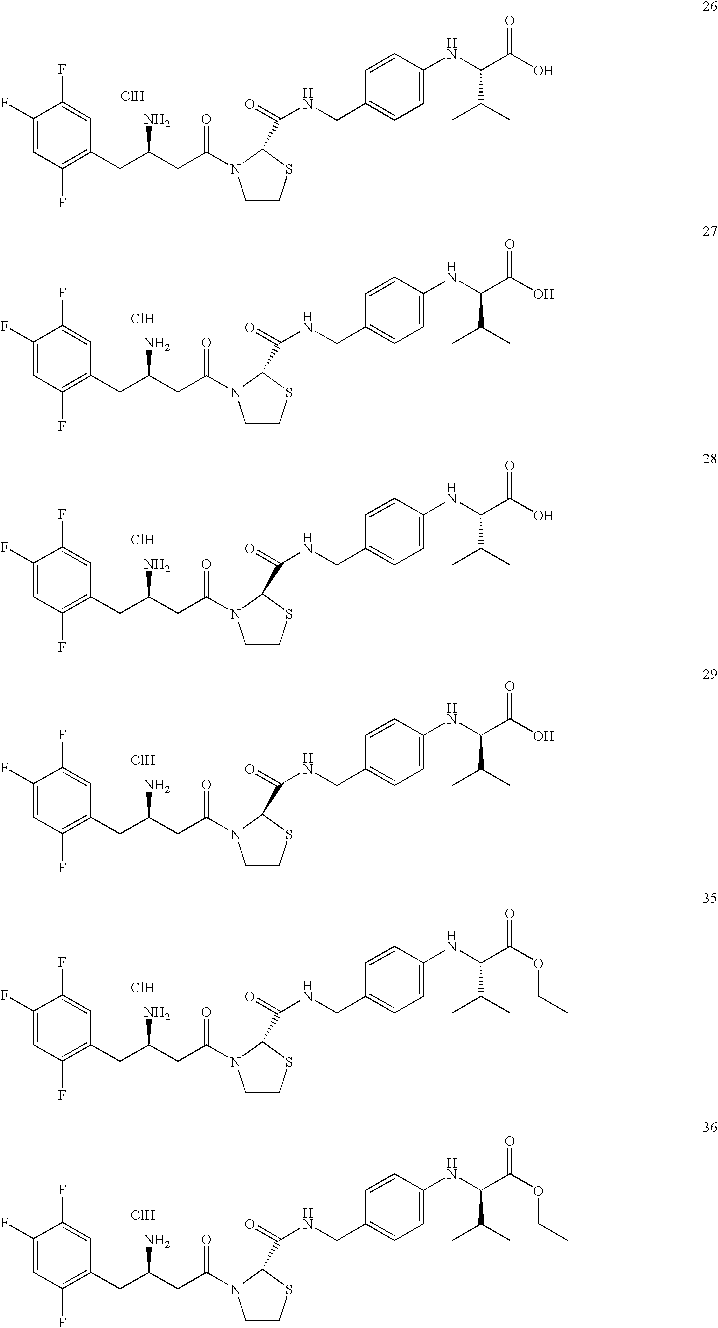

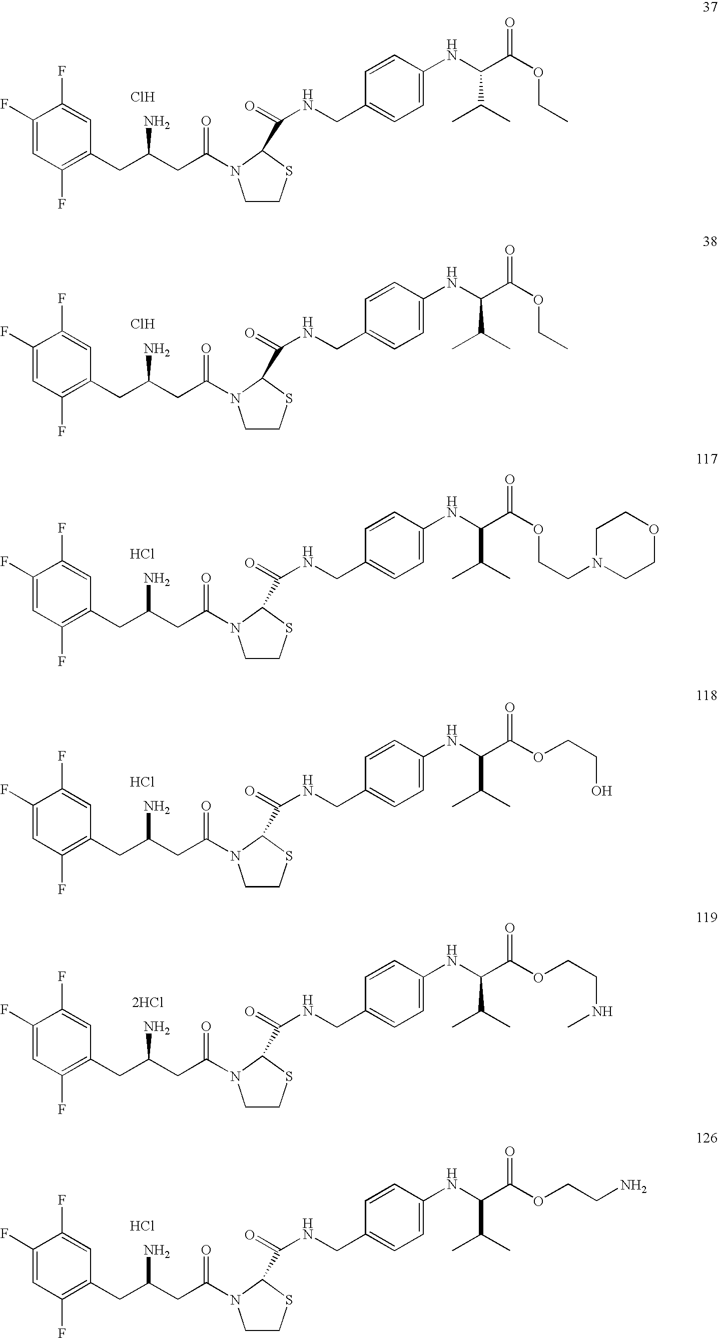

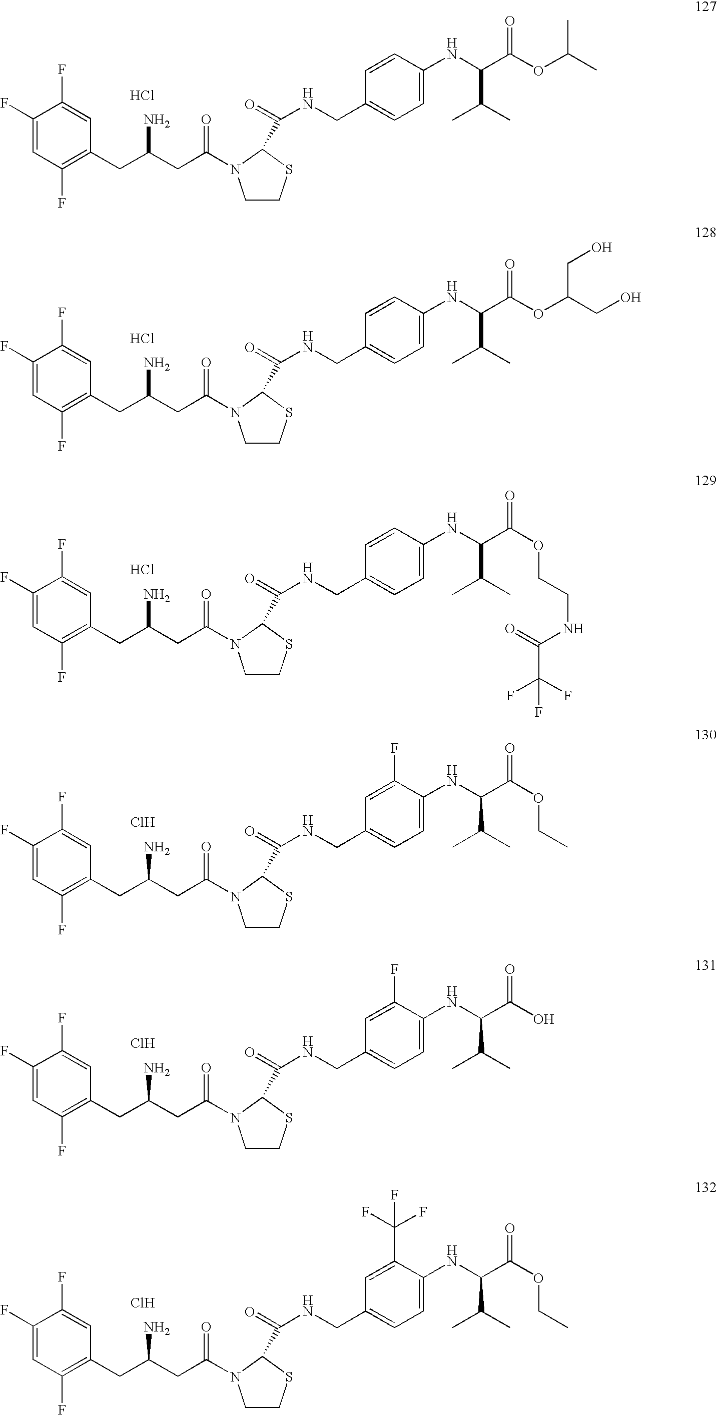

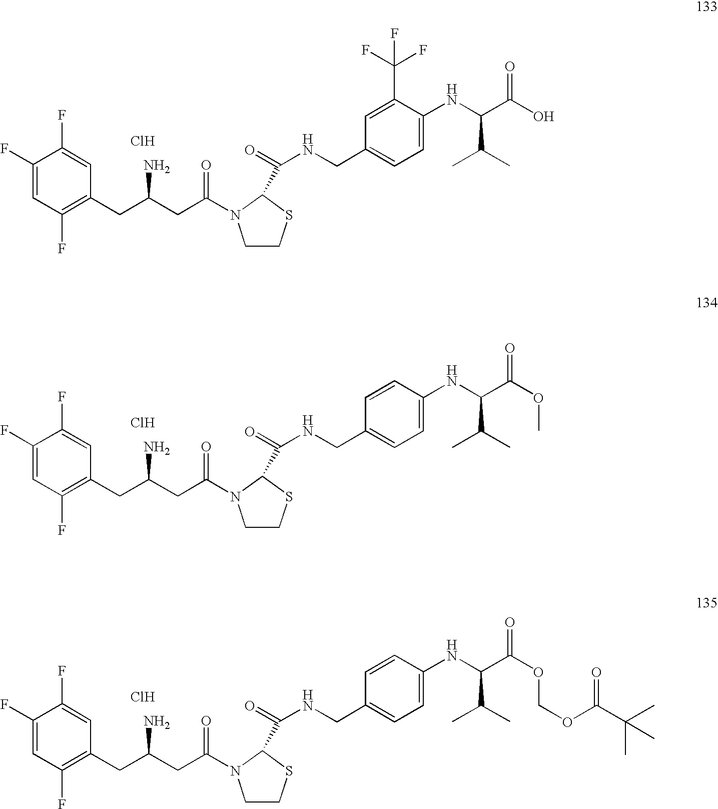

in free, salt or prodrug form.

The compound of formula 1 or formula (Q) according to the present invention may be prepared by various reaction routes.

In accordance with the first reaction route, the disclosed compound, for example, a compound of formula 1a (i.e., the compound of formula 1 wherein A is —OR^{b}) may be prepared by (i) subjecting an amino acid of formula 2 to a condensation reaction with a 2-carbonyl-1,3-thiazolidine-based compound of formula 3 to form a compound of formula 4; and (ii) deprotecting the compound of formula 4, as shown in Reaction Scheme 1.

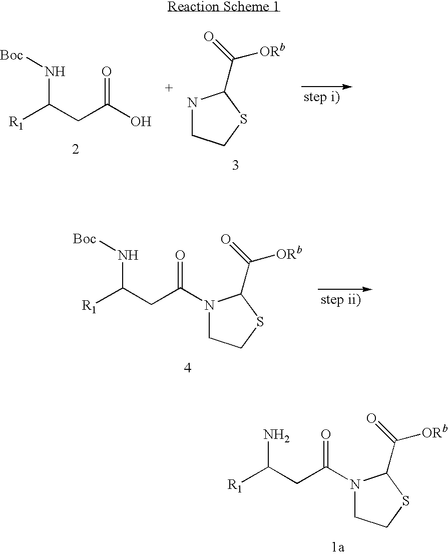

wherein, R₁, R^{b} and Boc are the same as defined above.

The amino acid of formula 2 used as a starting material in Reaction Scheme 1 may be prepared by a conventionally known method (see Ahn, J. H. et al., Bioorg. & Med. Chem. Lett. 2007, 17, 2622-2628).

The 2-carbonyl-1,3-thiazolidine-based compound of formula 3 may be commercially available, or may be prepared by a conventionally known method (see U.S. Pat. No. 6,867,211; and Johnson, R. L., Smissman, E. E., and Plolnikoff, N. P., J. Med. Chem. 1978, 21, 165) or by the method as shown below.

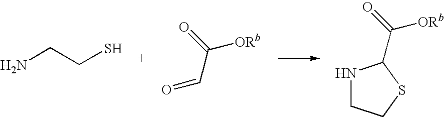

wherein, R^{b} is the same as defined above.

The compound of formula 3 may be subjected to crystallization by utilizing L- or D-tartaric acid to obtain a chiral stereoisomer of formula 3a or 3b. The crystallization is preferably conducted by utilizing dynamic kinetic resolution (DKR) so as to obtain the desired compound in a yield of 50% or higher selectively and quantitatively. The chiral stereoisomer obtained may be analyzed by high performance liquid chromatography (HPLC).

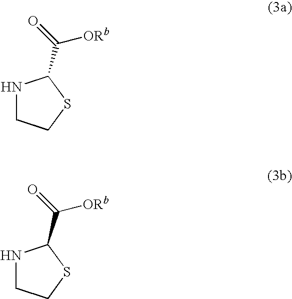

wherein, R^{b} is the same as defined above.

The crystallization by DKR may be conducted in a solvent of ethanol-diethyl ether mixture in the presence of 1 to 3 equivalents of L- or D-tartaric acid with the solvent being slowly evaporated. Further, the crystallization is preferably carried out at a temperature of 0 to 80° C. After crystallization, the filtrate may be concentrated and slowly evaporated for further recrystallization. The resultant obtained is a tartaric salt of the compound of formula 3, which may be further neutralized with 10% sodium bicarbonate or sodium carbonate and extracted with diethyl ether to produce the compound of formula 3a or 3b.

The stereoisomer of formula 3a or 3b thus obtained can be used as a starting material in Reaction Scheme 1 for the production of the compound of formula 1 in the form of a stereoisomer.

In step i) of Reaction Scheme 1, the amino acid of formula 2 is used in an amount of about 1 to 2 equivalents relative to the amount of the compound of formula 3.

Step i) (condensation reaction) may be conducted in the presence of a condensing agent in a solvent, e.g., an aliphatic hydrocarbon such as dichloromethane or chloroform. The condensing agent may be selected from the group consisting of 1,1′-carbonyldiimidazole (CDI), 1-(3-dimethylaminopropyl)-3-ethylcarbodiimide hydrochloride (EDCI), 1,3-dicyclohexylcarbodiimide (DCC) and a mixture thereof, and other condensing agent conventionally known in the art may be also used. The condensing agent may be used in an amount of about 1 to 2 equivalents relative to the amount of the compound of formula 3. Also, step i) may be conducted in the presence of a base such as an amine base (e.g., triethylamine or pyridine), the base being used in an amount of about 2 to 5 equivalents relative to the amount of the compound of formula 3. Such step i) is preferably conducted for 10 to 24 hours at a temperature of 20 to 70° C.

Step ii) of Reaction Scheme 1, deprotection, may be conducted in the presence of a deprotecting agent such as hydrochloric and trifluoroacetic acid in a solvent such as 1,4-dioxane, dichloromethane and ethyl acetate. The deprotecting agent is preferably used in an amount of 5 to 10 equivalents relative to the amount of the compound of formula 4. Step ii) is preferably conducted for 3 to 10 hours at a temperature of 20 to 40° C. The deprotection procedure is continued until the compound of formula 4 is wholly consumed, which may be confirmed by thin layer chromatography.

Meanwhile, the compound of formula 4 may be hydrolyzed to form a compound of formula 7, which may be deprotected to obtain the compound of formula 1 wherein A is OH.

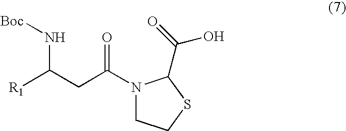

wherein, Boc and R₁ are the same as defined above.

The hydrolysis of the compound of formula 4 may be conducted in the presence of a base, e.g., an inorganic base such as sodium hydroxide (NaOH), potassium hydroxide (KOH) and lithium hydroxide (LiOH), in a solvent such as water, a lower alcohol, tetrahydrofuran (THF), dioxane and a mixture thereof. The base is preferably used in an amount of 1 to 20 equivalents relative to the amount of the compound of formula 4. The hydrolysis is preferably conducted for 1 to 12 hours at a temperature of 20 to 70° C.

In accordance with the second reaction route for preparing the compound of formula 1, a compound of formula 1b (i.e., the compound of formula 1 wherein A′ is

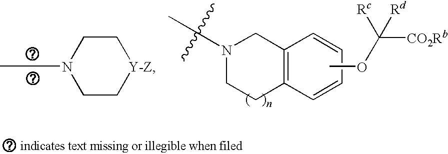

or —NR^{e} (CH₂)ₙR₂) may be prepared by (i) subjecting an amino acid of formula 2 to a condensation reaction with a 2-carbonyl-3-acyl-1,3-thiazolidine-based compound of formula 3 to form a compound of formula 4; (ii) forming a compound of formula 5 from the compound of formula 4; and (iii) deprotecting the compound of formula 5, as shown in Reaction Scheme 2.

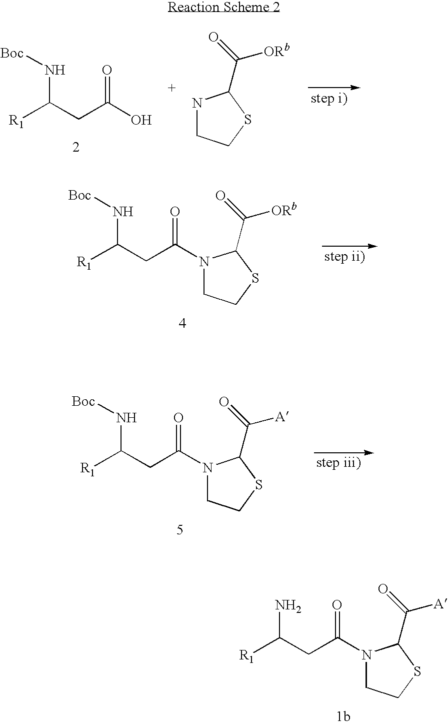

wherein, R₁, R^{b}, Boc and A′ are the same as defined above.

In Reaction Scheme 2, step i) is conducted by the same procedure as step i) of Reaction Scheme 1 for the first reaction route.

Step ii) of Reaction Scheme 2 may be conducted by a conventional nucleophilic substitution reaction or a hydrolyzing procedure followed by a condensation reaction, according to the types of the substituents —OR^{b} and A′.

For example, the compound of formula 4 may be hydrolyzed to form a compound of formula 7, which is then subjected to a condensation reaction with an A′-containing nucleophilic compound (e.g., HNR^{e}(CH₂)ₙR₂ or, HOR^{b}) to obtain the compound of formula 5.

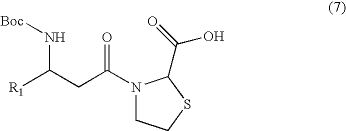

wherein, Boc and R₁ are the same as defined above.

The hydrolysis may be conducted by the procedure as disclosed in the first reaction route.

The condensation reaction with the A′-containing nucleophilic compound may be conducted in the presence of a condensing agent in a solvent, e.g., an aliphatic hydrocarbon such as dichloromethane or chloroform. The condensing agent may be selected from the group consisting of 1,1′-carbonyldiimidazole (CDI), 1-(3-dimethylaminopropyl)-3-ethylcarbodiimide hydrochloride (EDCI), 1,3-dicyclohexylcarbodiimide (DCC) and a mixture thereof, and other condensing agent conventionally known in the art may be also used. Each of the A′-containing nucleophilic compound and the condensing agent may be used in an amount of about 1 to 2 equivalents, relative to the amount of the compound of formula 7. Also, the condensation reaction may be conducted in the presence of a base such as an amine base (e.g., triethylamine or pyridine), the base being used in an amount of about 1 to 5 equivalents relative to the amount of the compound of formula 7. Such condensation reaction is preferably conducted for 1 to 24 hours at a temperature of 0 to 70° C.

The A′-containing nucleophilic compound may be substituted aniline compounds, substituted aryl compounds, methylene primary amines substituted with heteroaryl, ethylene primary amines substituted with heteroaryl or cyclized secondary amines, according to the type of A′, or it may be compounds having A′ being bonded with hydrogen or any other functional group.

Alternatively, the compound of formula 4 may be subjected to a conventional nucleophilic substitution reaction with the A′-containing compound, or other conventional methods in the art, to obtain the compound of formula 5.

Then, the compound of formula 5 may be deprotected to obtain the compound of formula 1b. The deprotection may be conducted in the presence of a deprotecting agent such as hydrochloric and trifluoroacetic acid in a solvent such as 1,4-dioxane, dichloromethane and ethyl acetate. The deprotecting agent is preferably used in an amount of 5 to 10 equivalents relative to the amount of the compound of formula 5. The deprotection is preferably conducted for 3 to 10 hours at a temperature of 20 to 40° C. The deprotection procedure is continued until the compound of formula 5 is wholly consumed, which may be confirmed by thin layer chromatography.

In accordance with the third reaction route for preparing the compound of formula 1, a compound of formula 1b-1 (i.e., the compound of formula 1 wherein A′ is —NR^{e}(CH₂)ₙR₂) may be prepared by (i) hydrolyzing a compound of formula 6 to form a compound of formula 7; (ii) subjecting the compound of formula 7 to a condensation reaction with a nucleophilic compound of formula 8 to form a compound of formula 9; and (iii) deprotecting the compound of formula 9, as shown in Reaction Scheme 3.

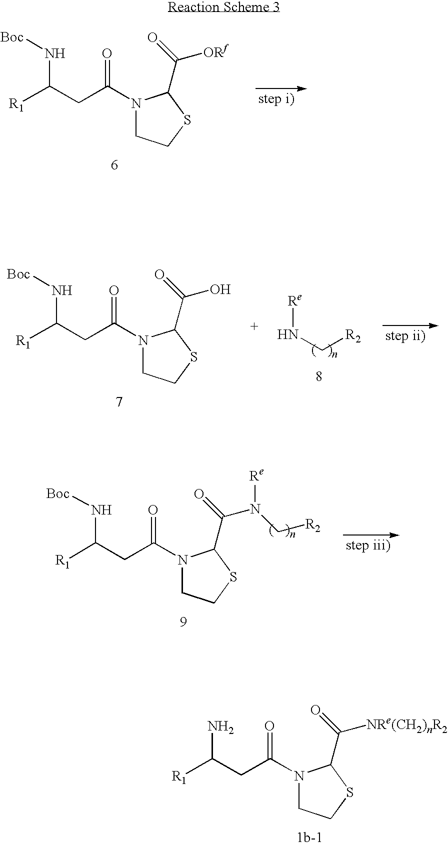

wherein, Boc, R₁, R₂, R^{e}, R^{f} and n are the same as defined above.

In accordance with the fourth reaction route for preparing the compound of formula 1, a compound of formula 1b-2 (i.e., the compound of formula 1 wherein A is

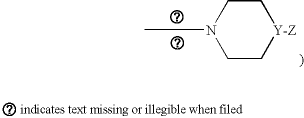

may be prepared by (i) subjecting a compound of formula 7 to a condensation reaction with a compound of formula 10 to form a compound of formula 5a; and (ii) deprotecting the compound of formula 5a, as shown in Reaction Scheme 4.

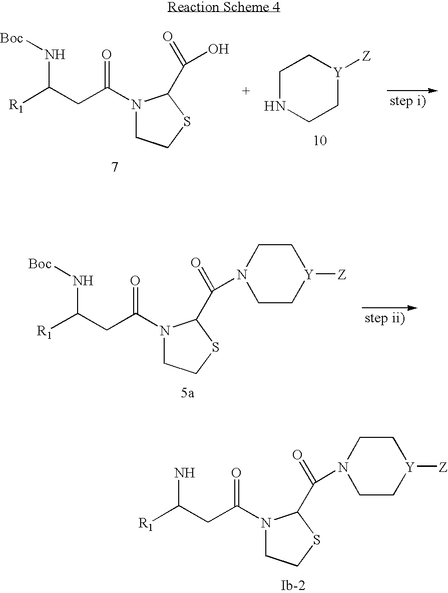

wherein, Boc, R₁, Y and Z are the same as defined above.

In Reaction Scheme 3, step i) (hydrolysis) may be conducted by the procedure as disclosed in the hydrolysis step of Reaction Scheme 1 or 2 (e.g., hydrolysis of a compound of formula 4 to compound of formula (7) using a base, e.g., an inorganic base such as sodium hydroxide (NaOH), potassium hydroxide (KOH) and lithium hydroxide (LiOH)). The nucleophilic compound of formula 8 may be substituted aniline compounds, substituted aryl compounds, aminomethyl or secondary amines substituted with heteroaryl, aminoethyl substituted with heteroaryl or cyclized secondary amines, or it may be compounds having R₂ being bonded with other functional groups.

Step ii) of Reaction Scheme 3 and step i) of Reaction Scheme 4, i.e., condensation reaction may be conducted in the presence of a condensing agent in a solvent, e.g., an aliphatic hydrocarbon such as dichloromethane or chloroform. The condensing agent may be selected from the group consisting of 1,1′-carbonyldiimidazole (CDI), 1-(3-dimethylaminopropyl)-3-ethylcarbodiimide hydrochloride (EDCI), 1,3-dicyclohexylcarbodiimide (DCC) and a mixture thereof, and other condensing agent conventionally known in the art may be also used. Each of the nucleophilic compound of formula 8 or the compound of formula 10, and the condensing agent may be used in an amount of about 1 to 2 equivalents, relative to the amount of the compound of formula 7. Also, the condensation reaction may be conducted in the presence of a base such as an amine base (e.g., triethylamine or pyridine) in an amount of about 1 to 5 equivalents relative to the amount of the compound of formula 7. Such condensation reaction is preferably conducted for 1 to 24 hours at a temperature of 0 to 70° C.

Step iii) of Reaction Scheme 3 and step ii) of Reaction Scheme 4, i.e., deprotection, may be conducted in the presence of a deprotecting agent such as hydrochloric and trifluoroacetic acid in a solvent such as 1,4-dioxane, dichloromethane and ethyl acetate. The deprotecting agent is preferably used in an amount of 5 to 10 equivalents relative to the amount of the compound of formula 5a or 9. The deprotection is preferably conducted for 3 to 10 hours at a temperature of 20 to 40° C. The deprotection procedure is continued until the compound of formula 5 is wholly consumed, which may be confirmed by thin layer chromatography.

In accordance with the fifth reaction route for preparing the compound of formula 1, a compound of formula 1b-3 (i.e., the compound of formula 1 wherein A is —NR^{e}(CH₂)ₙBCO₂H and BCO₂H is the same as defined above) may be prepared by (i) hydrolyzing a compound of formula 11 to form a compound of formula 12; and (ii) deprotecting the compound of formula 12, as shown in Reaction Scheme 5.

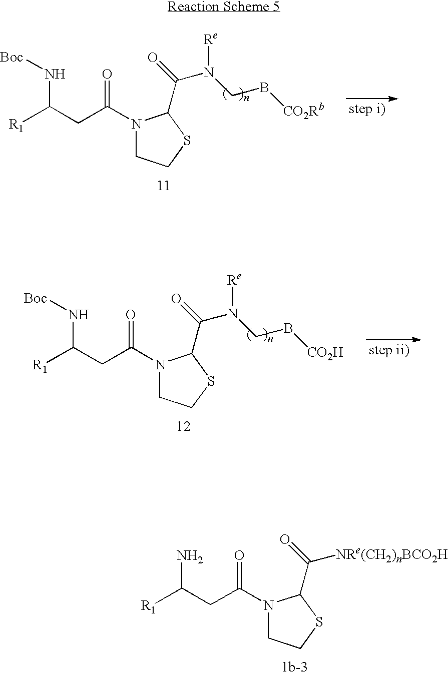

wherein, Boc, R₁, n and BCO₂H are the same as defined above.

The compound of formula 11 may be prepared by a process similar to that employed for preparing the compound of formula 9 in the third reaction route.

In Reaction Scheme 5, step i) (hydrolysis) may be conducted in the presence of a base, e.g., an inorganic base such as sodium hydroxide (NaOH), potassium hydroxide (KOH) and lithium hydroxide (LiOH) in a solvent such as water, a lower alcohol, tetrahydrofuran (THF), dioxane and a mixture thereof. The base is preferably used in an amount of 1 to 20 equivalents relative to the amount of the compound of formula 11. The hydrolysis is preferably conducted for 1 to 12 hours at a temperature of 20 to 70° C.

Then, step ii) of Reaction Scheme 5 (deprotection) may be conducted as disclosed above.

Similarly, compounds of formula (Q) or any of formula 1.1-1.75 may be prepared as hereinbefore described for compounds of formula 1 (e.g., Reaction Schemes 1-5) with the exception that the substituents P₁, R₁, R₂, and R^{a}-R^{h} are as defined in Methods (I)-(V) or formula (Q). Therefore, P₁ of compounds of formula Q-2, Q-4, Q-5, Q-9, Q-5a, or Q-12, may be any amine protecting group which is capable of preventing or reducing the reactivity of the amine group with other nucleophiles. P₁ therefore includes but is not limited to tert-butyloxycarbonyl (BOC), carbobenzyloxy (CBz), benzyl, Phthalimides (Pht), sulfonyl protecting groups (e.g., p-toluenesulfonyl) and other protecting groups well known in the art, including those found in “Protective Groups in Organic Synthesis” by Theodora Green (publisher: John Wiley & Sons), the disclosure of which is hereby incorporated by reference.

In deprotecting the amine of compounds of formula Q-4, Q-5, Q-9, Q-5a, or Q-12, appropriate deprotecting agent may be employed depending on the protecting agent used. For example, to removing a BOC or CBz protecting group, an acid or a combination of acids (e.g., trifluoroacetic acid, hydrobromic acid, acetic acid or hydrochloric acid) may be used. Benzyl protecting group may be removed by hydrogenation method (H₂ and palladium on carbon). Phthalimide protecting group may be removed by employing hydrazine. Sulfonyl protecting group may be removed by reduction method (e.g., using sodium or lithium in liquid ammonia). This list is not intended to be exhaustive and therefore does not exclue other deprotecting agents well known in the art such as those found in “Protective Groups in Organic Synthesis” by Theodora Green (publisher: John Wiley & Sons).

Other reactions for preparing compounds of formula (Q), e.g., condensation reaction and hydrolysis may be performed as described above in for compounds of formula 1.

The disclosed compounds of formula 1 and formula (Q) obtained thus show good inhibiting activity against DPP-IV.

Accordingly, the present invention provides a pharmaceutical composition comprising the compound of formula 1 in free or a pharmaceutically acceptable salt thereof and a pharmaceutically acceptable carrier, which is useful for preventing or treating DPP-IV-mediated diseases, such as insulin-dependent diabetes mellitus, insulin-independent diabetes mellitus, arthritis, obesity, osteoporosis and impaired glucose tolerance.

In another aspect, the invention provides a pharmaceutical composition comprising the compound of formula (Q) in free or pharmaceutically acceptable salt thereof and a pharmaceutically acceptable dilluent or carrier, which is useful for preventing or treating DPP-IV-mediated diseases, such as insulin-dependent diabetes mellitus, insulin-independent diabetes mellitus, arthritis, obesity, osteoporosis and impaired glucose tolerance.

The pharmaceutical composition may be formulated for oral or parenteral administration. The formulation for oral administration may take various forms such as tablet, pill, powder, soft and hard capsule, solution, suspension, emulsion, syrup, granule, elixir and the like, which may contain conventional additives such as a diluent (e.g., lactose, dextrose, sucrose, mannitol, sorbitol, cellulose and/or glycine), a lubricant (e.g., silica, talc, stearic acid or its magnesium or calcium salt, and/or polyethylene glycol).

A tablet form may also comprise a binder such as magnesium aluminum silicate, starch paste, gelatin, tragacanth, methyl cellulose, sodium carboxylmethyl cellulose and/or polyvinylpyrrolidone, and optionally a disintegrant such as starch, agar, alginic acid or its sodium salt, an effervescent mixture, an absorbent, a colorant, a flavor or a sweetener.

For parenteral administration, subcutaneous, intravenous, intramuscular or intraabdominal injection may be taken in the form of formulations such as solution and suspension which are contained in ample or vial.

Also, the pharmaceutical composition may be steriled, additionally include preservatives, stabilizers, wetting agents, emulsifying agents, osmotic pressure-adjusting agents, buffering agents and other therapeutically useful materials and may be formulated through a conventional mixing, granulating or coating procedures.

A typical daily dose of the compound of formula 1 ranges from about 0.1 to 500 mg/kg, preferably 0.1 to 100 mg/kg for mammals including a human being and can be orally or parenterally administered in a single dose or in divided doses.

Furthermore, the present invention provides a method for inhibiting DPP-IV in a mammal, comprising administering the compound of formula 1 in free or pharmaceutically acceptable salt thereof to the mammal in an amount effective for the inhibition of DPP-IV. The present invention also provides a method for inhibiting DPP-IV in a mammal, comprising administering the compound of formula (Q) in free or pharmaceutically acceptable salt thereof to the mammal in an amount effective for the inhibition of DPP-IV.

Also, the present invention provides a method for treating DPP-IV-mediated diseases in a mammal, comprising administering the compound of formula 1 in free or pharmaceutically acceptable salt thereof to the mammal in a therapeutically effective amount, the DPP-IV-mediated disease being insulin-dependent diabetes mellitus, insulin-independent diabetes mellitus, arthritis, obesity, osteoporosis or impaired glucose tolerance. Similarly, the present invention provides a method for treating DPP-IV-mediated diseases in a mammal, comprising administering the compound of formula (Q) in free or pharmaceutically acceptable salt thereof to the mammal in a therapeutically effective amount, the DPP-IV-mediated disease being insulin-dependent diabetes mellitus, insulin-independent diabetes mellitus, arthritis, obesity, osteoporosis or impaired glucose tolerance.

The administration route of the compound of formula 1 or formula (Q) or the therapeutically effective amount thereof will be determined depending on such various factors as the types of a mammal, diseases to be treated and a compound used, and the inhibiting activity against DPP-IV thereof.

In the present invention, it is intended that when a substituent is substituted with R^{a}, R^{a} may be substituted once or independently substituted more than once on said substituent. For example, where R₂ is

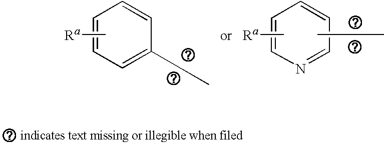

or any of the substituent selected from a group defined in formula (Q) or formula (1) and R^{a} is “one or more substitutents selected from the group consisting of hydrogen, C₁₋₆ alkyl (e.g., methyl), C₃₋₆ cycloalkyl, C₁₋₆ alkoxy, —OCF₃, hydroxy, —CH₂OH, halogen, —CN, —CF₃, —COOR^{b}, —CH₂COOR^{b}, —NR^{d}R^{e} and —OC(O)—C₁₋₆alkyl”, then R₂ may be:

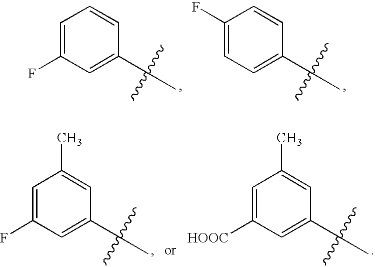

It is also intended that when R₂ is depicted as an aryl group substituted at an unspecified position, for example:

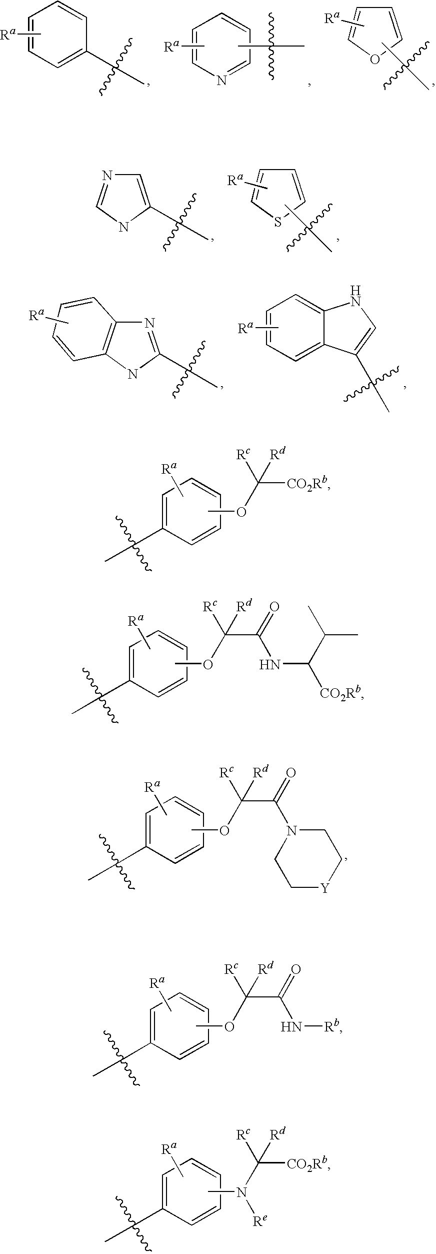

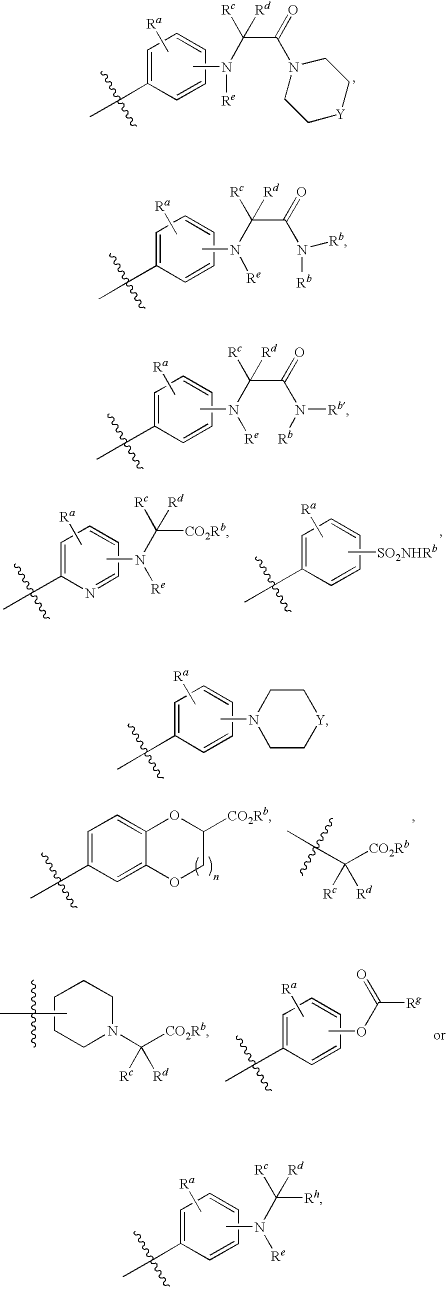

said substituents (e.g., R^{a} or —OC(O)R^{g},

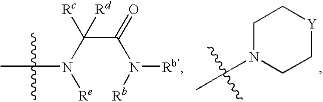

SO₂NHR^{b}, etc.) may be on any position of the ring.

The term “aryl” as used herein is a mono or bicyclic aromatic hydrocarbon, preferably phenyl.

The tarm “alkyl” as used herein is a saturated or unsaturated hydrocarbon moiety, preferably saturated, preferably one to four carbon atoms in length, which may be linear or branched, and may be optionally substituted, e.g., mono-, di-, or tri-substituted, e.g., with halogen (e.g., fluoro).

The present invention is further described and illustrated in Examples provided below, which are, however, not intended to limit the scope of the present invention.

### Example 1

### Preparation of methyl 3-((R)-3-amino-4-(2,4,5-trifluorophenyl) butanoyl)thiazolidine-2-carboxylate.HCl

### Step 1: Preparation of methyl 3-[(R)-3-t-butoxycarbonylamino-4-(2,4,5-trifluorophenyl)-butyryl]-thiazolidine-2-carboxylate

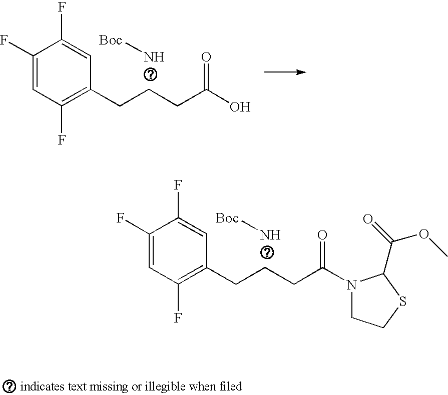

(R)-3-(tert-butoxycarbonylamino)-4-(2,4,5-trifluorophenyl)butanoic acid (5.13 g, 15.40 mmol) is dissolved in CH₂Cl₂. Thereto, 1-(3-dimethylaminopropyl)-3-ethylcarbodiimide hydrochloride (EDCI, 2.95 g, 15.4 mmol), dimethylaminopyridine (376 mg, 3.00 mmol), methyl thiazolidine-2-carboxylate.HCl (2.82 g, 15.40 mmol) and triethylamine (10.73 ml, 76.96 mmol) are added, followed by stirring for 12 hours at room temperature. The resulting mixture is washed with brine and extracted with CH₂Cl₂. The entire extracts are dried over MgSO₄. The organic layer is concentrated under a reduced pressure and separated by column chromatography (EtOAc:hexane=1:1) to obtain the compound, methyl 3-((R)-3-(tert-butoxycarbonylamino)-4-(2,4,5-trifluorophenyl)butanoyl)thiazolidine-2-carboxylate (5.48 g, 77%) as a white solid.

¹H NMR (CDCl₃, 300 MHz) δ 7.16-7.06 (m, 1H), 6.94-6.85 (m, 1H), 5.59 (d, J=3.3 Hz, 1H), 4.13-4.10 (m, 1H), 3.95-3.92 (m, 1H), 3.79 (s, 3H), 3.77-3.72 (m, 1H), 3.37-3.34 (m, 1H), 3.11-3.09 (m, 1H), 2.94-2.92 (m, 2H), 2.65-2.60 (m, 2H), 1.37 (s, 9H); LC-MS m/z (relative intensity) 463 (MH⁺).

### Step 2: Preparation of methyl 3-((R)-3-amino-4-(2,4,5-trifluorophenyl) butanoyl)thiazolidine-2-carboxylate.HCl

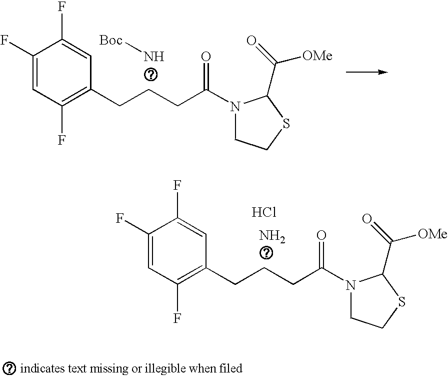

Methyl 3-((R)-3-(tert-butoxycarbonylamino)-4-(2,4,5-trifluorophenyl)butanoyl)thiazolidine-2-carboxylate (93 mg, 0.2 mmol) obtained in step 1 above is dissolved in EtOAc. Thereto, a 4 M HCl/1,4-dioxane mixture (0.1 ml) is added, followed by stirring for 12 hours at room temperature. The resulting mixture is concentrated under a reduced pressure to remove excessive solvent and crystallized with diethyl ether to obtain the desired compound, methyl 3-((R)-3-amino-4-(2,4,5-trifluorophenyl)butanoyl)thiazolidine-2-carboxylate HCl (77 mg, 97%) as a white solid.

### Example 2

### Preparation of 3-((R)-3-amino-4-(2,4,5-trifluorophenyl) butanoyl)thiazolidine-2-carboxylic acid.HCl

### Step 1: Preparation of 3-[(R)-3-t-butoxycarbonylamino-4-(2,4,5-trifluorophenyl)-butyryl]-thiazolidine-2-carboxylic acid

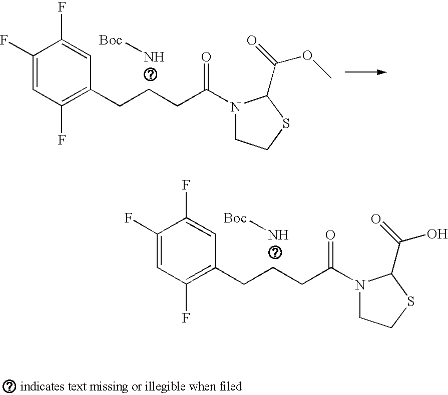

Methyl 3-[(R)-3-t-butoxycarbonylamino-4-(2,4,5-trifluorophenyl)-butyryl]-thiazolidine-2-carboxylate (1.26 g, 2.72 mmol) obtained in step 1 of Example 1 is dissolved in a mixture of tetrahydrofuran (10 ml) and methanol (10 ml). Thereto, LiOH.H₂O (579 mg, 13.62 mmol) dissolved in water (10 ml) is added, followed by stirring for 12 hours at room temperature. The resulting mixture is concentrated under a reduced pressure to remove excessive solvent. The concentrate is cooled to 0° C. and acidified to a pH of 4 by slow and dropwise addition of 1 N—HCl. The resultant is extracted with CH₂Cl₂. The entire extracts are washed with brine, dried over MgSO₄, concentrated under a reduced pressure, and filtered to obtain the compound, 3-[(R)-3-t-butoxycarbonylamino-4-(2,4,5-trifluorophenyl)-butyryl]-thiazolidine-2-carboxylic acid (1.08 g, 88%) as a white solid.

¹H NMR (CDCl₃, 300 MHz) δ 7.11-7.04 (m, 1H), 6.93-6.85 (m, 1H), 5.50 (brs, 1H), 4.16-4.09 (m, 1H), 3.96-3.85 (m, 1H), 3.82-3.74 (m, 1H), 3.43-3.36 (m, 1H), 3.13-3.08 (m, 1H), 2.94-2.92 (m, 2H), 2.67-2.50 (m, 2H), 2.00-1.94 (m, 1H), 1.37 (s, 9H).

### Step 2: Preparation of 3-((R)-3-amino-4-(2,4,5-trifluorophenyl) butanoyl)thiazolidine-2-carboxylic acid.HCl

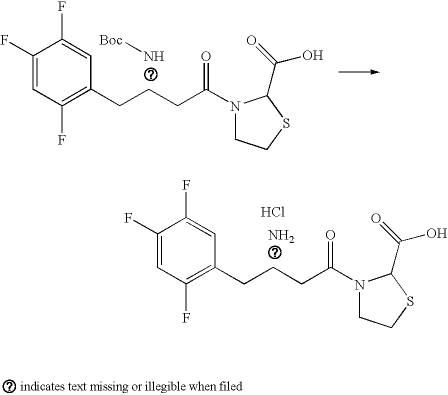

3-((R)-3-amino-4-(2,4,5-trifluorophenyl)butanoyl)thiazolidine-2-carboxylic acid.HCl is obtained according to the procedure used for Step 2, Example 1 (70 mg, 90%).

### Example 3

### Preparation of 3-((R)-3-amino-4-(2,4,5-trifluorophenyl) butanoyl)-N-benzylthiazolidine-2-carboxamide.HCl

### Step 1: Preparation of tert-butyl (R)-4-(2-(benzylcarbamoyl)thiazolidin-3-yl)-4-oxo-1-(2,4,5-trifluorophenyl)butan-2-ylcarbamate

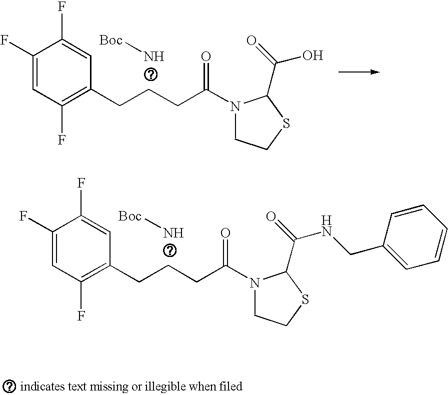

3-[(R)-3-t-butoxycarbonylamino-4-(2,4,5-trifluorophenyl)-butyryl]-thiazolidine-2-carboxylic acid (45 mg, 0.10 mmol) obtained in step 1 of Example 2 is dissolved in CH₂Cl₂ (1 ml). Thereto, benzylamine (11 μl, 0.20 mmol), EDCI (58 mg, 0.30 mmol) and Et₃N (70 μl, 0.50 mmol) are added, followed by stirring for 12 hours at room temperature. The resulting mixture is washed with brine and extracted with CH₂Cl₂. The entire extracts are dried over MgSO₄. The organic layer is concentrated under a reduced pressure and purified by column chromatography (EtOAc:hexane=1:1) to obtain the compound, tert-butyl (2R)-4-(2-(benzylcarbamoyl)thiazolidin-3-yl)-4-oxo-1-(2,4,5-trifluorophenyl)butan-2-ylcarbamate (15 mg, 28%).

¹H NMR (CDCl₃, 300 MHz) δ 7.60-7.28 (m, 5H), 7.12-7.07 (m, 1H), 6.91-6.86 (m, 1H), 6.30-6.15 (br, 1H), 5.53 (d, J=3.9 Hz, 1H), 4.44 (s, 2H), 4.13-4.11 (m, 1H), 4.00-3.91 (m, 1H), 3.77-3.75 (m, 1H), 3.51-3.44 (m, 1H), 3.20-3.00 (m, 2H), 2.92-2.90 (m, 2H), 2.65-2.60 (m, 2H), 1.37 (s, 9H).

### Step 2: Preparation of 3-((R)-3-amino-4-(2,4,5-trifluorophenyl) butanoyl)-N-benzylthiazolidine-2-carboxamide.HCl

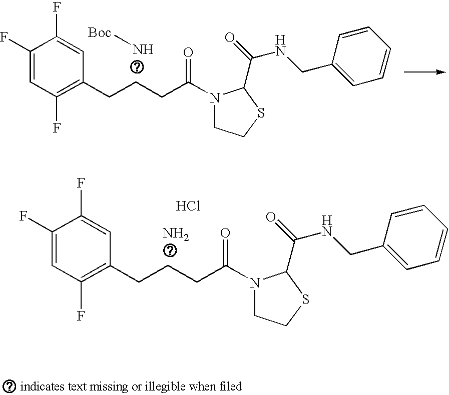

3-((R)-3-amino-4-(2,4,5-trifluorophenyl) butanoyl)-N-benzylthiazolidine-2-carboxamide.HCl is obtained according to the procedure used for Step 2, Example 1 (84%).

¹H NMR (CD₃OD, 300 MHz) δ 7.41-7.22 (m, 7H), 5.51 (d, J=10.8 Hz, 1H), 5.00-4.60 (m, 1H), 4.39 (s, 2H), 4.02-3.98 (m, 1H), 3.88-3.81 (m, 2H), 3.40-3.19 (m, 2H), 3.08-3.03 (m, 2H), 2.85-2.79 (m, 2H).

### Example 4

### Preparation of ethyl 2-(4-((3-((R)-3-amino-4-(2,4,5-trifluorophenyl)butanoyl)thiazolidine-2-carboxamido)methyl)phenoxy)acetate.HCl

### Step 1: Preparation of 4-hydroxy-benzaldehyde oxime

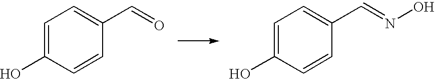

4-Hydroxy-benzaldehyde (5 g, 40.94 mmol) was dissolved in EtOH (100 ml). Thereto, hydroxyl amine.HCl (4.3 g, 61.41 mmol) and pyridine (9.9 ml, 122.82 mmol) are added. The mixture is refluxed for 1 hour. The resultant is concentrated under a reduced pressure, extracted with Et₂O. The entire extracts are washed with brine and dried over MgSO₄. The resulting organic solution is concentrated under a reduced pressure and purified by column chromatography (EtOAc:hexane=1:2) to obtain the compound, 4-hydroxy-benzaldehyde oxime (5.9 g, 100%).

¹H NMR (CDCl₃, 200 MHz) δ 9.23 (s, 1H), 8.15 (brs, 1H), 7.82 (s, 1H), 7.22 (d, J=8.8 Hz, 2H), 6.63 (d, J=8.8 Hz, 2H).

### Step 2: Preparation of 1-butyl (4-hydroxybenzyl)-carbamate

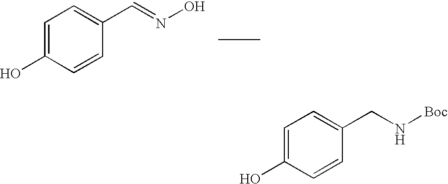

4-Hydroxy-benzaldehyde oxime (3.0 g, 21.88 mmol) obtained in step 1 above is dissolved in MeOH (70 ml). Thereto, 10% wt. Pd/C (300 mg) and Boc₂O (5.7 g, 26.25 mmol) are added, followed by stirring under H₂ pressure for 10 hours. After the remaining Pd is filtered out, the filtrate is concentrated under a reduced pressure and separated by column chromatography (EtOAc:hexane=1:2) to obtain the compound, t-butyl (4-hydroxybenzyl)-carbamate (3.0 g, 62%) as a white solid.

¹H NMR (CDCl₃, 200 MHz) δ 7.08 (d, J=8.2 Hz, 2H), 6.79 (s, 1H), 6.77 (d, J=8.2 Hz, 2H), 4.91 (brs, 1H), 4.21 (d, J=5.8 Hz, 2H), 1.46 (s, 9H).

### Step 3: Preparation of ethyl [4-(t-butoxycarbonylamino-methyl)-phenoxy]-acetate

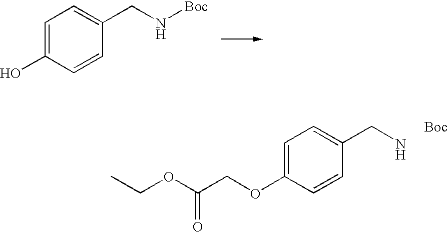

t-Butyl (4-hydroxybenzyl)-carbamate (223 mg, 1 mmol) obtained in step 2 above and bromo-acetic acid ethyl ester (0.11 ml, 1 mmol) are dissolved in acetone (3 ml). Thereto, K₂CO₃ (414 mg, 3 mmol) is added. The mixture is refluxed for 4 hours. The resultant is separated by column chromatography (EtOAc:hexane=1:5) to obtain the compound, ethyl [4-(t-butoxycarbonylamino-methyl)-phenoxy]-acetate (239 mg, 77%).

¹H NMR (CDCl₃, 300 MHz) δ 7.21 (d, J=8.7 Hz, 2H), 6.86 (d, J=8.7 Hz, 2H), 4.80 (brs, 1H), 4.60 (s, 2H), 4.26 (q, J=7.2 Hz, 2H), 4.23 (s, 2H), 1.45 (s, 9H), 1.30 (t, J=7.2 Hz, 3H).

### Step 4: Preparation of ethyl (4-aminomethyl-phenoxy)-acetate.HCl

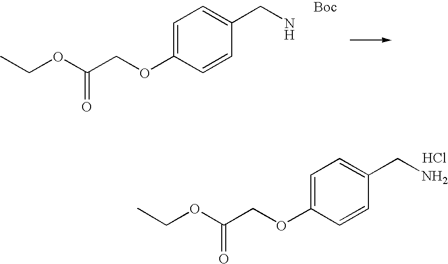

Ethyl [4-(t-butoxycarbonylamino-methyl)-phenoxy]-acetate (210 mg, 0.68 mmol) obtained in step 3 above is dissolved in EtOAc (3 ml). Thereto, a 4 M-HCl/1,4-dioxane mixture (1.7 ml) is added, followed by stirring for 16 hours at room temperature. The resulting mixture is concentrated under a reduced pressure to remove EtOAc and recrystallized with Et₂O to obtain the compound, ethyl (4-aminomethyl-phenoxy)-acetate.HCl (166 mg, 99%) as a white solid.

¹H NMR (DMSO-d₆, 300 MHz) δ 8.38 (brs, 3H), 7.42 (d, J=8.4 Hz, 2H), 6.96 (d, J=108.4 Hz, 2H), 4.79 (s, 2H), 4.16 (q, J=7.2 Hz, 2H), 3.93 (s, 2H), 1.21 (t, J=7.2 Hz, 3H); EI-MS m/z (relative intensive) 209 (M+, 23), 122 (100), 106 (72), 89 (38).

### Step 5: Preparation of ethyl 2-(4-((3-((R)-3-(tert-butoxycarbonylamino)-4-(2,4,5-trifluorophenyl)butanoyl)thiazolidine-2-carboxamido)methyl)-phenoxy)acetate

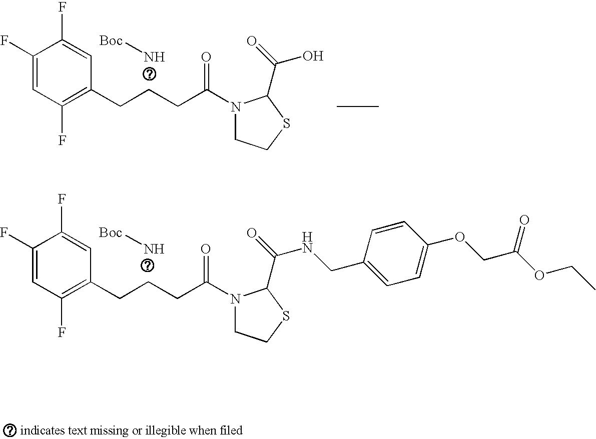

3-[(R)-3-t-butoxycarbonylamino-4-(2,4,5-trifluorophenyl)-butyryl]-thiazolidine-2-carboxylic acid (90 mg, 0.20 mmol) is dissolved in CH₂Cl₂ (2 ml). Thereto, ethyl (4-aminomethyl-phenoxy)-acetate.HCl (49 mg, 0.20 mmol) obtained in step 4 above, EDCI (77 mg, 0.40 mmol) and Et₃N (98 μl, 0.70 mmol) are added, followed by stirring for 12 hours at room temperature. The resulting mixture is extracted with CH₂Cl₂. The entire extracts are washed with brine and dried over MgSO₄. The resulting organic layer is concentrated under a reduced pressure and purified by column chromatography (EtOAc:hexane=1:1) to obtain the compound, ethyl (R)-{4-[({3-[3-t-butoxycarbonylamino-4-(2,4,5-trifluorophenyl)-butyryl]-thiazolidine-2-carbonyl}-amino)-methyl]-phenoxy}-acetate (34 mg, 27%).

¹H NMR (CDCl₃, 300 MHz) δ 7.72 (d, J=8.4 Hz, 2H), 7.20-7.00 (m, 1H), 7.00-6.87 (m, 1H), 6.86 (d, J=8.4 Hz, 2H), 6.20-6.10 (br, 1H), 5.51 (d, J=4.2 Hz, 1H), 4.60 (s, 2H), 4.38 (s, 2H), 4.27 (q, J=7.2 Hz, 2H), 4.13-4.11 (m, 1H), 4.00-3.80 (m, 1H), 3.75-3.73 (m, 1H), 3.60-3.40 (m, 1H), 3.15-3.00 (m, 1H), 2.95-2.80 (m, 2H), 2.64-3.63 (m, 2H), 1.32 (s, 9H), 1.28 (t, J=7.2 Hz, 3H).

### Step 6: Preparation of ethyl 2-(4-((3-((R)-3-amino-4-(2,4,5-trifluorophenyl)butanoyl)thiazolidine-2-carboxamido)methyl)phenoxy)acetate.HCl

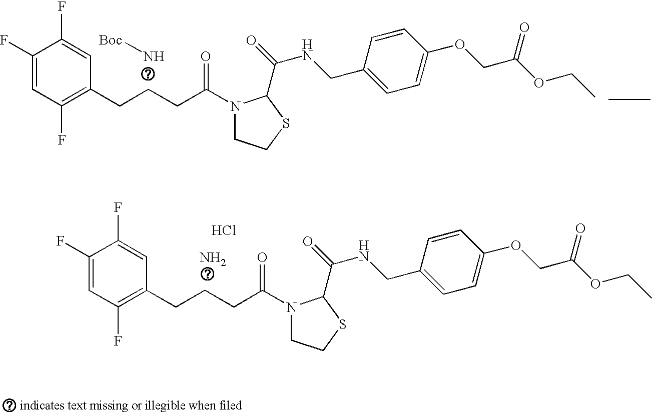

Ethyl 2-(4-((3-((R)-3-amino-4-(2,4,5-trifluorophenyl) butanoyl)thiazolidine-2-carboxamido)methyl)phenoxy)acetate.HCl is obtained according to the procedure used for Step 2, Example 1 (100%).

¹H NMR (CD₃OD, 300 MHz) δ 7.23-7.17 (m, 1H), 7.12-7.03 (m, 3H), 6.73-6.68 (m, 2H), 5.30 (d, J=13.3 Hz, 1H), 4.73-4.57 (m, 1H), 4.50 (s, 2H), 4.10 (s, 2H), 4.06 (q, J=7.2 Hz, 2H), 3.90-3.80 (m, 1H), 3.69-3.64 (m, 2H), 3.15-3.13 (m, 2H), 3.02-3.00 (m, 1H), 3.00-2.89 (m, 1H), 2.80-2.70 (m, 1H), 1.11 (t, J=7.2 Hz, 3H); LC-MS m/e 540 (MH⁺).

### Example 5

### Preparation of 2-(4-((3-((R)-3-amino-4-(2,4,5-trifluorophenyl) butanoyl)thiazolidine-2-carboxamido)methyl)phenoxy)acetic acid.HCl

### Step 1: Preparation of 2-(4-((3-((R)-3-(tert-butoxycarbonylamino)-4-(2,4,5-trifluorophenyl)butanoyl)thiazolidine-2-carboxamido)methyl)-phenoxy)acetic acid

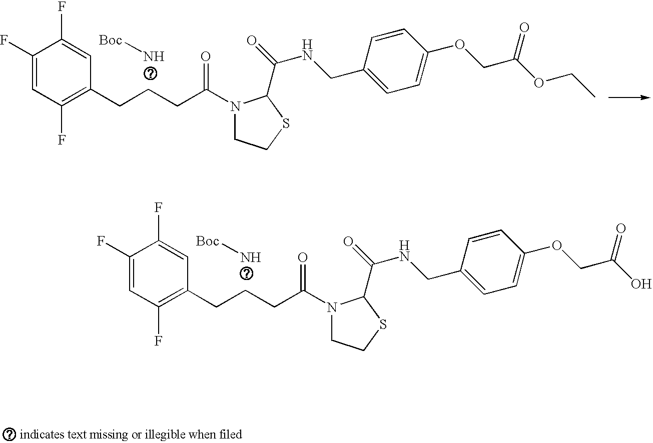

2-(4-((3-((R)-3-(tert-butoxycarbonylamino)-4-(2,4,5-trifluorophenyl)butanoyl)thiazolidine-2-carboxamido)methyl)-phenoxy)acetic acid is obtained according to the procedure used for Step 1, Example 2 (98%).

¹H NMR (CD₃OD, 300 MHz) δ 7.18 (d, J=8.4 Hz, 2H), 7.17-6.99 (m, 2H), 6.83 (d, J=8.4 Hz, 2H), 5.40 (d, J=6.0 Hz, 1H), 4.56 (s, 2H), 4.27 (s, 2H), 4.15-4.10 (m, 1H), 4.00-3.95 (m, 1H), 3.89-3.84 (m, 1H), 3.34-3.25 (m, 1H), 3.15-3.10 (m, 1H), 2.89-2.85 (m, 1H), 2.72-2.58 (m, 3H), 1.29 (s, 9H); LC-MS m/e 612 (MH⁺).

### Step 2: Preparation of 2-4-((3-((R)-3-amino-4-(2,4,5-trifluorophenyl) butanoyl)thiazolidine-2-carboxamido)methyl)phenoxy)acetic acid.HCl

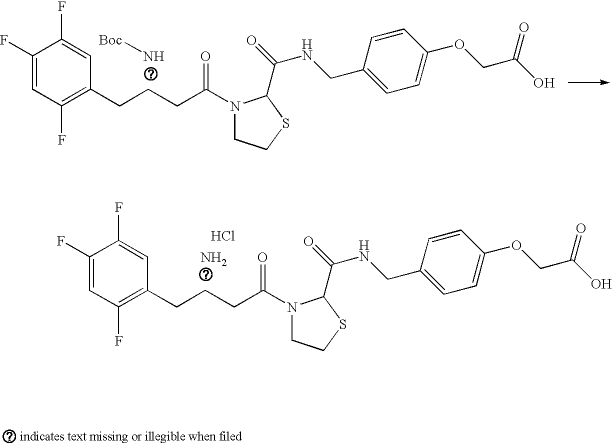

2-4-((3-((R)-3-amino-4-(2,4,5-trifluorophenyl)butanoyl) thiazolidine-2-carboxamido)methyl)phenoxy)acetic acid.HCl is obtained according to the procedure used for Step 2, Example 1 (81%).

¹H NMR (CD₃OD, 300 MHz) δ 7.40-7.20 (m, 1H), 7.18-7.13 (m, 3H), 6.83-6.80 (m, 2H), 5.40 (d, J=13.4 Hz, 1H), 4.56 (s, 2H), 4.24 (s, 2H), 4.00-3.80 (m, 1H), 3.80-3.70 (m, 2H), 3.25-3.23 (m, 1H), 3.20-3.05 (m, 1H), 2.99-2.97 (m, 2H), 2.80-2.60 (m, 1H); LC-MS m/e 511 (MH⁺).

### Example 6

### Preparation of ethyl 2-(4-(3-((R)-3-amino-4-(2,4,5-trifluorophenyl)butanoyl)thiazolidine-2-carboxamido)phenoxy)acetate.HCl

### Step 1: Preparation of t-butyl (4-hydroxyphenyl)-carbamate

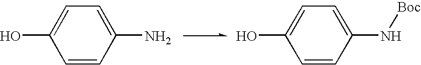

4-aminophenol (500 mg, 4.58 mmol) is dissolved in THF (15 ml). Thereto, Boc₂O (890 mg, 4.12 mmol) is added at 0° C., followed by stirring for 30 minutes at room temperature. The resulting mixture is concentrated under a reduced pressure and separated by column chromatography (EtOAc:hexane=1:2) to obtain the compound, t-butyl (4-hydroxyphenyl)-carbamate (710 mg, 82%) as a pink solid.

¹H NMR (CDCl₃, 300 MHz) δ 7.16 (d, J=8.7 Hz, 2H), 6.73 (d, J=8.7 Hz, 2H), 6.35 (brs, 1H), 5.43 (brs, 1H), 1.51 (s, 9H).

### Step 2: Preparation of ethyl [4-(t-butoxycarbonylamino)-phenoxy]-acetate

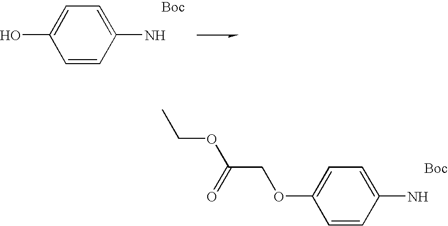

t-Butyl (4-hydroxyphenyl)-carbamate (300 mg, 1.43 mmol) obtained in step 1 above and ethyl bromoacetate (316 μl, 2.86 mmol) are dissolved in acetone (5 ml). Thereto, K₂CO₃ (593 mg, 4.29 mmol) is added. The mixture is refluxed for 4 hours, and separated by column chromatography (EtOAc:hexane=1:9) to obtain the compound, ethyl [4-(t-butoxycarbonylamino)-phenoxy]-acetate (422 mg, 99%).

¹H NMR (CDCl₃, 300 MHz) δ 7.27 (d, J=8.7 Hz, 2H), 6.85 (d, J=8.7 Hz, 2H), 6.38 (brs, 1H), 4.58 (s, 2H), 4.26 (q, J=7.2 Hz, 2H), 1.50 (s, 9H), 1.27 (t, J=7.2 Hz, 3H).

### Step 3: Preparation of ethyl (4-aminophenoxy)-acetate.HCl

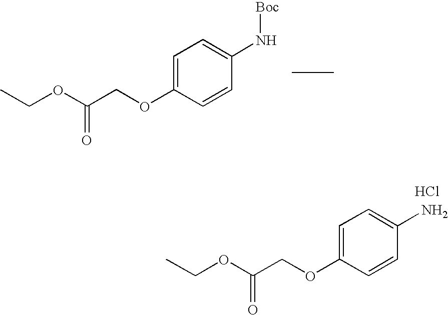

Ethyl (4-aminophenoxy)-acetate.HCl is obtained according to the procedure used for Step 2, Example 1 (82%) as a white solid.

¹H NMR (DMSO-d₆, 200 MHz) δ 10.23 (brs, 3H), 7.31 (d, J=8.8 Hz, 2H), 7.03 (d, J=8.8 Hz, 2H), 4.80 (s, 2H), 4.16 (q, J=7.2 Hz, 2H), 1.20 (t, J=7.2 Hz, 3H); LC-MS m/e 195 (MH⁺).

### Step 4: Preparation of ethyl 2-(4-(3-((R)-3-(tert-butoxycarbonylamino)-4-(2,4,5-trifluorophenyl)butanoyl)thiazolidine-2-carboxamido)phenoxy)acetate

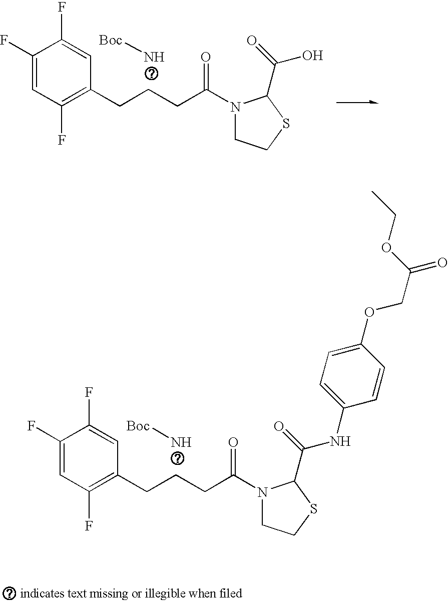

3-[(R)-3-t-butoxycarbonylamino-4-(2,4,5-trifluorophenyl)-butyryl]-thiazolidine-2-carboxylic acid (120 mg, 0.27 mmol) is dissolved in CH₂Cl₂ (2 ml). Thereto, ethyl (4-aminophenoxy)acetate.HCl (124 mg, 0.54 mmol) obtained in step 3 above, EDCI (154 mg, 0.80 mmol) and Et₃N (224 μl, 1.61 mmol) are added, followed by stirring for 12 hours at room temperature. The resulting mixture is extracted with CH₂Cl₂. The entire extracts are washed with brine and dried over MgSO₄. The resulting organic layer is concentrated under a reduced pressure and purified by column chromatography (EtOAc:hexane=1:1) to obtain the compound, ethyl 2-(4-(3-((R)-3-(tert-butoxycarbonylamino)-4-(2,4,5-trifluorophenyl)butanoyl)thiazolidine-2-carboxamido)phenoxy)acetate (76 mg, 45%).

¹H NMR (CDCl₃, 300 MHz) δ 7.43 (d, J=8.7 Hz, 2H), 7.15-7.05 (m, 1H), 6.90-6.84 (m, 1H), 6.85 (d, J=8.7 Hz, 2H), 5.71 (s, 1H), 5.48-5.45 (br, 1H), 4.58 (s, 2H), 4.26 (q, J=7.2 Hz, 2H), 4.15-4.09 (m, 1H), 3.94-3.91 (m, 1H), 3.83-3.78 (m, 1H), 3.52-3.49 (m, 1H), 3.15-3.11 (m, 1H), 2.97-2.93 (m, 2H), 2.70-2.50 (m, 2H), 1.36 (s, 9H), 1.29 (t, J=7.2 Hz, 3H); LC-MS m/e 625 (MH⁺).

### Step 5: Preparation of ethyl 2-(4-(3-((R)-3-amino-4-(2,4,5-trifluorophenyl)butanoyl)thiazolidine-2-carboxamido)phenoxy)acetate.HCl

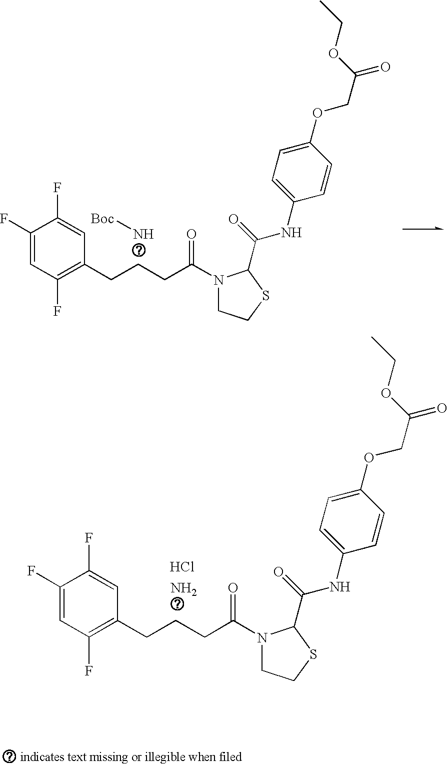

Ethyl 2-(4-(3-((R)-3-amino-4-(2,4,5-trifluorophenyl) butanoyl)thiazolidine-2-carboxamido)phenoxy)acetate.HCl is obtained according to the procedure used for Step 2, Example 1 (92%).

¹H NMR (CD₃OD, 300 MHz) δ 7.36 (d, J=9.0 Hz, 2H), 7.34-7.29 (m, 1H), 7.16-7.13 (m, 1H), 6.81 (d, J=9.0 Hz, 2H), 5.48 (d, J=14.0 Hz, 1H), 4.60 (s, 2H), 4.14 (q, J=7.2 Hz, 2H), 4.00-3.80 (m, 1H), 3.77-3.73 (m, 2H), 3.38-3.28 (m, 1H), 3.21-3.13 (m, 2H), 2.98-2.97 (m, 2H), 2.80-2.76 (m, 1H), 1.18 (t, J=7.2 Hz, 3H).

### Example 7

### Preparation of 2-(4-(3-((R)-3-amino-4-(2,4,5-trifluorophenyl) butanoyl)thiazolidine-2-carboxamido)phenoxy)acetic acid.HCl

### Step 1: Preparation of 2-(4-(3-((R)-3-(tert-butoxycarbonylamino)-4-(2,4,5-trifluorophenyl)butanoyl)thiazolidine-2-carboxamido)phenoxy)acetic acid

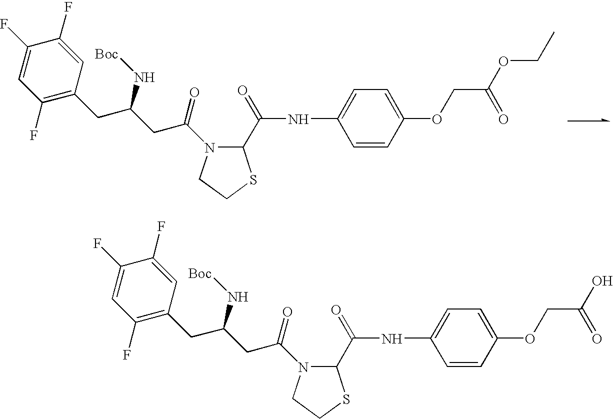

2-(4-(3-((R)-3-(tert-butoxycarbonylamino)-4-(2,4,5-trifluorophenyl)butanoyl)thiazolidine-2-carboxamido)phenoxy)acetic acid is obtained according to the procedure used for Step 1, Example 2 (72%).

¹H NMR (CD₃OD, 300 MHz) δ 7.48 (d, J=9.0 Hz, 2H), 7.16-7.13 (m, 1H), 6.96-6.89 (m, 1H), 6.88 (d, J=9.0 Hz, 2H), 5.61 (s, 1H), 4.58 (s, 2H), 3.80-3.79 (m, 2H), 3.60-3.40 (m, 1H), 3.15-3.12 (m, 2H), 3.00-2.90 (m, 2H), 2.69-2.64 (m, 2H), 1.36 (s, 9H).

### Step 2: Preparation of 2-(4-(3-((R)-3-amino-4-(2,4,5-trifluorophenyl) butanoyl)thiazolidine-2-carboxamido)phenoxy)acetic acid.HCl

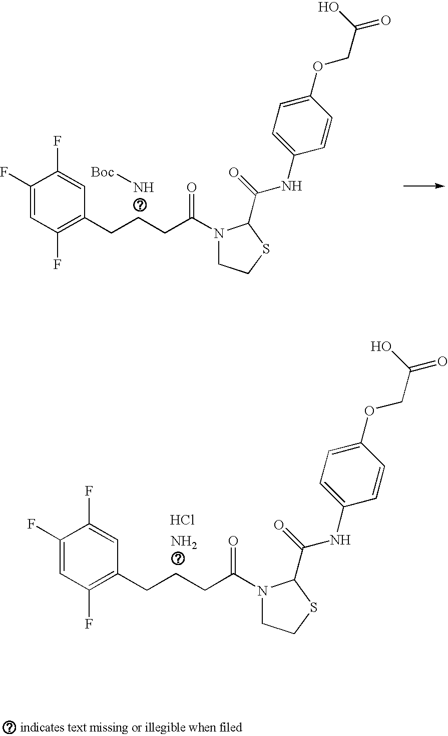

2-(4-(3-((R)-3-amino-4-(2,4,5-trifluorophenyl)butanoyl)thiazolidine-2-carboxamido)phenoxy)acetic acid hydrochloride is obtained according to the procedure used for Step 2, Example 1 (90%).

¹H NMR (DMSO-d₆, 300 MHz) δ 8.10 (brs, 3H), 7.56-7.51 (m, 2H), 7.46 (d, J=7.8 Hz, 2H), 6.88 (d, J=7.8 Hz, 2H), 5.52 (d, J=12.0 Hz, 1H), 4.72 (s, 2H), 4.01-3.69 (m, 4H), 2.98-2.64 (m, 5H).

### Example 8

### Preparation of ethyl 2-(4-((3-((R)-3-amino-4-(2,4,5-trifluorophenyl)butanoyl)thiazolidine-2-carboxamido)methyl)phenoxy)-3-methylbutanoate.HCl

### Step 1: Preparation of ethyl 2-(4-((3-((R)-3-(t-butoxycarbonylamino)-4-(2,4,5-trifluorophenyl)butanoyl)thiazolidine-2-carboxamido)methyl)phenoxy)-3-methylbutanoate

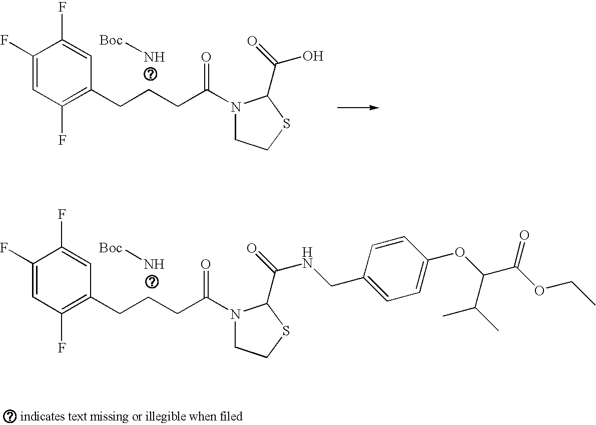

3-((R)-3-(tert-butoxycarbonylamino)-4-(2,4,5-trifluorophenyl)butanoyl)thiazolidine-2-carboxylic acid (1.77 g, 3.95 mmol) is dissolved in CH₂Cl₂. Thereto, EDCI (1.51 g, 7.89 mmol), ethyl 2-(4-aminomethyl-phenoxy)-3-methyl-butyrate.HCl (5.92 g, 1.49 mmol) and triethylamine (2.75 ml, 19.734 mmol) are added, followed by stirring for 12 hours at room temperature. The resulting mixture is washed with brine and extracted with CH₂Cl₂. The entire extracts are dried over MgSO₄. The resulting organic layer is concentrated under a reduced pressure and purified by column chromatography (EtOAc:hexane=1:1) to obtain the desired compound, ethyl 2-(4-((3-((R)-3-(t-butoxycarbonylamino)-4-(2,4,5-trifluorophenyl)butanoyl)thiazolidine-2-carboxamido)methyl)phenoxy)-3-methylbutanoate (2.03 g, 82%) as a white solid.

¹H NMR (CDCl₃, 300 MHz) δ 7.20-7.07 (m, 3H), 6.92-6.82 (m, 3H), 6.15 (br, 1H), 5.51 (br, 2H), 4.37-4.30 (m, 3H), 4.24-4.17 (m, 3H), 3.95-3.85 (m, 1H), 3.80-3.70 (m, 1H), 3.50-3.40 (m, 1H), 3.10-3.00 (m, 1H), 2.91-2.80 (m, 2H), 2.70-2.62 (m, 2H), 2.30-2.26 (m, 1H), 1.37 (s, 9H), 1.28-1.23 (m, 3H), 1.09-1.04 (m, 6H).

### Step 2: Preparation of ethyl 2-(4-((3-((R)-3-amino-4-(2,4,5-trifluorophenyl)butanoyl)thiazolidine-2-carboxamido)methyl)phenoxy)-3-methylbutanoate.HCl

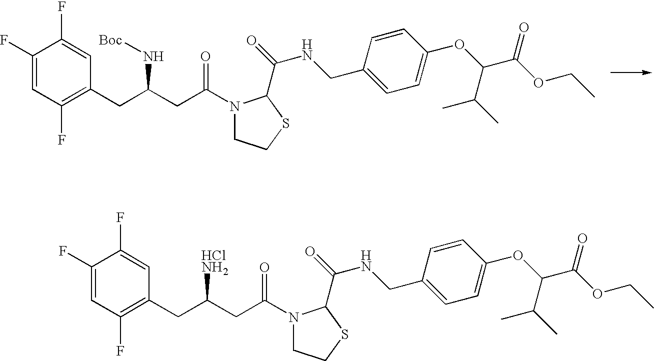

Ethyl 2-(4-((3-((R)-3-amino-4-(2,4,5-trifluorophenyl)butanoyl)thiazolidine-2-carboxamido)methyl)phenoxy)-3-methylbutanoate.HCl is obtained according to the procedure used for Step 2, Example 1 (99%) as a white solid.

¹H NMR (DMSO-d₆, 300 MHz) δ 8.59-8.51 (m, 1H), 8.21 (brs, 3H), 7.63-7.50 (m, 2H), 7.17-7.13 (m, 2H), 6.87-6.78 (m, 2H), 5.47-5.35 (m, 2H), 4.54-4.50 (m, 1H), 4.21-4.10 (m, 4H), 4.00-3.71 (m, 3H), 3.23-2.76 (m, 5H), 2.30-2.00 (m, 1H), 1.17 (t, J=7.1 Hz, 3H), 1.00-0.98 (m, 6H).

### Example 9

### Preparation of 2-(4-((3-((R)-3-amino-4-(2,4,5-trifluorophenyl) butanoyl)thiazolidine-2-carboxamido)methyl)phenoxy)-3-methylbutanoic acid.HCl

### Step 1: Preparation of 2-(4-((3-((R)-3-(tert-butoxycarbonylamino)-4-(2,4,5-trifluorophenyl)butanoyl)thiazolidine-2-carboxamido)methyl)phenoxy)-3-methylbutanoic acid

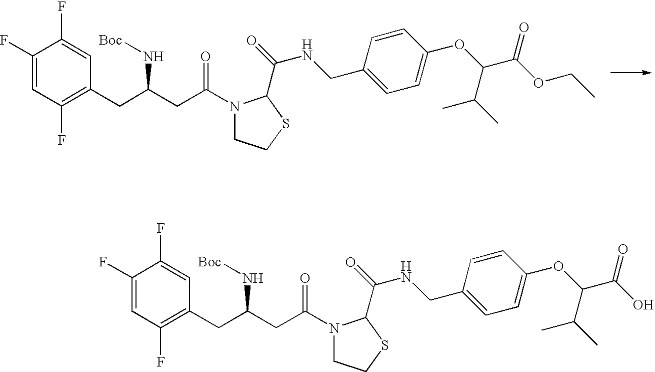

2-(4-((3-((R)-3-(tert-butoxycarbonylamino)-4-(2,4,5-trifluorophenyl)butanoyl)thiazolidine-2-carboxamido)methyl)phenoxy)-3-methylbutanoic acid is obtained according to the procedure used for Step 1, Example 2 (98%) as a white solid.

¹H NMR (CDCl₃, 300 MHz) δ 7.15-7.13 (m, 3H), 6.92-6.82 (m, 3H), 6.58 (br, 1H), 5.50 (br, 2H), 4.39-4.32 (m, 3H), 4.13-4.05 (m, 1H), 3.89-3.68 (m, 4H), 3.50-3.40 (m, 1H), 3.10-2.92 (m, 1H), 2.89-2.87 (m, 1H), 2.60-2.46 (m, 1H), 2.40-2.20 (m, 1H), 1.99-1.87 (m, 1H), 1.36 (s, 9H), 1.11-1.08 (m, 6H).

### Step 2: Preparation of 2-(4-((3-((R)-3-amino-4-(2,4,5-trifluorophenyl) butanoyl)thiazolidine-2-carboxamido)methyl)phenoxy)-3-methylbutanoic acid.HCl

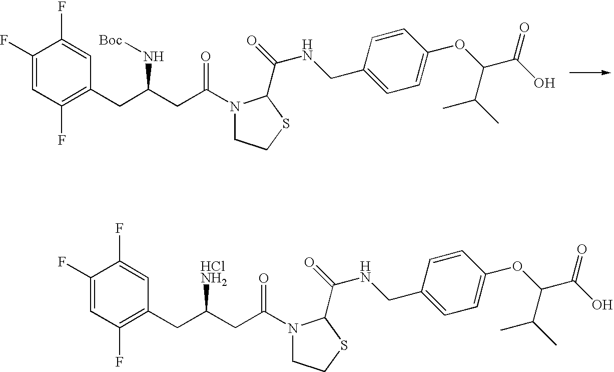

2-(4-((3-((R)-3-amino-4-(2,4,5-trifluorophenyl)butanoyl)thiazolidine-2-carboxamido)methyl)phenoxy)-3-methylbutanoic acid.HCl is obtained according to the procedure used for Step 2, Example 1 (86%) as a white solid.

¹H NMR (DMSO-d₆, 300 MHz) δ 12.91 (br, 1H), 8.59 (br, 1H), 7.98 (brs, 3H), 7.53-7.50 (m, 2H), 7.13-7.11 (m, 2H), 6.80-6.75 (m, 2H), 5.37-5.33 (m, 1H), 4.40-4.38 (m, 1H), 4.20-4.12 (m, 3H), 3.83-3.68 (m, 3H), 2.92-2.85 (m, 2H), 2.69-2.60 (m, 1H), 2.24-2.14 (m, 1H), 0.97 (d, J=6.6 Hz, 6H).

### Example 10

### Preparation of pivaloyloxymethyl 2-(4-((3-((R)-3-amino-4-(2,4,5-trifluorophenyl)butanoyl)thiazolidine-2-carboxamido)methyl)phenoxy)-3-methylbutanoate.HCl

### Step 1: Preparation of pivaloyloxymethyl 2-(4-((3-((R)-3-(tert-butoxycarbonylamino)-4-(2,4,5-trifluorophenyl)butanoyl)thiazolidine-2-carboxamido)methyl)phenoxy)-3-methylbutanoate

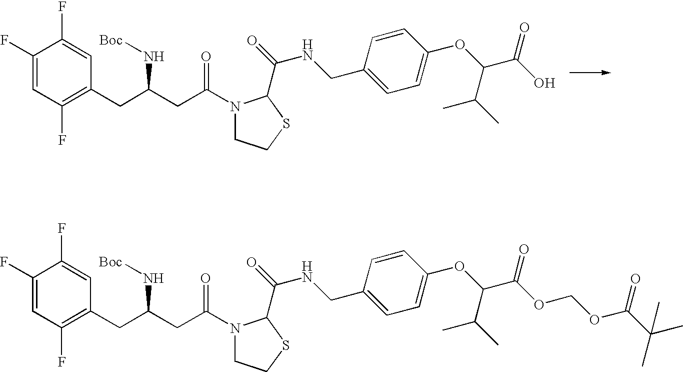

2-(4-((3-((R)-3-(tert-butoxycarbonylamino)-4-(2,4,5-trifluorophenyl)butanoyl)thiazolidine-2-carboxamido)methyl)phenoxy)-3-methylbutanoic acid (200 mg, 0.31 mmol) obtained in step 1 of Example 9 is dissolved in DMA. Thereto, K₂CO₃ (127 mg, 0.92 mmol) and iodomethylpivalate (89 mg, 0.37 mmol) are added, followed by stirring for 3 hours at room temperature. The resulting mixture is washed with brine and extracted with EtOAc. The entire extracts are dried over MgSO₄. The resulting organic layer is concentrated under a reduced pressure and purified by column chromatography (EtOAc:hexane=1:1) to obtain the compound, pivaloyloxymethyl 2-(4-((3-((R)-3-(tert-butoxycarbonylamino)-4-(2,4,5-trifluorophenyl)butanoyl)thiazolidine-2-carboxamido)methyl)phenoxy)-3-methylbutanoate (180 mg, 77%) as a white solid.

¹H NMR (CDCl₃, 300 MHz) δ 7.20-7.09 (m, 3H), 6.91-6.81 (m, 3H), 6.20 (br, 1H), 5.81 (d J=4.2 Hz, 1H), 5.78 (d J=4.2 Hz, 1H), 5.60-5.51 (m, 2H), 4.40-4.37 (m, 3H), 4.20-4.11 (m, 2H), 4.00-3.80 (m, 1H), 3.77-3.75 (m, 1H), 3.50-3.40 (m, 1H), 3.11-2.91 (m, 2H), 2.70-2.62 (m, 2H), 2.29-2.27 (m, 1H), 1.38 (s, 9H), 1.18 (s, 9H), 1.08-1.06 (m, 6H).

### Step 2: Preparation of pivaloyloxymethyl 2-(4-((3-((R)-3-amino-4-(2,4,5-trifluorophenyl)butanoyl)thiazolidine-2-carboxamido)methyl)phenoxy)-3-methylbutanoate.HCl

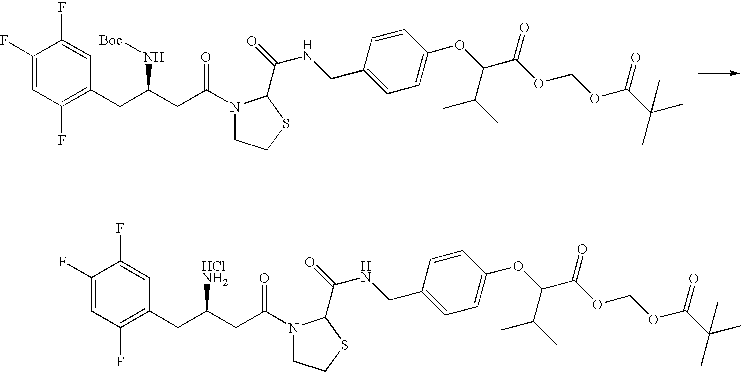

Pivaloyloxymethyl 2-(4-((3-((R)-3-amino-4-(2,4,5-trifluorophenyl)butanoyl)thiazolidine-2-carboxamido)methyl)phenoxy)-3-methylbutanoate.HCl is obtained according to the procedure used for Step 2, Example 1 (100%) as a white solid.

¹H NMR (DMSO-d₆, 300 MHz) δ 8.55-8.49 (m, 1H), 8.13 (brs, 3H), 7.59-7.53 (m, 3H), 7.16-7.12 (m, 3H), 5.81 (d J=5.8 Hz, 1H), 5.73 (d J=5.8 Hz, 1H), 5.40-5.36 (m, 1H), 4.72-4.63 (m, 2H), 4.19-4.15 (m, 3H), 4.00-3.71 (m, 3H), 3.20-3.17 (m, 2H), 3.00-2.93 (m, 1H), 2.79-2.76 (m, 1H), 2.30-2.17 (m, 1H), 1.12 (s, 9H), 1.00-0.98 (m, 6H); LC-MS m/z (relative intensity) 669 (MH⁺).

### Example 11

### Preparation of ethyl 1-(3-((R)-3-amino-4-(2,4,5-trifluorophenyl)butanoyl)thiazolidine-2-carbonyl)piperidine-4-carboxylate.HCl

### Step 1: Preparation of ethyl 1-(3-((R)-3-(tert-butoxycarbonylamino)-4-(2,4,5-trifluorophenyl)butanoyl)thiazolidine-2-carbonyl)piperidine-4-carboxylate

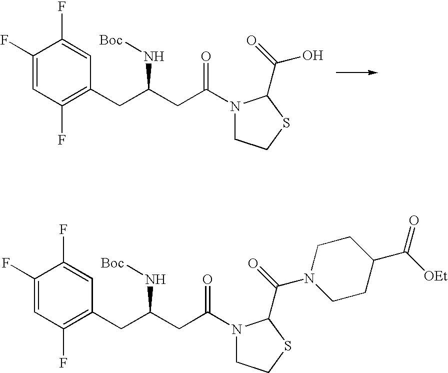

3-((R)-3-(tert-butoxycarbonylamino)-4-(2,4,5-trifluorophenyl)butanoyl)thiazolidine-2-carboxylic acid (150 mg, 0.34 mmol) is dissolved in CH₂Cl₂. Thereto, EDCI (128 mg, 0.67 mmol), DMAP (8 mg, 0.07 mmol) ethyl isonipecotate (62 μl, 0.40 mmol) and triethylamine (233 μl, 1.67 mmol) are added, followed by stirring for 12 hours at room temperature. The resulting mixture is washed with brine and extracted with CH₂Cl₂. The entire extracts are dried over MgSO₄. The resulting organic layer is concentrated under a reduced pressure and purified by column chromatography (MeOH:EtOAc:hexane=1:4:4) to obtain the compound, ethyl 1-(3-((R)-3-(tert-butoxycarbonylamino)-4-(2,4,5-trifluorophenyl)butanoyl)thiazolidine-2-carbonyl)piperidine-4-carboxylate (50 mg, 25%) as a white solid.

¹H NMR (CDCl₃, 300 MHz) δ 7.18-7.06 (m, 1H), 6.92-6.84 (m, 1H), 5.91 (br, 1H), 5.63-5.58 (m, 1H), 4.45-4.30 (m, 1H), 4.16 (q, J=7.2 Hz, 2H), 3.96-3.76 (m, 4H), 3.50-3.35 (m, 1H), 3.14-2.89 (m, 6H), 2.65-2.56 (m, 3H), 2.00-1.96 (m, 1H), 1.37 (s, 9H), 1.27 (t, J=7.2 Hz, 3H).

### Step 2: Preparation of ethyl 1-(3-((R)-3-amino-4-(2,4,5-trifluorophenyl)butanoyl)thiazolidine-2-carbonyl)piperidine-4-carboxylate.HCl

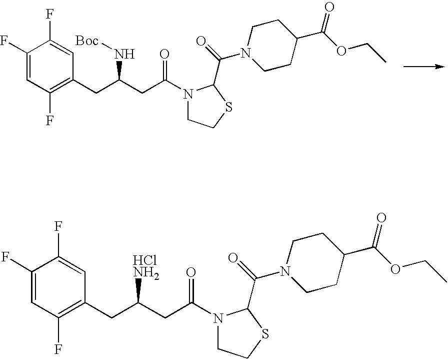

Ethyl 1-(3-((R)-3-amino-4-(2,4,5-trifluorophenyl)butanoyl)thiazolidine-2-carbonyl)piperidine-4-carboxylate.HCl was obtained according to the procedure used for Step 2, Example 1 (90%) as a white solid.

¹H NMR (DMSO-d₆, 300 MHz) δ 7.81 (brs, 3H), 7.46-7.37 (m, 2H), 6.37 (br, 1H), 4.26 (q, J=7.0 Hz, 2H), 3.89-3.30 (m, 4H), 3.05-2.58 (m, 13H), 1.23 (t, J=7.0 Hz, 3H).

### Example 12

### Preparation of 1-(3-((R)-3-amino-4-(2,4,5-trifluorophenyl) butanoyl)thiazolidine-2-carbonyl)piperidine-4-carboxylic acid.HCl

### Step 1: Preparation of 1-(3-((R)-3-(tert-butoxycarbonylamino)-4-(2,4,5-trifluorophenyl)butanoyl)thiazolidine-2-carbonyl)piperidine-4-carboxylic acid

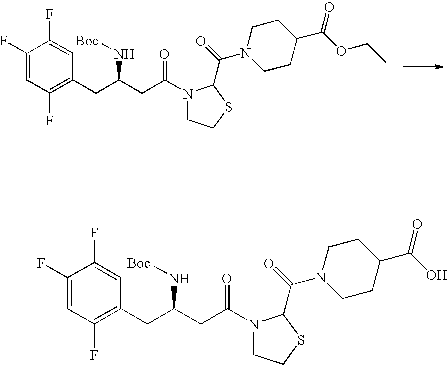

1-(3-((R)-3-(tert-butoxycarbonylamino)-4-(2,4,5-trifluorophenyl)butanoyl)thiazolidine-2-carbonyl)piperidine-4-carboxylic acid is obtained according to the procedure used for Step 1, Example 2 (97%) as a white solid.

¹H NMR (CDCl₃, 300 MHz) δ 7.23-7.19 (m, 1H), 6.93-6.84 (m, 1H), 5.92-5.90 (m, 1H), 4.11-3.71 (m, 10H), 3.20-3.00 (m, 2H), 2.80-2.70 (m, 2H), 2.10-1.88 (m, 4H), 1.36 (s, 9H).

### Step 2: Preparation of 1-(3-((R)-3-amino-4-(2,4,5-trifluorophenyl) butanoyl)thiazolidine-2-carbonyl)piperidine-4-carboxylic acid.HCl

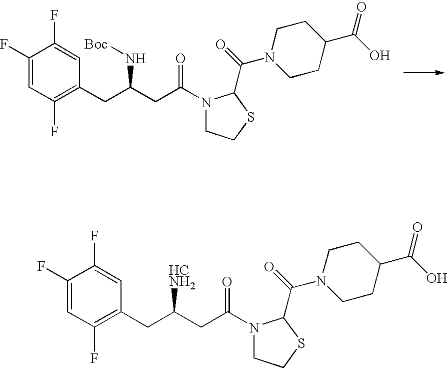

1-(3-((R)-3-amino-4-(2,4,5-trifluorophenyl)butanoyl)thiazolidine-2-carbonyl)piperidine-4-carboxylic acid.HCl is obtained according to the procedure used for Step 2, Example 1 (90%) as a white solid.

¹H NMR (DMSO-d₆, 300 MHz) δ 8.09 (brs, 3H), 7.69-7.60 (m, 2H), 6.03-6.00 (m, 1H), 4.20-4.15 (m, 1H), 3.94-3.79 (m, 2H), 3.41-3.30 (m, 4H), 3.29-2.82 (m, 8H), 2.11-1.99 (m, 1H), 1.80-1.30 (m, 1H).

### Example 13

### Preparation of 2-(4-((3-((R)-3-amino-4-(2,4,5-trifluorophenyl) butanoyl)thiazolidine-2-carboxamido)methyl)phenyl)acetic acid.HCl

### Step 1: Preparation of ethyl 2-(4-((3-((R)-3-(tert-butoxycarbonylamino)-4-(2,4,5-trifluorophenyl)butanoyl)thiazolidine-2-carboxamido)methyl)phenyl)acetate

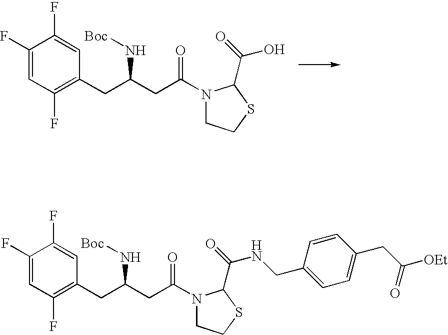

3-((R)-3-(tert-butoxycarbonylamino)-4-(2,4,5-trifluorophenyl)butanoyl)thiazolidine-2-carboxylic acid (150 mg, 0.34 mmol) is dissolved in CH₂Cl₂. Thereto, EDCI (128 mg, 0.67 mmol), DMAP (8 mg, 0.07 mmol), ethyl 4-aminomethyl-phenyl acetate.HCl (115 mg, 0.51 mmol) and triethylamine (233 μl, 1.67 mmol) are added, followed by stirring for 12 hours at room temperature. The resulting mixture is washed with brine and extracted with CH₂Cl₂. The entire extracts are dried over MgSO₄. The resulting organic layer is concentrated under a reduced pressure and purified by column chromatography (MeOH:EtOAc:hexane=1:4:4) to obtain the compound, ethyl 2-(4-((3-((R)-3-(tert-butoxycarbonylamino)-4-(2,4,5-trifluorophenyl)butanoyl)thiazolidine-2-carboxamido)methyl)phenyl)acetate (33 mg, 16%) as a white solid.

¹H NMR (CDCl₃, 300 MHz) δ 7.32-7.24 (m, 4H), 7.17-7.06 (m, 1H), 6.90-6.87 (m, 1H), 6.38-6.33 (m, 1H), 5.53-5.52 (m, 1H), 4.48-4.41 (m, 2H), 4.00-3.91 (m, 1H), 3.80-3.74 (m, 2H), 3.60-3.57 (m, 2H), 3.11-3.00 (m, 1H), 2.90-2.80 (m, 2H), 2.64-2.62 (m, 2H), 2.00-1.80 (m, 1H), 1.37-1.23 (m, 12H).

### Step 2: Preparation of 2-(4-((3-((R)-3-(tert-butoxycarbonylamino)-4-(2,4,5-trifluorophenyl)butanoyl)thiazolidine-2-carboxamido)methyl)phenyl)acetic acid

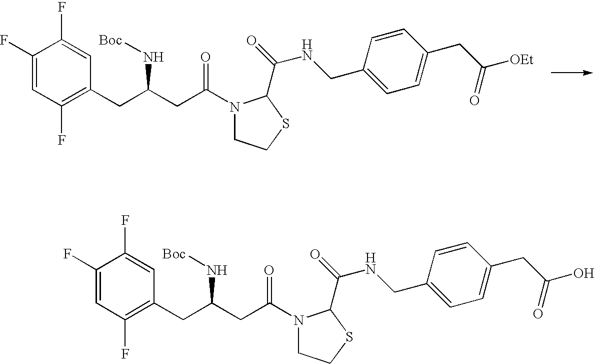

2-(4-((3-((R)-3-(tert-butoxycarbonylamino)-4-(2,4,5-trifluorophenyl)butanoyl)thiazolidine-2-carboxamido)methyl)phenyl)acetic acid is obtained according to the procedure used for Step 1, Example 2 (77%) as a white solid.

¹H NMR (CDCl₃, 300 MHz) δ 12.23 (br, 1H), 8.53-8.51 (m, 1H), 7.52-7.49 (m, 2H), 7.35-7.27 (m, 1H), 6.84-6.79 (m, 2H), 5.55-5.45 (m, 1H), 4.32-4.30 (m, 2H), 4.12-3.87 (m, 6H), 3.58-3.57 (m, 2H), 3.00-2.80 (m, 2H), 2.70-2.65 (m, 1H), 2.00-1.60 (m, 1H), 1.34 (s, 9H).

### Step 3: Preparation of 2-(4-((3-((R)-3-amino-4-(2,4,5-trifluorophenyl) butanoyl)thiazolidine-2-carboxamido)methyl)phenyl)acetic acid.HCl

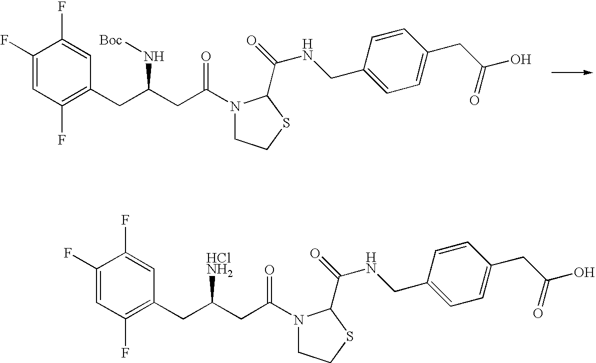

2-(4-((3-((R)-3-amino-4-(2,4,5-trifluorophenyl)butanoyl)thiazolidine-2-carboxamido)methyl)phenyl)acetic acid.HCl is obtained according to the procedure used for Step 2, Example 1 (92%) as a white solid.

¹H NMR (DMSO-d₆, 300 MHz) δ 8.54 (br, 1H), 8.01 (brs, 3H), 7.60-7.51 (m, 2H), 7.21-7.18 (m, 4H), 4.32-4.25 (m, 3H), 3.80-3.53 (m, 7H), 3.00-2.80 (m, 2H), 2.74-2.73 (m, 2H).

### Example 14

### Preparation of ethyl 2-(2-(3-((R)-3-amino-4-(2,4,5-trifluorophenyl)butanoyl)thiazolidine-2-carbonyl)-1,2,3,4-tetrahydroisoquinolin-7-yloxy)-3-methylbutanoate.HCl

### Step 1: Preparation ethyl 2-(2-(3-((R)-3-(tert-butoxycarbonylamino)-4-(2,4,5-trifluorophenyl)butanoyl)thiazolidine-2-carbonyl)-1,2,3,4-tetrahydroisoquinolin-7-yloxy)-3-methylbutanoate

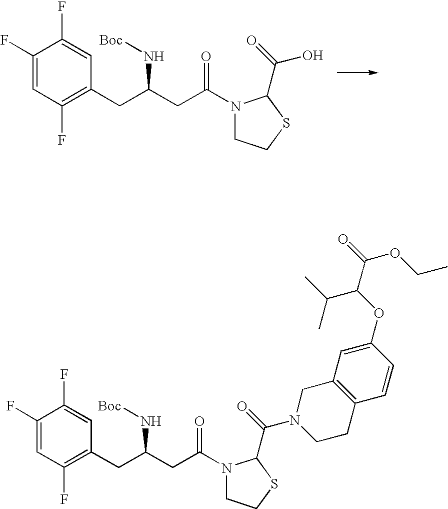

3-((R)-3-(tert-butoxycarbonylamino)-4-(2,4,5-trifluorophenyl)butanoyl)thiazolidine-2-carboxylic acid (120 mg, 0.27 mmol) is dissolved in CH₂Cl₂. Thereto, EDCI (103 mg, 0.54 mmol), DMAP (3.3 mg, 0.03 mmol), 3-methyl-2-(1,2,3,4-tetrahydroisoquinolin-7-yloxy)-butyric acid ethyl ester.HCl (100 mg, 0.32 mmol) and triethylamine (186 μl, 1.34 mmol) are added, followed by stirring for 12 hours at room temperature. The resulting mixture is washed with brine and extracted with CH₂Cl₂. The entire extracts are dried over MgSO₄. The resulting organic layer is concentrated under a reduced pressure and purified by column chromatography (EtOAc:hexane=1:1) to obtain the compound, ethyl 2-(2-(3-((R)-3-(tert-butoxycarbonylamino)-4-(2,4,5-trifluorophenyl)butanoyl)thiazolidine-2-carbonyl)-1,2,3,4-tetrahydroisoquinolin-7-yloxy)-3-methylbutanoate (58 mg, 31%) as a white solid.

¹H NMR (CDCl₃, 300 MHz) δ 7.20-7.03 (m, 2H), 6.90-6.84 (m, 1H), 6.75-6.73 (m, 1H), 6.66 (s, 1H), 5.99-5.97 (m, 1H), 5.80-5.60 (m, 1H), 4.74-4.50 (m, 2H), 4.33-4.11 (m, 3H), 4.00-3.69 (m, 4H), 3.45-3.30 (m, 1H), 3.21-3.12 (m, 1H), 3.00-2.89 (m, 4H), 2.80-2.65 (m, 2H), 2.26-2.20 (m, 1H), 1.37 (s, 9H), 1.28 (t, J=7.2 Hz, 3H), 1.09-1.05 (m, 6H).

### Step 2: Preparation of ethyl 2-(2-(3-((R)-3-amino-4-(2,4,5-trifluorophenyl)butanoyl)thiazolidine-2-carbonyl)-1,2,3,4-tetrahydroisoquinolin-7-yloxy)-3-methylbutanoate.HCl

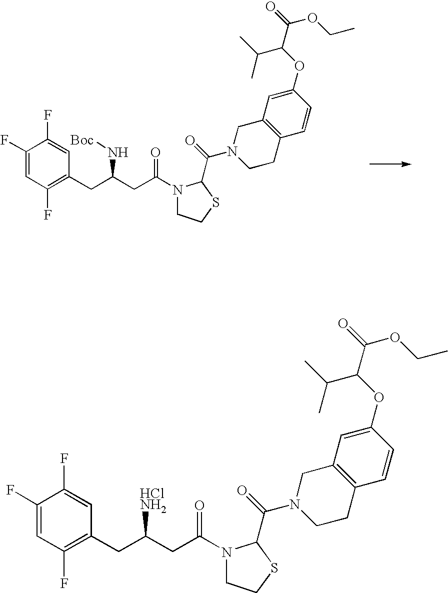

ethyl 2-(2-(3-((R)-3-amino-4-(2,4,5-trifluorophenyl)butanoyl)thiazolidine-2-carbonyl)-1,2,3,4-tetrahydroisoquinolin-7-yloxy)-3-methylbutanoate.HCl is obtained according to the procedure used for Step 2, Example 1 (92%) as a white solid.

¹H NMR (MeOH-d₄, 300 MHz) δ 7.41-7.19 (m, 2H), 7.05-7.02 (m, 1H), 6.72-6.63 (m, 2H), 6.00-5.96 (m, 1H), 4.87-4.41 (m 5H), 4.17-4.14 (m, 2H), 3.89-3.61 (m, 6H), 3.25-2.66 (m, 7H), 2.21-2.10 (m, 1H), 1.99 (t, J=7.2 Hz, 3H), 0.83-0.80 (m, 6H).

### Example 15

### Preparation of 2-(2-(3-((R)-3-amino-4-(2,4,5-trifluorophenyl) butanoyl)thiazolidine-2-carbonyl)-1,2,3,4-tetrahydroisoquinolin-7-yloxy)-3-methylbutanoic acid.HCl

### Step 1: Preparation of 2-(2-(3-((R)-3-(tert-butoxycarbonylamino)-4-(2,4,5-trifluorophenyl)butanoyl)thiazolidine-2-carbonyl)-1,2,3,4-tetrahydroisoquinolin-7-yloxy)-3-methylbutanoic acid

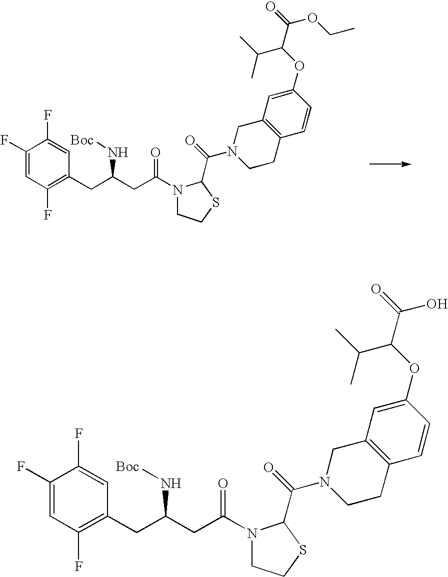

2-(2-(3-((R)-3-(tert-butoxycarbonylamino)-4-(2,4,5-trifluorophenyl)butanoyl)thiazolidine-2-carbonyl)-1,2,3,4-tetrahydroisoquinolin-7-yloxy)-3-methylbutanoic acid is obtained according to the procedure used for Step 1, Example 2 (97%) as a white solid.

¹H NMR (CDCl₃, 300 MHz) δ 7.07-7.05 (m, 2H), 6.89-6.86 (m, 1H), 6.79-6.76 (m, 1H), 6.69-6.60 (m, 1H), 5.99-5.97 (m, 1H), 4.80-4.60 (m, 2H), 4.41 (br, 1H), 3.91-3.67 (m, 5H), 3.60-3.50 (m, 2H), 3.20-3.00 (m, 2H), 2.99-2.80 (m, 2H), 2.70-2.50 (m, 2H), 1.96-1.88 (m, 2H), 1.70-1.60 (m, 1H), 1.36 (s, 9H), 1.12-1.09 (m, 6H).

### Step 2: Preparation of 2-(2-(3-((R)-3-amino-4-(2,4,5-trifluorophenyl) butanoyl)thiazolidine-2-carbonyl)-1,2,3,4-tetrahydroisoquinolin-7-yloxy)-3-methylbutanoic acid.HCl

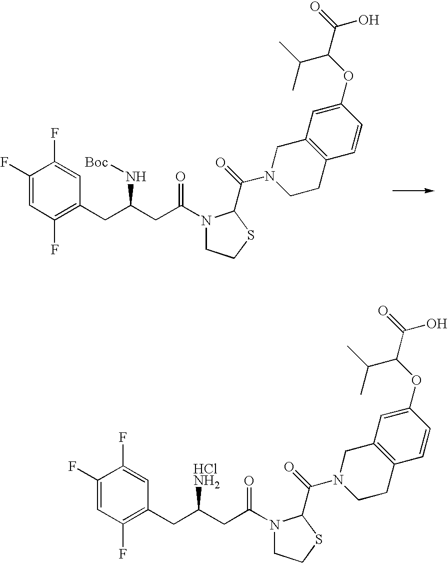

2-(2-(3-((R)-3-amino-4-(2,4,5-trifluorophenyl) butanoyl)thiazolidine-2-carbonyl)-1,2,3,4-tetrahydroisoquinolin-7-yloxy)-3-methylbutanoic acid.HCl is obtained according to the procedure used for Step 2, Example 1 (93%) as a white solid.

¹H NMR (DMSO-d₆, 300 MHz) δ 12.93 (br, 1H), 8.05 (brs, 3H), 7.61-7.54 (m, 2H), 7.10-7.08 (m, 1H), 6.73-6.71 (m, 2H), 6.18-5.99 (m, 1H), 4.53-4.45 (m, 4H), 3.86-3.57 (m, 6H), 3.20-2.74 (m, 6H), 2.20-2.00 (m, 1H), 1.07-0.99 (m, 6H); LC-MS m/z (relative intensity) 581 (MH⁺).

### Example 16

### Preparation of ethyl 6-((3-((R)-3-amino-4-(2,4,5-trifluorophenyl)butanoyl)thiazolidine-2-carboxamido)methyl)-2,3-dihydrobenzo[b][1,4]dioxin-2-carboxylate.HCl

### Step 1: Preparation of ethyl 6-((3-((R)-3-(tert-butoxycarbonylamino)-4-(2,4,5-trifluorophenyl)butanoyl)thiazolidine-2-carboxamido)methyl)-2,3-dihydrobenzo[b][1,4]dioxine-2-carboxylate

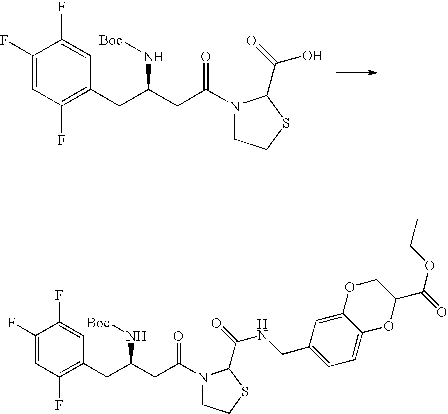

3-((R)-3-(tert-butoxycarbonylamino)-4-(2,4,5-trifluorophenyl)butanoyl)thiazolidine-2-carboxylic acid (120 mg, 0.27 mmol) is dissolved in CH₂Cl₂. Thereto, EDCI (103 mg, 0.54 mmol), ethyl 6-aminomethyl-2,3-dihydrobenzo[1,4]dioxin-2-carboxylate.HCl (88 mg, 0.32 mmol) and triethylamine (186 μl, 1.338 mmol) are added, followed by stirring for 12 hours at room temperature. The resulting mixture is washed with brine and extracted with CH₂Cl₂. The entire extracts are dried over MgSO₄. The resulting solution is concentrated under a reduced pressure and purified by column chromatography (MeOH:EtOAc:hexane=1:4:8) to obtain the compound, ethyl 6-((3-((R)-3-(tert-butoxycarbonylamino)-4-(2,4,5-trifluorophenyl)butanoyl)thiazolidine-2-carboxamido)methyl)-2,3-dihydrobenzo[b][1,4]dioxine-2-carboxylate (92 mg, 50%) as a white solid.

¹H NMR (CDCl₃, 300 MHz) δ 7.11-7.00 (m, 1H), 6.97-6.80 (m, 4H), 6.25 (br, 1H), 5.53-5.50 (m, 1H), 4.80-4.77 (m, 1H), 4.37-4.23 (m, 5H), 4.16-4.09 (m, 1H), 4.00-3.91 (m, 1H), 3.85-3.69 (m, 1H), 3.50-3.48 (m, 1H), 3.19-3.11 (m, 1H), 3.00-2.92 (m, 2H), 2.65-2.61 (m, 2H), 1.37 (s, 9H), 1.27 (t, J=7.2 Hz, 3H).

### Step 2: Preparation of ethyl 6-((3-((R)-3-amino-4-(2,4,5-trifluorophenyl)butanoyl)thiazolidine-2-carboxamido)methyl)-2,3-dihydrobenzo[b][1,4]dioxin-2-carboxylate.HCl

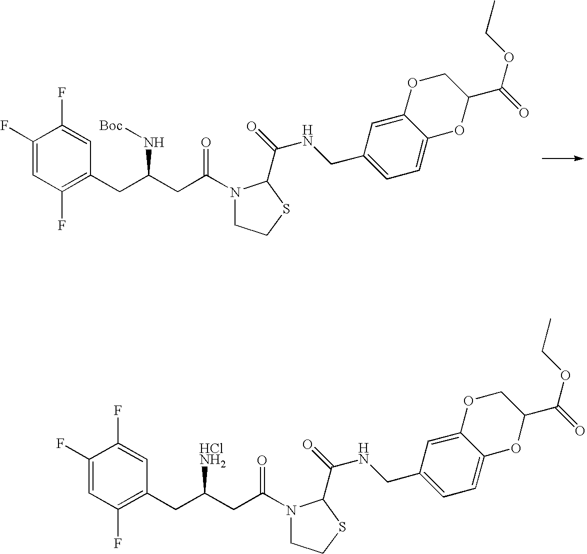

Ethyl 6-((3-((R)-3-amino-4-(2,4,5-trifluorophenyl) butanoyl)thiazolidine-2-carboxamido)methyl)-2,3-dihydrobenzo[b][1,4]dioxin-2-carboxylate.HCl is obtained according to the procedure used for Step 2, Example 1 (99%) as a white solid.

¹H NMR (MeOH-d₄, 300 MHz) δ 7.33-7.19 (m, 2H), 6.86-6.73 (m, 3H), 4.89-4.74 (m, 7H), 4.35-4.30 (m, 1H), 4.27-4.15 (m, 4H), 4.00-3.90 (m, 1H), 3.79-3.62 (m, 2H), 3.21-3.00 (m, 2H), 2.80-2.60 (m, 2H), 1.22 (t, J=7.1 Hz, 3H).

### Example 17

### Preparation of 6-((3-((R)-3-amino-4-(2,4,5-trifluorophenyl) butanoyl)thiazolidine-2-carboxamido)methyl)-2,3-dihydrobenzo[b][1,4]dioxin-2-carboxylic acid.HCl

### Step 1: Preparation of 6-((3-((R)-3-(tert-butoxycarbonylamino)-4-(2,4,5-trifluorophenyl)butanoyl)thiazolidine-2-carboxamido)methyl)-2,3-dihydrobenzo[b][1,4]dioxine-2-carboxylic acid

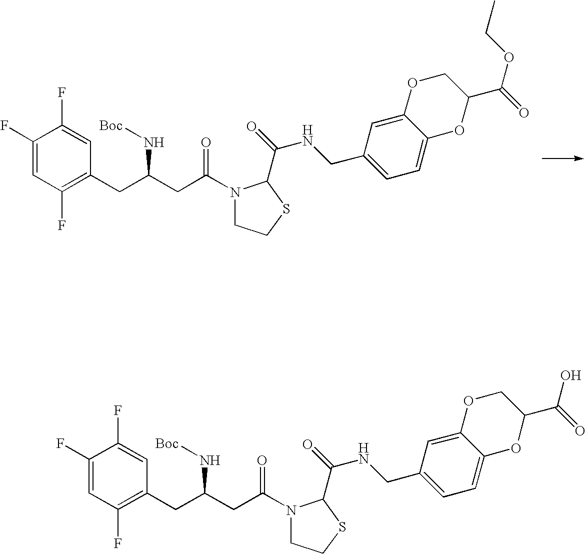

6-((3-((R)-3-(tert-butoxycarbonylamino)-4-(2,4,5-trifluorophenyl)butanoyl)thiazolidine-2-carboxamido)methyl)-2,3-dihydrobenzo[b][1,4]dioxine-2-carboxylic acid is obtained according to the procedure used for Step 1, Example 2 (97%) as a white solid.

¹H NMR (CDCl₃, 300 MHz) δ 7.10-7.00 (m, 1H), 6.93-6.79 (m, 4H), 5.53-5.49 (m, 1H), 4.90-4.79 (m, 1H), 4.40-4.25 (m, 3H), 4.11-3.70 (m, 5H), 3.10-2.90 (m, 2H), 2.70-2.60 (m, 2H), 2.04-1.90 (m, 2H), 1.26 (s, 9H).

### Step 2: Preparation of 6-((3-((R)-3-amino-4-(2,4,5-trifluorophenyl) butanoyl)thiazolidine-2-carboxamido)methyl)-2,3-dihydrobenzo[b][1,4]dioxin-2-carboxylic acid.HCl

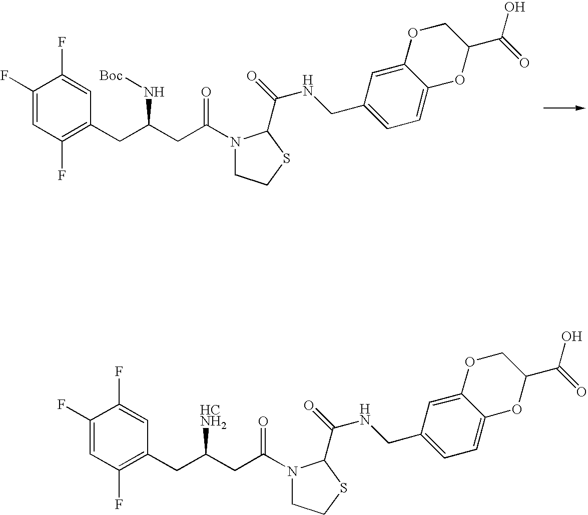

6-((3-((R)-3-amino-4-(2,4,5-trifluorophenyl) butanoyl)thiazolidine-2-carboxamido)methyl)-2,3-dihydrobenzo[b][1,4]dioxin-2-carboxylic acid.HCl is obtained according to the procedure used for Step 2, Example 1 (55 mg, 94%) as a white solid.

¹H NMR (DMSO-d₆, 300 MHz) δ 13.30 (br, 1H), 8.08 (br, 3H), 7.58-7.52 (m, 2H), 6.87-6.73 (m, 3H), 5.41-5.37 (m, 1H), 5.02-5.00 (m, 1H), 4.40-4.30 (m, 1H), 4.23-3.57 (m, 8H), 3.20-3.00 (m, 2H), 2.99-2.80 (m, 2H).

### Example 18

### Preparation of (S)-3-((R)-3-amino-4-(2,4,5-trifluorophenyl)butanoyl)thiazolidine-2-carboxylic acid.HCl

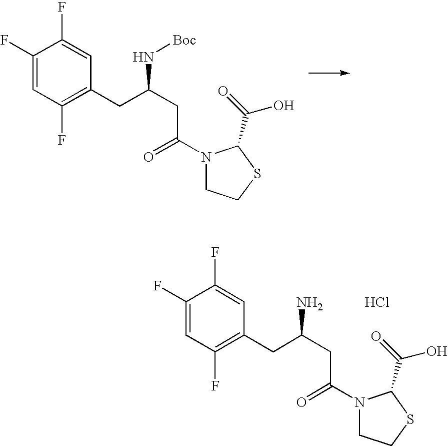

(S)-3-((R)-3-amino-4-(2,4,5-trifluorophenyl)butanoyl)thiazolidine-2-carboxylic acid.HCl is obtained according to the procedure used for Step 2, Example 1 (90%) as a white solid from (S)-3-((R)-3-(tert-butoxycarbonylamino)-4-(2,4,5-trifluorophenyl)butanoyl)thiazolidine-2-carboxylic acid in Example 22.

¹H NMR (DMSO-d₆ 300 MHz) δ 13.08 (br, 1H), 8.06 (br, 3H), 7.61-7.48 (m, 2H), 5.28 (s, 1H), 3.95-3.59 (m, 3H), 3.23-3.16 (m, 2H), 3.08-2.67 (m, 4H). LC-MS m/z (relative intensity) 349 (M+H)⁺.

### Example 19

### Preparation of ethyl 2-(4-((3-((R)-3-((1-acetoxyethoxy)carbonylamino)-4-(2,4,5-trifluorophenyl)butanoyl)thiazolidine-2-carboxamido)methyl)phenoxy)-3-methylbutanoate

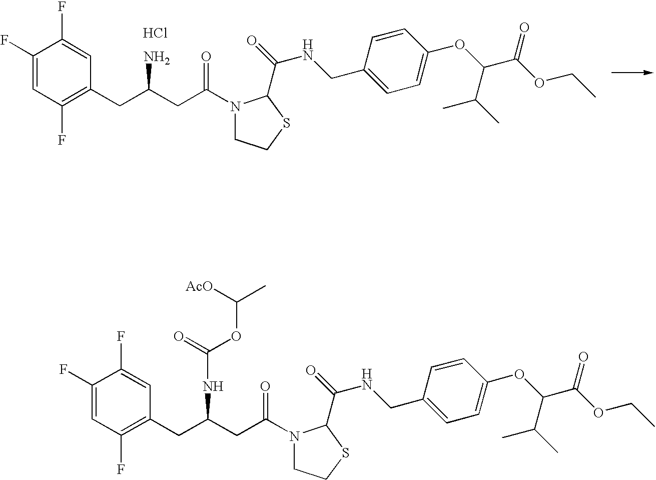

(1-(Acetoxy)ethyl)-(4-nitrophenyl)carbonate and ethyl 2-(4-((3-((R)-3-amino-4-(2,4,5-trifluorophenyl)butanoyl)thiazolidine-2-carboxamido)methyl)phenoxy)-3-methylbutanoate.HCl (155 mg, 0.25 mmol) are dissolved in CH₂Cl₂. Thereto, triethylamine (42 μl, 0.30 mmol) is added, followed by stirring for 2 days at room temperature. The resulting mixture is washed with 0.3 M KHSO₄, NaHCO₃ and brine and extracted with CH₂Cl₂. The entire extracts are dried over MgSO₄. The resulting organic layer is concentrated under a reduced pressure and purified by column chromatography (MeOH:CH₂Cl₂=1:10 and EtOAc:hexane=1:1) to obtain the compound, ethyl 2-(4-((3-((R)-3-((1-acetoxyethoxy)carbonylamino)-4-(2,4,5-trifluorophenyl)butanoyl)thiazolidine-2-carboxamido)methyl)phenoxy)-3-methylbutanoate (120 mg, 67%) as a white solid.

¹H NMR (CDCl₃, 300 MHz) δ 7.21-7.09 (m, 3H), 6.91-6.82 (m, 3H), 6.72-6.69 (m, 1H), 6.25 (br, 1H), 6.00-5.92 (m, 1H), 5.49 (d, J=6.3 Hz, 1H), 4.37-4.17 (m, 6H), 4.00-3.83 (m, 1H), 3.80-3.65 (m, 1H), 3.55-3.40 (m, 1H), 3.26-2.82 (m, 3H), 2.75-2.50 (m, 2H), 2.40-2.20 (m, 1H), 2.03 (s, 3H), 1.43-1.40 (m, 3H), 1.25 (t, J=7.2 Hz, 3H), 1.07-1.04 (m, 6H); LC-MS m/z (relative intensity) 712 (MH⁺).

### Example 20

### Preparation of (3R)-3-amino-1-(2-(morpholine-4-carbonyl)thiazolidin-3-yl)-4-(2,4,5-trifluorophenyl)butan-1-one.HCl

### Step 1: Preparation of tert-butyl (R)-4-(2-(morpholine-4-carbonyl)thiazolidin-3-yl)-4-oxo-1-(2,4,5-trifluorophenyl)butan-2-ylcarbamate

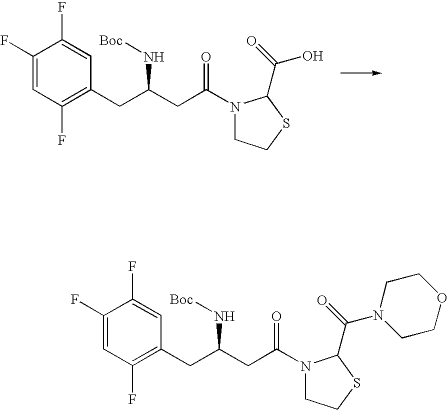

3-((R)-3-(tert-butoxycarbonylamino)-4-(2,4,5-trifluorophenyl)butanoyl)thiazolidine-2-carboxylic acid (30 mg, 0.067 mmol) is dissolved in CH₂Cl₂ (1 ml). Thereto, morpoline (20 μl, 0.22 mmol), EDC (63 mg, 0.33 mmol) and Et₃N (77 μl, 0.55 mmol) are added, followed by stirring for 12 hours at room temperature. The resulting mixture is extracted with CH₂Cl₂. The entire extracts are washed with brine and dried over MgSO₄. The resulting organic layer is concentrated under a reduced pressure and purified by column chromatography (EtOAc:hexane=1:1) to obtain the compound, tert-butyl (R)-4-(2-(morpholine-4-carbonyl)thiazolidin-3-yl)-4-oxo-1-(2,4,5-trifluorophenyl)butan-2-ylcarbamate (17 mg, 50%).

¹H NMR (CDCl₃, 300 MHz) δ 7.27-7.05 (m, 1H), 6.93-6.84 (m, 1H), 5.87 (d, J=3.9 Hz, 1H), 5.58-5.47 (br, 1H), 4.15-4.10 (m, 1H), 3.98-3.94 (m, 1H), 3.80-3.51 (m, 8H), 3.43-3.37 (m, 1H), 3.14-3.12 (m, 1H), 2.95-2.89 (m, 2H), 2.66-2.62 (m, 2H), 1.80-1.75 (m, 1H), 1.37 (s, 9H).

### Step 2: Preparation of (3R)-3-amino-1-(2-(morpholin-4-carbonyl) thiazolidin-3-yl)-4-(2,4,5-trifluorophenyl)butan-1-one.HCl

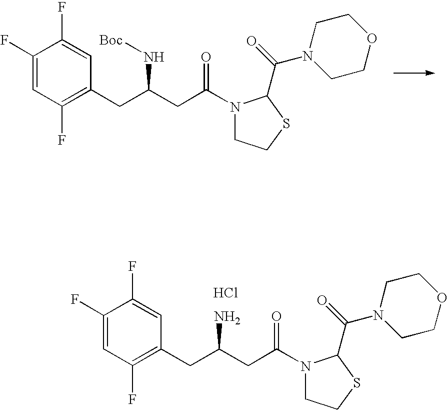

(3R)-3-amino-1-(2-(morpolin-4-carbonyl)thiazolidin-3-yl)-4-(2,4,5-trifluorophenyl)butan-1-on.HCl is obtained according to the procedure used for Step 2, Example 1 (80%).

¹H NMR (CD₃OD, 300 MHz) δ 7.35-7.30 (m, 1H), 7.24-7.18 (m, 1H), 5.89 (d, J=14.0 Hz, 1H), 3.86-3.80 (m, 2H), 3.66-3.40 (m, 7H), 3.29-3.25 (m, 4H), 3.06-3.00 (m, 2H), 2.84-2.64 (m, 2H).

### Example 21

### Preparation of N-(2-(1H-imidazol-4-yl)ethyl)-3-((R)-3-amino-4-(2,4,5-trifluorophenyl)butanoyl)thiazolidine-2-carboxamide.2HCl

### Step 1: Preparation of tert-butyl (2R)-4-(2-(2-(1H-imidazol-4-yl)ethylcarbamoyl)thiazolidin-3-yl)-4-oxo-1-(2,4,5-trifluorophenyl)butan-2-ylcarbamate

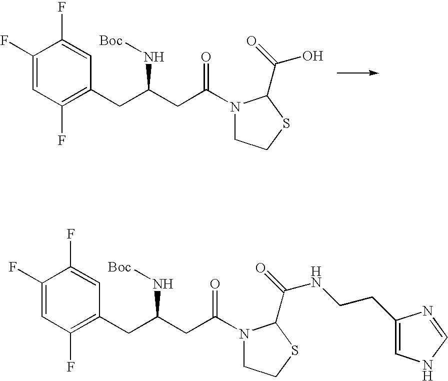

3-((R)-3-(tert-butoxycarbonylamino)-4-(2,4,5-trifluorophenyl)butanoyl)thiazolidine-2-carboxylic acid (45 mg, 0.10 mmol) is dissolved in CH₂Cl₂ (1 ml). Thereto, histamine.2HCl (55 mg, 0.30 mmol), EDCI (58 mg, 0.30 mmol), HOBT (3 mg, 0.02 mmol) and DIEA (174 μl, 1.00 mmol) are added, followed by stirring for 12 hours at room temperature. The resulting mixture is extracted with CH₂Cl₂. The entire extracts are washed with brine and dried over MgSO₄. The resulting organic layer is concentrated under a reduced pressure and purified by column chromatography (EtOAc:hexane=1:1) to obtain the compound tert-butyl (2R)-4-(2-(2-(1H-imidazol-4-yl)ethylcarbamoyl)thiazolidin-3-yl)-4-oxo-1-(2,4,5-trifluorophenyl)butan-2-ylcarbamate (8 mg, 15%).

¹H NMR (CDCl₃, 300 MHz) δ 7.61 (s, 1H), 7.18-7.06 (m, 1H), 6.93-6.85 (m, 1H), 6.83 (s, 1H), 5.58 (brs, 1H), 5.46 (s, 1H), 4.16-4.02 (m, 2H), 3.76-3.37 (m, 4H), 3.09-3.07 (m, 1H), 2.83-2.62 (m, 6H), 1.36 (s, 9H).

### Step 2: Preparation of N-(2-(1H-imidazol-5-yl)ethyl)-3-((R)-3-amino-4-(2,4,5-trifluorophenyl)butanoyl)thiazolidine-2-carboxamide.2HCl

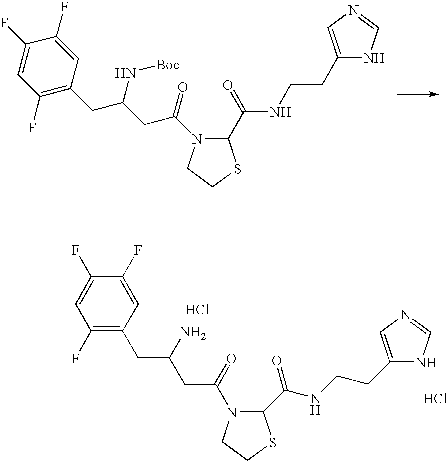

N-(2-(1H-imidazol-5-yl)ethyl)-3-((R)-3-amino-4-(2,4,5-trifluorophenyl)butanoyl)thiazolidine-2-carboxamide.2HCl is obtained according to the procedure used for Step 2, Example 1 (92%).

¹H NMR (DMSO-d₆, 300 MHz) δ 9.01 (s, 1H), 8.33-8.07 (m, 1H), 7.64-7.49 (m, 1H), 7.40 (s, 1H), 5.25 (d, J=11.7 Hz, 1H), 3.71-3.57 (m, 1H), 3.16-3.14 (m, 2H), 3.02-2.78 (m, 8H).

### Example 22

### Preparation of (S)-2-(4-(((S)-3-((R)-3-amino-4-(2,4,5-trifluorophenyl)butanoyl)thiazolidine-2-carboxamido)methyl)phenoxy)-3-methylbutanoic acid.HCl

### Step 1: Preparation of (S)-ethyl thiazolidine-2-carboxylate

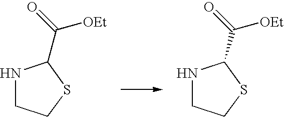

L-tartaric acid (18.91 g, 0.126 mol) is dissolved in anhydrous ethanol (103 ml) while heated in an opened flask. Thereto, ethyl thiazolidine-2-carboxylate (20.316 g, 0.126 mol) dissolved in diethyl ether (35 ml) is added and placed at room temperature. As crystals begins to precipitate, the mixture is repeatedly subjected to heating and cooling for 10 days until about 30% of the reaction solvent is slowly evaporated. The precipitated crystals are filtered and collected. The filtrate is washed with diethyl ether and dried to obtain an L-tartaric acid salt of (S)-ethyl thiazolidine-2-carboxylate (α_{D}=−65°, >99% ee, HPLC t_{R}=6.5 min) (31.38 g, 80%) as a white solid. Similarly, the filtrate is repeatedly subjected to heating and cooling for evaporation of the solvent, which procedure is repeated 2 to 3 times to obtain the L-tartaric acid salt quantitatively in its total yield. The L-tartaric acid salt of(S)-ethyl thiazolidine-2-carboxylate (16.55 g, 50 mmol) thus obtained is added to a 10% sodium bicarbonate solution maintained at 10° C. or less, followed by stirring for 30 minutes. The resultant is extracted with diethyl ether twice, the entire extracts are washed with distilled water. The organic layer is separated, dried over MgSO₄, filtered and concentrated, to obtain (S)-ethyl thiazolidine-2-carboxylate (6.12 g, 76%, 99% ee, HPLC t_{R}=6.5 min).

¹H NMR (300 MHz, CDCl₃) 4.93 (brs, 1H), 4.26 (q, J=7.1 Hz, 2H), 3.72-3.63 (m, 1H), 3.13-2.98 (m, 2H), 2.90-2.81 (m, 1H), 2.33 (br, 1H), 1.32 (t, J=7.1 Hz, 3H).

HPLC analysis: Daicel OD column 4.6*250 mm, EtOH/n-Hexane (1/9) with 0.1% diethylamine, 1.0 ml/min, 254 nm UV detector; (S-form, 6.5 min), (R-form, 7.4 min).

### Step 2: Preparation of (S)-ethyl 3-((R)-3-(tert-butoxycarbonylamino)-4-(2,4,5-trifluorophenyl)butanoyl)thiazolidine-2-carboxylate

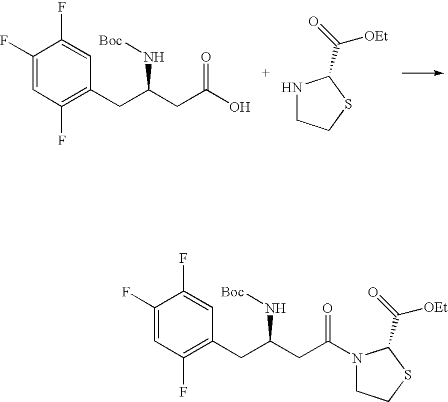

(R)-3-t-butoxycarbonylamino-4-(2,4,5-trifluorophenyl)-butyric acid (20 g, 60 mmol), (S)-ethyl thiazolidine-2-carboxylate (9.7 g, 60 mmol) obtained in step 1 above, EDC (14 g, 73 mmol) and DMAP (7.4 g, 60 mmol) are suspended in CH₂Cl₂ (500 ml). Thereto, triethylamine (17 g) is added, followed by stirring for 12 hours at room temperature. The resulting mixture was washed with brine and extracted with CH₂Cl₂. The entire extracts are dried over anhydrous sodium sulfate and concentrated. The residue is purified by silica gel column chromatography to obtain the compound, (S)-ethyl 3-((R)-3-(tert-butoxycarbonylamino)-4-(2,4,5-trifluorophenyl)butanoyl)thiazolidine-2-carboxylate (20 g, 70%).

¹H NMR (300 MHz, CDCl₃) 7.12-7.03 (m, 1H), 6.93-6.84 (m, 1H), 5.59 (brd, 1H), 5.47 (s, 1H), 4.24 (q, J=7.1 Hz, 2H), 4.16-4.09 (m, 1H), 3.98-3.82 (m, 1H), 3.77-3.68 (m, 1H), 3.40-3.31 (m, 1H), 3.11-3.05 (m, 1H), 2.93 (d, J=7.2 Hz, 2H), 2.64 (d, J=5.1 Hz, 2H), 1.38 (s, 9H), 1.30 (t, J=7.1 Hz, 3H).

### Step 3: Preparation of (S)-3-((R)-3-(tert-butoxycarbonylamino)-4-(2,4,5-trifluorophenyl)butanoyl)thiazolidine-2-carboxylic acid

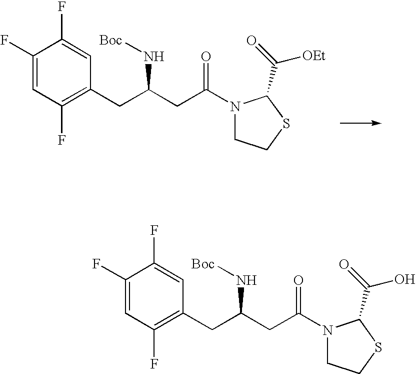

(S)-ethyl 3-((R)-3-(tert-butoxycarbonylamino)-4-(2,4,5-trifluorophenyl)butanoyl)thiazolidine-2-carboxylate (3.2 g, 6.7 mmol) obtained in step 2 above is dissolved in a mixture of THF (30 ml) and MeOH (30 ml). Thereto, LiOH.H₂O (1.42 g, 34 mmol) dissolved in distilled water (30 ml) is added, followed by stirring for 3 hours at room temperature. The resulting mixture is concentrated, cooled with ice water and acidified to a pH of 3.0 with 2 N HCl. The resultant is extracted with ethyl acetate and the entire extracts are dried over anhydrous sodium sulfate and concentrated to obtain the compound, (S)-3-((R)-3-(tert-butoxycarbonylamino)-4-(2,4,5-trifluorophenyl)butanoyl)thiazolidine-2-carboxylic acid (2.99 g, 99%).

¹H NMR (300 MHz, CDCl₃) 7.12-7.04 (m, 1H), 6.93-6.85 (m, 1H), 5.51 (s, 1H), 4.17-4.04 (m, 1H), 3.99-3.93 (m, 1H), 3.79-3.70 (m, 1H), 3.43-3.34 (m, 1H), 3.14-3.07 (m, 1H), 2.93 (d, J=6.9 Hz, 2H), 2.67 (d, J=4.7 Hz, 2H), 1.36 (s, 9H).

### Step 4: Preparation of (R)-ethyl 2-hydroxy-3-methylbutanoate

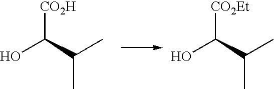

(R)-2-hydroxy-3-methyl-butyric acid (1 g, 8.4 mmol) is dissolved in acetone (50 ml). Thereto, K₂CO₃ (1.4 g, 10 mmol) and ethyl iodide (2.67 g, excess) are added, and the resulting mixture is refluxed for 4 hours. Then, the mixture is extracted with diethyl ether. The entire extracts are dried over anhydrous MgSO₄ and concentrated. The residue is purified by silica gel column chromatography to obtain the compound, (R)-ethyl 2-hydroxy-3-methylbutanoate (0.88 g, 72%).

### Step 5: Preparation of (S)-ethyl 2-(4-formylphenoxy)-3-methylbutanoate

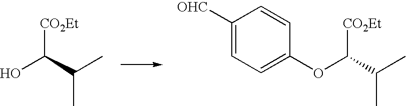

(R)-ethyl 2-hydroxy-3-methylbutanoate (1.425 g, 9.74 mmol) obtained in step 4 above, 4-hydroxybenzaldehyde (1.064 g, 9.74 mmol) and triphenylphosphin (2.556 g, 9.74 mmol) are dissolved in tetrahydrofuran (30 ml) and cooled to 0° C. with ice water. Thereto, diisopropyl azodicarboxylate (1.970 g, 9.74 mmol) is slowly added dropwise, followed by stirring for 12 hours. The resulting mixture is washed with brine and extracted with diethyl ether. The organic layer is dried over anhydrous MgSO₄ and concentrated. The residue is purified by silica gel column chromatography to obtain the compound, (S)-ethyl 2-(4-formylphenoxy)-3-methylbutanoate (1.237 g, 51%).

¹H NMR (300 MHz, CDCl₃) 9.88 (s, 1H), 7.82 (dt, J=8.8 Hz, 2H), 6.90 (dt, J=8.8 Hz, 2H), 4.48 (d, J=5.3 Hz, 1H), 4.23 (q, J=7.1 Hz, 2H), 2.39-2.28 (m, 1H), 1.24 (t, J=7.1 Hz, 3H), 1.11 (d, J=5.1 Hz, 3H), 1.09 (d, J=5.1 Hz, 3H).

### Step 6: Preparation of (S)-ethyl 2-(4-((hydroxyimino)methyl)phenoxy)-3-methylbutanoate

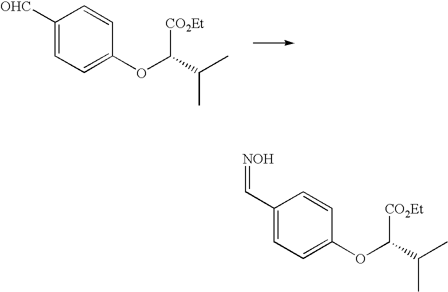

(S)-Ethyl 2-(4-formylphenoxy)-3-methylbutanoate (1.102 g, 4.4 mmol) obtained in step 5 above is dissolved in ethanol (70 ml). Thereto, NH₂OH.HCl (918 mg, 13.2 mmol) and pyridine (1.04 g, 13.2 mmol) are added, and the resulting mixture is refluxed for 3 hours. Then, the mixture is concentrated and extracted with ethyl acetate, and the entire extracts are washed with dilute HCl. The organic layer is dried over anhydrous MgSO₄ and concentrated. The residue is purified by silica gel column chromatography to obtain the compound, (S)-ethyl 2-(4-((hydroxyimino)methyl)phenoxy)-3-methylbutanoate (0.821 g, 71%).

¹H NMR (300 MHz, CDCl₃) 8.07 (s, 1H), 7.49 (dt, J=8.8 Hz, 2H), 6.89 (dt, J=8.8 Hz, 2H), 4.39 (d, J=5.5 Hz, 1H), 4.22 (q, J=7.1 Hz, 2H), 2.34-2.27 (m, 1H), 1.24 (t, J=7.1 Hz, 3H), 1.09 (d, J=6.8 Hz, 3H), 1.07 (d, J=6.8 Hz, 3H)

### Step 7: Preparation of (S)-ethyl 2-(4-((tert-butoxycarbonylamino)methyl)phenoxy)-3-methylbutanoate

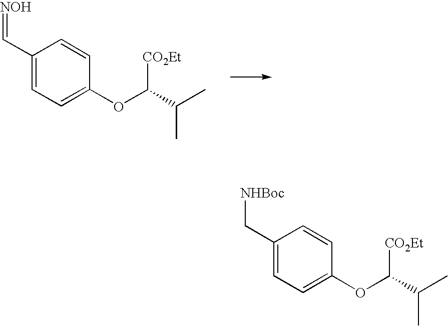

(S)-Ethyl 2-(4-((hydroxyimino)methyl)phenoxy)-3-methylbutanoate (492 g, 1.85 mmol) obtained in step 6 above is dissolved in ethanol (40 ml). Thereto, di-tert-butyl dicarbonate (484 mg, 2.22 mmol) and 10%-Pd/C (99 mg, 5 mol %) is added and reacted for 12 hours under hydrogen (1 atm). The reaction mixture is filtered through celite and concentrated. The residue is separated by silica gel column chromatography to obtain the compound, (S)-ethyl 2-(4-((tert-butoxycarbonylamino)methyl)phenoxy)-3-methylbutanoate (454 mg, 70%).

¹H NMR (300 MHz, CDCl₃) 7.18 (dt, J=8.5 Hz, 2H), 6.84 (dt, J=8.5 Hz, 2H), 4.33 (d, J=5.6 Hz, 1H), 4.25-4.17 (m, 4H), 2.32-2.21 (m, 1H), 1.25 (t, J=7.1 Hz, 3H), 1.09 (d, J=6.8 Hz, 3H), 1.06 (d, J=6.8 Hz, 3H).

### Step 8: Preparation of (S)-ethyl 2-(4-(aminomethyl)phenoxy)-3-methylbutanoate.HCl

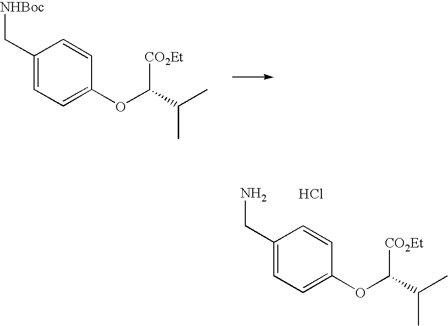

(S)-ethyl 2-(4-((tert-butoxycarbonylamino)methyl)phenoxy)-3-methylbutanoate (351 mg, 1 mmol) obtained in step 7 above is dissolved in CH₂Cl₂ (30 ml). Thereto, a 4 M HCl/dioxane mixture (1 ml) is added, followed by stirring for 12 hours at room temperature. The resulting mixture is concentrated and dried to obtain the compound, (S)-ethyl 2-(4-(aminomethyl)phenoxy)-3-methylbutanoate.HCl (274 mg, 95%) as a white solid.

### Step 9: Preparation of (S)-ethyl 2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoate

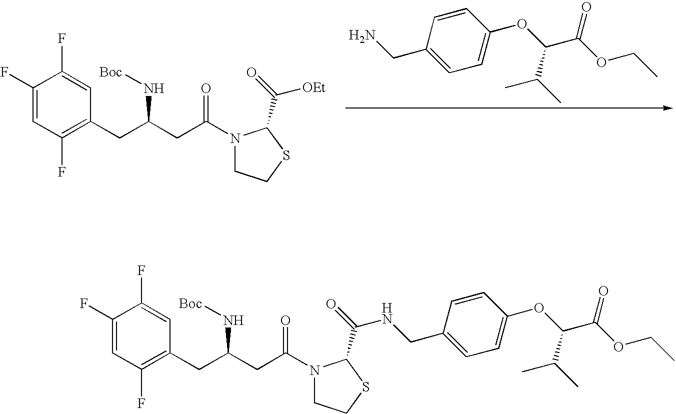

(S)-3-((R)-3-(tert-butoxycarbonylamino)-4-(2,4,5-trifluorophenyl)butanoyl)thiazolidine-2-carboxylic acid (160 mg, 0.35 mmol) obtained in step 3 above and (S)-ethyl 2-(4-(aminomethyl)phenoxy)-3-methylbutanoate.HCl (123 mg, 0.42 mmol) obtained in step 8 above are suspended in CH₂Cl₂ (100 ml). Thereto, EDC (164 mg, 0.85 mmol) is added, followed by stirring for 3 hours at room temperature. The resulting mixture is washed with brine and extracted with CH₂Cl₂. The entire extracts are dried over anhydrous sodium sulfate and concentrated. The residue is purified by silica gel column chromatography to obtain the compound, (S)-ethyl 2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoate (161 mg, 68%).

¹H NMR (300 MHz, CDCl₃) 7.19 (d, J=8.6 Hz, 2H), 7.18-7.03 (m, 1H), 6.93-6.80 (m, 1H), 6.83 (d, J=8.6 Hz, 2H), 6.32 (br, 1H, NH), 5.58 (brd, 1H, NH), 5.50 (s, 1H), 4.48-4.08 (m, 6H), 3.96-3.90 (m, 1H), 3.76-3.68 (m, 1H), 3.52-3.43 (m, 1H), 3.11-3.05 (m, 1H), 2.89 (d, J=5.7 Hz, 2H), 2.62 (d, J=5.0 Hz, 2H), 2.30-2.23 (m, 1H), 1.37 (s, 9H), 1.24 (t, J=7.1 Hz, 3H), 1.08 (d, J=6.8 Hz, 3H), 1.05 (d, J=6.8 Hz, 3H).

### Step 10: Preparation of (S)-2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoic acid

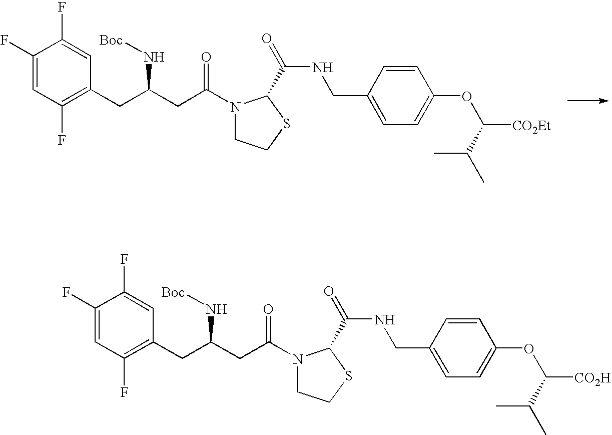

(S)-ethyl 2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoate (100 mg, 0.146 mmol) is dissolved in a mixture of THF (5 ml) and MeOH (5 ml). Thereto, LiOH.H₂O (125 mg, 2.94 mmol) dissolved in distilled water (5 ml) is added, followed by stirring for 24 hours at room temperature. The resulting mixture is concentrated, cooled with ice water and acidified to a pH of 3 with 2 N HCl. The resultant is extracted with ethyl acetate. The entire extracts are dried over anhydrous sodium sulfate and concentrated to obtain the compound, (S)-2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoic acid (83 mg, 87%).

¹H NMR (300 MHz, CDCl₃) 7.16-7.02 (m, 3H), 6.93-6.82 (m, 3H), 6.59 (br, 1H, NH), 5.54 (brd, 1H, NH), 5.47 (s, 1H), 4.40-4.28 (m, 2H), 4.14-4.04 (m, 1H), 3.91-3.80 (m, 1H), 3.74-3.64 (m, 1H), 3.50-3.40 (m, 1H), 3.09-3.00 (m, 1H), 2.90-2.82 (m, 2H), 2.62-2.56 (m, 2H), 2.36-2.26 (m, 1H), 1.37 (s, 9H), 1.11 (d, J=6.5 Hz, 3H), 1.09 (d, J=6.5 Hz, 3H).

### Step 11: Preparation of (S)-2-(4-(((S)-3-((R)-3-amino-4-(2,4,5-trifluorophenyl)butanoyl)thiazolidine-2-carboxamido)methyl)phenoxy)-3-methylbutanoic acid.HCl

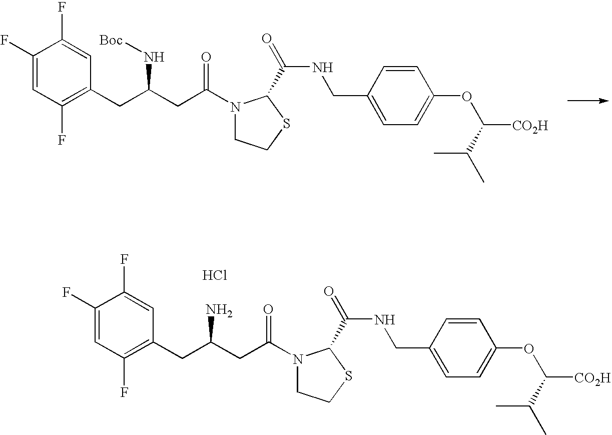

(S)-2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoic acid (73 mg, 0.11 mmol) is dissolved in CH₂Cl₂ (5 ml). Thereto, a 4 M-HCl/dioxane mixture (0.2 ml) is added, followed by stirring for 12 hours at room temperature. The resulting mixture is completely concentrated and recrystallized with diethyl ether added in a small amount. After the supernatant is separated out, the white solid formed is dried to obtain the desired compound, (S)-2-(4-(((S)-3-((R)-3-amino-4-(2,4,5-trifluorophenyl)butanoyl)thiazolidine-2-carboxamido)methyl)phenoxy)-3-methylbutanoic acid.HCl (55 mg, 85%).

¹H NMR (300 MHz, DMSO-d₆) 12.96 (brs, 1H), 8.48 (brt, 1H, NH), 8.07 (brs, 3H), 7.61-7.51 (m, 2H), 7.19-7.12 (m, 2H), 6.86-6.77 (m, 2H), 5.40 (s, 1H), 4.45-4.39 (m, 1H), 4.24-4.16 (m, 2H), 3.99-3.92 (m, 1H), 3.80-3.66 (m, 2H), 3.24-3.16 (m, 2H), 3.00-2.94 (m, 2H), 2.78-2.72 (m, 2H), 2.22-2.14 (m, 1H), 1.00 (d, J=6.7 Hz, 6H).

### Example 23

### Preparation of (R)-2-(4-(((S)-3-((R)-3-amino-4-(2,4,5-trifluorophenyl)butanoyl)thiazolidine-2-carboxamido)methyl)phenoxy)-3-methylbutanoic acid.HCl

### Step 1: Preparation of (S)-ethyl 2-hydroxy-3-methylbutanoate

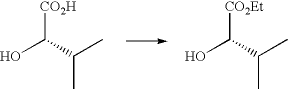

(S)-ethyl 2-hydroxy-3-methylbutanoate is obtained according to the procedure used for Step 4, Example 22 (70%) except (S)-2-hydroxy-3-methyl-butyric acid is used instead of (R)-2-hydroxy-3-methyl-butyric acid (70%).

### Step 2: Preparation of (R)-ethyl 2-(4-formylphenoxy)-3-methylbutanoate

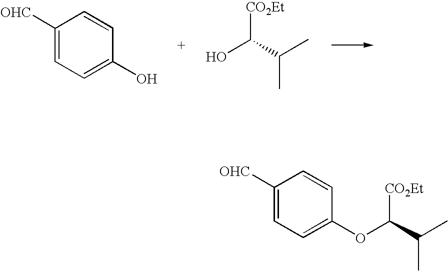

(R)-ethyl 2-(4-formylphenoxy)-3-methylbutanoate is obtained according to the procedure used for Step 5, Example 22 except (S)-ethyl 2-hydroxy-3-methylbutanoate is used instead of (R)-ethyl 2-hydroxy-3-methylbutanoate (50%).

¹H NMR (300 MHz, CDCl₃) 9.88 (s, 1H), 7.82 (dt, J=8.8 Hz, 2H), 6.90 (dt, J=8.8 Hz, 2H), 4.48 (d, J=5.3 Hz, 1H), 4.23 (q, J=7.1 Hz, 2H), 2.39-2.28 (m, 1H), 1.24 (t, J=7.1 Hz, 3H), 1.11 (d, J=5.1 Hz, 3H), 1.09 (d, J=5.1 Hz, 3H).

### Step 3: Preparation of (R)-ethyl 2-(4-((hydroxyimino)methyl)phenoxy)-3-methylbutanoate

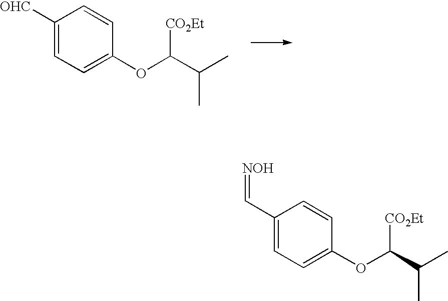

(R)-ethyl 2-(4-((hydroxyimino)methyl)phenoxy)-3-methylbutanoate is obtained according to the procedure used for Step 6, Example 22 except (R)-Ethyl 2-(4-formylphenoxy)-3-methylbutanoate instead of (S)-Ethyl 2-(4-formylphenoxy)-3-methylbutanoate (88%).

¹H NMR (300 MHz, CDCl₃) 8.07 (s, 1H), 7.49 (dt, J=8.8 Hz, 2H), 6.89 (dt, J=8.8 Hz, 2H), 4.39 (d, J=5.5 Hz, 1H), 4.22 (q, J=7.1 Hz, 2H), 2.34-2.27 (m, 1H), 1.24 (t, J=7.1 Hz, 3H), 1.09 (d, J=6.8 Hz, 3H), 1.07 (d, J=6.8 Hz, 3H).

### Step 4: Preparation of (R)-ethyl 2-(4-((tert-butoxycarbonylamino)methyl)phenoxy)-3-methylbutanoate

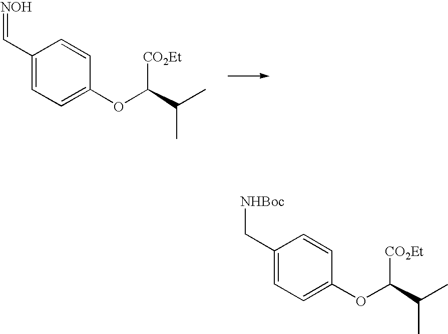

(R)-ethyl 2-(4-((tert-butoxycarbonylamino)methyl)phenoxy)-3-methylbutanoate is obtained according to the procedure used for Step 7, Example 22 except (R)-Ethyl 2-(4-((hydroxyimino)methyl)phenoxy)-3-methylbutanoate is used instead of (S)-Ethyl 2-(4-((hydroxyimino)methyl)phenoxy)-3-methylbutanoate (69%).

¹H NMR (300 MHz, CDCl₃) 7.18 (dt, J=8.5 Hz, 2H), 6.84 (dt, J=8.5 Hz, 2H), 4.33 (d, J=5.6 Hz, 1H), 4.25-4.17 (m, 4H), 2.32-2.21 (m, 1H), 1.25 (t, J=7.1 Hz, 3H), 1.09 (d, J=6.8 Hz, 3H), 1.06 (d, J=6.8 Hz, 3H).

### Step 5: Preparation of (R)-ethyl 2-(4-(aminomethyl)phenoxy)-3-methylbutanoate.HCl

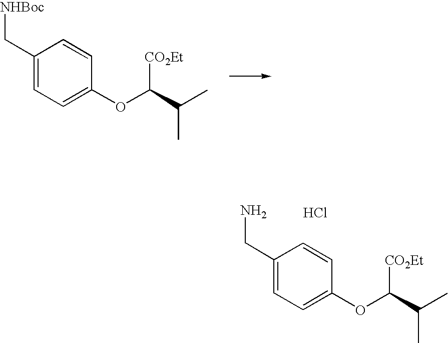

(R)-ethyl 2-(4-(aminomethyl)phenoxy)-3-methylbutanoate.HCl is obtained according to the procedure used for Step 8, Example 22 except (R)-ethyl 2-(4-((tert-butoxycarbonylamino)methyl)phenoxy)-3-methylbutanoate is used instead of (S)-ethyl 2-(4-((tert-butoxycarbonylamino)methyl)phenoxy)-3-methylbutanoate (92%) as a white solid.

### Step 6: Preparation of (R)-ethyl 2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoate

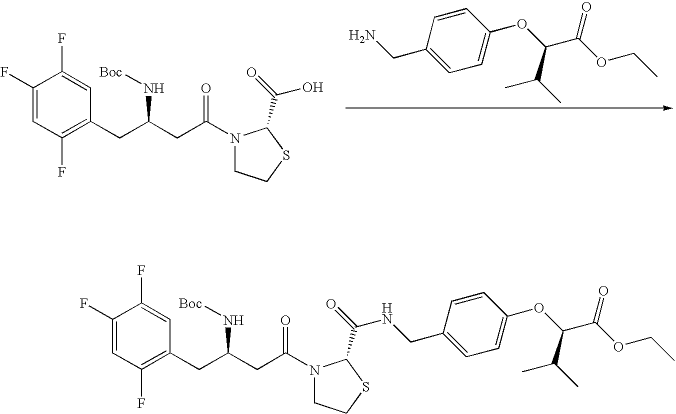

(R)-ethyl 2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoate is obtained according to the procedure used for Step 9, Example 22 except (R)-ethyl 2-(4-(aminomethyl)phenoxy)-3-methylbutanoate.HCl is used instead of (S)-ethyl 2-(4-(aminomethyl)phenoxy)-3-methylbutanoate.HCl (67%).

¹H NMR (300 MHz, CDCl₃) 7.19 (d, J=8.6 Hz, 2H), 7.16-7.03 (m, 1H), 6.93-6.82 (m, 1H), 6.83 (d, J=8.6 Hz, 2H), 6.20 (btr, 1H, NH), 5.57 (brd, 1H, NH), 5.50 (s, 1H), 4.46-4.29 (m, 3H), 4.21 (q, J=7.1 Hz, 2H), 4.16-4.08 (m, 1H), 3.96-3.89 (m, 1H), 3.76-3.68 (m, 1H), 3.52-3.43 (m, 1H), 3.12-3.05 (m, 1H), 2.90 (d, J=5.5 Hz, 2H), 2.63 (d, J=4.9 Hz, 2H), 2.32-2.21 (m, 1H), 1.37 (s, 9H), 1.25 (t, J=7.1 Hz, 3H), 1.08 (d, J=6.9 Hz, 3H), 1.05 (d, J=6.9 Hz, 3H).

### Step 7: Preparation of (R)-2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoic acid

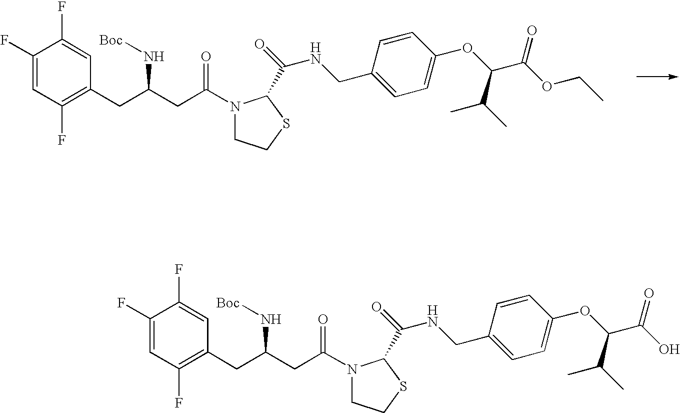

(R)-2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoic acid is obtained according to the procedure used for Step 10, Example 22 except (R)-ethyl 2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoate is used instead of (S)-ethyl 2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoate (97%).

¹H NMR (300 MHz, CDCl₃) 7.12 (d, J=8.6 Hz, 2H), 7.09-6.98 (m, 1H), 6.93-6.80 (m, 1H), 6.80 (d, J=8.6 Hz, 2H), 6.72 (br, 1H, NH), 5.54 (s, 1H), 5.47 (brd, 1H, NH), 4.38 (d, J=5.1 Hz, 1H), 4.33-4.27 (m, 1H), 4.12-4.04 (m, 1H), 3.97-3.89 (m, 1H), 3.74-3.64 (m, 1H), 3.51-3.42 (m, 1H), 3.08-3.00 (m, 1H), 2.82 (d, 2H), 2.59 (d, 2H), 2.32-2.21 (m, 1H), 1.37 (s, 9H), 1.25 (t, J=7.1 Hz, 3H), 1.08 (d, J=6.9 Hz, 3H), 1.05 (d, J=6.9 Hz, 3H).

### Step 8: Preparation of (R)-2-(4-(((S)-3-((R)-3-amino-4-(2,4,5-trifluorophenyl)butanoyl)thiazolidine-2-carboxamido)methyl)phenoxy)-3-methylbutanoic acid.HCl

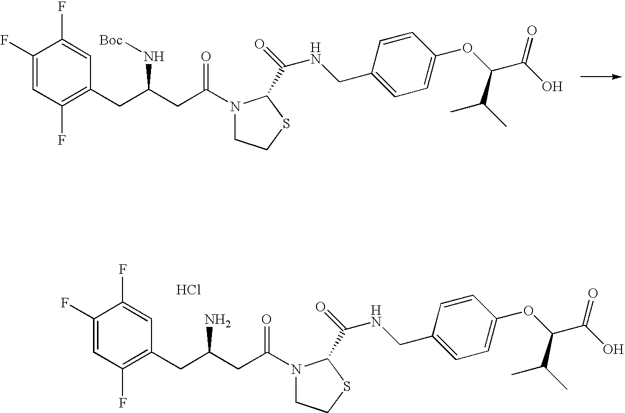

(R)-2-(4-(((S)-3-((R)-3-amino-4-(2,4,5-trifluorophenyl)butanoyl)thiazolidine-2-carboxamido)methyl)phenoxy)-3-methylbutanoic acid.HCl is obtained according to the procedure used for Step 11, Example 22 except (R)-2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoic acid is used instead of (S)-2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoic acid (95%).

¹H NMR (300 MHz, DMSO-d₆) 12.93 (brs, 1H), 8.48 (brt, 1H, NH), 8.08 (brs, 3H), 7.61-7.51 (m, 2H), 7.19-7.12 (m, 2H), 6.86-6.78 (m, 2H), 5.40 (s, 1H), 4.45-4.40 (m, 1H), 4.24-4.16 (m, 2H), 3.99-3.92 (m, 1H), 3.80-3.66 (m, 2H), 3.24-3.16 (m, 2H), 3.00-2.94 (m, 2H), 2.78-2.72 (m, 2H), 2.22-2.14 (m, 1H), 1.00 (d, J=6.7 Hz, 6H).

### Example 24

### Preparation of (S)-2-(4-(((R)-3-((R)-3-amino-4-(2,4,5-trifluorophenyl)butanoyl)thiazolidine-2-carboxamido)methyl)phenoxy)-3-methylbutanoic acid.HCl

### Step 1: Preparation (R)-ethyl thiazolidine-2-carboxylate

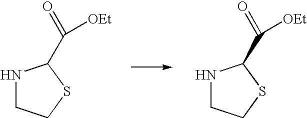

(R)-ethyl thiazolidine-2-carboxylate is obtained according to the procedure used for Step 1, Example 22 except D-tartaric acid is used instead of L-tartaric acid (99% ee, HPLC t_{R}=7.4 min).

¹H NMR (300 MHz, CDCl₃) 4.93 (brs, 1H), 4.26 (q, J=7.1 Hz, 2H), 3.72-3.63 (m, 1H), 3.13-2.98 (m, 2H), 2.90-2.81 (m, 1H), 2.33 (br, 1H), 1.32 (t, J=7.1 Hz, 3H).

HPLC analysis: Daicel OD column 4.6*250 mm, EtOH/n-Hexane (1/9) with 0.1% diethylamine, 1.0 ml/min, 254 nm UV detector; (S-form, 6.5 min), (R-form, 7.4 min).

### Step 2: Preparation of (R)-ethyl 3-((R)-3-(tert-butoxycarbonylamino)-4-(2,4,5-trifluorophenyl)butanoyl)thiazolidine-2-carboxylate

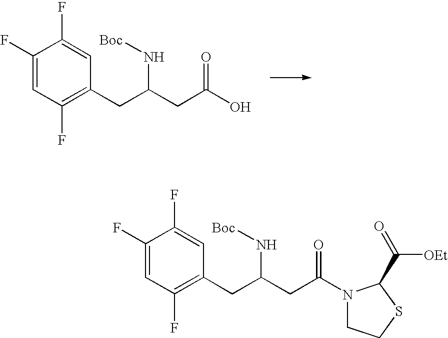

(R)-ethyl 3-((R)-3-(tert-butoxycarbonylamino)-4-(2,4,5-trifluorophenyl)butanoyl)thiazolidine-2-carboxylate is obtained according to the procedure used for Step 2, Example 22 except (R)-ethyl thiazolidine-2-carboxylate is used instead of (S)-ethyl thiazolidine-2-carboxylate (60%).

¹H NMR (300 MHz, CDCl₃) 7.19-7.10 (m, 1H), 6.94-6.85 (m, 1H), 5.64 (brd, 1H), 5.46 (s, 1H), 4.24 (q, J=7.1 Hz, 2H), 4.15-4.07 (m, 1H), 3.96-3.89 (m, 1H), 3.80-3.72 (m, 1H), 3.40-3.31 (m, 1H), 3.12-3.05 (m, 1H), 2.97-2.89 (m, 2H), 2.63-2.60 (m, 2H), 1.36 (s, 9H), 1.31 (t, J=7.1 Hz, 3H).

### Step 3: Preparation of (R)-3-((R)-3-(tert-butoxycarbonylamino)-4-(2,4,5-trifluorophenyl)butanoyl)thiazolidine-2-carboxylic acid

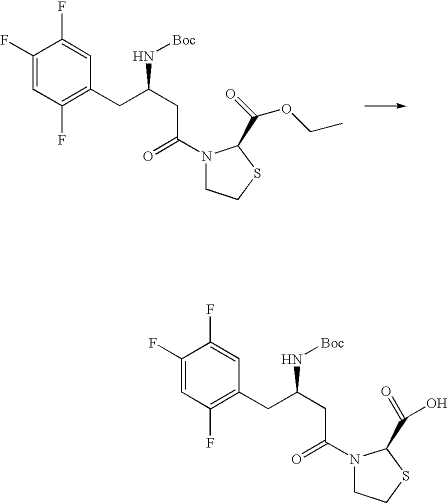

(R)-3-((R)-3-(tert-butoxycarbonylamino)-4-(2,4,5-trifluorophenyl)butanoyl)thiazolidine-2-carboxylic acid is obtained according to the procedure used for Step 3, Example 22 except (R)-ethyl 3-((R)-3-(tert-butoxycarbonylamino)-4-(2,4,5-trifluorophenyl)butanoyl)thiazolidine-2-carboxylate is used instead of (S)-ethyl 3-((R)-3-(tert-butoxycarbonylamino)-4-(2,4,5-trifluorophenyl)butanoyl)thiazolidine-2-carboxylate (95%).

¹H NMR (300 MHz, CDCl₃) 7.14-7.05 (m, 1H), 6.93-6.84 (m, 1H), 5.55 (brd, 1H), 5.49 (s, 1H), 4.17-4.03 (m, 1H), 3.99-3.92 (m, 1H), 3.81-3.73 (m, 1H), 3.41-3.32 (m, 1H), 3.13-3.06 (m, 1H), 3.01-2.87 (m, 2H), 2.74-2.55 (m, 2H), 1.36 (s, 9H).

### Step 4: Preparation of (S)-ethyl 2-(4-(((R)-3-((R)-3-(tert-butoxycarbonylamino)-4-(2,4,5-trifluorophenyl)butanoyl)thiazolidine-2-carboxamido)methyl)phenoxy)-3-methylbutanoate

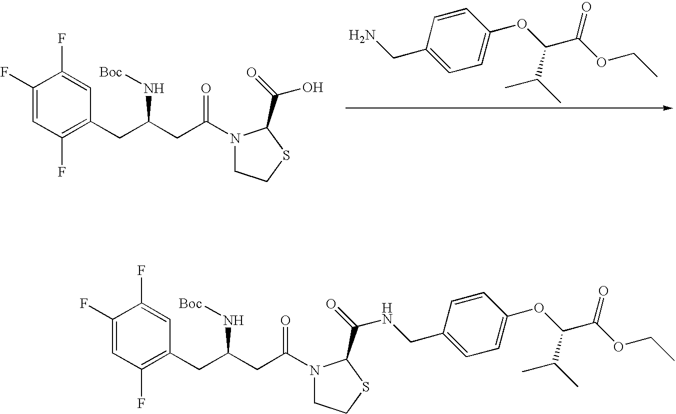

(S)-ethyl 2-(4-(((R)-3-((R)-3-(tert-butoxycarbonylamino)-4-(2,4,5-trifluorophenyl)butanoyl)thiazolidine-2-carboxamido)methyl)phenoxy)-3-methylbutanoate is obtained according to the procedure used for Step 9, Example 22 except (R)-3-((R)-3-(tert-butoxycarbonylamino)-4-(2,4,5-trifluorophenyl)butanoyl)thiazolidine-2-carboxylic acid is used instead of (S)-3-((R)-3-(tert-butoxycarbonylamino)-4-(2,4,5-trifluorophenyl)butanoyl)thiazolidine-2-carboxylic acid (75%).

¹H NMR (300 MHz, CDCl₃) 7.19 (d, J=8.6 Hz, 2H), 7.18-7.08 (m, 1H), 6.92-6.82 (m, 1H), 6.82 (d, J=8.6 Hz, 2H), 6.29 (brt, 1H, NH), 5.55 (brd, 1H, NH), 5.51 (s, 1H), 4.49-4.29 (m, 3H), 4.20 (q, J=7.1 Hz, 2H), 4.14-4.05 (m, 1H), 3.93-3.86 (m, 1H), 3.79-3.70 (m, 1H), 3.51-3.42 (m, 1H), 3.13-3.06 (m, 1H), 2.94-2.85 (m, 2H), 2.65-2.58 (m, 2H), 2.31-2.20 (m, 1H), 1.35 (s, 9H), 1.24 (t, J=7.1 Hz, 3H), 1.07 (d, J=7.0 Hz, 3H), 1.04 (d, J=7.0 Hz, 3H).

### Step 5: Preparation of (S)-2-(4-(((R)-3-((R)-3-(tert-butoxycarbonylamino)-4-(2,4,5-trifluorophenyl)butanoyl)thiazolidine-2-carboxamido)methyl)phenoxy)-3-methylbutanoic acid

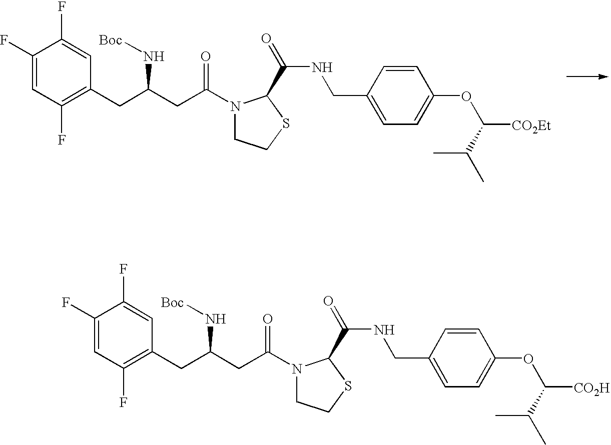

(S)-2-(4-(((R)-3-((R)-3-(tert-butoxycarbonylamino)-4-(2,4,5-trifluorophenyl)butanoyl)thiazolidine-2-carboxamido)methyl)phenoxy)-3-methylbutanoic acid is obtained according to the procedure used for Step 10, Example 22 except (S)-ethyl 2-(4-(((R)-3-((R)-3-(tert-butoxycarbonylamino)-4-(2,4,5-trifluorophenyl)butanoyl)thiazolidine-2-carboxamido)methyl)phenoxy)-3-methylbutanoate is used instead of (S)-ethyl 2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoate (96%).

¹H NMR (300 MHz, CDCl₃) 7.14-7.03 (m, 3H), 6.92-6.76 (m, 4H), 5.52 (s, 1H), 5.43 (brd, 1H, NH), 4.34 (d, J=7.8 Hz, 2H), 4.32-4.20 (m, 2H), 4.10-4.00 (m, 1H), 3.96-3.88 (m, 1H), 3.76-3.64 (m, 1H), 3.49-3.40 (m, 1H), 3.08-3.01 (m, 1H), 2.87-2.74 (m, 2H), 2.60-2.52 (m, 2H), 2.33-2.23 (m, 1H), 1.34 (s, 9H), 1.08 (d, J=6.5 Hz, 3H), 1.07 (d, J=6.5 Hz, 3H).

### Step 6: Preparation of (S)-2-(4-(((R)-3-((R)-3-amino-4-(2,4,5-trifluorophenyl)butanoyl)thiazolidine-2-carboxamido)methyl)phenoxy)-3-methylbutanoic acid.HCl

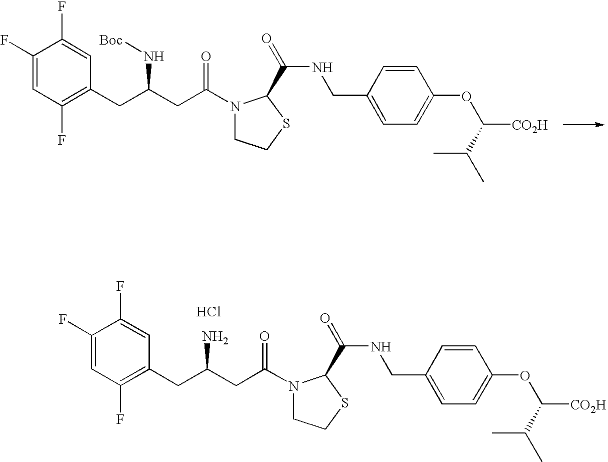

(S)-2-(4-(((R)-3-((R)-3-amino-4-(2,4,5-trifluorophenyl)butanoyl)thiazolidine-2-carboxamido)methyl)phenoxy)-3-methylbutanoic acid.HCl is obtained according to the procedure used for Step 11, Example 22 except (S)-2-(4-(((R)-3-((R)-3-(tert-butoxycarbonylamino)-4-(2,4,5-trifluorophenyl)butanoyl)thiazolidine-2-carboxamido)methyl)phenoxy)-3-methylbutanoic acid is used instead of (S)-2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoic acid (64%).

¹H NMR (300 MHz, DMSO-d₆) 12.94 (brs, 1H), 8.54 (brt, 1H, NH), 8.15 (brs, 3H, NH₂.HCl), 7.62-7.50 (m, 2H), 7.15 (d, J=8.6 Hz, 2H), 6.81 (d, J=8.6 Hz, 2H), 5.35 (s, 1H), 4.42 (d, J=5.0 Hz, 1H), 4.26-4.09 (m, 2H), 3.93-3.65 (m, 3H), 3.28-2.84 (m, 4H), 2.76-2.70 (m, 2H), 2.23-2.12 (m, 1H), 1.00 (d, J=6.8 Hz, 6H); LC-MS; 554 (M⁺+1).

### Example 25

### Preparation of (R)-2-(4-(((R)-3-((R)-3-amino-4-(2,4,5-trifluorophenyl)butanoyl)thiazolidine-2-carboxamido)methyl)phenoxy)-3-methylbutanoic acid.HCl

### Step 1: Preparation of (R)-ethyl 2-(4-(((R)-3-((R)-3-(tert-butoxycarbonylamino)-4-(2,4,5-trifluorophenyl)butanoyl)thiazolidine-2-carboxamido)methyl)phenoxy)-3-methylbutanoate

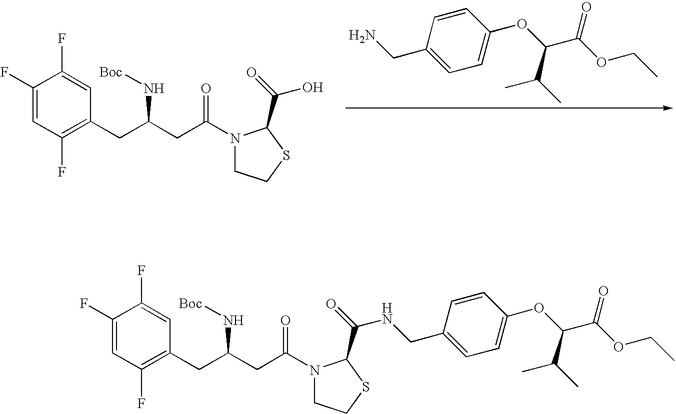

(R)-ethyl 2-(4-(((R)-3-((R)-3-(tert-butoxycarbonylamino)-4-(2,4,5-trifluorophenyl)butanoyl)thiazolidine-2-carboxamido)methyl)phenoxy)-3-methylbutanoate is obtained according to the procedure used for Step 9, Example 22 except (R)-3-((R)-3-(tert-butoxycarbonylamino)-4-(2,4,5-trifluorophenyl)butanoyl)thiazolidine-2-carboxylic acid is used instead of (S)-3-((R)-3-(tert-butoxycarbonylamino)-4-(2,4,5-trifluorophenyl)butanoyl)thiazolidine-2-carboxylic acid (75%).

¹H NMR (300 MHz, CDCl₃) 7.19 (d, J=8.6 Hz, 2H), 7.18-7.08 (m, 1H), 6.92-6.82 (m, 1H), 6.82 (d, J=8.6 Hz, 2H), 6.32 (brt, 1H, NH), 5.55 (brd, 1H, NH), 5.52 (s, 1H), 4.48-4.29 (m, 3H), 4.20 (q, J=7.1 Hz, 2H), 4.13-4.06 (m, 1H), 3.93-3.86 (m, 1H), 3.79-3.71 (m, 1H), 3.51-3.42 (m, 1H), 3.13-3.06 (m, 1H), 2.92-2.87 (m, 2H), 2.63-2.60 (m, 2H), 2.31-2.20 (m, 1H), 1.36 (s, 9H), 1.24 (t, J=7.1 Hz, 3H), 1.07 (d, J=7.0 Hz, 3H), 1.04 (d, J=7.0 Hz, 3H).

### Step 2: Preparation of (R)-2-(4-(((R)-3-((R)-3-(tert-butoxycarbonylamino)-4-(2,4,5-trifluorophenyl)butanoyl)thiazolidine-2-carboxamido)methyl)phenoxy)-3-methylbutanoic acid

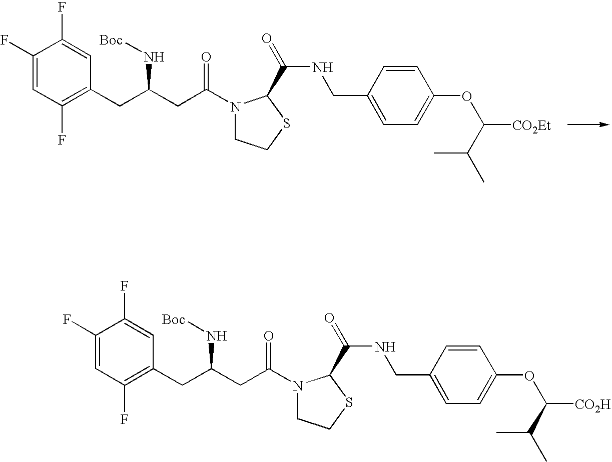

(R)-2-(4-(((R)-3-((R)-3-(tert-butoxycarbonylamino)-4-(2,4,5-trifluorophenyl)butanoyl)thiazolidine-2-carboxamido)methyl)phenoxy)-3-methylbutanoic acid is obtained according to the procedure used for Step 10, Example 22 except (R)-ethyl 2-(4-(((R)-3-((R)-3-(tert-butoxycarbonylamino)-4-(2,4,5-trifluorophenyl)butanoyl)thiazolidine-2-carboxamido)methyl)phenoxy)-3-methylbutanoate is used instead of (S)-ethyl 2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoate (96%).

¹H NMR (300 MHz, CDCl₃) 7.13-7.02 (m, 3H), 6.92-6.76 (m, 3H), 6.71 (brt, 1H), 5.48 (br, 1H), 5.47 (s, 1H), 4.40-4.24 (m, 3H), 4.10-4.00 (m, 1H), 3.89-3.80 (m, 1H), 3.73-3.63 (m, 1H), 3.47-3.37 (m, 1H), 3.06-2.99 (m, 1H), 2.88-2.72 (m, 2H), 2.56-2.50 (m, 2H), 2.35-2.24 (m, 1H), 1.34 (s, 9H), 1.10 (d, J=6.5 Hz, 3H), 1.08 (d, J=6.5 Hz, 3H).

### Step 3: Preparation of (R)-2-(4-(((R)-3-((R)-3-amino-4-(2,4,5-trifluorophenyl)butanoyl)thiazolidine-2-carboxamido)methyl)phenoxy)-3-methylbutanoic acid.HCl

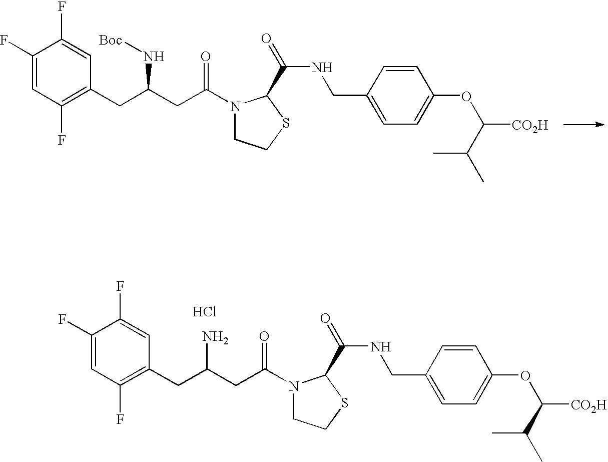

(R)-2-(4-(((R)-3-((R)-3-amino-4-(2,4,5-trifluorophenyl)butanoyl) thiazolidine-2-carboxamido)methyl)phenoxy)-3-methylbutanoic acid.HCl is obtained according to the procedure used for Step 11, Example 22 except (R)-2-(4-(((R)-3-((R)-3-(tert-butoxycarbonylamino)-4-(2,4,5-trifluorophenyl)butanoyl)thiazolidine-2-carboxamido)methyl)phenoxy)-3-methylbutanoic acid is used instead of (S)-2-(4-(((S)-3-((R)-3-(tert-butoxycarbonylamino)-4-(2,4,5-trifluorophenyl)butanoyl)thiazolidine-2-carboxamido)methyl)phenoxy)-3-methylbutanoic acid (79%).

¹H NMR (300 MHz, DMSO-d₆) 12.94 (brs, 1H), 8.54 (brt, 1H, NH), 8.15 (brs, 3H, NH₂.HCl), 7.62-7.50 (m, 2H), 7.16 (d, J=8.6 Hz, 2H), 6.83 (d, J=8.6 Hz, 2H), 5.36 (s, 1H), 4.44 (d, J=5.0 Hz, 1H), 4.27-4.10 (m, 2H), 3.93-3.66 (m, 3H), 3.28-2.84 (m, 4H), 2.76-2.70 (m, 2H), 2.23-2.12 (m, 1H), 1.01 (d, J=6.8 Hz, 6H); LC-MS; 554 (MH⁺).

### Example 26

### Preparation of (S)-2-(4-(((S)-3-((R)-3-amino-4-(2,4,5-trifluorophenyl)butanoyl)thiazolidine-2-carboxamido)methyl)phenylamino)-3-methylbutanoic acid.HCl

### Step 1: Preparation of (S)-ethyl 2-(4-cyanophenylamino)-3-methylbutanoate

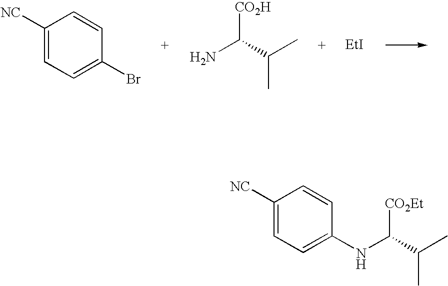

4-Bromobenzonitrile (1 g, 5.5 mmol), L-valine (773 mg, 6.6 mmol), K₃PO₄ (1.749 g, 8.25 mmol) or K₂CO₃ (1.139 g, 8.25 mmol) and copper (I) iodide (210 mg, 20 mol %) are added to dimethylacetamide (15 ml) in a pressure tube, followed by being reacted for 48 hours at 90° C. under nitrogen atmosphere. The reaction mixture is placed in a round flask, to which acetone (30 ml), K₂CO₃ (1.139 g, 8.25 mmol) and ethyl iodide (EtI, 1.716 g, 11 mmol) are added. The mixture is stirred for 2 hours while heated. The resultant is cooled and filtered. The filtrate is neutralized with dilute HCl, washed with brine and extracted with ethyl acetate twice. The entire extracts are dried over anhydrous MgSO₄ and concentrated. The residue is purified by column chromatography to obtain the compound, (S)-ethyl 2-(4-cyanophenylamino)-3-methylbutanoate (1.083 g, 80%).

### Step 2: Preparation of (S)-ethyl 2-(4-((tert-butoxycarbonylamino)methyl)phenylamino)-3-methylbutanoate

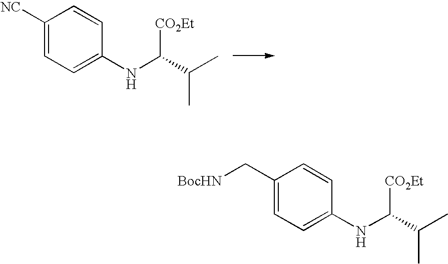

(S)-ethyl 2-(4-cyanophenylamino)-3-methylbutanoate (791 mg, 3.2 mmol) obtained in step 1 above is dissolved in ethanol (20 ml) in a 100 ml round flask. Thereto, nickel (II) chloride (879 mg, 3.2 mmol) is added and cooled with ice water. The reaction mixture is vigorously stirred with slow addition of NaBH₄ (FW; 37.83, 364 mg, 9.63 mmol). The resulting mixture is stirred for 20 minutes at room temperature, filtered through celite and concentrated. The residue is suspended in a mixture of acetone (20 ml) and water (10 ml). Thereto, NaHCO₃ (809 g, 9.63 mmol) and di-t-butyldicarbonate (840 mg, 3.85 mmol) are added, followed by stirring for 3 hours at room temperature. The resulting mixture is extracted with ethyl acetate. The organic layer is dried over anhydrous MgSO₄ and concentrated. The residue is purified by column chromatography to obtain the compound, (S)-ethyl 2-(4-((tert-butoxycarbonylamino)methyl)phenylamino)-3-methylbutanoate (867 mg, 77%) as a pale yellow solid.

¹H NMR (300 MHz, CDCl₃) 7.08 (d, J=8.3 Hz, 2H), 6.59 (d, J=8.3 Hz, 2H), 4.70 (br, 1H), 4.24-4.11 (m, 4H), 3.82 (dd, J=9.5, 5.8 Hz, 1H), 2.16-2.05 (m, 1H), 1.45 (s, 9H), 1.25 (t, J=7.1 Hz, 3H), 1.04 (d, J=6.8 Hz, 3H), 1.01 (d, J=6.8 Hz, 3H).

### Step 3: Preparation of (S)-ethyl 2-(4-(aminomethyl)phenylamino)-3-methylbutanoate.HCl

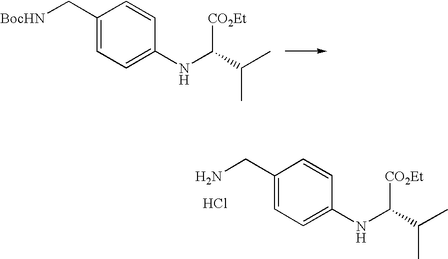

(S)-ethyl 2-(4-((tert-butoxycarbonylamino)methyl)phenylamino)-3-methylbutanoate (350 mg, 1 mmol) obtained in step 2 above is dissolved in CH₂Cl₂ (20 ml). Thereto, a 4 M HCl/dioxane mixture (1 ml) is added, followed by stirring for 12 hours at room temperature. The resulting mixture is concentrated, to which diethyl ether (5 ml) and n-hexane (20 ml) are added. The mixture is subjected to sonication and left at room temperature. After the supernatant is separated out, the precipitate is dried to obtain the compound, (S)-ethyl 2-(4-(aminomethyl)phenylamino)-3-methylbutanoate.HCl.

### Step 4: Preparation of (S)-ethyl 2-(4-(((S)-3-((R)-3-(tert-butoxycarbonylamino)-4-(2,4,5-trifluorophenyl)butanoyl)thiazolidine-2-carboxamido)methyl)phenylamino)-3-methylbutanoate

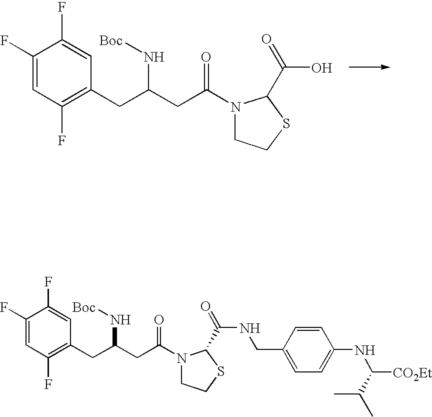

(S)-3-((R)-3-(tert-butoxycarbonylamino)-4-(2,4,5-trifluorophenyl)butanoyl)thiazolidine-2-carboxylic acid (580 mg, 1.29 mmol) and (S)-ethyl 2-(4-(aminomethyl)phenylamino)-3-methylbutanoate.HCl (480 mg, 1.5 mmol) obtained in step 3 above are suspended in CH₂Cl₂ (20 ml). Thereto, EDCI (523 mg, 2.72 mmol) and triethylamine (544 mg, 5.38 mmol) are slowly added, followed by stirring for 10 hours at room temperature. The resulting mixture, to which distilled water is added, extracted with CH₂Cl₂ twice. The entire extracts are dried over anhydrous MgSO₄ and concentrated. The residue is purified by column chromatography to obtain the compound, (S)-ethyl 2-(4-(((S)-3-((R)-3-(tert-butoxycarbonylamino)-4-(2,4,5-trifluorophenyl)butanoyl)thiazolidine-2-carboxamido)methyl)phenylamino)-3-methylbutanoate (497 mg, 70%).

¹H NMR (300 MHz, CDCl₃) 7.13-7.03 (m, 3H), 6.94-6.84 (m, 1H), 6.59 (d, J=8.4 Hz, 2H), 6.05 (brt, 1H), 5.58 (brd, 1H), 5.48 (s, 1H), 4.43-4.08 (m, 5H), 3.97-3.89 (m 1H), 3.82 (dd, J=9.3, 5.7 Hz, 1H), 3.76-3.68 (m, 1H), 3.53-3.44 (m, 1H), 3.13-3.06 (m, 1H), 2.90 (d, J=6.5 Hz, 2H), 2.63 (d, J=5.1 hz, 2H), 2.16-2.07 (m, 1H), 1.38 (s, 9H), 1.25 (t, J=7.1 Hz, 3H), 1.04 (d, J=6.9 Hz, 3H), 1.01 (d, J=6.9 Hz, 3H).

### Step 5: Preparation of (S)-2-(4-(((S)-3-((R)-3-(tert-butoxycarbonylamino)-4-(2,4,5-trifluorophenyl)butanoyl)thiazolidine-2-carboxamido)methyl)phenylamino)-3-methylbutanoic acid

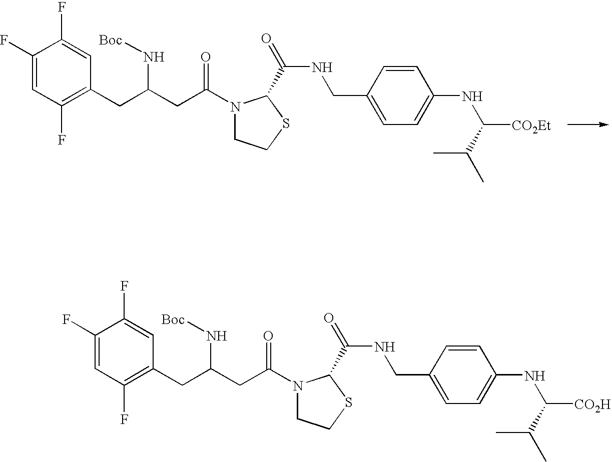

(S)-ethyl 2-(4-(((S)-3-((R)-3-(tert-butoxycarbonylamino)-4-(2,4,5-trifluorophenyl)butanoyl)thiazolidine-2-carboxamido)methyl)phenylamino)-3-methylbutanoate (661 mg, 1 mmol) obtained in step 4 above is dissolved in a mixture of THF (10 ml) and MeOH (10 ml). Thereto, LiOH.H₂O (420 mg) dissolved in distilled water (10 ml) is added, followed by stirring for 24 hours at room temperature. The resulting mixture is concentrated, cooled with ice water and acidified to a pH of 3 with 2 N HCl. The resultant is extracted with ethyl acetate. The entire extracts are dried over anhydrous sodium sulfite and concentrated to obtain the compound, (S)-2-(4-(((S)-3-((R)-3-(tert-butoxycarbonylamino)-4-(2,4,5-trifluorophenyl)butanoyl)thiazolidine-2-carboxamido)methyl)phenylamino)-3-methylbutanoic acid (620 mg, 95%).

¹H NMR (300 MHz, CDCl₃) 7.10-6.99 (m, 3H), 6.92-6.83 (m, 1H), 6.58 (d, J=8.4 Hz, 2H), 6.36 (br, 1H), 5.55 (brd, 1H), 5.46 (s, 1H), 4.36-4.18 (m, 2H), 4.13-4.01 (m, 1H), 3.92-3.85 (m, 1H), 3.80 (d, J=5.6 Hz, 1H), 3.72-3.64 (m, 1H), 3.49-3.40 (m, 1H), 3.07-3.00 (m, 1H), 2.98-2.70 (m, 2H), 2.60-2.47 (m, 2H), 2.11-2.10 (m, 1H), 1.36 (s, 9H), 1.06 (d, J=6.8 Hz, 3H), 1.05 (d, J=6.8 Hz, 3H).

### Step 6: Preparation of (S)-2-(4-(((S)-3-((R)-3-amino-4-(2,4,5-trifluorophenyl)butanoyl)thiazolidine-2-carboxamido)methyl)phenylamino)-3-methylbutanoic acid.HCl

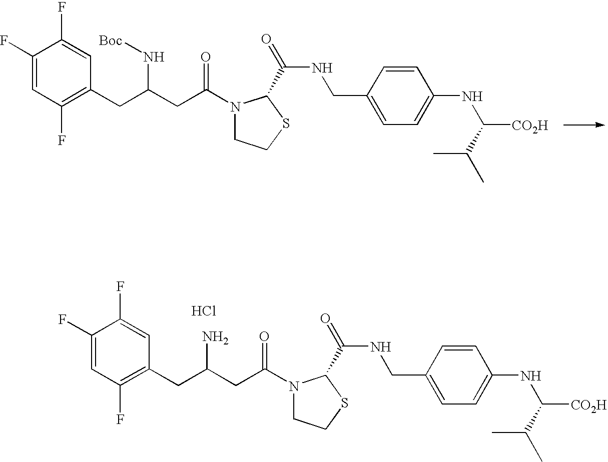

(S)-2-(4-(((S)-3-((R)-3-(tert-butoxycarbonylamino)-4-(2,4,5-trifluorophenyl)butanoyl)thiazolidine-2-carboxamido)methyl)phenylamino)-3-methylbutanoic acid (652 mg, 1 mmol) obtained in step 4 above is dissolved in CH₂Cl₂ (20 ml). Thereto, a 4 M-HCl/dioxane mixture (1.5 ml) is added, followed by stirring for 12 hours at room temperature. The resulting mixture is completely concentrated and recrystallized with diethyl ether added in a small amount. After the supernatant is separated out, the resulting white solid is dried to obtain the desired compound, (S)-2-(4-(((S)-3-((R)-3-amino-4-(2,4,5-trifluorophenyl)butanoyl)thiazolidine-2-carboxamido)methyl)phenylamino)-3-methylbutanoic acid.HCl (472 mg, 80%).

¹H NMR (300 MHz, DMSO-d₆) 8.33 (brt, 1H), 8.08 (brs, 3H), 7.60-7.48 (m, 2H), 6.98-6.91 (m, 2H), 6.61-6.54 (m, 2H), 5.37 (s, 1H), 4.12-4.05 (m, 2H), 3.95-3.87 (m, 1H), 3.78-3.55 (m, 3H), 3.24-3.11 (m, 2H), 3.04-2.91 (m, 2H), 2.79-2.69 (m, 2H), 2.06-1.96 (m, 1H), 0.97 (d, J=6.7 Hz, 3H), 0.94 (d, J=6.7 Hz, 3H).

### Example 27

### Preparation of (R)-2-(4-(((S)-3-((R)-3-amino-4-(2,4,5-trifluorophenyl)butanoyl)thiazolidine-2-carboxamido)methyl)phenylamino)-3-methylbutanoic acid.HCl

### Step 1: Preparation of (R)-ethyl 2-(4-(aminomethyl)phenylamino)-3-methylbutanoate.HCl

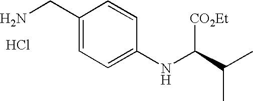

(R)-ethyl 2-(4-(aminomethyl)phenylamino)-3-methylbutanoate.HCl is obtained according to the procedure used for Step 1 to 3, Example 26.

### Step 2: Preparation of (R)-ethyl 2-(4-(((S)-3-((R)-3-(tert-butoxycarbonylamino)-4-(2,4,5-trifluorophenyl)butanoyl)thiazolidine-2-carboxamido)methyl)phenylamino)-3-methylbutanoate

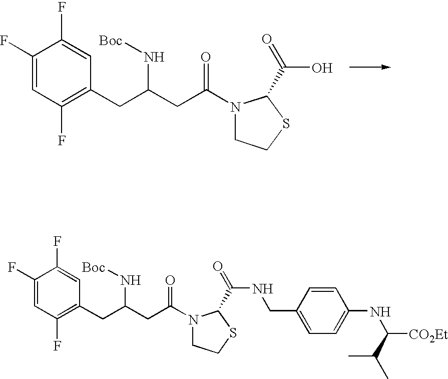

(R)-ethyl 2-(4-(((S)-3-((R)-3-(tert-butoxycarbonylamino)-4-(2,4,5-trifluorophenyl)butanoyl)thiazolidine-2-carboxamido)methyl)phenylamino)-3-methylbutanoate is obtained according to the procedure used for Step 4, Example 26 (67%) except (R)-ethyl 2-(4-(aminomethyl)phenylamino)-3-methylbutanoate.HCl is used instead of (S)-ethyl 2-(4-(aminomethyl)phenylamino)-3-methylbutanoate.HCl.

¹H NMR (300 MHz, CDCl₃) 7.12-7.03 (m, 3H), 6.93-6.84 (m, 1H), 6.59 (d, J=8.4 Hz, 2H), 6.01 (brt, 1H), 5.58 (brd, 1H), 5.48 (s, 1H), 4.43-4.08 (m, 5H), 3.97-3.90 (m 1H), 3.83 (dd, J=9.3, 5.7 Hz, 1H), 3.77-3.66 (m, 1H), 3.53-3.44 (m, 1H), 3.13-3.06 (m, 1H), 2.91 (d, J=6.5 Hz, 2H), 2.63 (d, J=5.1 hz, 2H), 2.16-2.07 (m, 1H), 1.38 (s, 9H), 1.25 (t, J=7.1 Hz, 3H), 1.04 (d, J=6.9 Hz, 3H), 1.01 (d, J=6.9 Hz, 3H).

### Step 3: Preparation of (R)-2-(4-(((S)-3-((R)-3-(tert-butoxycarbonylamino)-4-(2,4,5-trifluorophenyl)butanoyl)thiazolidine-2-carboxamido)methyl)phenylamino)-3-methylbutanoic acid

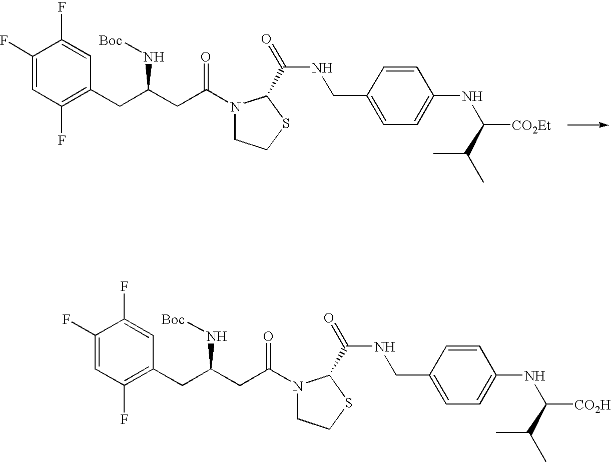

(R)-2-(4-(((S)-3-((R)-3-(tert-butoxycarbonylamino)-4-(2,4,5-trifluorophenyl)butanoyl)thiazolidine-2-carboxamido)methyl)phenylamino)-3-methylbutanoic acid is obtained according to the procedure used for Step 5, Example 26 except (R)-ethyl 2-(4-(((S)-3-((R)-3-(tert-butoxycarbonylamino)-4-(2,4,5-trifluorophenyl)butanoyl)thiazolidine-2-carboxamido)methyl)phenylamino)-3-methylbutanoate is used instead of (S)-ethyl 2-(4-(((S)-3-((R)-3-(tert-butoxycarbonylamino)-4-(2,4,5-trifluorophenyl)butanoyl)thiazolidine-2-carboxamido)methyl)phenylamino)-3-methylbutanoate (99%).

### Step 4: Preparation of (R)-2-(4-(((S)-3-((R)-3-amino-4-(2,4,5-trifluorophenyl)butanoyl)thiazolidine-2-carboxamido)methyl)phenylamino)-3-methylbutanoic acid.HCl

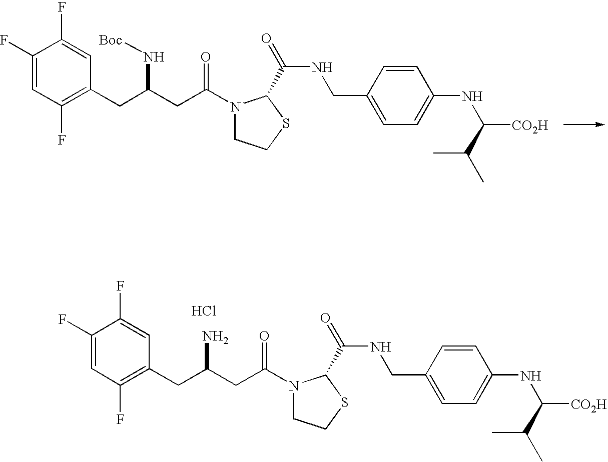

(R)-2-(4-(((S)-3-((R)-3-amino-4-(2,4,5-trifluorophenyl)butanoyl)thiazolidine-2-carboxamido)methyl)phenylamino)-3-methylbutanoic acid.HCl is obtained according to the procedure used for Step 6, Example 26 except (R)-2-(4-(((S)-3-((R)-3-(tert-butoxycarbonylamino)-4-(2,4,5-trifluorophenyl)butanoyl)thiazolidine-2-carboxamido)methyl)phenylamino)-3-methylbutanoic acid is used instead of (S)-2-(4-(((S)-3-((R)-3-(tert-butoxycarbonylamino)-4-(2,4,5-trifluorophenyl)butanoyl)thiazolidine-2-carboxamido)methyl)phenylamino)-3-methylbutanoic acid (96%).

¹H NMR (300 MHz, DMSO-d₆) 8.36 (brt, 1H, NH), 8.15 (brs, 3H, NH₂.HCl), 7.61-7.46 (m, 2H), 6.99-6.93 (m, 2H), 6.63-6.56 (m, 2H), 5.37 (s, 1H), 4.13-4.05 (m, 2H), 3.96-3.89 (m, 1H), 3.78-3.55 (m, 3H), 3.23-3.13 (m, 2H), 3.03-2.95 (m, 2H), 2.80-2.72 (m, 2H), 2.07-1.97 (m, 1H), 0.97 (d, J=6.8 Hz, 3H), 0.94 (d, J=6.8 Hz, 3H).

Various 2-thiazolidine derivatives having β-amino group represented by formula 1 were obtained by the procedures of Examples 1 to 27, and their structures and characteristic properties (NMR or Mass spectrum data) are shown in Table 1.

TABLE 1

| Ex | Structure | Data |
|---|---|---|
| 1 | 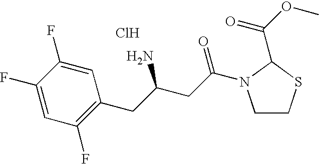 | LC-MS m/e 363 (MH⁺). |
| 2 | 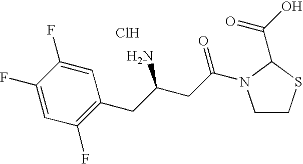 | LC-MS m/e 349 (MH⁺). |
| 3 | 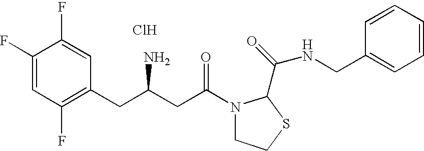 | (CD₃OD, 300 MHz) δ 7.41-7.22 (m, 7H), 5.51 (d, J = 10.8 Hz, 1H), 5.00-4.60 (m, 1H), 4.39 (s, 2H), 4.02-3.98 (m, 1H), 3.88-3.81 (m, 2H), 3.40-3.19 (m, 2H), 3.08-3.03 (m, 2H), 2.85-2.79 (m, 2H). |
| 4 | 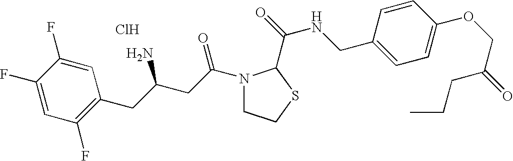 | (CD₃OD, 300 MHz) δ 7.23-7.17 (m, 1H), 7.12-7.03 (m, 3H), 6.73-6.68 (m, 2H), 5.30 (d, J = 13.3 Hz, 1H), 4.73-4.57 (m, 1H), 4.50 (s, 2H), 4.10 (s, 2H), 4.06 (q, J = 7.2 Hz, 2H), 3.90-3.80 (m, 1H), 3.69-3.64 (m, 2H), 3.15-3.13 (m, 2H), 3.02-3.00 (m, 1H), 3.00- 2.89 (m, 1H), 2.80-2.70 (m, 1H), 1.11 (t, J = 7.2 Hz, 3H) |
| 5 | 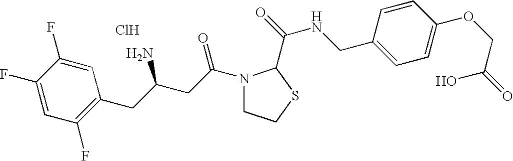 | (CD₃OD, 300 MHz) δ 7.40-7.20 (m, 1H), 7.18-7.13 (m, 3H), 6.83-6.80 (m, 2H), 5.40 (d, J = 13.4 Hz, 1H), 4.56 (s, 2H), 4.24 (s, 2H), 4.00-3.80 (m, 1H), 3.80-3.70 (m, 2H), 3.25-3.23 (m, 1H), 3.20-3.05 (m, 1H), 2.99-2.97 (m, 2H), 2.80-2.60 (m, 1H) |
| 6 | 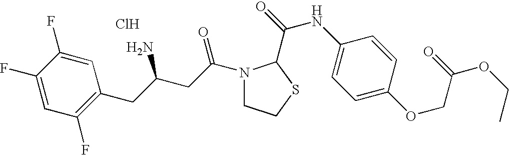 | (CD₃OD, 300 MHz) δ 7.36 (d, J = 9.0 Hz, 2H), 7.34-7.29 (m, 1H), 7.16-7.13 (m, 1H), 6.81 (d, J = 9.0 Hz, 2H), 5.48 (d, J = 14.0 Hz, 1H), 4.60 (s, 2H), 4.14 (q, J = 7.2 Hz, 2H), 4.00-3.80 (m, 1H), 3.77- 3.73 (m, 2H), 3.38-3.28 (m, 1H), 3.21-3.13 (m, 2H), 2.98-2.97 (m, 2H), 2.80-2.76 (m, 1H), 1.18 (t, J = 7.2 Hz, 3 H) |
| 7 | 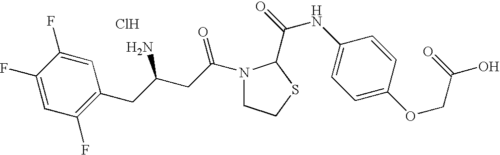 | (DMSO-d₆, 300 MHz) δ 8.10 (brs, 3H), 7.56-7.51 (m, 2H), 7.46 (d, J = 7.8 Hz, 2H), 6.88 (d, J = 7.8 Hz, 2H), 5.52 (d, J = 12.0 Hz, 1H), 4.72 (s, 2H), 4.01-3.69 (m, 4H), 2.98-2.64 (m, 5H) |
| 8 | 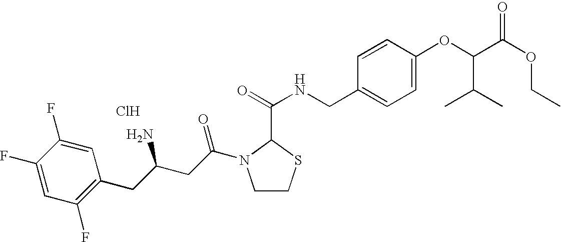 | (DMSO-d₆, 300 MHz) δ 8.59~ 8.51 (m, 1H), 8.21 (brs, 3H), 7.63~7.50 (m, 2H), 7.17~7.13 (m, 2H), 6.87~6.78 (m, 2H), 5.47~5.35 (m, 2H), 4.54~ 4.50 (m, 1H), 4.21~4.10 (m, 4H), 4.00~3.71 (m, 3H), 3.23~2.76 (m, 5H), 2.30~2.00 (m, 1H), 1.17 (t, J = 7.1 Hz, 3H), 1.00~0.98 (m, 6H) |
| 9 | 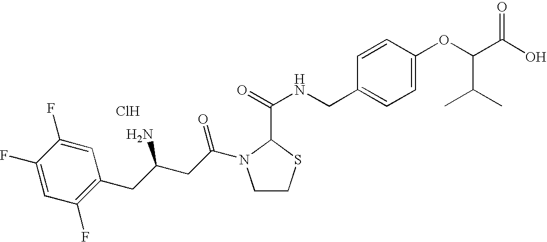 | (DMSO-d₆, 300 MHz) δ 12.91 (br, 1H), 8.59 (br, 1H), 7.98 (brs, 3H), 7.53~7.50 (m, 2H), 7.13~7.11 (m, 2H), 6.80~6.75 (m, 2H), 5.37~5.33 (m, 1H), 4.40~4.38 (m, 1H), 4.20~4.12 (m, 3H), 3.83~3.68 (m, 3H), 2.92~2.85 (m, 2H), 2.69~2.60 (m, 1H), 2.24~2.14 (m, 1H), 0.97 (d, J = 6.6Hz, 6H) |
| 10 | 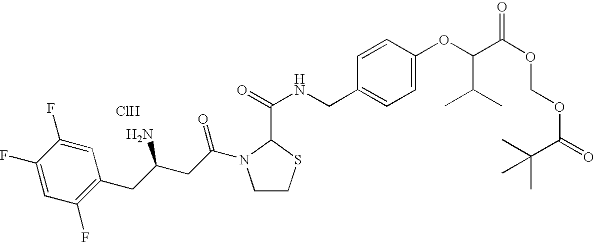 | (DMSO-d₆, 300 MHz) δ 8.55~ 8.49 (m, 1H), 8.13 (brs, 3H), 7.59~7.53 (m, 3H), 7.16~7.12 (m, 3H), 5.81 (d, J = 5.8Hz, 1H), 5.73 (d, J = 5.8 Hz, 1H), 5.40~5.36 (m, 1H), 4.72~4.63 (m, 2H), 4.19~4.15 (m, 3H), 4.00~3.71 (m, 3H), 3.20~3.17 (m, 2H), 3.00~2.93 (m, 1H), 2.79~2.76 (m, 1H), 2.30~2.17 (m, 1H), 1.12 (s, 9H), 1.00~0.98 (m, 6H) |
| 11 | 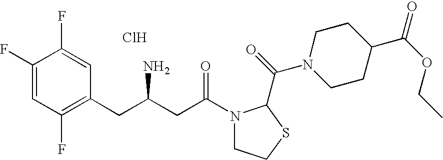 | (DMSO-d₆, 300 MHz) δ 7.81 (brs, 3H), 7.46~7.37 (m, 2H), 6.37 (br, 1H), 4.26 (q, J = 7.0 Hz, 2H), 3.89~3.30 (m, 4H), 3.05~2.58 (m, 13H), 1.23 (t, J = 7.0 Hz, 3H) |
| 12 | 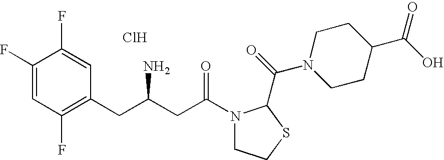 | (DMSO-d₆, 300 MHz) δ 8.09 (brs, 3H), 7.69~7.60 (m, 2H), 6.03~6.00 (m, 1H), 4.20~4.15 (m, 1H), 3.94~3.79 (m, 2H), 3.41~3.30 (m, 4H), 3.29~2.82 (m, 8H), 2.11~1.99 (m, 1H), 1.80~1.30 (m, 1H) |
| 13 | 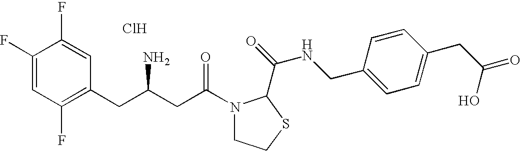 | (DMSO-d₆, 300 MHz) δ 8.54 (br, 1H), 8.01 (brs, 3H), 7.60~ 7.51 (m, 2H), 7.21~7.18 (m, 4H), 4.32~4.25 (m, 3H), 3.80~ 3.53 (m, 7H), 3.00~2.80 (m, 2H), 2.74~2.73 (m, 2H) |
| 14 | 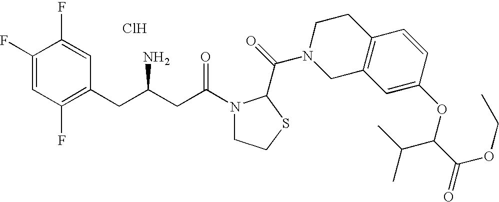 | (CD₃OD, 300 MHz) δ 7.41~ 7.19 (m, 2H), 7.05~7.02 (m, 1H), 6.72~6.63 (m, 2H), 6.00~ 5.96 (m, 1H), 4.87~4.41 (m 5H), 4.17~4.14 (m, 2H), 3.89~3.61 (m, 6H), 3.25~2.66 (m, 7H), 2.21~2.10 (m, 1 H), 1.99 (t, J = 7.2 Hz, 3H), 0.83~ 0.80 (m, 6H) |
| 15 | 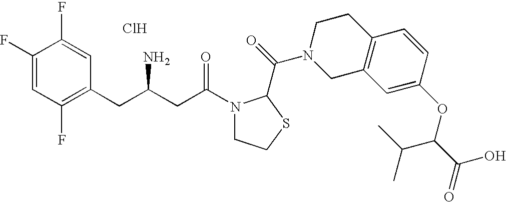 | (DMSO-d₆, 300 MHz) δ 12.93 (br, 1H), 8.05 (brs, 3H), 7.61~ 7.54 (m, 2H), 7.10~7.08 (m, 1H), 6.73~6.71 (m, 2H), 6.18~5.99 (m, 1H), 4.53~4.45 (m, 4H), 3.86~3.57 (m, 6H), 3.20~2.74 (m, 6H), 2.20~2.00 (m, 1H), 1.07~0.99 (m, 6H) |
| 16 | 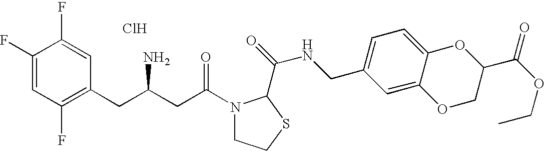 | (CD₃OD, 300 MHz) δ 7.33~ 7.19 (m, 2H), 6.86~6.73 (m, 3H), 4.89~4.74 (m, 7 H), 4.35~ 4.30 (m, 1H), 4.27~4.15 (m, 4H), 4.00~3.90 (m, 1H), 3.79~ 3.62 (m, 2H), 3.21~3.00 (m, 2H), 2.80~2.60 (m, 2H), 1.22 (t, J = 7.1 Hz, 3H) |
| 17 | 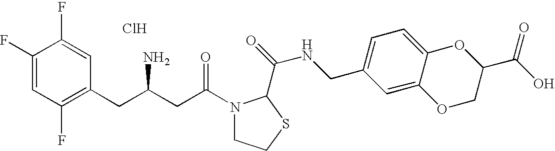 | (DMSO-d₆, 300 MHz) δ 13.30 (br, 1H), 8.08 (br, 3H), 7.58~ 7.52 (m, 2H), 6.87~6.73 (m, 3H), 5.41~5.37 (m, 1H), 5.02~5.00 (m, 1H), 4.40~4.30 (m, 1H), 4.23~3.57 (m, 8H), 3.20~3.00 (m, 2H), 2.99~2.80 (m, 2H) |
| 18 | 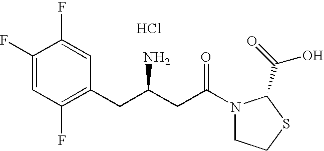 | (DMSO-d₆, 300 MHz) δ 13.08 (br, 1H), 8.06 (br, 3H), 7.61-7.48 (m, 2H), 5.28 (s, 1H), 3.95-3.59 (m, 3H), 3.23-3.16 (m, 2H), 3.08-2.67 (m, 4H). |
| 19 | 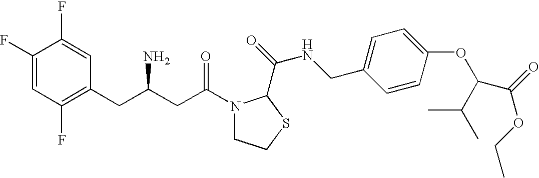 | (CDCl₃ 300 MHz) δ 7.21~7.09 (m, 3H), 6.91~6.82 (m, 3H), 6.72~6.69 (m, 1H), 6.25 (br, 1H), 6.00~5.92 (m, 1H), 5.49 (d, J = 6.3 Hz, 1H), 4.37~4.17 (m, 6H), 4.00~3.83 (m, 1H), 3.80~3.65 (m, 1H), 3.55~3.40 (m, 1H), 3.26~2.82 (m, 3H), 2.75~2.50 (m, 2H), 2.40~2.20 (m, 1H), 2.03 (s, 3H), 1.43~1.40 (m, 3H), 1.25 (t, J = 7.2 Hz, 3H), 1.07~1.04 (m, 6H) |
| 20 | 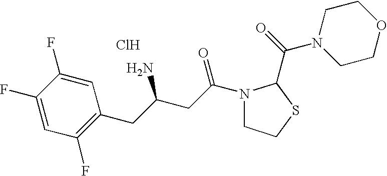 | (CD₃OD, 300 MHz) δ 7.35-7.30 (m, 1H), 7.24-7.18 (m, 1H), 5.89 (d, J = 14.0 Hz, 1H), 3.86- 3.80 (m, 2H), 3.66-3.40 (m, 7H), 3.29-3.25 (m, 4H), 3.06-3.00 (m, 2H), 2.84-2.64 (m, 2H) |
| 21 | 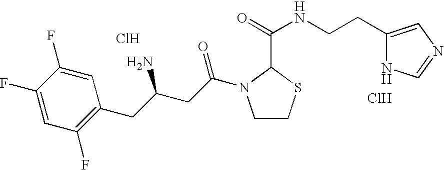 | (DMSO-d₆, 300 MHz) δ 9.01 (s, 1H), 8.33-8.07 (m, 1H), 7.64-7.49 (m, 1H), 7.40 (s, 1H), 5.25 (d, J = 11.7 Hz, 1H), 3.71-3.57 (m, 1H), 3.16-3.14 (m, 2H), 3.02-2.78 (m, 8H) |
| 22 | 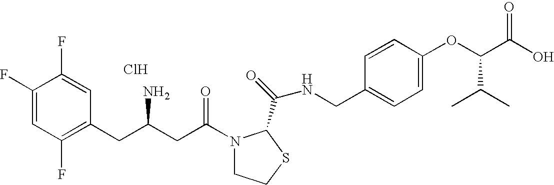 | (DMSO-d₆, 300 MHz) δ 12.96 (brs, 1H), 8.48 (brt, 1H, NH), 8.07 (brs, 3H, NH₂•HCl), 7.61- 7.51 (m, 2H), 7.13 (d, J = 8.6 Hz, 2H), 6.79 (d, J = 8.6 Hz, 2H), 5.40 (s, 1H), 4.40 (d, J = 5.0 Hz, 1H), 4.24-4.12 (m, 2H), 3.99-3.92 (m, 1H), 3.80- 3.66 (m, 2H), 3.24-3.16 (m, 2H), 3.00-2.94 (m, 2H), 2.78-2.72 (m, 2H), 2.22-2.14 (m, 1H), 1.00 (d, J = 6.7 Hz, 6H) |
| 23 | 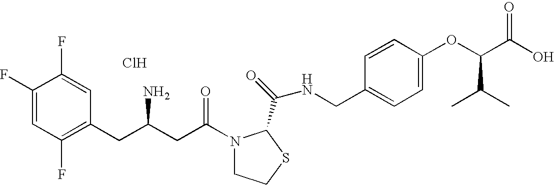 | (DMSO-d₆, 300 MHz) δ 8.48 (brt, 1H, NH), 8.07 (brs, 3H, NH₂•HCl), 7.61-7.51 (m, 2H), 7.13 (d, J = 8.6 Hz, 2H), 6.79 (d, J = 8.6 Hz, 2H), 5.40 (s, 1H), 4.40 (d, J = 5.0 Hz, 1H), 4.24-4.12 (m, 2H), 3.99- 3.92 (m, 1H), 3.80-3.66 (m, 2H), 3.24-3.16 (m, 2H), 3.00-2.94 (m, 2H), 2.78-2.72 (m, 2H), 2.22-2.14 (m, 1H), 1.00 (d, J = 6.7 Hz, 6H) |
| 24 | 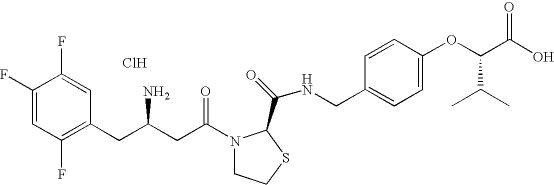 | ((DMSO-d₆, 300 MHz) δ 12.94 (brs, 1H), 8.54 (brt, 1H, NH), 8.15 (brs, 3H, NH₂•HCl), 7.62- 7.50 (m, 2H), 7.15 (d, J = 8.6 Hz, 2H), 6.81 (d, J = 8.6 Hz, 2H), 5.35 (s, 1H), 4.42 (d, J = 5.0 Hz, 1H), 4.26-4.09 (m, 2H), 3.93-3.65 (m, 3H), 3.28-2..84 (m, 4H), 2.76-2.70 (m, 2H), 2.23-2.12 (m, 1H), 1.00 (d, J = 6.8 Hz, 6H) |
| 25 | 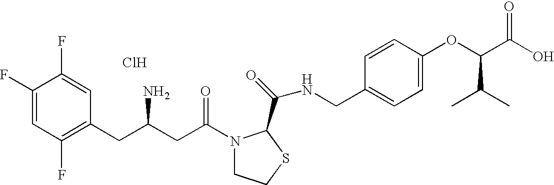 | ((DMSO-d₆, 300 MHz) δ 12.94 (brs, 1H), 8.54 (brt, 1H, NH), 8.15 (brs, 3H, NH₂•HCl), 7.62-7.50 (m, 2H), 7.16 (d, J = 8.6 Hz, 2H), 6.83 (d, J = 8.6 Hz, 2H), 5.36 (s, 1H), 4.44 (d, J = 5.0 Hz, 1H), 4.27-4.10 (m, 2H), 3.93-3.66 (m, 3H), 3.28-2.84 (m, 4H), 2.76-2.70 (m, 2H), 2.23-2.12 (m, 1H), 1.01 (d, J = 6.8 Hz, 6H) |
| 26 | 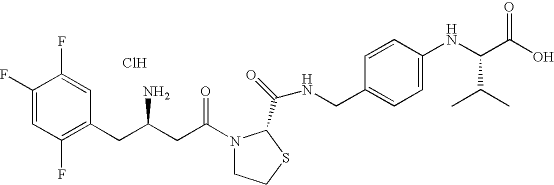 | (DMSO-d₆, 300 MHz) δ 8.36 (brt, 1H, NH), 8.15 (brs, 3H, NH₂•HCl), 7.61-7.46 (m, 2H), 6.99-6.93 (m, 2H), 6.63-6.56 (m, 2H), 5.37 (s, 1H), 4.13-4.05 (m, 2H), 3.96-3.89 (m, 1H), 3.78-3.55 (m, 3H), 3.23-3.13 (m, 2H), 3.03-2.95 (m, 2H), 2.80-2.72 (m, 2H), 2.07-1.97 (m, 1H), 0.98 (d, J = 6.8 Hz, 3H), 0.94 (d, J = 6.8 Hz, 3H). |
| 27 | 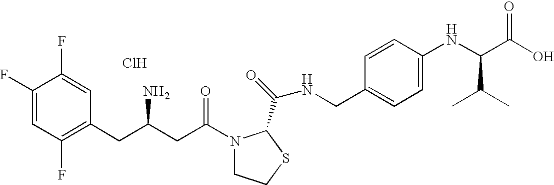 | (DMSO-d₆, 300 MHz) δ 8.36 (brt, 1H, NH), 8.15 (brs, 3H, NH₂•HCl), 7.61-7.46 (m, 2H), 6.99-6.93 (m, 2H), 6.63-6.56 (m, 2H), 5.37 (s, 1H), 4.13-4.05 (m, 2H), 3.96-3.89 (m, 1H), 3.78-3.55 (m, 3H) 3.23-3.13 (m, 2H), 3.03-2.95 (m, 2H), 2.80-2.72 (m, 2H), 2.07-1.97 (m, 1H), 0.97 (d, J = 6.8 Hz, 3H), 0.94 (d, J = 6.8 Hz, 3H). |
| 28 | 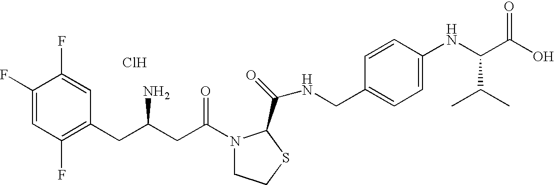 | (DMSO-d₆, 300 MHz) δ 8.40 (brt, 1H), 8.18 (br, 3H), 7.61- 7.48 (m, 2H), 6.96 (d, J = 8.4 Hz, 2H), 6.61-6.57 (m, 2H), 5.39 (br, 1H), 5.33 (s, 1H), 4.07 (qd, J = 15.7, 5.8 Hz, 2H), 3.94- 3.60 (m, 4H), 3.25-3.08 (m, 2H), 3.07-2.84 (m, 2H), 2.80- 2.67 (m, 2H), 2.07-1.97 (m, 1H), 0.98 (d, J = 6.8 Hz, 3H), 0.94 (d, J = 6.8 Hz, 3H). |
| 29 | 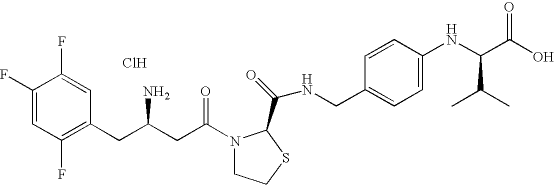 | (DMSO-d₆, 300 MHz) δ 8.41 (brt, 1H), 8.18 (brs, 3H), 7.61- 7.48 (m, 2H), 6.96 (d, J = 8.3 Hz, 2H), 6.59 (d, J = 8.3 Hz, 2H), 5.39 (br, 1H), 5.32 (s, 1H), 4.15-3.44 (m, 6H), 3.24-2.84 (m, 4H), 2.76-2.69 (m, 2H), 2.06-1.97 (m, 1H), 0.98 (d, J = 6.8 Hz, 3H), 0.95 (d, J = 6.8 Hz, 3H). |
| 30 | 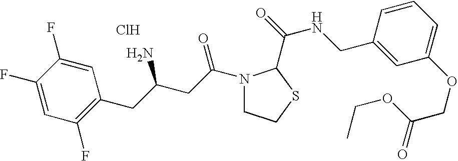 | (CD₃OD, 300 MHz) δ 7.48- 7.43 (m, 1H), 7.32-7.30 (m, 2H), 7.01-6.98 (m, 2H), 6.91- 6.89 (m, 1H), 5.08 (d, J = 11.7 Hz, 1H), 4.87 (s, 2H), 4.79-4.72 (m, 2H), 4.53 (s, 2H), 4.33 (q, J = 7.2 Hz, 2H), 4.10- 4.06 (m, 1H), 3.95-3.90 (m, 2H), 3.40-3.34 (m, 2H), 3.20-3.16 (m, 2H), 1.37 (t, J = 7.2 Hz, 3H) |
| 31 | 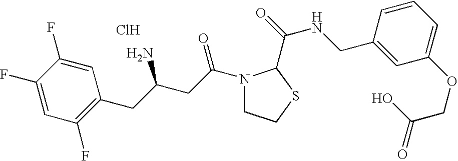 | (CD₃OD, 300 MHz) δ 7.98 (brs, 3H), 7.49-7.40 (m, 2H), 7.14-7.07 (m, 1H), 6.75-6.64 (m, 3H), 5.29 (d, J = 12.6 Hz, 1H), 4.62 (s, 2H), 4.09 (s, 2H), 3.66-3.58 (m, 4H), 3.15-3.07 (m, 2H), 2.88-2.86 (m, 1H), 2.65-2.61 (m, 2H). |
| 32 | 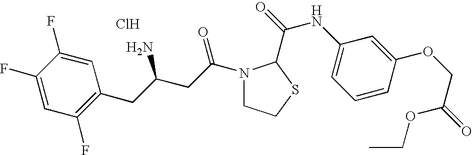 | (CD₃OD, 300 MHz) δ 7.15- 7.09 (m, 1H), 7.06-7.01 (m, 1H), 7.00-6.90 (m, 1H), 6.54- 6.44 (m, 1H), 5.38 (d, J = 12.8 Hz, 1H), 4.47 (s, 2H), 4.04 (q, J = 7.2 Hz, 2H), 3.85- 3.81 (m, 1H), 3.70-3.60 (m, 2H), 3.38-3.28 (m, 2H), 2.91- 2.57 (m, 4H), 1.08 (t, J = 7.2 Hz, 3H) |
| 33 | 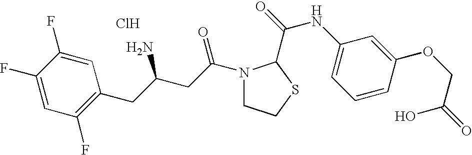 | (DMSO-d₆, 300 MHz) δ 8.11 (brs, 3H), 7.69-7.52 (m, 2H), 7.29-7.19 (m, 2H), 7.15-7.12 (m, 1H), 6.64-6.61 (m, 1H), 5.54 (d, J = 12.9 Hz, 1H), 4.63 (s, 2H), 4.12-4.05 (m, 2H), 3.83-3.70 (m, 2H), 3.01-2.74 (m, 5H). |
| 34 | 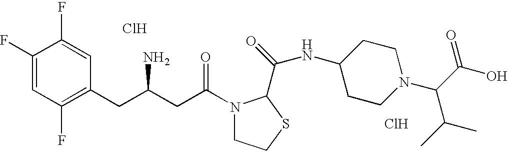 | (DMSO-d₆, 300 MHz) δ 8.05 (s, 1H), 7.56~7.53 (m, 2H), 5.32 (s, 1H), 4.57~4.55 (m, 1H), 3.84~3.64 (m, 8H), 3.18~3.16 (m, 1H), 2.98~2.89 (m, 8H), 2.72~2.70 (m, 1H), 2.20~2.00 (m, 1H), 1.80~1.60 (m, 2H), 0.94~0.87 (m, 6H) |
| 35 | 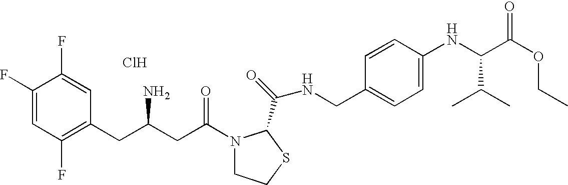 | (DMSO-d₆, 300 MHz) δ 8.37 (brt, 1H), 8.14 (brs, 3H), 7.62-7.51 (m, 2H), 7.00-6.93 (m, 2H), 6.60-6.53 (m, 2H), 5.37 (s, 1H), 4.08 (q, J = 7.1 Hz, 2H), 4.13-3.55 (m, 6H), 3.23-3.13 (m, 2H), 3.04-2.95 (m, 2H), 2.81-2.71 (m, 2H), 2.08-1.98 (m, 1H), 1.15 (t, J = 7.1 Hz, 3H), 0.99 (d, J = 6.7 Hz, 3H), 0.93 (d, J = 6.7 Hz, 3H) |
| 36 | 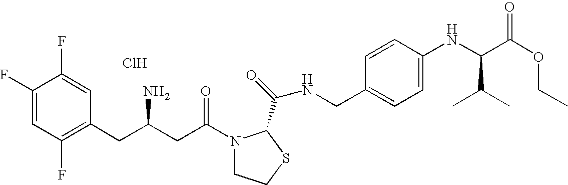 | (DMSO-d₆, 300 MHz) δ 8.33 (brt, 1H), 8.13 (brs, 3H), 7.61- 7.49 (m, 2H), 6.93-6.92 (m, 2H), 6.60-6.52 (m, 2H), 5.38 (s, 1H), 4.10-4.03 (m, 4H), 3.96-3.89 (m, 1H), 3.79-3.62 (m, 3H), 3.21-3.13 (m, 2H), 3.01-2.93 (m, 2H), 2.78-2.72 (m, 2H), 2.04-1.95 (m, 1H), 1.14 (t, J =7.1 Hz, 3H), 0.97 (d, J = 6.7 Hz, 3H), 0.92 (d, J = 6.7 Hz, 3H) |
| 37 | 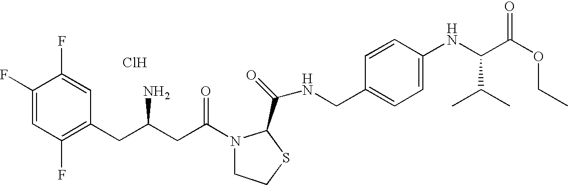 | (DMSO-d₆, 300 MHz) δ 8.37 (brt, 1H), 8.07 (br, 3H), 7.59- 7.48 (m, 2H), 6.94 (d, J = 8.4 Hz, 2H), 8.56-8.51 (m, 2H), 5.36 (br, 1H), 5.33 (s, 1H), 4.16 3.96 (m, 4H), 3.86-3.44 (m, 4H), 3.24-2.80 (m, 4H), 2.74- 2.66 (m, 2H), 2.06-1.96 (m, 1H), 0.97 (d, J = 6.7 Hz, 3H), 0.92 (d, J = 6.7 Hz, 3H) |
| 38 | 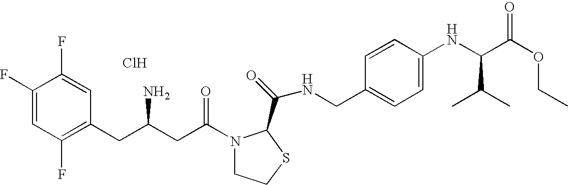 | (CD₃OD, 300 MHz) δ 7.34-7.24 (m, 1H), 7.19-7.09 (m, 3H), 6.95-6.84 (m, 2H), 5.34 (s, 1H), 4.24 (q. J =15 Hz, 2H), 4.12-4.04 (m, 2H), 3.94- 3.86 (m, 2H), 3.78-3.60 (m, 2H), 3.28-3.08 (m, 2H), 3.00- 2.88 (m, 2H), 2.76-2.62 (m, 2H), 2.14-2.04 (m, 1H), 1.12 (t, J = 7.1 Hz, 3H), 1.02 (d, 6.9 Hz, 3H), 0.96 (d, J = 6.9 Hz, 3H) |
| 39 | 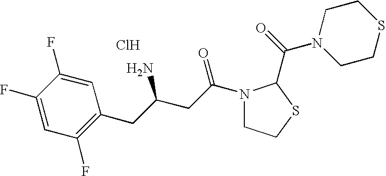 | (CD₃OD, 300 MHz) δ 7.38-7.15 (m, 2H), 5.90 (d, J = 14.6 Hz, 1H), 3.90-3.78 (m, 8H), 3.21- 3.02 (m, 1H), 3.00-2.78 (m, 2H), 2.70-2.57 (m, 6H) |
| 40 | 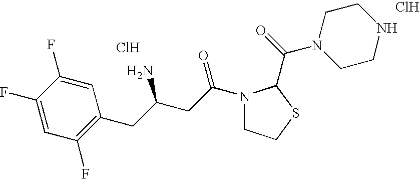 | (DMSO-d₆, 300 MHz) δ 8.14 (brs, 3H), 7.61-7.54 (m, 2H), 4.40-4.10 (m, 8H), 3.21-3.02 (m, 2H), 3.89-3.57 (m, 5H), 3.41-3.24 (m, 5H), 3.00-2.86 (m, 4H), 2.79 (s, 3H), 2.78-2.73 (m, 2H) |
| 41 | 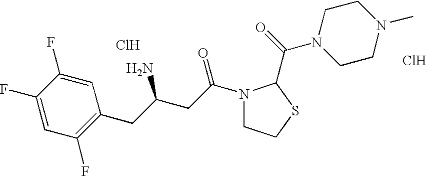 | (DMSO-d₆, 300 MHz) δ 8.21 (brs, 3H), 7.61-7.52 (m, 2H), 5.97 (d, J = 8.2 Hz, 1H), 3.84- 3.67 (m, 8H), 3.23-2.73 (m, 9H) |
| 42 | 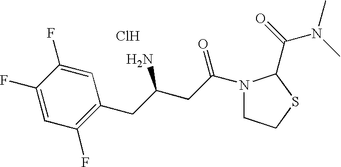 | (DMSO-d₆, 300 MHz) δ 8.13 (brs, 3H), 7.61-7.52 (m, 2H), 5.76 (s, 1H), 4.15-4.10 (m, 1H), 3.88-3.82 (m, 3H), 3.23-3.17 (m, 2H), 3.00 (s, 6H), 2.92-2.71 (m, 3H) |
| 43 | 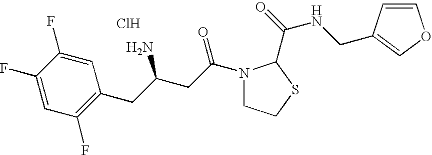 | (DMSO-d₆, 300 MHz) δ 8.14 (brs, 3H), 7.57 (s, 1H), 7.58- 7.54 (m, 2H), 6.39 (s, 1H), 7.23 (d, J = 7.8 Hz, 1H), 5.38 (d, J = 13.8 Hz, 1H), 4.24 (s, 2H), 3.93-3.70 (m, 3H), 3.22- 3.18 (m, 2H), 3.06-2.90 (m, 2H), 2.78-2.75 (m, 2H) |
| 44 | 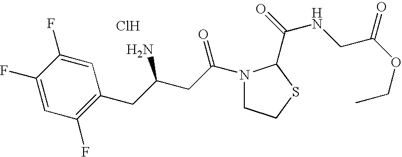 | (CD₃OD, 300 MHz) δ 8.19 (brs, 3H), 7.62-7.53 (m, 2H), 5.42 (d, J = 15.0 Hz, 1H), 4.08 (q, J = 7.2 Hz, 2H), 4.00-3.70 (m, 5H), 3.57 (s, 2H), 3.20-3.18 (m, 1H), 3.05-2.85 (m, 2H), 2.77-2.75 (m, 1H), 1.19 (t, J = 7.2 Hz, 3H) |
| 45 | 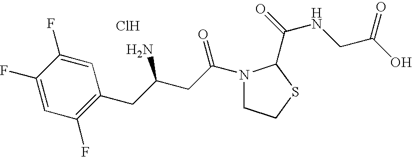 | (CD₃OD, 300 MHz) δ 7.32-7.24 (m, 1H), 7.19-7.10 (m, 1H), 5.42 (s, 2H), 4.09-3.83 (m, 4H), 3.09-2.93 (m, 3H), 2.76-2.69 (m, 2H) |
| 46 | 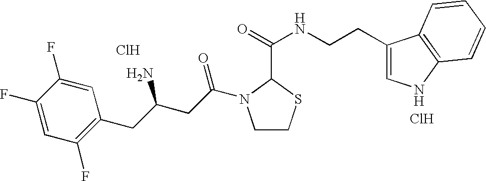 | (CD₃OD, 300 MHz) δ 7.52-7.50 (m, 1H), 7.50-7.17 (m, 3H), 7.05-6.91 (m, 3H), 5.38 (d, J = 14.1 Hz, 1H), 3.76-3.60 (m, 4H), 3.25-3.16 (m, 1H), 3.06- 2.81 (m, 8H) |
| 47 | 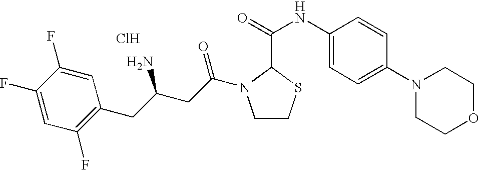 | (CD₃OD, 300 MHz) δ 7.75 (d, J = 9.0 Hz, 2H), 7.56 (d, J = 9.0 Hz, 2H), 7.20-7.00 (m, 2H), 4.86-4.73 (m, 1H), 4.03- 7.01 (m, 7H), 3.64-3.62 (m, 8H), 3.27-3.20 (m, 2H) |
| 48 | 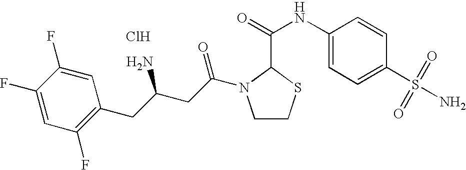 | (DMSO-d₆, 300 MHz) δ 8.10 (brs, 3H), 7.80-7.78 (m, 1H), 7.52-7.48 (m, 3H), 6.61-6.56 (m, 2H), 5.30 (d, J = 18.0 Hz, 1H), 3.70-3.64 (m, 3H), 3.00- 2.71 (m, 6H) |
| 49 | 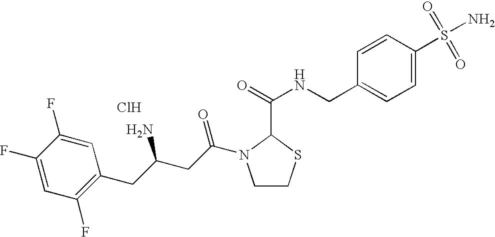 | (DMSO-d₆, 300 MHz) δ 8.11 (brs, 3H), 7.78-7.74 (m, 2H), 7.61-7.51 (m, 2H), 7.43-7.20 (m, 2H), 5.40 (d, J = 15.8 Hz, 1H), 4.32 (s, 2H), 3.93-3.70 (m, 3H), 3.22-3.20 (m, 2H), 3.00-2.98 (m, 3H), 2.77-2.75 (m, 2H) |
| 50 | 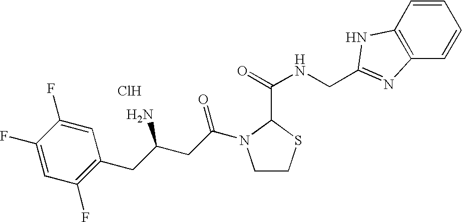 | (DMSO-d₆, 300 MHz) δ 8.19 (brs, 3H), 7.79-7.73 (m, 2H), 7.59-7.48 (m, 4H), 5.40 (s, 1H), 4.69 (s, 2H), 4.10-3.55 (m, 5H), 3.40-3.27 (m, 1H), 3.19-3.17 (m, 1H), 2.99-2.65 (m, 1H) |
| 51 | 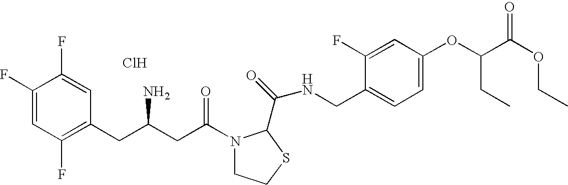 | (DMSO-d₆, 300 MHz) δ 8.55- 8.42 (m, 1H), 8.02 (br, 3H), 7.59-7.49 (m, 2H), 7.33-7.14 (m, 1H), 6.78-6.64 (m, 2H), 5.40-5.34 (s, 1H), 4.80 (br, 1H), 4.24-4.06 (m, 4H), 3.94- 3.60 (m, 4H), 3.20-3.14 (m, 2H), 2.94-2.80 (m, 2H), 2.77- 2.70 (m, 2H), 1.90-1.78 (m, 1H), 1.13 (t, J = 7.1 Hz, 3H), 0.92 (t, J = 7.3 Hz, 3H) |
| 52 | 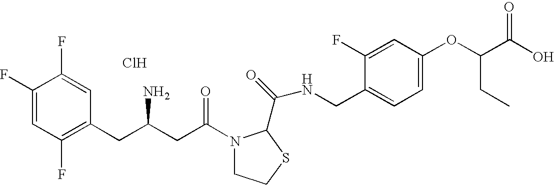 | (DMSO-d₆, 300 MHz) δ 12.89 (br, 1H), 8.53-8.41 (m, 1H), 8.02 (br, 3H). 7.55-7.47 (m, 2H), 7.25-7.13 (m, 1H), 6.76- 6.62 (m, 2H), 5.36-5.32 (s, 1H), 4.47 (brm, 1H), 4.19-4.08 (m, 2H), 3.88-3.59 (m, 4H), 3.20- 2.88 (m, 4H), 2.75-2.68 (m, 2H), 1.89-1.77 (m, 1H), 0.93 (t, J = 7.3 Hz, 3H) |
| 53 | 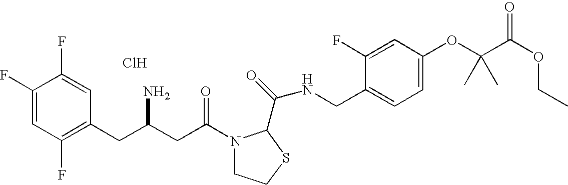 | (DMSO-d₆, 300 MHz) δ 8.58- 8.45 (m, 1H), 8.10 (br, 2H), 7.59-7.48 (m, 2H), 7.24-7.13 (m, 1H), 6.63-5.51 (m, 2H), 5.41-5.33 (s, 1H), 4.24- 4.07 (m, 4H), 3.94-3.59 (m, 3H), 3.26- 3.11 (m, 2H), 3.06-2.69 (m, 4H), 1.49-1.48 (s, 6H), 1.16-1.10 (m, J = 7.1 Hz, 3H) |
| 54 | 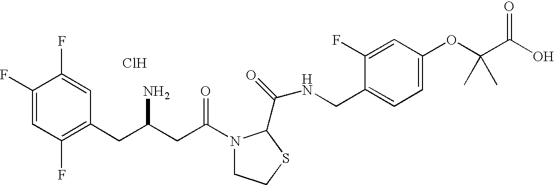 | (DMSO-d₆, 300 MHz) δ 13.13 (br, 1H), 8.55-8.43 (br, 1H), 8.06 (br, 2H), 7.58-7.48 (m, 2H), 7.21-7.12 (m, 1H), 6.64- 6.55 (m, 2H), 5.49-5.33 (s, 1H), 4.25-4.07 (m, 2H), 3.94-3.59 (m, 3H), 3.28-3.12 (m, 2H), 3.06-2.66 (m, 4H), 1.47 (s, 6H) |
| 55 | 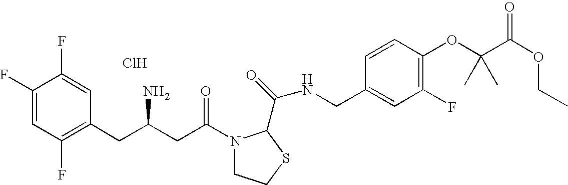 | (DMSO-d₆, 300 MHz) δ 8.65- 8.52 (m, 1H), 8.07 (br, 2H), 7.57-7.48 (m, 2H), 7.09-6.81 (m, 3H), 5.43 + 5.33 (s, 1H), 4.25-4.07 (m, 4H), 3.96-3.85 (m, 1H), 3.80-3.59 (m, 2H), 3.25-2.70 (m, 6H), 1.44 (s, 6H), 1.18-1.13 (m, 3H) |
| 56 | 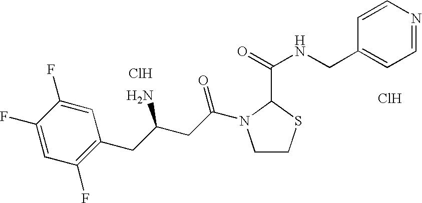 | (DMSO-d₆, 300 MHz) δ 8.83 (d, J = 4.5 Hz, 2H), 8.31 (brs, 3H), 7.83 (d, J = 4.5 Hz, 2H), 7.60-7.51 (m, 2H), 5.40 (s, 1H), 4.49 (s, 2H), 4.04-4.01 (m, 1H), 3.81-3.75 (m, 2H), 3.25- 3.21 (m, 1H), 3.09-2.65 (m, 5H) |
| 57 | 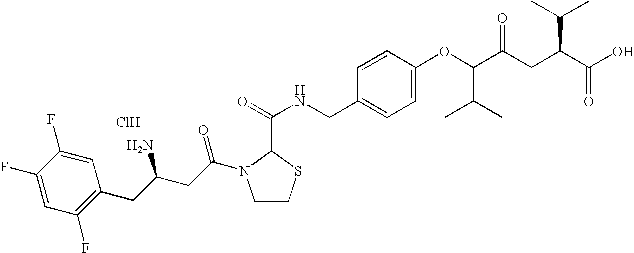 | (DMSO-d₆, 300 MHz) δ 8.26~ 8.21 (m, 1H), 7.78 (br, 3H), 7.36~7.24 (m, 2H), 6.91~6.89 (m, 2H), 6.66~6.62 (m, 2H), 5.17~5.14 (m, 1H), 4.25~4.19 (m, 1H), 4.03~3.91 (m, 6H), 3.55~3.40 (m, 2H), 3.00~2.80 (m, 2H), 2.71~2.60 (m, 2H), 2.50~2.28 (m, 2H), 1.78~1.68 (m, 2H), 0.77~0.73 (m, 6H), 0.67~0.55 (m, 6H) |
| 58 | 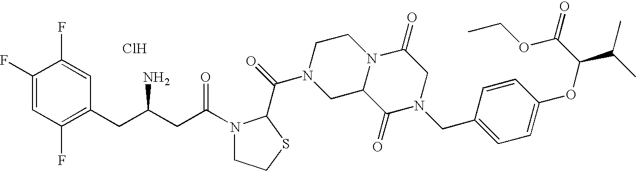 | (CD₃OD, 300 MHz) δ 7.42~ 7.30 (m, 1H), 7.23~7.20 (m, 3H), 6.85~6.83 (m, 2H), 4.88~ 4.74 (m, 4H), 4.51~4.45 (m, 6H), 4.16 (q, J = 7.1 Hz, 2H), 4.00~3.70 (m, 6H), 3.05~2.95 (m, 2H), 2.80~2.60 (m, 4H), 2.23~2.21 (m, 1H), 1.19 (t, J = 7.1 Hz, 3H), 1.04~1.01 (m, 6H) |
| 59 | 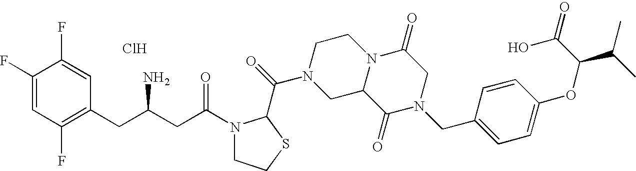 | (DMSO-d₆, 300 MHz) δ 7.56~ 7.54 (m, 2H), 7.22~7.20 (m, 2H), 6.86~6.83 (m, 2H), 4.80~ 4.60 (m, 1H), 4.46~4.26 (m, 8H), 4.16~4.00 (m, 2H), 3.99~ 3.75 (m, 3H), 2.94~2.70 (m, 8H), 2.30~2.00 (m, 2H), 1.00 (d, J = 6.6 Hz, 6H) |
| 60 | 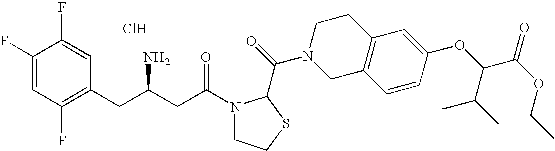 | (CD₃OD, 300 MHz) δ 7.32~7.19 (m, 2H), 7.04~7.00 (m, 1H), 6.72~6.67 (m, 2H), 6.00~ 5.96 (m, 1H), 4.78~4.42 (m, 3H), 4.16 (q, J = 7.1 Hz, 2H), 3.89~3.62 (m, 6H), 3.03~2.79 (m, 8H), 2.22~2.20 (m, 1H), 1.23 (t, J = 7.1 Hz, 3H), 1.04~1.01 (m, 6H) |
| 61 | 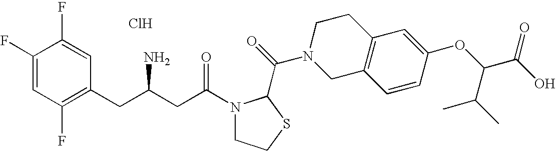 | (DMSO-d₆, 300 MHz) δ 12.92 (br, 1H), 8.05 (brs, 3H), 7.57~7.52 (m, 2H), 7.12~7.09 (m, 1H), 6.76~6.72 (m, 2H), 6.17~5.98 (m, 1H), 4.65~4.44 (m, 3H), 3.85~3.57 (m, 6H), 3.22~2.73 (m, 6H), 2.20~2.00 (m, 1H), 1.00 (t, J = 6.9Hz, 6H) |
| 62 | 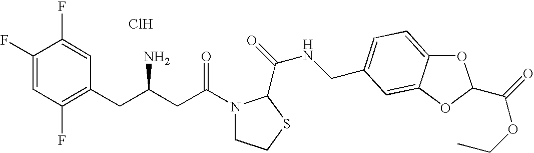 | (DMSO-d₆, 300 MHz) δ 8.75 (br, 1H), 8.23 (brs, 3H), 7.75- 7.63 (m, 2H), 7.11-6.90 (m, 3H), 6.79-6.76 (br, 1H), 5.55- 5.50 (s, 1H), 4.41-3.86 (m, 7H), 3.57-2.65 (m, 7H), 1.38 (t, J = 7.1 Hz, 3H) |
| 63 | 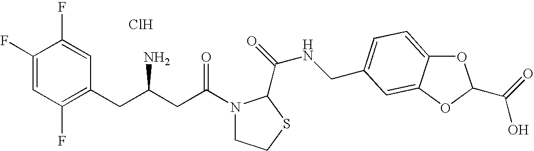 | (DMSO-d₆, 300 MHz) δ 8.70 (br, 1H), 8.08 (brs, 3H), 7.57- 7.54 (m, 2H), 6.92-6.67 (m, 3H), 6.47 (br, 1H), 5.43-5.40 (s, 1H), 4.30-3.86 (m, 5H), 3.76-2.97 (m, 7H), 2.74-2.51 (m, 2H) |
| 64 | 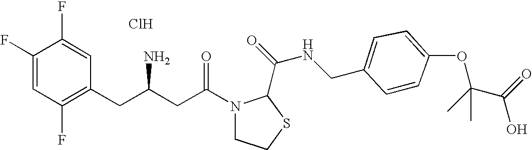 | (DMSO-d₆, 300 MHz) δ 8.53 (brs, 1H) 7.68~7.41 (m, 2H) 7.23~7.09 (m, 2H) 6.85~6.63 (m, 2H) 5.43~5.38 (m, 1H) 4.33~4.06 (m, 2H) 4.00~3.91 (m, 2H) 3.42~3.23 (m, 2H) 3.15~2.95 (m, 2H) 2.83~2.74 (m, 2H) 1.48 (s, 6H) |
| 65 | 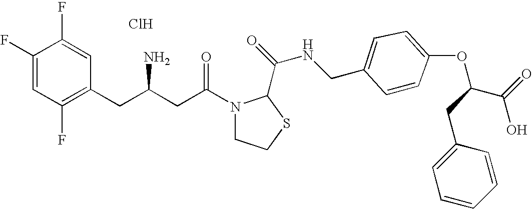 | (DMSO-d₆, 300 MHz) δ 8.53 (brs, 1H) 7.64~7.45 (m, 2H) 7.38~7.19 (m, 5H) 7.18~7.11 (m, 2H) 6.88~6.73 (m, 2H) 5.43~5.36 (m, 1H) 5.08~4.82 (m, 1H) 4.21~4.00 (m, 3H) 3.98~3.63 (m, 4H) 3.28~3.16 (m, 2H) 3.09~2.85 (m, 2H) 2.80~2.69 (m, 2H) |
| 66 | 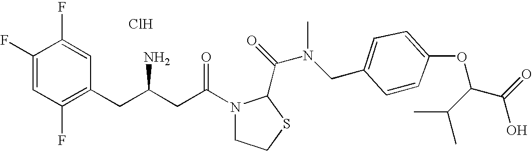 | (DMSO-d₆, 300 MHz) δ 8.16 (brs, 2H) 7.77~7.57 (m, 2H) 8.26~7.12 (m, 2H) 6.84~6.61 (m, 2H) 6.11~5.88 (m, 1H) 4.74~4.45 (m, 2H) 3.87~3.70 (m, 3H) 3.34~2.70 (m, 6H) 2.20~2.04 (m, 1H) 1.02~0.99 (m, 6H) |
| 67 | 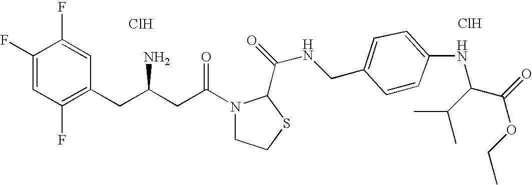 | (DMSO-d₆, 300 MHz) δ 8.16 (brs, 3H) 7.65~7.46 (m, 2H) 7.08~6.92 (m, 2H) 6.63~6.48 (m, 2H) 5.43~5.36 (m, 1H) 4.21~4.03 (m, 4H) 3.96~3.43 (m, 4H) 3.33~3.28 (m, 2H) 3.15~2.96 (m, 2H) 2.81~2.71 (m, 2H) 2.19~1.97 (m, 2H) 1.16 (t, J = 6.85 Hz, 3H) 1.01~ 0.92 (m, 6H) |
| 68 | 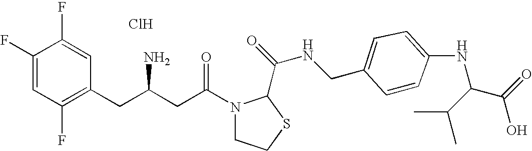 | (DMSO-d₆, 300 MHz) δ 8.19 (brs, 3H) 7.63~7.41 (m, 2H) 7.08~6.93 (m, 2H) 6.70~6.49 (m, 2H) 5.47~5.36 (m, 1H) 4.02~3.62 (m, 4H) 3.53~3.36 (m, 2H) 3.12~2.96 (m, 2H) 2.83~2.71 (m, 2H) 2.18~1.98 (m, 5H) 1.05~0.96 (m, 6H) |
| 69 | 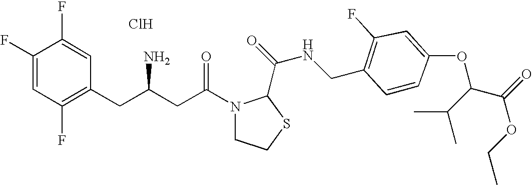 | (DMSO-d₆, 300 MHz) δ 8.16 (brs, 3H) 7.67~7.49 (m, 2H) 7.31-7.13 (m, 1H) 6.83~6.59 (m, 2H) 7.31~7.13 (m, 1H) 6.83~6.59 (m, 2H) 5.51~5.36 (m, 1H) 4.71~4.61 (m, 1H) 4.38~4.03 (m, 5H) 4.00~3.61 (m, 5H) 3.35~3.29 (m, 2H) 3.11~2.95 (m, 2H) 2.83~2.72 (m, 2H) 2.38~2.13 (m, 1H) 1.23~1.11 (m, 3H) 1.08~0.96 (m, 6H) |
| 70 | 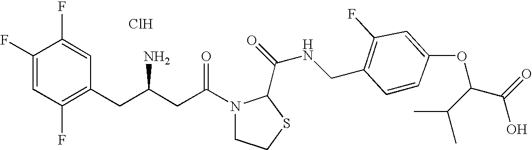 | (DMSO-d₆, 300 MHz) δ 8.50 (brs, 1H) 7.63~7.41 (m, 2H) 7.28~7.12 (m, 1H) 6.80~6.59 (m, 2H) 5.42~5.36 (m, 1H) 4.58~4.42 (m, 1H) 4.31~4.10 (m, 3H) 3.91~3.63 (m, 2H) 3.38~3.30 (m, 2H) 3.13~2.83 (m, 2H) 2.81~2.73 (m, 2H) 2.31~2.16 (m, 1H) 1.08~0.97 (m, 6H) |
| 71 | 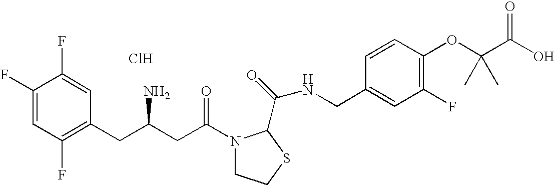 | (DMSO-d₆, 300 MHz) δ 8.65- 8.50 (m, 1H), 8.07 (br, 2H), 7.57-7.48 (m, 2H), 7.10-6.84 (m, 3H), 5.43-5.32 (s, 1H), 4.27-4.06 (m, 2H), 3.97-3.59 (m, 3H), 3.25-2.66 (m, 6H), 1.44-1.43 (s, 3H) |
| 72 | 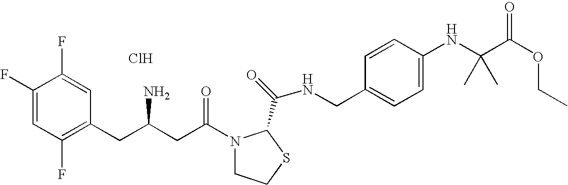 | (DMSO-d₆, 300 MHz) δ 8.41 (brt, 1H), 8.07 (br, 3H), 7.59- 7.49 (m, 2H), 7.06-6.96 (m, 2H), 6.64-5.54 (m, 2H), 5.38 (s, 1H), 4.15-3.59 (m, 5H), 3.22- 3.09 (m, 2H), 3.04-2.89 (m, 2H), 2.80-2.67 (m, 2H), 1.41 (s, 6H), 1.10 (t, J = 7.1 Hz, 3H). |
| 73 | 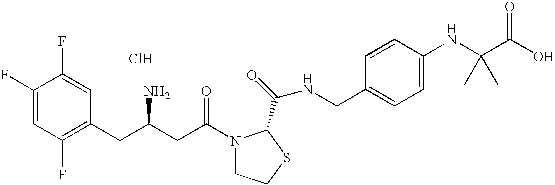 | (DMSO-d₆, 300 MHz) δ 8.55 (brs, 1H), 8.14 (brs, 3H), 7.62-7.49 (m, 2H), 7.17-7.15 (m, 2H), 7.01-6.97 (m, 2H), 5.40 (s, 1H), 4.22-4.18 (m, 2H), 3.97-3.90 (m, 1H), 3.86-3.56 (m, 2H), 3.25-3.16 (m, 2H), 3.04-2.96 (m, 2H), 2.78-2.74 (m, 2H), 1.41 (s, 6H) |
| 74 | 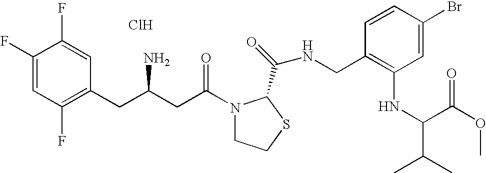 | (DMSO-d₆, 300 MHz) δ 8.59 (brs, 1H), 8.08 (brs, 3H), 7.59-7.49 (m, 2H), 7.10-6.95 (m, 1H), 6.79-6.68 (m, 1H), 6.65-6.57 (m, 1H), 5.49-5.40 (m, 1H), 5.39 (s, 1H), 4.22-4.06 (m, 2H), 3.98-3.90 (m, 1H), 3.82-3.57 (m, 6H), 3.20-3.13 (m, 2H), 3.04-2.88 (m, 2H), 2.78-2.69 (m, 2H), 2.14- 2.04 (m, 1H), 0.98 (d, J = 6.7 Hz, 3H), 0.91 (d, J = 6.7 Hz, 3H) |
| 75 | 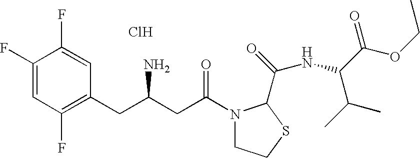 | (DMSO-d₆, 300 MHz) δ 8.73 (brs, 1H), 8.48 (brs, 2H), 7.39~7.10 (m, 1H), 6.96~6.91 (m, 1H), 6.28 (s, 1H), 4.37~4.15 (m, 3H), 3.87~3.64 (m, 3H), 3.49~2.94 (m, 5H), 2.80~2.60 (m, 1H), 2.40~2.09 (m, 1H), 1.88 (brs, 1H), 1.31~1.21 (m, 3H), 1.07~0.88 (m, 6H) |
| 76 | 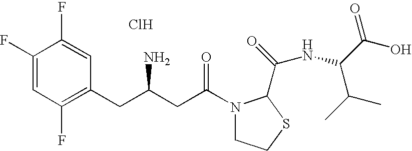 | (CDCl₃ 300 MHz) δ 8.55 (br, 1H), 8.30 (br, 2H), 7.32~ 7.27 (m, 1H), 6.94~6.80 (m, 1H) 6.06 (s, 1H), 4.34~3.78 (m, 4H), 3.68~3.61 (m, 1H), 3.40~3.20 (m, 1H), 3.08 (brs, 1H), 2.89~2.06 (m, 4H), 1.30~1.24 (m, 2H), 1.00~0.86 (m, 6H) |
| 77 | 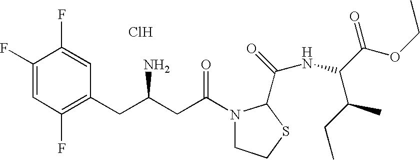 | (DMSO-d₆, 300 MHz) δ 8.76 (brs, 1H), 8.48 (brs, 2H), 7.40~7.10 (m, 1H), 6.97~6.91 (m, 1H), 6.26 (s, 1H), 4.40~4.36 (m, 1H), 4.25~4.11 (m, 2H), 3.90~3.64 (m, 3H), 3.50~3.25 (m, 2H), 3.20~2.90 (m, 4H), 2.80~2.60 (m, 1H), 2.25~2.00 (m, 1H), 1.88 (brs, 1H), 1.50~1.48 (m, 1H), 1.20~1.10 (m, 3H), 1.07~0.86 (m, 6H) |
| 78 | 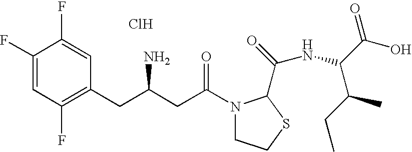 | (CDCl₃ 300 MHz) δ 8.55 (br, 1H), 8.10 (brs, 2H), 7.58~ 7.30 (m, 1H), 7.10~6.94 (m, 1H), 6.10 (s, 1H), 4.20~3.80 (m, 3H), 3.70~3.60 (m, 2H), 3.50~3.20 (m, 2H), 2.95~2.90 (m, 2H), 2.80~2.40 (m, 4H), 2.00~1.80 (m, 1H), 1.60~1.50 (m, 1H), 1.00~0.84 (m, 6H) |
| 79 | 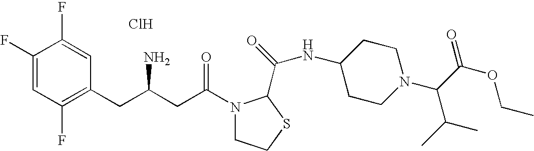 | (CDCl₃ 300 MHz) δ 8.34 (br, 2H), 7.41~7.28 (m, 1H), 7.00~6.90 (m, 1H), 5.65 (brs, 1H), 5.00~4.95 (m, 1H), 4.80~4.70 (m, 1H), 4.20~3.60 (m, 7H), 3.40~3.20 (m, 2H), 3.12~2.84 (m, 4H), 2.80~2.60 (m, 1H), 2.45~2.40 (m, 1H), 2.30~2.20 (m, 2H), 2.00~1.80 (m, 3H), 1.68~1.51 (m, 2H), 1.23~1.21 (m, 3H), 0.99~0.95 (m, 6H) |
| 80 | 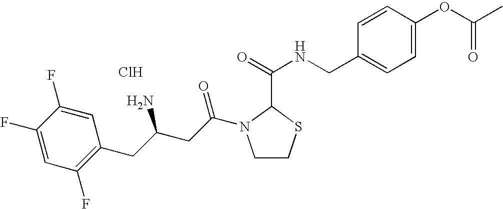 | (CD₃OD, 300 MHz) δ 7.43-7.35 (m, 1H), 7.38 (d, J = 8.4 Hz, 2H), 7.30-7.23 (m, 1H), 7.09 (d, J = 8.4 Hz, 2H), 4.92 (d, J = 4.5 Hz, 1H), 4.86-4.80 (m, 1H), 4.42 (s, 2H), 4.02-4.00 (m, 1H), 3.88-3.85 (m, 2H), 3.42-3.40 (m, 1H), 3.29-3.24 (m, 2H), 3.10-3.04 (m, 2H), 2.30 (s, 3H) |
| 81 | 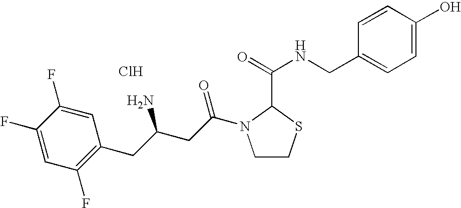 | (DMSO-d₆, 300 MHz) δ 7.61- 7.47 (m, 2H), 7.05 (d, J = 8.4 Hz, 2H), 6.70 (d, J = 8.4 Hz, 2H), 5.38 (d, J = 12.6 Hz, 1H), 4.14 (s, 2H), 3.73-3.70 (m, 2H), 3.24-3.21 (m, 1H), 3.02-2.91 (m, 2H), 2.76-2.64 (m, 2H), 2.51-2.44 (m, 2H) |
| 82 | 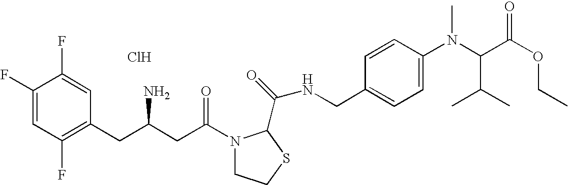 | (DMSO-d₆, 300 MHz) δ 8.60- 8.40 (m, 1H), 8.02 (br, 3H), 7.60-7.51 (m, 2H), 7.08-7.04 (m, 2H), 6.80-6.75 (m, 2H), 5.40-5.37 (s, 1H), 4.18-3.65 (m, 8H), 3.49 (s, 3H), 3.49-3.19 (m, 2H), 2.90-2.78 (m, 2H), 2.70-2.51 (m, 2H), 2.01-1.90 (m, 1H), 1.12 (t, 3H), 1.00-0.79 (m, 6H) |
| 83 | 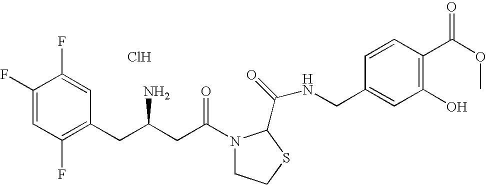 | (DMSO-d₆, 300 MHz) δ 8.59- 8.52 (m, 1H), 8.06 (brd, 3H), 7.66-7.34 (m, 4H), 6.94-6.89 (m, 1H), 5.44-5.33 (s, 1H), 4.22-4.08 (m, 2H), 3.90 (s, 3H), 3.95-3.60 (m, 3H), 3.32- 3.12 (m, 2H), 3.06-2.67 (m, 4H) |
| 84 | 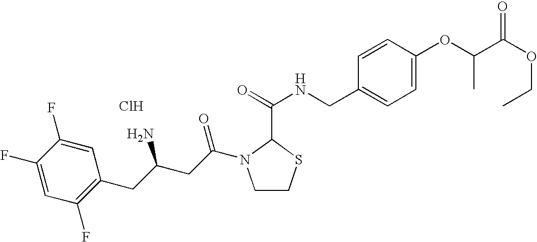 | (CD₃OD, 300 MHz) δ 7.38-7.32 (m, 1H), 7.25-7.19 (m, 1H), 7.17 (d, J = 8.4 Hz, 2H), 6.80 (d, J = 8.4 Hz, 2H), 5.42 (s, 1H), 4.80-4.74 (m, 1H), 4.28 (s, 2H),4.16 (q, J = 7.2 Hz, 2H), 3.98-3.95 (m, 1H), 3.83-3.78 (m, 2H), 3.35-3.28 (m, 2H), 3.23-3.14 (m, 1H), 3.05- 3.00 (m, 2H), 2.81-2.75 (m, 1H), 1.52 (q, J = 6.7 Hz, 3H), 1.21 (t, J = 7.2 Hz, 3H) |
| 85 | 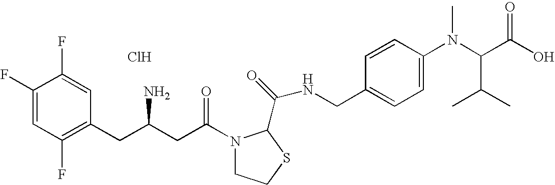 | (DMSO-d₆, 300 MHz) δ 8.60- 8.40 (m, 1H), 8.04 (br, 3H), 7.66-7.51 (m, 2H), 7.33-6.99 (m, 2H), 6.92-6.72 (m, 2H), 5.40-5.37 (s, 1H), 4.24-3.60 (m, 6H), 3.49 (s, 3H), 3.32-3.12 (m, 2H), 3.06-2.87 (m, 2H), 2.78-2.67 (m, 2H), 2.00-1.79 (m, 1H), 0.90-0.81 (m, 6H) |
| 86 | 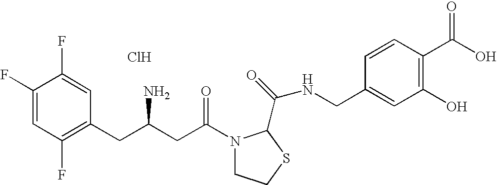 | (DMSO-d₆, 300 MHz) δ 8.58- 8.49 (m, 1H), 7.95 (br, 3H), 7.65-7.33 (m, 4H), 6.91-6.86 (m, 1H), 5.39-5.34 (s, 1H), 4.23-4.10 (m, 2H), 3.92-3.68 (m, 4H), 3.30-3.10 (m, 2H), 2.98-2.70 (m, 4H) |
| 87 | 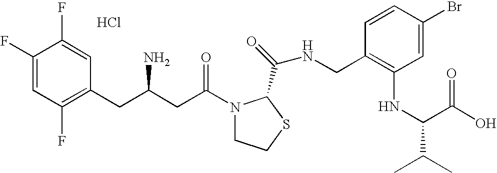 | (DMSO-d₆, 300 MHz) δ 12.6 (br, 1H), 8.54 (brt, 1H), 8.05 (br, 3H), 7.59-7.49 (m, 2H), 7.06-6.96 (m, 1 H), 6.78-6.60 (m, 2H), 5.39 (s, 1H), 5.28 (br, 1H), 4.25-4.03 (m, 2H, NH—CH₂-Ph), 3.97-3.89 (m, 1H), 3.78-3.61 (m, 3H), 3.55 (s, 3H), 3.23-3.11 (m, 2H), 3.02-2.89 (m, 2H), 2.79-2.68 (m, 2H), 2.14-2.02 (m, 1H), 0.99 (d, J = 6.7 Hz, 3H), 0.95 (d, J = 6.7 Hz, 3H). |
| 88 | 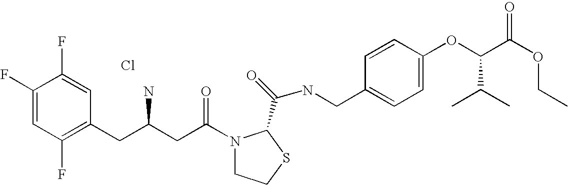 | (DMSO-d₆, 300 MHz) δ 8.50 (brt, 1H, NH), 8.08 (brs, 3H, NH₂•HCl), 7.61-7.52 (m, 2H), 7.14 (d, J = 8.6 Hz, 2H), 6.80 (d, J = 8.6 Hz, 2H), 5.40 (s, 1H), 4.53 (d, J = 5.2 Hz, 1H), 4.24-4.09 (m, 4H), 3.99-3.92 (m, 1H), 3.81- 3.70 (m, 2H), 3.24-3.16 (m, 2H), 3.03- 2.93 (m, 2H), 2.80-2.72 (m, 2H), 2.23-2.12 (m, 1H), 1.17 (t, J = 7.1 Hz, 3H), 1.00 (d, J = 6.7 Hz, 3H), 0.99 (d, J = 6.7 Hz, 3H) |
| 89 | 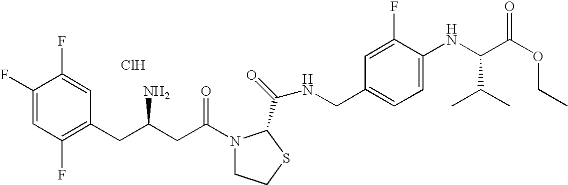 | (DMSO-d₆, 300 MHz) δ 7.96 (br, 1H), 7.60-7.48 (m, 1H), 7.07-6.56 (m, 4H), 5.86 (s, 1H), 5.37-5.22 (m, 2H), 4.30-4.10 (m, 2H), 3.97-3.43 (m, 4H), 3.60 (s, 3H), 3.30-3.10 (m, 2H), 2.98-2.90 (m, 2H), 2.76-2.68 (m, 2H), 2.18-2.06 (m, 1H), 1.00-0.88 (m, 6H). |
| 90 | 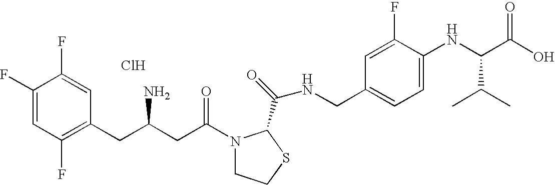 | (DMSO-d₆, 300 MHz) δ 12.75- 7.96 (br, 3H), 7.58-7.50 (m, 1H), 7.06-6.60 (m, 4H), 5.87 (s, 1H), 5.06 (br, 2H), 4.55-3.62 (m, 6H), 3.48-3.28 (m, 2H), 2.98-2.90 (m, 2H), 2.76-2.69 (m, 2H), 2.16-2.05 (m, 1H), 1.00-0.90 (m, 6H). |
| 91 | 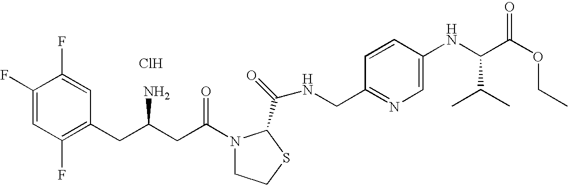 | (DMSO-d₆, 300 MHz) δ 8.86 (br, 1H), 8.14-8.02 (m, 4H), 7.63-7.34 (m, 4H), 5.39 (s, 1H), 4.47-4.32 (m, 2H), 4.17-4.06 (m, 2H), 4.02-3.92 (m, 1H), 3.82-3.50 (m, 3H), 3.24-3.16 (m, 2H), 3.09-2.71 (m, 4H), 2.20-2.09 (m, 1H), 1.18 (t, J = 7.1 Hz, 3H), 0.98 (d, J = 7.0 Hz, 3H), 0.95 (d, J = 7.0 Hz, 3H). |
| 92 | 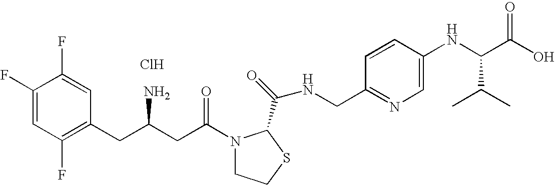 | (DMSO-d₆, 300 MHz) δ 12.95 (br, 1H), 8.85 (br, 1H), 8.16- 8.00 (m, 4H), 7.66-7.31 (m, 4H), 5.39 (s, 1H), 4.47-4.32 (m, 2H), 4.02-3.50 (m, 4H), 3.24-3.16 (m, 2H), 3.09-2.71 (m, 4H), 2.21-2.10 (m, 1H), 0.99 (d, J = 7.0 Hz, 3H), 0.96 (d, J = 7.0 Hz, 3H). |
| 93 | 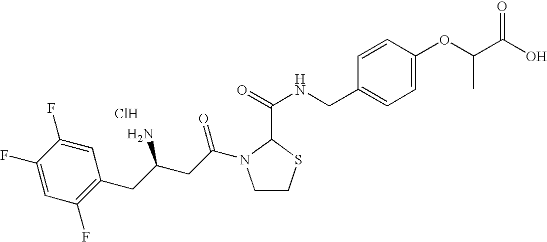 | (CD₃OD, 300 MHz) δ 7.22 (d, J = 8.4 Hz, 2H), 7.21-7.19 (m, 1H), 7.13-7.07 (m, 1H), 6.84 (d, J = 8.4 Hz, 2H), 5.46 (d, J = 6.9 Hz, 1H), 4.81- 4.84 (m, 1H), 4.32 (s, 2H), 4.29- 4.20 (m, 1H), 4.07-3.97 (m, 1H), 3.91-3.86 (m, 2H), 3.17- 3.16 (m, 1H), 2.96-2.94 (m, 1H), 2.80-2.64 (m, 3H), 1.57 (d, J = 6.6 Hz, 3H), 1.34 (s, 9H) |
| 94 | 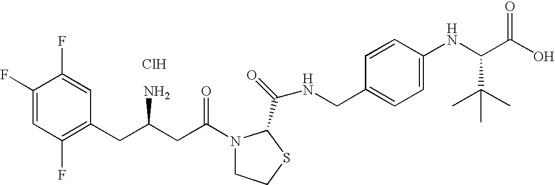 | (DMSO-d₆, 300 MHz) δ 8.33 (br, 1H), 8.07 (br, 3H), 7.60- 7.47 (m, 2H), 6.98-6.89 (m, 2H), 6.67-6.57 (m, 2H), 5.37 (s, 1H), 4.13-4.01 (m, 2H), 3.95-3.85 (m, 1H), 3.79-3.42 (m, 3H), 3.24-3.10 (m, 2H), 3.01-2.83 (m, 2H), 2.77-2.67 (m, 2H), 1.00 (s, 9H) |
| 95 | 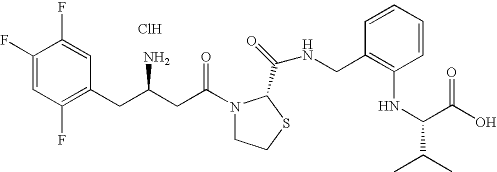 | (DMSO-d₆, 300 MHz) δ 8.58 (brt, 1H), 8.19 (brs, 3H), 7.70-7.40 (m, 3H), 7.19-7.09 (m, 2H), 6.73-6.53 (m, 2H), 5.50 (s, 1H), 4.43-3.95 (m, 3H), 3.90-3.42 (m, 3H), 3.34-3.20 (m, 2H), 3.14-2.98 (m, 2H), 2.92-2.77 (m, 2H), 2.24-2.12 (m, 1H), 1.10 (d, J = 6.6 Hz, 3H), 1.06 (d, J = 6.6 Hz, 3H). |
| 96 | 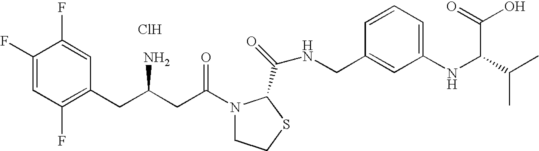 | (DMSO-d₆, 300 MHz) δ 8.43 (brt, 1H), 8.07 (br, 3H), 7.70- 7.24 (m, 3H), 7.03-6.90 (m, 1H), 6.55-6.40 (m, 2H), 5.39 (s, 1H), 4.18-4.03 (m, 2H), 3.98-3.87 (m, 1H), 3.79-3.42 (m, 3H), 3.25-3.10 (m, 2H), 3.04-2.89 (m, 2H), 2.80-2.64 (m, 2H), 2.09-1.96 (m, 1H), 0.96 (d, J = 7.0 Hz, 3H), 0.93 (d, J = 7.0 Hz, 3H). |
| 97 | 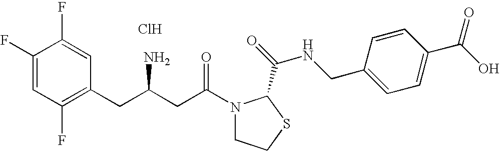 | LC-MS m/e 482 (MH⁺) |
| 98 | 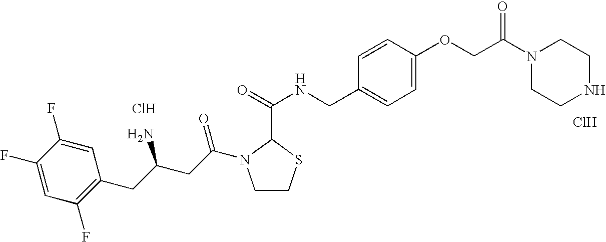 | LC-MS m/e 580 (MH⁺) |
| 99 | 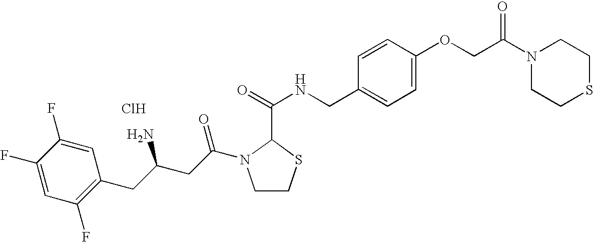 | LC-MS m/e 597 (MH⁺) |
| 100 | 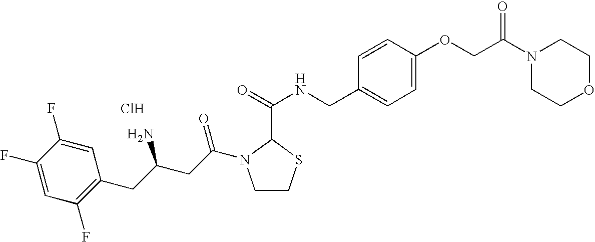 | LC-MS m/e 581 (MH⁺) |
| 101 | 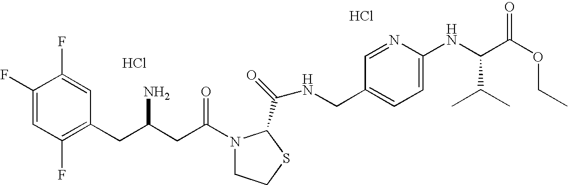 | (DMSO-d6, 300 MHz) δ 8.70 (br, 1H), 8.12 (brs, 3H), 7.81-7.71 (m, 2H), 7.60-7.49 (m, 2H), 7.23-7.11 (brm, 1H), 5.34 (s, 1H), 4.20-4.07 (m, 4H), 3.90-3.91 (m, 1H), 3.76-3.44 (m, 3H), 3.19-3.15 (m, 2H), 3.04-2.94 (m, 2H), 2.76-2.75 (m, 2H), 2.27-2.22 (m, 1H), 1.18 (t, J = 7.1 Hz, 3H), 0.96 (d, J = 6.7 Hz, 3H), 0.94 (J = 6.7 Hz, 3H) |
| 102 | 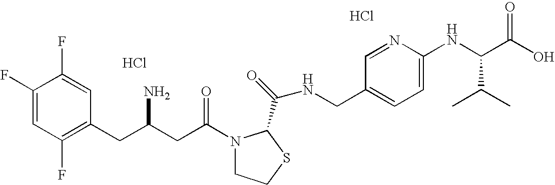 | ((DMSO-d6, 300 MHz) δ 8.67 (brt, 1H), 8.21-8.17 (br, 3H), 7.79-7.74 (brm, 2H), 7.61-7.48 (m, 2H), 7.25-7.21 (m, 1H), 5.34 (s, 1H), 4.15-4.13 (m, 2H), 3.99-3.92 (m, 1H), 3.77-3.44 (m, 3H), 3.21-3.15 (m, 2H) 3.17-2.89 (m, 2H), 2.78-2.73 (m, 2H), 2.16-2.05 (m, 1H), 0.97 (d, J = 6.7 Hz, 3H), 0.94 (J = 6.7 Hz, 3H) |
| 103 | 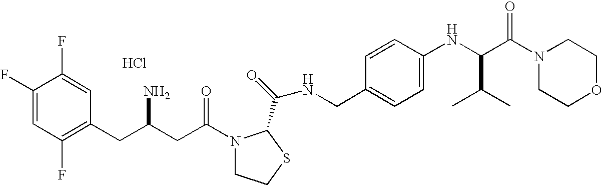 | (DMSO-d6, 300 MHz) δ 8.43- 8.37 (brt, 1H), 8.16 (br, 3H), 7.61-7.54 (m, 2H), 6.99-6.90 (m, 2H), 6.71-6.68 (m, 2H), 5.40 (s, 1H), 4.25 (d, J = 6.5 Hz, 1H), 4.12-4.07 (m, 2H), 3.98-3.91 (m, 1H), 3.80-3.37 (m, 10H), 3.27-3.12 (m, 2H), 3.09-2.91 (m, 2H), 2.83-2.68 (m, 2H), 2.04-1.91 (m, 1H), 0.95 (d, J = 6.6 Hz, 3H), 0.93 (d, J = 6.6 Hz, 3H) |
| 104 | 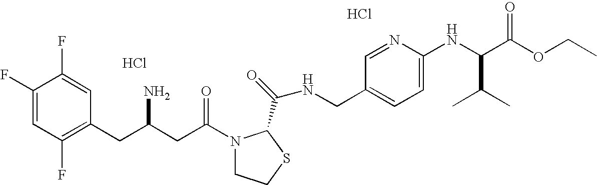 | (DMSO-d6, 300 MHz) δ 8.69 (brt, 1H), 8.14 (brs, 3H), 7.75-7.70 (m, 2H), 7.59-7.46 (m, 2H), 7.19-7.14 (brm, 1H), 5.32 (s, 1H), 4.19-4.06 (m, 4H), 3.95-3.90 (m, 1H), 3.71-3.45 (m, 3H), 3.17-3.11 (m, 2H), 3.02-2.88 (m, 2H), 2.75-2.73 (m, 2H), 2.26-2.20 (m, 1H), 1.16 (t, J = 7.1 Hz, 3H), 0.95 (d, J = 6.7 Hz, 3H), 0.91 (d, J = 6.7 Hz, 3H) |
| 105 | 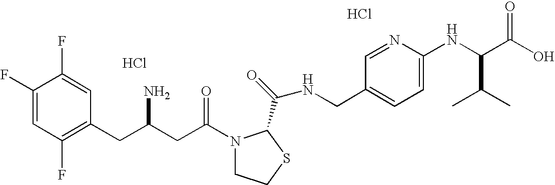 | (DMSO-d6, 300 MHz) δ 8.67 (brt, 1H), 8.21-8.17 (brm, 3H), 7.80-7.73 (brm, 2H), 7.60-7.49 (m, 2H), 7.25-7.20 (m, 1H), 5.32 (s, 1H), 4.16-4.13 (m, 2H), 4.00-3.92 (m, 1H), 3.77-3.44 (m, 3H), 3.21-3.15 (m, 2H) 3.17-2.88 (m, 2H), 2.78-2.73 (m, 2H), 2.17-2.03 (m, 1H), 0.97 (d, J = 6.7 Hz, 3H), 0.94 (J = 6.7 Hz, 3H) |
| 106 | 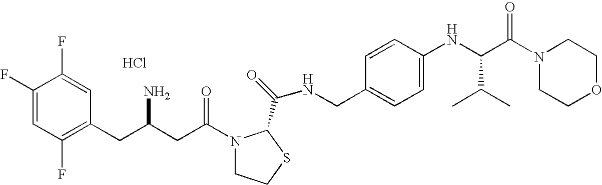 | (DMSO-d6, 300 MHz) δ 8.43- 8.35 (brt, 1H), 8.10 (br, 3H), 7.62-7.53 (m, 2H), 7.01-6.90 (m, 2H), 6.71-6.65 (m, 2H), 5.40 (s, 1H), 4.23 (d, J = 6.5 Hz, 1H), 4.15-4.04 (m, 2H), 3.98-3.90 (m, 1H), 3.80-3.37 (m, 10H), 3.26-3.12 (m, 2H), 3.08-2.91 (m, 2H), 2.83-2.68 (m, 2H), 2.03-1.91 (m, 1H), 0.95 (d, J = 6.6 Hz, 3H), 0.93 (d, J = 6.6 Hz, 3H) |
| 107 | 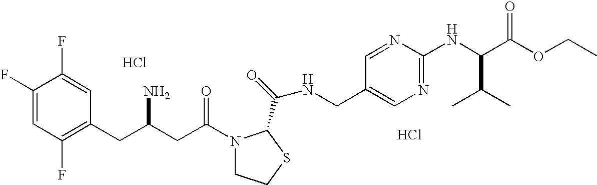 | (DMSO-d6, 300 MHz) δ 8.55 (brt, 1H), 8.23 (m, 4H), 7.56- 7.42 (m, 3H), 5.31 (s, 1H), 4.19-3.89 (m, 4H), 3.74-3.50 (m, 4H), 3.17-3.12 (m, 2H), 3.01-2.91 (m, 2H), 2.72-2.69 (m, 2H), 2.13-2.04 (m, 1H), 1.12 (t, J = 7.1 Hz, 3H), 0.92 (d, J = 6.7 Hz, 3H), 0.86 (J = 6.7 Hz, 3H) |
| 108 | 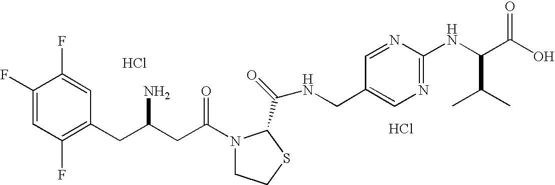 | (DMSO-d6, 300 MHz) δ 8.53 (brt, 1H), 8.26-8.02 (m, 4H), 7.60-7.47 (m, 3H), 5.31 (s, 1H), 4.19-3.89 (m, 4H), 3.72-3.62 (m, 2H), 3.17-3.12 (m, 2H), 3.01-2.86 (m, 2H), 2.73-2.69 (m, 2H), 2.15-2.10 (m, 1H), 1.12 (t, J = 7.1 Hz, 3H), 0.93 (d, J = 6.7 Hz, 3H), 0.91 (J = 6.7 Hz, 3H) |
| 109 | 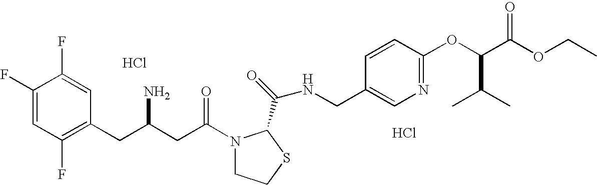 | (DMSO-d6, 300 MHz) δ 8.54 (brt, 1H), 8.06 (brs, 3H), 7.89 (d, J =2.1 Hz, 1H), 7.56-7.47 (m, 3H), 6.82-6.79 (m, 1H), 5.33 (s, 1H), 4.23-4.04 (m, 4H), 3.95-3.90 (m, 1H), 3.74-3.67 (m, 3H), 3.18-3.12 (m, 2H), 3.18-2.91 (m, 2H), 2.74-2.69 (m, 2H), 2.20-2.11 (m, 1H), 1.15 (t, J = 7.1 Hz, 3H), 2.98 (d, J = 6.7 Hz, 3H), 0.95 (J = 6.7 Hz, 3H) |
| 110 | 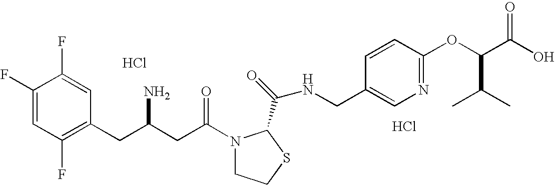 | (DMSO-d6, 300 MHz) δ 8.54 (brt, 1H), 8.06 (br, 3H), 7.90- 7.89 (m, 1H), 7.55-7.50 (m, 3H), 6.79-6.77 (m, 1H), 5.33 (s, 1H), 4.19-4.14 (m, 2H), 3.91-3.70 (m, 4H), 3.18-3.12 (m, 2H), 2.95-2.90 (m, 2H), 2.73-2.71 (m, 2H), 2.16-2.14 (m, 1H), 0.98 (d, J = 6.7 Hz, 3H), 0.95 (J = 6.7 Hz, 3H) |
| 111 | 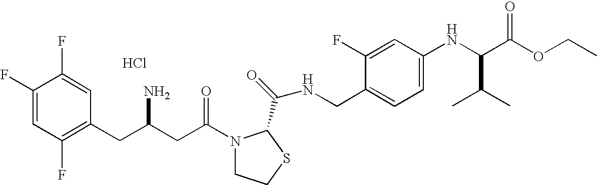 | (DMSO-d6, 300 MHz) δ 8.33- 8.28 (brt, 1H), 7.79 (br, 3H), 7.59-7.47 (m, 2H), 7.02-6.82 (m, 1H), 6.42-6.32 (m, 2H), 5.36 (s, 1H), 4.15-3.96 (m, 4H), 3.94-3.87 (m, 1H), 3.78-3.61 (m, 3H), 3.22-3.09 (m, 2H), 3.04-2.81 (m, 2H), 2.76-2.64 (m, 2H), 2.06-1.95 (m, 1H), 1.14 (t, J = 7.1 Hz, 3H), 0.95 (d, J = 6.7 Hz, 3H), 0.90 (d, J = 6.7 Hz, 3H) |
| 112 | 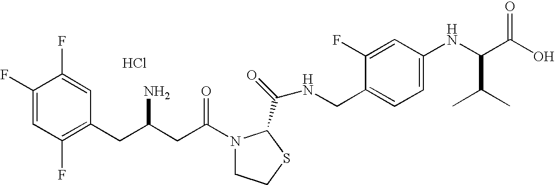 | (DMSO-d6, 300 MHz) δ δ 8.33- 8.28 (brt, 1H), 8.11 (br, 3H), 7.60-7.47 (m, 2H), 7.02-6.82 (m, 1H), 6.42-6.32 (m, 2H), 5.35 (s, 1H), 4.18-3.86 (m, 2H), 3.93-3.86 (m, 1H), 3.76-3.58 (m, 3H), 3.21-3.09 (m, 2H), 3.03-2.89 (m, 2H), 2.81-2.64 (m, 2H), 2.05-1.93 (m, 1H), 0.94 (d, J = 6.7 Hz, 3H), 0.91 (d, J = 6.7 Hz, 3H) |
| 113 | 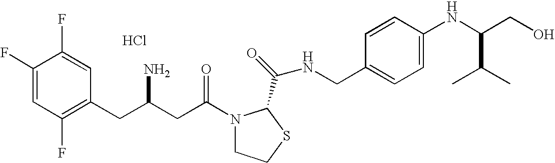 | (DMSO-d6, 300 MHz) δ 8.48 (brt, 1H), 8.12 (br, 3H), 7.54- 7.47 (m, 2H), 7.13-6.93 (m, 4H), 5.37 (s, 1H), 4.18-4.11 (m, 2H), 3.92-3.41 (m, 6H), 3.20-3.13 (m, 2H), 3.00-2.93 (m, 2H), 2.76-2.70 (m, 2H), 1.92-1.87 (m, 1H), ), 0.91 (d, J = 6.7 Hz, 3H), 0.89 (d, J = 6.7 Hz, 3H) |
| 114 | 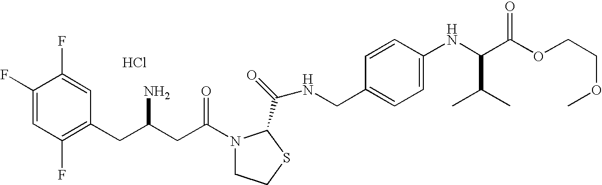 | (DMSO-d6, 300 MHz) δ 8.34 (brt, 1H), 8.15 (br, 3H), 7.59- 7.47 (m, 2H), 6.96-6.89 (m, 2H), 6.57-6.49 (m, 2H), 5.35 (s, 1H), 4.47 (br, 2H), 4.14-4.02 (m, 4H), 3.92-3.87 (m, 1H), 3.76-3.63 (m, 3H), 3.46-3.43 (m, 2H), 3.19 (s, 3H), 3.16-3.11 (m, 2H), 3.02-2.94 (m, 2H), 2.83-2.70 (m, 2H), 2.02-1.96 (m, 1H), 0.94 (d, J = 6.7 Hz, 3H), 0.90 (d, J = 6.7 Hz, 3H) |
| 115 | 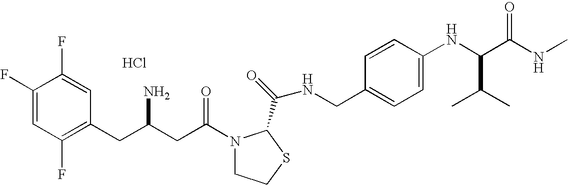 | (DMSO-d6, 300 MHz) δ 8.32 (brt, 1H), 8.00 (brm, 3H), 7.97- 7.81 (m, 2H), 7.57-7.47 (m, 2H), 6.95-6.89 (m, 2H), 5.35 (s, 1H), 4.07-4.03 (m, 2H), 3.95- 3.82 (m, 1H), 3.74-3.66 (m, 1H), 3.41-3.31 (m, 2H), 3.22-3.05 (m, 2H), 3.00-2.88 (m, 2H), 2.73-2.66 (m, 2H), 2.51 (s, 3H), 1.97-1.86 (m, 1H), 0.91 (d, J = 6.7 Hz, 3H), 0.86 (d, J = 6.7 Hz, 3H) |
| 116 | 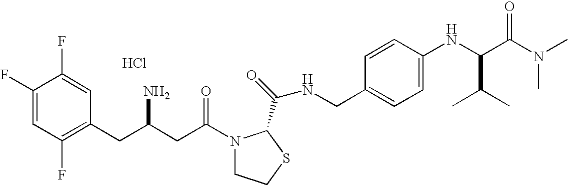 | (DMSO-d6, 300 MHz) δ 8.43- 8.37 (brt, 1H), 8.16 (br, 3H), 7.60-7.44 (m, 2H), 6.99-6.90 (m, 2H), 6.73-6.69 (m, 2H), 5.36 (s, 1H), 4.23 (d, J = 6.6 Hz, 1H), 4.14-4.01 (m, 2H), 3.96-3.87 (m, 1H), 3.78-3.62 (m, 2H), 3.22-3.09 (m, 2H), 3.05-2.92 (m, 2H), 3.03 (s, 3H), 2.83-2.65 (m, 2H), 2.76 (s, 3H), 2.05-1.92 (m, 1 H), 0.92 (d, J = 6.6 Hz, 3H), 0.90 (d, J = 6.6 Hz, 3H) |
| 117 | 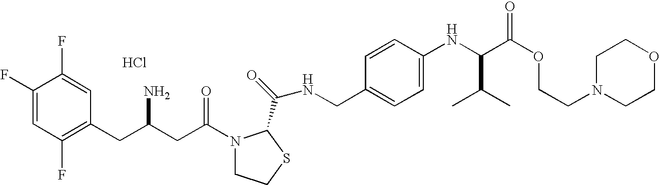 | (DMSO-d6, 300 MHz) δ 8.36 (brt, 1H), 8.14 (br, 3H), 7.60- 7.49 (m, 2H), 6.97-6.90 (m, 2H), 6.64-6.57 (m, 2H), 5.36 (s, 1H), 4.51-4.27 (m, 9H), 4.12-3.62 (m, 9H), 3.30-2.90 (m, 4H), 2.86-2.68 (m, 2H), 2.12-1.99 (m, 1H), 0.97 (d, J = 6.6 Hz, 3H), 0.92 (d, J = 6.6 Hz, 3H) |
| 118 | 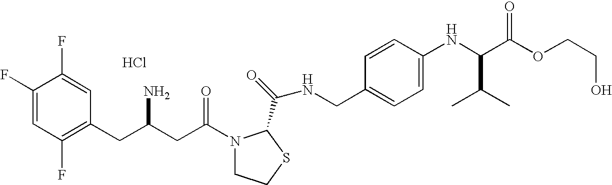 | (DMSO-d6, 300 MHz) δ 8.33 (brt, 1H), 8.15 (brs, 3H), 7.56- 7.50 (m, 2H), 6.96-6.89 (m, 2H), 6.59-6.51 (m, 2H), 5.35 (s, 1H), 4.09-3.87 (m, 5H), 3.72-3.66 (m, 3H), 3.52-3.49 (m, 2H), 3.19-3.11 (m, 2H), 2.98-2.95 (m, 2H), 2.75-2.70 (m, 2H), 2.05-1.95 (m, 1H), 1.02 (d, J = 6.7 Hz, 3H), 0.90 (d, J = 6.7 Hz, 3H) |
| 119 | 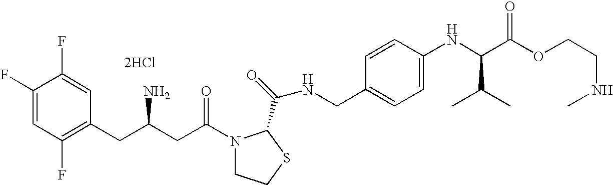 | (DMSO-d6, 300 MHz) δ 9.31 (br, 2H), 8.52 (brt, 1H), 8.26 (brs, 3H), 7.74-7.63 (m, 2H), 7.11-7.05 (m, 2H), 6.79-6.74 (m, 2H), 5.51 (s, 1H), 4.44-3.48 (m, 13H), 3.33-3.25 (m, 2H), 3.14-3.05 (m, 2H), 3.92-2.85 (m, 2H), 2.28-2.19 (m, 1H), 1.12-1.07 (m, 6H) |
| 120 | 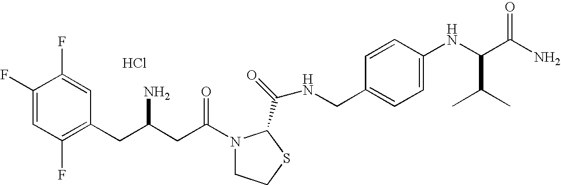 | (DMSO-d6, 300 MHz) δ 8.34 (brt, 1H), 7.99 (br, 3H), 7.60- 7.47 (m, 2H), 7.33 (m, 1H), 6.99 (br, 1H), 6.99-6.92 (m, 2H), 6.61-6.55 (m, 2H), 5.39 (s, 1H), 4.11-4.04 (m, 2H), 3.95-3.10 (m, 6H), 3.03-2.90 (m, 2H), 2.74-2.69 (m, 2H), 2.00-1.89 (m, 1H), 0.96 (d, J = 6.7 Hz, 3H), 0.93 (d, J = 6.7 Hz, 3H) |
| 121 | 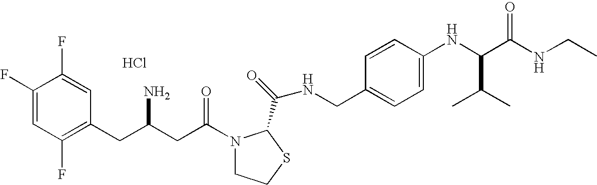 | (DMSO-d6, 300 MHz) δ 8.37 (brt, 1H), 8.13 (brs, 3H), 7.96 (br, 1H), 7.62-7.49 (m, 2H), 7.01-6.94 (m, 2H), 6.67- 6.60 (m, 2H), 5.39 (s, 1H), 4.15-4.08 (m, 2H), 3.97-3.90 (m, 1H), 3.80-3.65 (m, 3H), 3.51-3.44 (m, 2H), 3.22-2.96 (m, 4H), 2.82-2.68 (m, 2H), 2.02-1.92 (m, 1H), 0.98-0.90 (m, 9H) |
| 122 | 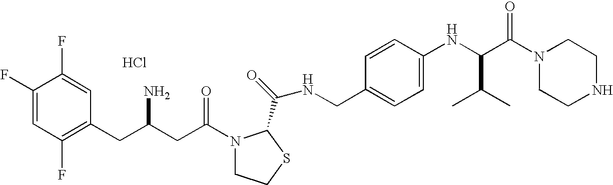 | (DMSO-d6, 300 MHz) δ 9.51 (br, 2H), 8.40 (brt, 1H), □ 8.17 (br, 3H), 7.63-7.50 (m, 2H), 7.01-6.95 (m, 2H), 6.72-6.67 (m, 2H), 5.40 (s, 1H), 4.25 (d, J = 6.7 Hz,, 1H), 4.13-4.06 (m, 2H), 3.98-3.60 (m, 7H), 3.26-3.14 (m, 2H), 3.08-2.89 (m, 6H), 2.86-2.68 (m, 2H), 2.05-1.92 (m, 1H), 0.93 (d, J = 6.7 Hz, 6H) |
| 123 | 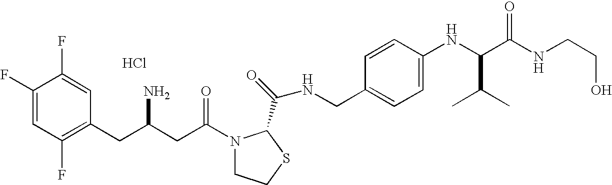 | (DMSO-d6, 300 MHz) δ 8.36 (br, 1H), 8.16 (br, 3H), 7.99 (br, 1H), 7.62-7.49 (m, 2H), 7.02-6.96 (m, 2H), 6.69-6.64 (m, 2H), 5.40 (s, 1H), 4.13-4.04 (m, 2H), 3.97-3.89 (m, 1H), 3.80-3.63 (m, 3H), 3.36-3.33 (m, 2H), 3.20-2.94 (m, 6H), 2.80-2.27 (m, 2H), 2.06-1.95 (m, 1H), 0.96 (d, J = 6.7 Hz, 3H), 0.92 (d, J = 6.7 Hz, 3H) |
| 124 | 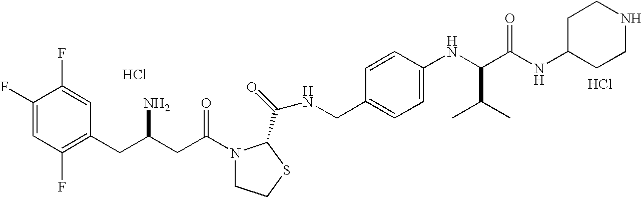 | (DMSO-d6, 300 MHz) δ 8.98 (brs, 2H), 8.50-8.20 (m, 4H), 7.60-7.45 (m, 2H), 6.96 (d, J = 8.4 Hz, 2H), 6.68 (d, J = 8.4 Hz, 2H), 5.38 (s, 1H), 4.25-3.45 (m, 11H), 3.20-2.73 (m, 6H), 2.08-1.93 (m, 1H), 1.85-1.50 (m, 4H), 0.96 (d, J = 6.7 Hz, 3H), 0.89 (d, J = 6.7 Hz, 3H) |
| 125 | 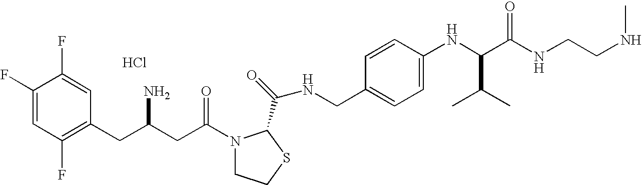 | (DMSO-d6, 300 MHz) δ 8.88 (brs, 2H), 7.42-7.30 (m, 2H), 8.05 (brs, 3H), 7.58-7.45 (m, 2H), 6.96 (d, J = 8.2 Hz, 2H), 6.56 (d, J = 8.2 Hz, 2H), 5.36 (s, 1H), 4.20-3.90 (m, 3H), 3.80-3.45 (m, 5H), 3.40-3.10 (m, 4H), 3.05-2.73 (m, 7H), 2.06-1.95 (m, 1H), 0.92 (d, J = 6.6 Hz, 3H), 0.88 (d, J = 6.6 Hz, 3H) |
| 126 | 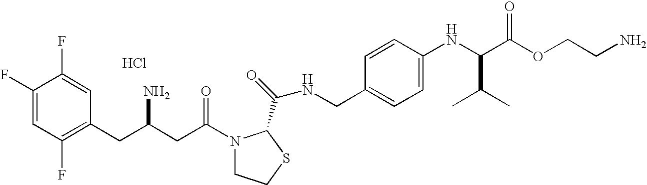 | (DMSO-d6, 300 MHz) δ 8.36 (brt, 1H), 8.18-8.05- (brm, 6H), 7.60-7.50 (m, 2H), 6.96- 6.92 (m, 2H), 6.65-6.58 (m, 2H), 5.38 (s, 1H), 4.23-4.04 (m, 4H), 3.90 (m, 1H), 3.86-3.72 (m, 3H), 3.21-3.15 (m, 2H), 3.17-2.95 (m, 4H), 2.75-2.70 (m, 2H), 2.15-2.05 (m, 1H), 0.96 (d, J = 6.7 Hz, 3H), 0.93 (d, J = 6.7 Hz, 3H) |
| 127 | 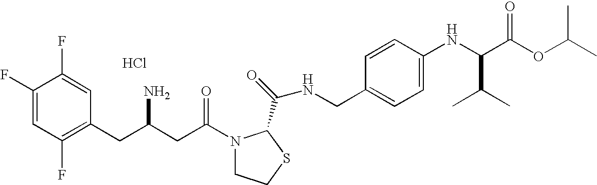 | (DMSO-d6, 300 MHz) δ 8.34 (brt, 1H), 8.12 (brs, 3H), 7.59-7.44 (m, 2H), 6.91 (d, J = 7.5 Hz, 2H), 6.52 (d, J = 7.5 Hz, 2H), 5.38 (s, 1H), 4.89 (m, 1H), 4.16 (m, 2H), 3.98-3.84 (m, 1H), 3.81-3.65 (m, 3H), 3.22-3.10 (m, 2H), 3.02-2.94 (m, 2H), 2.81-2.70 (m, 2H), 2.03-1.95 (m, 1H), 1.25-1.15 (m, 6H), 0.95 (d, J = 6.7 Hz, 3H), 0.92 (d, J = 6.7 Hz, 3H) |
| 128 | 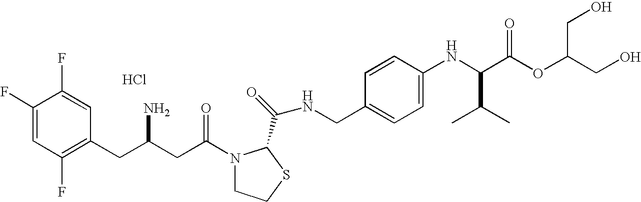 | (DMSO-d6, 300 MHz) δ 8.33 (brt, 1H), 8.03 (brs, 3H), 7.61-7.49 (m, 2H), 6.92 (d, J = 8.4 Hz, 2H), 6.55 (d, J = 8.4 Hz, 2H), 5.37 (s, 1H), 4.71 (m, 1H), 4.18-3.66 (m, 6H), 3.48-3.40 (m, 4H), 3.27-3.15 (m, 2H), 2.95-2.90 (m, 2H), 2.75-2.70 (m, 2H), 2.07-1.97 (m, 1H), 0.96 (d, J = 6.7 Hz, 3H), 0.92 (d, J = 6.7 Hz, 3H) |
| 129 | 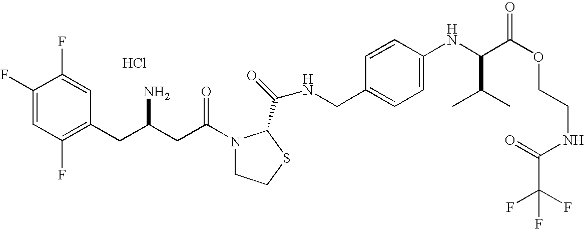 | (DMSO-d6, 300 MHz) δ 9.56 (brs, 1H), 8.40-8.10 (brm, 4H), 7.65-7.55 (m, 2H), 7.00-6.92 (m, 2H), 6.60-6.52 (m, 2H), 5.36 (s, 1H), 4.20-3.45 (m, 10H), 3.20-3.10 (m, 2H), 3.05- 2.96 (m, 2H), 2.78-2.70 (m, 2H), 2.03-1.98 (m, 1H), 1.00- 0.90 (m, 6H) |
| 130 | 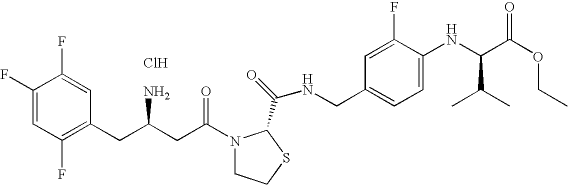 | (DMSO-d6, 300 MHz) δ 8.50- 8.46 (brt, 1H), 8.13 (br, 3H), 7.61-7.49 (m, 2H), 6.99-6.81 (m, 2H), 6.71-6.59 (m, 1H), 5.38 (s, 1H), 5.14 (br, 1H), 4.19-4.04 (m, 4H), 3.98-3.92 (m, 1H), 3.83-3.66 (m, 3H), 3.26-3.13 (m, 2H), 3.08-2.92 (m, 2H), 2.88-2.72 (m, 2H), 2.17-2.06 (m, 1H), 1.15 (t, J = 7.1 Hz, 3H), 0.98 (d, J = 6.6 Hz, 3H), 0.92 (d, J = 6.6 Hz, 3H) |
| 131 | 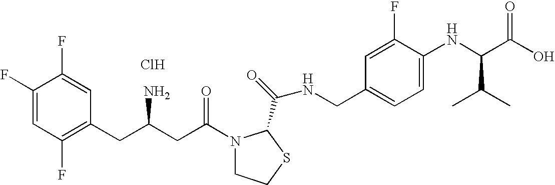 | (DMSO-d6, 300 MHz) δ 8.49- 8.45 (brt, 1H), 8.11 (br, 3H), 7.61-7.49 (m, 2H), 6.99-6.81 (m, 2H), 6.71-6.59 (m, 1H), 5.38 (s, 1H), 4.96 (br, 1H), 4.19-4.05 (m, 2H), 3.98-3.92 (m, 1H), 3.80-3.65 (m, 3H), 3.26-3.13 (m, 2H), 3.08-2.92 (m, 2H), 2.88-2.72 (m, 2H), 2.17-2.06 (m, 1H), 0.98 (d, J = 6.6 Hz, 3H), 0.92 (d, J = 6.6 Hz, 3H) |
| 132 | 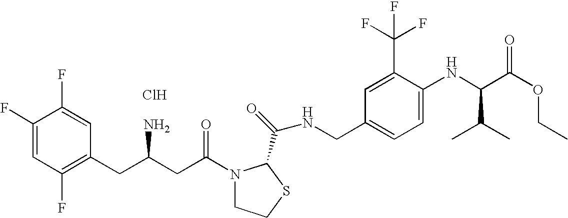 | (DMSO-d6, 300 MHz) δ 8.54- 8.50 (brt, 1H), 8.09 (br, 3H), 7.65-7.26 (m, 4H), 6.90-6.75 (m, 1H), 5.38 (s, 1H), 4.81 (brd, 1H), 4.25-4.04 (m, 4H), 3.99- 3.92 (m, 1H), 3.80-3.64 (m, 3H), 3.22-3.15 (m, 2H), 3.07- 2.91 (m, 2H), 2.85-2.67 (m, 2H), 2.20-2.09 (m, 1H), 1.18 (t, J = 7.1 Hz, 3H), 0.98-0.89 (m, 6H) |
| 133 | 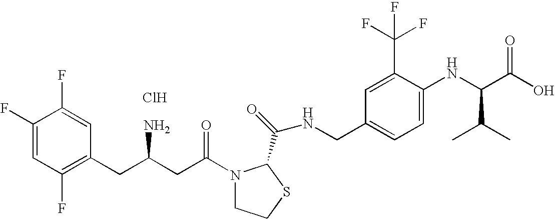 | (DMSO-d6, 300 MHz) δ 13.08 (br, 1H), 8.53-8.49 (brt, 1H), 8.10 (br, 3H), 7.60-7.51 (m, 2H), 7.38-7.26 (m, 2H), 6.85- 6.75 (m, 1H), 5.38 (s, 1H), 4.85 (brd, 1H), 4.24-3.92 (m, 4H), 3.79-3.66 (m, 2H), 3.24-3.14 (m, 2H), 3.07-2.88 (m, 2H), 2.85-2.68 (m, 2H), 2.18-2.09 (m, 1H), 0.98-0.89 (m, 6H) |
| 134 | 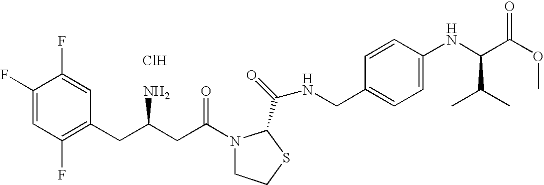 | (DMSO-d6, 300 MHz) δ 8.36 (brt, 1H), 8.17 (brs, 3H), 7.62- 7.49 (m, 2H), 7.00-6.93 (m, 2H), 6.60-6.52 (2H), 5.39 (s, 3H), 4.15-4.01 (m, 2H), 3.98-3.91 (1H), 3.80-3.67 (m, 3H), 3.60 (s, 3H), 3.25-3.15 (m, 2H), 3.05-2.97 (m, 2H), 2.86-2.68 (m, 2H), 2.08-2.19 (m, 1H), 0.98 (d, J = 6.7 Hz, 3H), 0.92 (d, J = 6.7 Hz, 3H) |
| 135 | 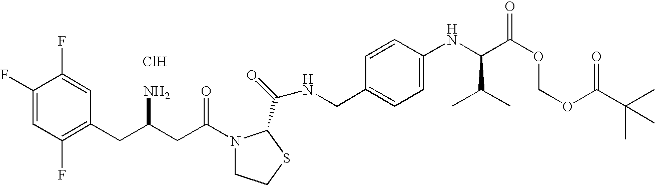 | (DMSO-d6) δ 8.36 (brt, 1H), 8.23 (brs, 3H), 7.60-7.45 (m, 2H), 6.96-6.88 (m, 2H), 6.56-6.49 (m, 2H), 5.72 (d, J = 5.9 Hz, 1H), 5.66 (d, J = 5.9 Hz, 1H), 5.35 (s, 1H), 4.12-3.87 (m, 3H), 3.76-3.63 (m, 3H), 3.20-3.08 (m, 2H) 3.04-2.64 (m, 4H), 2.06-1.95 (m, 1H), 1.05 (s, 9H), 0.94 (d, J = 6.7 Hz, 3H), 0.90 (d, J = 6.7 Hz, 3H) |

### Formulation Example 1

### Preparation of Syrup

A syrup comprising 2 w/v % of a 2-carbonyl-3-acyl-1,3-thiazolidine derivative having β-amino group according to formula 1 or formula (Q) in free or pharmaceutically acceptable salt form may be prepared as follows.

2 g of (R)-ethyl 2-(4-(((S)-3-((R)-3-amino-4-(2,4,5-trifluorophenyl)-butanoyl)-thiazolidin-2-carboxamido)methyl)phenylamino)-3-methylbutanoate.HCl (Compound 36 in Table 1), 25.4 g of sugar and 0.8 g of saccharine are dissolved in 80 g of warm distilled water, and the resulting solution is cooled. Thereto is added a solution of 8.0 g of glycerin, 4.0 g of ethanol, 0.04 g of a flavoring agent, 0.4 g of sorbic acid, and, then, the total volume of the resulting solution is adjusted to 100 ml with addition of distilled water. The components and their amounts used in the above procedure are shown in Table 2.

TABLE 2

| Components | Amount (g) |
|---|---|
| (R)-ethyl 2-(4-(((S)-3-((R)-3-amino-4-(2,4,5-trifluoro- | 2 |
| phenyl)butanoyl)-thiazolidin-2-carboxamido)methyl)- | |
| phenylamino)-3-methylbutanoate•HCl | |
| Saccharin | 0.8 |
| Sugar | 25.4 |
| Glycerine | 8.0 |
| Favoring agent | 0.04 |
| Ethanol | 4.0 |
| Sorbic acid | 0.4 |
| Distilled water | Balanced amount |
| | to 100 ml |

### Formulation Example 2

### Preparation of Tablet

A tablet comprising 15 mg of a 2-carbonyl-3-acyl-1,3-thiazolidine derivative having β-amino group on the acyl chain according to formula 1 or formula (Q) in free or pharmaceutically acceptable salt form may be prepared as follows.

250 g of (R)-ethyl 2-(4-(((S)-3-((R)-3-amino-4-(2,4,5-trifluorophenyl)-butanoyl)thiazolidin-2-carboxamido)methyl)phenylamino)-3-methylbutanoate.HCl (Compound 36 in Table 1) is mixed with 175.9 g of lactose, 180 g of potato starch, and 32 g of colloidal silica. To the resulting mixture, 10 wt % aqueous gelatin solution is added, and the resultant is pulverized, screened through a 14 mesh sieve, and dried. To the powder thus obtained are added 160 g of potato starch, 50 g of talc, and 5 g of magnesium stearate, and the resultant is pressed to form tablets. The components and their amounts used in the above procedure are shown in Table 3.

TABLE 3

| Components | Amount (g) |
|---|---|
| (R)-ethyl 2-(4-(((S)-3-((R)-3-amino-4-(2,4,5-trifluoro- | 250 |
| phenyl)butanoyl)thiazolidin-2-carboxamido)methyl) | |
| phenylamino)-3-methylbutanoate•HCl | |
| Lactose | 175.9 |
| Potato starch | 340 |
| Colloidal silica | 32 |
| 10% Gelatin solution | |
| Talc | 50 |
| Magnesium stearate | 5 |

### Formulation Example 2A

### Preparation of Tablet

A tablet comprising 15 mg of a compound of formula (Q), e.g., 1.1-1.75, or compound of formula 1 in free or pharmaceutically acceptable salt form may be prepared as follows.

15 mg of (R)-ethyl 2-(4-(((S)-3-((R)-3-amino-4-(2,4,5-trifluorophenyl)-butanoyl)thiazolidin-2-carboxamido)methyl)phenylamino)-3-methylbutanoate.HCl (Compound 36 in Table 1), 26 mg of Lactose (granular, 12-mesh), 20 mg of starch, 20 mg of Talc and 0.3 mg of magnesium stearate are mixed thoroughly. The resulting mixture is compressed into slugs, then ground and screened to 14- to 16-mesh granules. The granules are re-compressed into tablets using a 9/32-inch concave punch. The components and their amounts used in this procedure are shown in Table 3A.

TABLE 3A

| Components | Amount (mg) |
|---|---|
| (R)-ethyl 2-(4-(((S)-3-((R)-3-amino-4-(2,4,5-trifluoro- | 15 |
| phenyl)butanoyl)thiazolidin-2-carboxamido)methyl) | |
| phenylamino)-3-methylbutanoate•HCl | |
| Lactose (granular, 12-mesh) | 26 |
| starch | 20 |
| Talc | 20 |
| Magnesium stearate | 0.3 |

### Formulation Example 3

### Preparation of Injective Solution

A solution for injection comprising 10 mg of a 2-thiazolidine derivative having β-amino group according to formula 1 or formula (Q) or its salt may be prepared as follows.

1 g of (R)-ethyl 2-(4-(((S)-3-((R)-3-amino-4-(2,4,5-trifluorophenyl)-butanoyl)thiazolidin-2-carboxamido)methyl)phenylamino)-3-methylbutanoate.HCl obtained in Compound 36, 0.6 g of sodium chloride, and 0.1 g of ascorbic acid are dissolved in distilled water to make 100 ml of the resulting solution. The resulting solution is charged into a vessel, which is heated at 20° C. for 30 minutes to sterilize it. The components and their amounts used in the above procedure are shown in Table 4.

TABLE 4

| Components | Amount (g) |
|---|---|
| (R)-ethyl 2-(4-(((S)-3-((R)-3-amino-4-(2,4,5-trifluoro- | 1 |
| phenyl)butanoyl)thiazolidin-2-carboxamido)methyl)- | |
| phenylamino)-3-methylbutanoate•HCl | |
| Sodium chloride | 0.6 |
| Ascorbic acid | 0.1 |
| Distilled water | Balanced amount |
| | to 100 ml |

### Experimental Example

### Effectiveness in Inhibiting DPP-IV

The effectiveness in inhibiting DPP-IV by the compound of formula 1 or formula (Q) (e.g., Compound 27 or 36) may be evaluated using the extract of human colon carcinoma cells (Caco-2).

Human colon carcinoma cells (Caco-2) obtained from the American Type Culture Collection (ATCC) are cultured for 20 days. The cells are treated with 1 ml of a lysis solution (10 mM Tris, 0.15 M NaCl, 1% Triton® X 100, 10% glycerol) and subjected to centrifugation at a rotation speed of 12,000 rpm for 10 minutes at 4° C. Then, the supernatant is separated. 20 μl of the cell lysate, 10 μl of the test compounds (Example 27 and 36) and 150 μl of incubation buffer solution are added to 96-well microtiter plate, to which 20 μl of Ala-Pro-AFC (final concentration, 40 μM) is added. MK-0431 Sitagliptin is used as a positive control. After incubating for 1 hour at room temperature, the concentrations of the control and test compound that reduce the DPP-IV activity by 50%, i.e., IC₅₀ value are measured. The results are shown in Table 5.

TABLE 5

| Compound | IC₅₀ |
|---|---|
| 27 | 1 nM |
| 36 | 17 nM |
| MK-0431 | 20 nM |

As shown in Table 5, the Compound 27 and 36 exhibited good DPP-IV inhibition activity, thereby activating a hormone such as glucagon-like peptide 1 (GLP-1, GLP-2) to promote insulin secretion from the beta-cell of pancreas and inhibit glucagon secretion from the alpha-cell thereof, which is useful for treating diabetes. Other compounds of the invention also show good DPP-IV inhibition activities. For example, Compounds 26, 27, 28, 29, 35, 36, 37 and 38 all show IC50 value of less than 50 nM.

Thus, the disclosed compounds of formula 1 or formula (Q) can be advantageously used for preventing or treating DPP-IV-mediated diseases such as Type 1 diabetes (insulin-dependent diabetes mellitus), Type 2 diabetes (insulin-independent diabetes mellitus), arthritis, obesity, osteoporosis and impaired glucose tolerance.

While the invention has been described with respect to the above specific embodiments, it should be recognized that various modifications and changes may be made and also fall within the scope of the invention as defined by the claims that follow.

1. A 2-carbonyl-3-acyl-1,3-thiazolidine having a β-amino group on the acyl chain, in free, salt or prodrug form.

2. The compound according to claim 1, wherein said compound is a Compound of formula (Q): 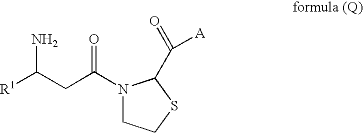
in free, salt or prodrug form, wherein:
A is 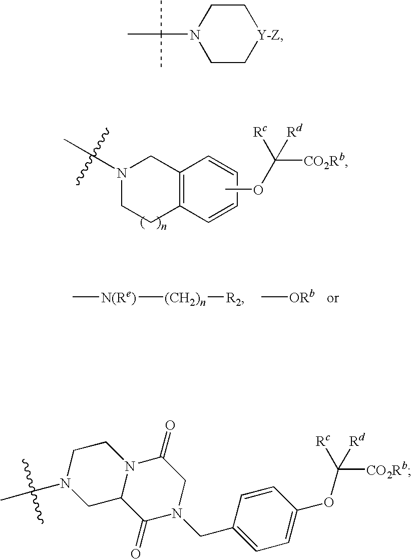
R₁ is 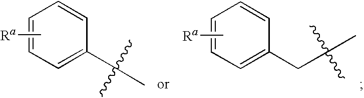
R₂ is C₁₋₆alkyl (e.g., methyl), 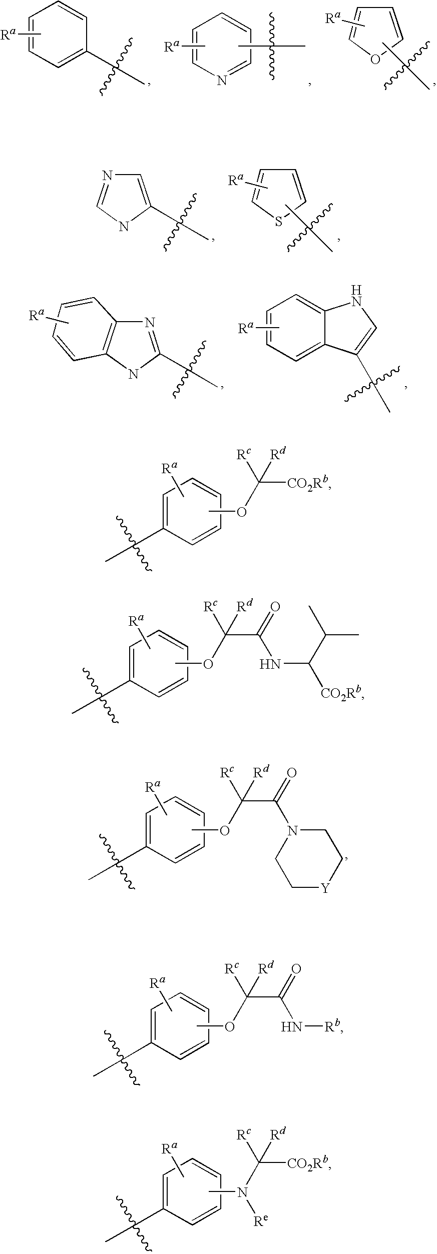 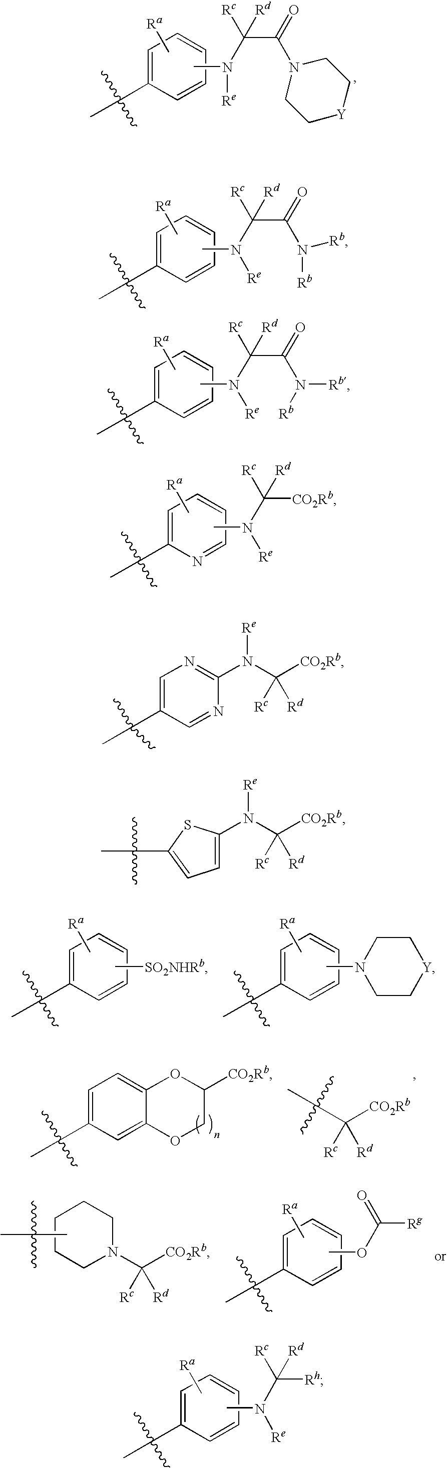
R^{a} is one or more substitutents selected from the group consisting of hydrogen, C₁₋₆ alkyl, C₃₋₆ cycloalkyl, C₁₋₆ alkoxy, —OCF₃, hydroxy, halogen, —CN, —CF₃—COOR^{b}, —CH₂COOR^{b}, and —NR^{d}R^{e};
R^{b} and R^{b′} are independently selected from a group consisting of hydrogen, C₁₋₆ alkyl, C₃₋₆ cycloalkyl or —C₁₋₆alkylC₃₋₆cycloalkyl wherein said cycloalkyl optionally contains one or more heteroatom selected from a group consisting of N, O, or S (e.g., piperazinyl, morpholinyl, morpholin-4-ylethyl, piperidinyl, —CH₂CH₂OH, —CH₂CH₂NH₂, —CH₂CH₂N(CH₂CH₂)₂O, —CH₂CH₂N(CH₂CH₃)₂ or —CH₂CH₂NHCOCH₃; CH₂CH₂NHCOCF₃; CH(CH₂OH)₂; CH₂CH₂OCH₃; CH₂CH₂NHCH₃; CH(CH₂CH₂)₂NH and CH₂OCOC(CH₃)₃;
R^{c} is hydrogen, C₁₋₆ alkyl, C₃₋₆ cycloalkyl, or arylC₁₋₆alkyl-;
R^{d} and R^{e} are each independently hydrogen, C₁₋₆ alkyl or C₃₋₆ cycloalkyl;
R^{g} is C₁₋₆ alkyl;
R^{h} is a substituent selected from the group consisting of hydrogen, C₁₋₆ alkyl, hydroxyC₁₋₆alkyl;
Y is C, O, S or N;
Z is hydrogen, C₁₋₆ alkyl, C₃₋₄ cycloalkyl or —CO₂R^{b} with the proviso that when Y is O or S, Z is absent; and
n is an integer of 0, 1 or 2.

3. The compound according to claim 1, selected from a group consisting of:
(1) methyl 3-((R)-3-amino-4-(2,4,5-trifluorophenyl)butanoyl)thiazolidine-2-carboxylate,
(2) 3-((R)-3-amino-4-(2,4,5-trifluorophenyl)butanoyl)thiazolidine-2-carboxylic acid,
(3) 3-((R)-3-amino-4-(2,4,5-trifluorophenyl)butanoyl)-N-benzylthiazolidine-2-carboxamide,
(4) ethyl 2-(4-((3-((R)-3-amino-4-(2,4,5-trifluorophenyl)butanoyl)thiazolidine-2-carboxamido)methyl)phenoxy)acetate,
(5) 2-4-((3-((R)-3-amino-4-(2,4,5-trifluorophenyl)butanoyl)thiazolidine-2-carboxamido)methyl)phenoxy)acetic acid,
(6) ethyl 2-(4-(3-((R)-3-amino-4-(2,4,5-trifluorophenyl)butanoyl)thiazolidine-2-carboxamido)phenoxy)acetate,
(7) 2-(4-(3-((R)-3-amino-4-(2,4,5-trifluorophenyl)butanoyl)thiazolidine-2-carboxamido)phenoxy)acetic acid,
(8) ethyl 2-(4-((3-((R)-3-amino-4-(2,4,5-trifluorophenyl)butanoyl)thiazolidine-2-carboxamido)methyl)phenoxy)-3-methylbutanoate,
(9) 2-(4-((3-((R)-3-amino-4-(2,4,5-trifluorophenyl)butanoyl)thiazolidine-2-carboxamido)methyl)phenoxy)-3-methylbutanoic acid,
(10) pivaloyloxymethyl 2-(4-((3-((R)-3-amino-4-(2,4,5-trifluorophenyl) butanoyl)thiazolidine-2-carboxamido)methyl)phenoxy)-3-methylbutanoate,
(11) ethyl 1-(3-((R)-3-amino-4-(2,4,5-trifluorophenyl)butanoyl)thiazolidine-2-carbonyl)piperidine-4-carboxylate,
(12) 1-(3-((R)-3-amino-4-(2,4,5-trifluorophenyl)butanoyl)thiazolidine-2-carbonyl)piperidine-4-carboxylic acid,
(13) 2-(4-((3-((R)-3-amino-4-(2,4,5-trifluorophenyl)butanoyl)thiazolidine-2-carboxamido)methyl)phenyl)acetic acid,
(14) ethyl 2-(2-(3-((R)-3-amino-4-(2,4,5-trifluorophenyl)butanoyl)thiazolidine-2-carbonyl)-1,2,3,4-tetrahydroisoquinolin-7-yloxy)-3-methylbutanoate,
(15) 2-(2-(3-((R)-3-amino-4-(2,4,5-trifluorophenyl)butanoyl)thiazolidine-2-carbonyl)-1,2,3,4-tetrahydroisoquinolin-7-yloxy)-3-methylbutanoic acid,
(16) ethyl 6-((3-((R)-3-amino-4-(2,4,5-trifluorophenyl)butanoyl)thiazolidine-2-carboxamido)methyl)-2,3-dihydrobenzo[b][1,4]dioxin-2-carboxylate,
(17) 6-((3-((R)-3-amino-4-(2,4,5-trifluorophenyl)butanoyl)thiazolidine-2-carboxamido)methyl)-2,3-dihydrobenzo[b][1,4]dioxin-2-carboxylic acid,
(18) (S)-3-((R)-3-amino-4-(2,4,5-trifluorophenyl)butanoyl)thiazolidine-2-carboxylic acid,
(19) ethyl 2-(4-((3-((R)-3-((1-acetoxyethoxy)carbonylamino)-4-(2,4,5-trifluorophenyl)butanoyl)thiazolidine-2-carboxamido)methyl)phenoxy)-3-methylbutanoate,
(20) (3R)-3-amino-1-(2-(morpolin-4-carbonyl)thiazolidin-3-yl)-4-(2,4,5-trifluorophenyl)butan-1-one,
(21) N-(2-(1H-imidazol-5-yl)ethyl)-3-((R)-3-amino-4-(2,4,5-trifluorophenyl) butanoyl)thiazolidine-2-carboxamide,
(22) (S)-2-(4-(((S)-3-((R)-3-amino-4-(2,4,5-trifluorophenyl)butanoyl) thiazolidine-2-carboxamido)methyl)phenoxy)-3-methylbutanoic acid,
(23) (R)-2-(4-(((S)-3-((R)-3-amino-4-(2,4,5-trifluorophenyl)butanoyl) thiazolidine-2-carboxamido)methyl)phenoxy)-3-methylbutanoic acid,
(24) (S)-2-(4-(((R)-3-((R)-3-amino-4-(2,4,5-trifluorophenyl)butanoyl) thiazolidine-2-carboxamido)methyl)phenoxy)-3-methylbutanoic acid,
(25) (R)-2-(4-(((R)-3-((R)-3-amino-4-(2,4,5-trifluorophenyl)butanoyl) thiazolidine-2-carboxamido)methyl)phenoxy)-3-methylbutanoic acid,
(26) (S)-2-(4-(((S)-3-((R)-3-amino-4-(2,4,5-trifluorophenyl)butanoyl) thiazolidine-2-carboxamido)methyl)phenylamino)-3-methylbutanoic acid,
(27) (R)-2-(4-(((S)-3-((R)-3-amino-4-(2,4,5-trifluorophenyl)butanoyl) thiazolidine-2-carboxamido)methyl)phenylamino)-3-methylbutanoic acid,
(28) (S)-2-(4-(((R)-3-((R)-3-amino-4-(2,4,5-trifluorophenyl)butanoyl) thiazolidine-2-carboxamido)methyl)phenylamino)-3-methylbutanoic acid,
(29) (R)-2-(4-(((R)-3-((R)-3-amino-4-(2,4,5-trifluorophenyl)butanoyl) thiazolidine-2-carboxamido)methyl)phenylamino)-3-methylbutanoic acid,
(30) ethyl 2-(3-((3-((R)-3-amino-4-(2,4,5-trifluorophenyl)butanoyl) thiazolidine-2-carboxamido)methyl)phenoxy)acetate,
(31) 2-(3-((3-((R)-3-amino-4-(2,4,5-trifluorophenyl)butanoyl)thiazolidine-2-carboxamido)methyl)phenoxy)acetic acid,
(32) ethyl 2-(3-(3-((R)-3-amino-4-(2,4,5-trifluorophenyl)butanoyl) thiazolidine-2-carboxamido)phenoxy)acetate,
(33) 2-(3-(3-((R)-3-amino-4-(2,4,5-trifluorophenyl)butanoyl)thiazolidine-2-carboxamido)phenoxy)acetic acid,
(34) 2-(4-(3-((R)-3-amino-4-(2,4,5-trifluorophenyl)butanoyl)thiazolidine-2-carboxamido)piperidine-1-yl)-3-methylbutanoic acid,
(35) (S)-ethyl 2-(4-(((S)-3-((R)-3-amino-4-(2,4,5-trifluorophenyl)butanoyl) thiazolidine-2-carboxamido)methyl)phenylamino)-3-methylbutanoate,
(36) (R)-ethyl 2-(4-(((S)-3-((R)-3-amino-4-(2,4,5-trifluorophenyl)butanoyl) thiazolidine-2-carboxamido)methyl)phenylamino)-3-methylbutanoate,
(37) (S)-ethyl 2-(4-(((R)-3-((R)-3-amino-4-(2,4,5-trifluorophenyl)butanoyl) thiazolidine-2-carboxamido)methyl)phenylamino)-3-methylbutanoate,
(38) (R)-ethyl 2-(4-(((R)-3-((R)-3-amino-4-(2,4,5-trifluorophenyl)butanoyl) thiazolidine-2-carboxamido)methyl)phenylamino)-3-methylbutanoate,
(39) (3R)-3-amino-1-(2-(thiomorpolin-4-carbonyl)thiazolidin-3-yl)-4-(2,4,5-trifluorophenyl)butan-1-one,
(40) (3R)-3-amino-1-(2-(piperazine-1-carbonyl)thiazolidin-3-yl)-4-(2,4,5-trifluorophenyl)butan-1-one,
(41) (3R)-3-amino-1-(2-(4-methylpiperazine-1-carbonyl)thiazolidin-3-yl)-4-(2,4,5-trifluorophenyl)butan-1-one,
(42) 3-((R)-3-amino-4-(2,4,5-trifluorophenyl)butanoyl)-N,N-dimethyl thiazolidine-2-carboxamide,
(43) 3-((R)-3-amino-4-(2,4,5-trifluorophenyl)butanoyl)-N-(furan-3-yl)methyl) thiazolidine-2-carboxamide,
(44) ethyl 2-(3-((R)-3-amino-4-(2,4,5-trifluorophenyl)butanoyl)thiazolidine-2-carboxamido)acetate,
(45) 2-(3-((R)-3-amino-4-(2,4,5-trifluorophenyl)butanoyl)thiazolidine-2-carboxamido)acetic acid,
(46) N-(2-(1H-indol-3-yl)ethyl)-3-((R)-3-amino-4-(2,4,5-trifluorophenyl) butanoyl)thiazolidine-2-carboxamide,
(47) 3-((R)-3-amino-4-(2,4,5-trifluorophenyl)butanoyl)-N-(4-morpholinophenyl) thiazolidine-2-carboxamide,
(48) 3-((R)-3-amino-4-(2,4,5-trifluorophenyl)butanoyl)-N-(4-sulfamoylphenyl) thiazolidine-2-carboxamide,
(49) 3-((R)-3-amino-4-(2,4,5-trifluorophenyl)butanoyl)-N-(4-sulfamoylbenzyl) thiazolidine-2-carboxamide,
(50) N-((1H-benzo[d]imidazol-2-yl)methyl)-3-((R)-3-amino-4-(2,4,5-trifluorophenyl)butanoyl)thiazolidine-2-carboxamide,
(51) ethyl 2-(4-((3-((R)-3-amino-4-(2,4,5-trifluorophenyl)butanoyl) thiazolidine-2-carboxamido)methyl)-3-fluorophenoxy)butanoate,
(52) 2-(4-((3-((R)-3-amino-4-(2,4,5-trifluorophenyl)butanoyl)thiazolidine-2-carboxamido)methyl)-3-fluorophenoxy)butanoic acid,
(53) ethyl 2-(4-((3-((R)-3-amino-4-(2,4,5-trifluorophenyl)butanoyl)thiazolidine-2-carboxamido)methyl)-3-fluorophenoxy)-2-methylpropanoate,
(54) 2-(4-((3-((R)-3-amino-4-(2,4,5-trifluorophenyl)butanoyl)thiazolidine-2-carboxamido)methyl)-3-fluorophenoxy)-2-methylpropanoic acid,
(55) ethyl 2-(4-((3-((R)-3-amino-4-(2,4,5-trifluorophenyl)butanoyl)thiazolidine-2-carboxamido)methyl)-2-fluorophenoxy)-2-methylpropanoate,
(56) 3-((R)-3-amino-4-(2,4,5-trifluorophenyl)butanoyl)-N-(pyridin-4-yl methyl)thiazolidine-2-carboxamide,
(57) (S)-2-(2-(4-((3-((R)-3-amino-4-(2,4,5-trifluorophenyl)butanoyl)thiazolidine-2-carboxamido)methyl)phenoxy)-3-methylbutanamido)-3-methylbutanoic acid,
(58) (R)-ethyl 2-(4-((8-(3-((R)-3-amino-4-(2,4,5-trifluorophenyl)butanoyl) thiazolidine-2-carbonyl)-1,4-dioxo-hexahydro-1,1-pyrazino[1,2-a]pyrazin-2(6H)-yl)methyl)phenoxy)-3-methylbutanoate,
(59) (R)-2-(4-((8-(3-((R)-3-amino-4-(2,4,5-trifluorophenyl)butanoyl)thiazolidine-2-carbonyl)-1,4-dioxo-hexahydro-1H-pyrazino[1,2-a]pyrazin-2(6H)-yl)methyl)phenoxy)-3-methylbutanoic acid,
(60) ethyl 2-(2-(3-((R)-3-amino-4-(2,4,5-trifluorophenyl)butanoyl)thiazolidine-2-carbonyl)-1,2,3,4-tetrahydroisoquinoline-6-yloxy)-3-methylbutanoate,
(61) 2-(2-(3-((R)-3-amino-4-(2,4,5-trifluorophenyl)butanoyl)thiazolidine-2-carbonyl)-1,2,3,4-tetrahydroisoquinoline-6-yloxy)-3-methylbutanoic acid,
(62) ethyl 5-((3-((R)-3-amino-4-(2,4,5-trifluorophenyl)butanoyl)thiazolidine-2-carboxamido)methyl)benzo[d][1,3]dioxol-2-carboxylate,
(63) 5-((3-((R)-3-amino-4-(2,4,5-trifluorophenyl)butanoyl)thiazolidine-2-carboxamido)methyl)benzo[d][1,3]dioxol-2-carboxylic acid,
(64) 2-(4-((3-((R)-3-amino-4-(2,4,5-trifluorophenyl)butanoyl)thiazolidine-2-carboxamido)methyl)phenoxy)-2-methylpropanoic acid,
(65) (R)-2-(4-((3-((R)-3-amino-4-(2,4,5-trifluorophenyl)butanoyl)thiazolidine-2-carboxamido)methyl)phenoxy)-3-phenylpropanoic acid,
(66) 2-(4-((3-((R)-3-amino-4-(2,4,5-trifluorophenyl)butanoyl)-N-methyl thiazolidine-2-carboxamido)methyl)phenoxy)-3-methylbutanoic acid,
(67) ethyl 2-(4-((3-((R)-3-amino-4-(2,4,5-trifluorophenyl)butanoyl)thiazolidine-2-carboxamido)methyl)phenylamino)-3-methylbutanoate,
(68) 2-(4-((3-((R)-3-amino-4-(2,4,5-trifluorophenyl)butanoyl)thiazolidine-2-carboxamido)methyl)phenylamino)-3-methylbutanoic acid,
(69) ethyl 2-(4-((3-((R)-3-amino-4-(2,4,5-trifluorophenyl)butanoyl)thiazolidine-2-carboxamido)methyl)-3-fluorophenoxy)-3-methylbutanoate,
(70) 2-(4-((3-((R)-3-amino-4-(2,4,5-trifluorophenyl)butanoyl)thiazolidine-2-carboxamido)methyl)-3-fluorophenoxy)-3-methylbutanoic acid,
(71) 2-(4-((3-((R)-3-amino-4-(2,4,5-trifluorophenyl)butanoyl)thiazolidine-2-carboxamido)methyl)-2-fluorophenoxy)-2-methylpropanoic acid,
(72) ethyl 2-(4-(((S)-3-((R)-3-amino-4-(2,4,5-trifluorophenyl)butanoyl) thiazolidine-2-carboxamido)methyl)phenylamino)-2-methylpropanoate,
(73) 2-(4-(((S)-3-((R)-3-amino-4-(2,4,5-trifluorophenyl)butanoyl)thiazolidine-2-carboxamido)methyl)phenylamino)-2-methylpropanoic acid,
(74) (S)-methyl 2-(2-(((S)-3-((R)-3-amino-4-(2,4,5-trifluorophenyl)butanoyl)thiazolidine-2-carboxamido)methyl)-5-bromophenylamino)-3-methylbutanoate,
(75) (S)-ethyl 2-(3-((R)-3-amino-4-(2,4,5-trifluorophenyl)butanoyl)thiazolidine-2-carboxamido)-3-methylbutanoate,
(76) (S)-2-(3-((R)-3-amino-4-(2,4,5-trifluorophenyl)butanoyl)thiazolidine-2-carboxamido)-3-methylbutanoic acid,
(77) (2S,3S)-ethyl 2-(3-((R)-3-amino-4-(2,4,5-trifluorophenyl)butanoyl) thiazolidine-2-carboxamido)-3-methylpentanoate,
(78) (2S,3S)-2-(3-((R)-3-amino-4-(2,4,5-trifluorophenyl)butanoyl)thiazolidine-2-carboxamido)-3-methylpentanoic acid,
(79) ethyl 2-(4-(3-((R)-3-amino-4-(2,4,5-trifluorophenyl)butanoyl) thiazolidine-2-carboxamido)piperidine-1-yl)-3-methylbutanoate,
(80) 4-((3-((R)-3-amino-4-(2,4,5-trifluorophenyl)butanoyl)thiazolidine-2-carboxamido)methyl)phenyl acetate,
(81) 3-((R)-3-amino-4-(2,4,5-trifluorophenyl)butanoyl)-N-(4-hydroxybenzyl) thiazolidine-2-carboxamide,
(82) ethyl 2-((4-((3-((R)-3-amino-4-(2,4,5-trifluorophenyl)butanoyl)thiazolidine-2-carboxamido)methyl)phenyl)(methyl)amino)-3-methylbutanoate,
(83) methyl 4-((3-((R)-3-amino-4-(2,4,5-trifluorophenyl)butanoyl)thiazolidine-2-carboxamido)methyl)-2-hydroxybenzoate,
(84) ethyl 2-(4-((3-((R)-3-amino-4-(2,4,5-trifluorophenyl)butanoyl)thiazolidine-2-carboxamido)methyl)phenoxy)propanoate,
(85) 2-((4-((3-((R)-3-amino-4-(2,4,5-trifluorophenyl)butanoyl)thiazolidine-2-carboxamido)methyl)phenyl)(methyl)amino)-3-methylbutanoic acid,
(86) 4-((3-((R)-3-amino-4-(2,4,5-trifluorophenyl)butanoyl)thiazolidine-2-carboxamido)methyl)-2-hydroxybenzoic acid,
(87) (S)-2-(2-(((S)-3-((R)-3-amino-4-(2,4,5-trifluorophenyl)butanoyl)thiazolidine-2-carboxamido)methyl)-5-bromophenylamino)-3-methylbutanoic acid,
(88) (S)-ethyl 2-(4-(((S)-3-((R)-3-amino-4-(2,4,5-trifluorophenyl)butanoyl) thiazolidine-2-carboxamido)methyl)phenoxy)-3-methylbutanoate,
(89) (S)-ethyl 2-(4-(((S)-3-((R)-3-amino-4-(2,4,5-trifluorophenyl)butanoyl) thiazolidine-2-carboxamido)methyl)-2-fluorophenylamino)-3-methylbutanoate,
(90) (S)-2-(4-(((S)-3-((R)-3-amino-4-(2,4,5-trifluorophenyl)butanoyl) thiazolidine-2-carboxamido)methyl)-2-fluorophenylamino)-3-methylbutanoic acid,
(91) (S)-ethyl 2-(6-(((S)-3-((R)-3-amino-4-(2,4,5-trifluorophenyl)butanoyl) thiazolidine-2-carboxamido)methyl)pyridin-3-ylamino)-3-methylbutanoate,
(92) (S)-2-(6-(((S)-3-((R)-3-amino-4-(2,4,5-trifluorophenyl)butanoyl) thiazolidine-2-carboxamido)methyl)pyridin-3-ylamino)-3-methylbutanoic acid,
(93) 2-(4-((3-((R)-3-amino-4-(2,4,5-trifluorophenyl)butanoyl)thiazolidine-2-carboxamido)methyl)phenoxy)propanoic acid,
(94) (S)-2-(4-(((S)-3-((R)-3-amino-4-(2,4,5-trifluorophenyl)butanoyl) thiazolidine-2-carboxamido)methyl)phenylamino)-3,3-dimethylbutanoic acid,
(95) (S)-2-(2-(((S)-3-((R)-3-amino-4-(2,4,5-trifluorophenyl)butanoyl) thiazolidine-2-carboxamido)methyl)phenylamino)-3-methylbutanoic acid,
(96) (S)-2-(3-(((S)-3-((R)-3-amino-4-(2,4,5-trifluorophenyl)butanoyl) thiazolidine-2-carboxamido)methyl)phenylamino)-3-methylbutanoic acid,
(97) 4-((3-((R)-3-amino-4-(2,4,5-trifluorophenyl)butanoyl)thiazolidine-2-carboxamido)methyl)benzoic acid,
(98) 3-((R)-3-amino-4-(2,4,5-trifluorophenyl)butanoyl)-N-(4-(2-oxo-2-(piperazine-1-yl)ethoxy)benzyl)thiazolidine-2-carboxamide,
(99) 3-((R)-3-amino-4-(2,4,5-trifluorophenyl)butanoyl)-N-(4-(2-oxo-2-thiomorpholinoethoxy)benzyl)thiazolidine-2-carboxamide,
(100) 3-((R)-3-amino-4-(2,4,5-trifluorophenyl)butanoyl)-N-(4-(2-morpholino-2-oxoethoxy)benzyl)thiazolidine-2-carboxamide,
(101) (S)-ethyl 2-(5-(((S)-3-((R)-3-amino-4-(2,4,5-trifluorophenyl)butanoyl) thiazolidine-2-carboxamido)methyl)pyridin-2-ylamino)-3-methylbutanoate,
(102) (S)-2-(5-(((S)-3-((R)-3-amino-4-(2,4,5-trifluorophenyl)butanoyl) thiazolidine-2-carboxamido)methyl)pyridin-2-ylamino)-3-methylbutanoic acid,
(103) (S)-3-((R)-3-amino-4-(2,4,5-trifluorophenyl)butanoyl)-N-(4-((R)-3-methyl-1-morpholino-1-oxobutan-2-ylamino)benzyl)thiazolidine-2-carboxamide,
(104) (R)-ethyl 2-(5-(((S)-3-((R)-3-amino-4-(2,4,5-trifluorophenyl)butanoyl) thiazolidine-2-carboxamido)methyl)pyridin-2-ylamino)-3-methylbutanoate,
(105) (R)-2-(5-(((S)-3-((R)-3-amino-4-(2,4,5-trifluorophenyl)butanoyl) thiazolidine-2-carboxamido)methyl)pyridin-2-ylamino)-3-methylbutanoic acid,
(106) (S)-3-((R)-3-amino-4-(2,4,5-trifluorophenyl)butanoyl)-N-(4-((S)-3-methyl-1-morpholino-1-oxobutan-2-ylamino)benzyl)thiazolidine-2-carboxamide,
(107) (R)-ethyl 2-(5-(((S)-3-((R)-3-amino-4-(2,4,5-trifluorophenyl)butanoyl) thiazolidine-2-carboxamido)methyl)pyrimidin-2-ylamino)-3-methyl butanoate,
(108) (R)-2-(5-(((S)-3-((R)-3-amino-4-(2,4,5-trifluorophenyl)butanoyl) thiazolidine-2-carboxamido)methyl)pyrimidin-2-ylamino)-3-methylbutanoic acid,
(109) (R)-ethyl 2-(5-(((S)-3-((R)-3-amino-4-(2,4,5-trifluorophenyl)butanoyl) thiazolidine-2-carboxamido)methyl)pyridin-2-yloxy)-3-methylbutanoate,
(110) (R)-2-(5-(((S)-3-((R)-3-amino-4-(2,4,5-trifluorophenyl)butanoyl) thiazolidine-2-carboxamido)methyl)pyridin-2-yloxy)-3-methylbutanoic acid,
(111) (R)-ethyl 2-(4-(((S)-3-((R)-3-amino-4-(2,4,5-trifluorophenyl)butanoyl) thiazolidine-2-carboxamido)methyl)-3-fluorophenylamino)-3-methylbutanoate,
(112) (R)-2-(4-(((S)-3-((R)-3-amino-4-(2,4,5-trifluorophenyl)butanoyl) thiazolidine-2-carboxamido)methyl)-3-fluorophenylamino)-3-methylbutanoic acid,
(113) (S)-3-((R)-3-amino-4-(2,4,5-trifluorophenyl)butanoyl)-N-(4-((R)-1-hydroxy-3-methylbutan-2-ylamino)benzyl)thiazolidine-2-carboxamide,
(114) (R)-2-methoxyethyl 2-(4-(((S)-3-((R)-3-amino-4-(2,4,5-trifluorophenyl) butanoyl)thiazolidine-2-carboxamido)methyl)phenylamino)-3-methylbutanoate,
(115) (S)-3-((R)-3-amino-4-(2,4,5-trifluorophenyl)butanoyl)-N-(4-((R)-3-methyl-1-(methylamino)-1-oxobutan-2-ylamino)benzyl)thiazolidine-2-carboxamide,
(116) (S)-3-((R)-3-amino-4-(2,4,5-trifluorophenyl)butanoyl)-N-(4-((R)-1-(dimethylamino)-3-methyl-1-oxobutan-2-ylamino)benzyl)thiazolidine-2-carboxamide,
(117) (R)-2-morpholinoethyl 2-(4-(((S)-3-((R)-3-amino-4-(2,4,5-trifluorophenyl) butanoyl)thiazolidine-2-carboxamido)methyl)phenylamino)-3-methylbutanoate,
(118) (R)-2-hydroxyethyl 2-(4-(((S)-3-((R)-3-amino-4-(2,4,5-trifluorophenyl) butanoyl)thiazolidine-2-carboxamido)methyl)phenylamino)-3-methylbutanoate,
(119) (R)-2-(methylamino)ethyl 2-(4-(((S)-3-((R)-3-amino-4-(2,4,5-trifluorophenyl)butanoyl)thiazolidine-2-carboxamido)methyl)phenylamino)-3-methylbutanoate,
(120) (S)—N-(4-((R)-1-amino-3-methyl-1-oxobutan-2-ylamino)benzyl)-3-((R)-3-amino-4-(2,4,5-trifluorophenyl)butanoyl)thiazolidine-2-carboxamide,
(121) (S)-3-((R)-3-amino-4-(2,4,5-trifluorophenyl)butanoyl)-N-(4-((R)-1-(ethylamino)-3-methyl-1-oxobutan-2-ylamino)benzyl)thiazolidine-2-carboxamide,
(122) (S)-3-((R)-3-amino-4-(2,4,5-trifluorophenyl)butanoyl)-N-(4-((R)-3-methyl-1-oxo-1-(piperazin-1-yl)butan-2-ylamino)benzyl)thiazolidine-2-carboxamide,
(123) (S)-3-((R)-3-amino-4-(2,4,5-trifluorophenyl)butanoyl)-N-(4-((R)-1-(2-hydroxyethylamino)-3-methyl-1-oxobutan-2-ylamino)benzyl)thiazolidine-2-carboxamide,
(124) (S)-3-((R)-3-amino-4-(2,4,5-trifluorophenyl)butanoyl)-N-(4-((R)-3-methyl-1-oxo-1-(piperidin-4-ylamino)butan-2-ylamino)benzyl)thiazolidine-2-carboxamide,
(125) (S)-3-((R)-3-amino-4-(2,4,5-trifluorophenyl)butanoyl)-N-(4-((R)-3-methyl-1-(2-(methylamino)ethylamino)-1-oxobutan-2-ylamino)benzyl)thiazolidine-2-carboxamide,
(126) (R)-2-aminoethyl 2-(4-(((S)-3-((R)-3-amino-4-(2,4,5-trifluorophenyl) butanoyl)thiazolidine-2-carboxamido)methyl)phenylamino)-3-methylbutanoate,
(127) (R)-isopropyl 2-(4-(((S)-3-((R)-3-amino-4-(2,4,5-trifluorophenyl) butanoyl)thiazolidine-2-carboxamido)methyl)phenylamino)-3-methylbutanoate,
(128) (R)-1,3-dihydroxypropan-2-yl 2-(4-(((S)-3-((R)-3-amino-4-(2,4,5-trifluorophenyl)butanoyl)thiazolidine-2-carboxamido) methyl)phenylamino)-3-methylbutanoate,
(129) (R)-2-(2,2,2-trifluoroacetamido)ethyl 2-(4-(((S)-3-((R)-3-amino-4-(2,4,5-trifluorophenyl)butanoyl) thiazolidine-2-carboxamido)methyl)phenylamino)-3-methylbutanoate,
(130) (R)-ethyl 2-(4-(((S)-3-((R)-3-amino-4-(2,4,5 trifluorophenyl)butanoyl)thiazolidine-2-carboxamido) methyl)-2-fluorophenylamino)-3-methylbutanoate,
(131) (R)-2-(4-(((S)-3-((R)-3-amino-4-(2,4,5-trifluorophenyl)butanoyl)thiazolidine-2-carboxamido)methyl)-2-fluorophenylamino)-3-methylbutanoic acid,
(132) (R)-ethyl 2-(4-(((S)-3-((R)-3-amino-4-(2,4,5-trifluorophenyl)butanoyl)thiazolidine-2-carboxamido)methyl)-2-(trifluoromethyl)phenylamino)-3-methylbutanoate,
(133) (R)-2-(4-(((S)-3-((R)-3-amino-4-(2,4,5-trifluorophenyl)butanoyl)thiazolidine-2-carboxamido)methyl)-2-(trifluoromethyl)phenylamino)-3-methylbutanoic acid,
(134) (R)-ethyl 2-(4-(((S)-3-((R)-3-amino-4-(2,4,5-trifluorophenyl)butanoyl)thiazolidine-2-carboxamido)methyl)-3-fluorophenylamino)-3-methylbutanoate, and
(135) (R)-2-(4-(((S)-3-((R)-3-amino-4-(2,4,5-trifluorophenyl)butanoyl)thiazolidine-2-carboxamido)methyl)-3-fluorophenylamino)-3-methylbutanoic acid,
in free, salt or prodrug form.

4. The compound according to claim 1 wherein said compound is 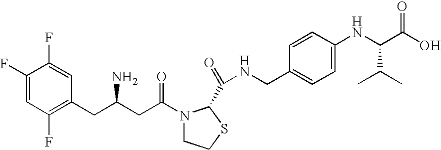
in free, salt or prodrug form.

5. The compound according to claim 1 wherein said compound is 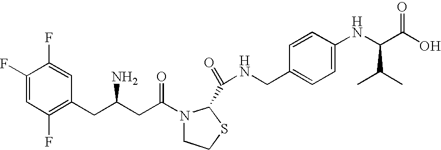
in free, salt or prodrug form.

6. The compound according to claim 1, wherein said compound is 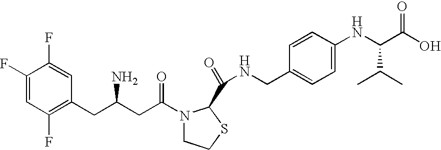
in free, salt or prodrug form.

7. The compound according to claim 1, wherein said compound is 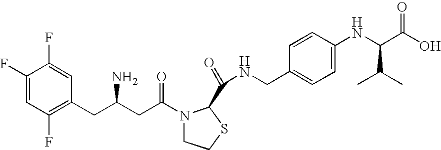
in free, salt or prodrug form.

8. The compound according to claim 1, wherein said compound is 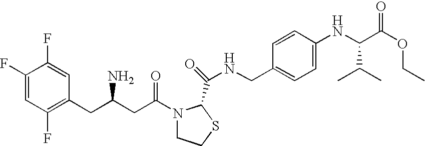
in free, salt or prodrug form.

9. The compound according to claim 1, wherein said compound is 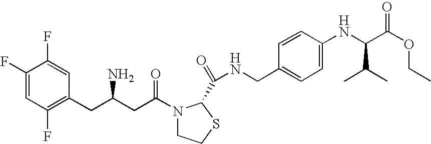
in free, salt or prodrug form.

10. The compound according to claim 1, wherein said compound is 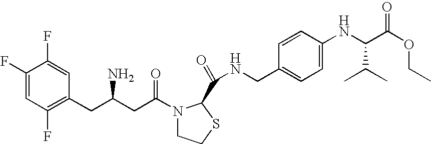
in free, salt or prodrug form.

11. The compound according to claim 1, wherein said compound is 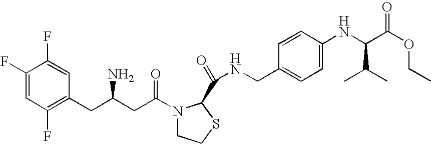
in free, salt or prodrug form.

12. The compound according to claim 1, wherein said compound is selected from: 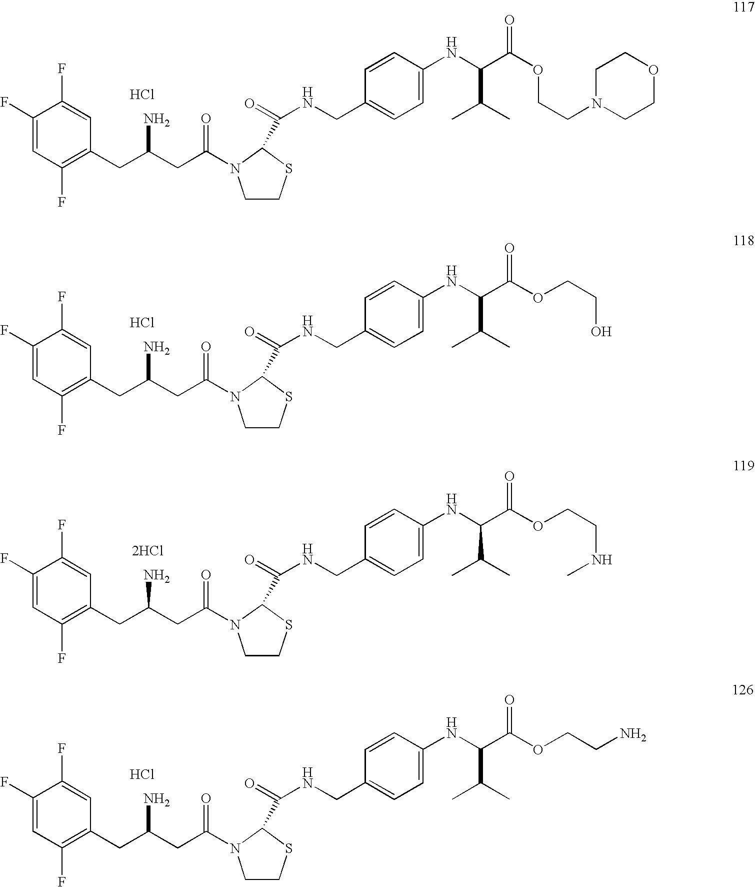 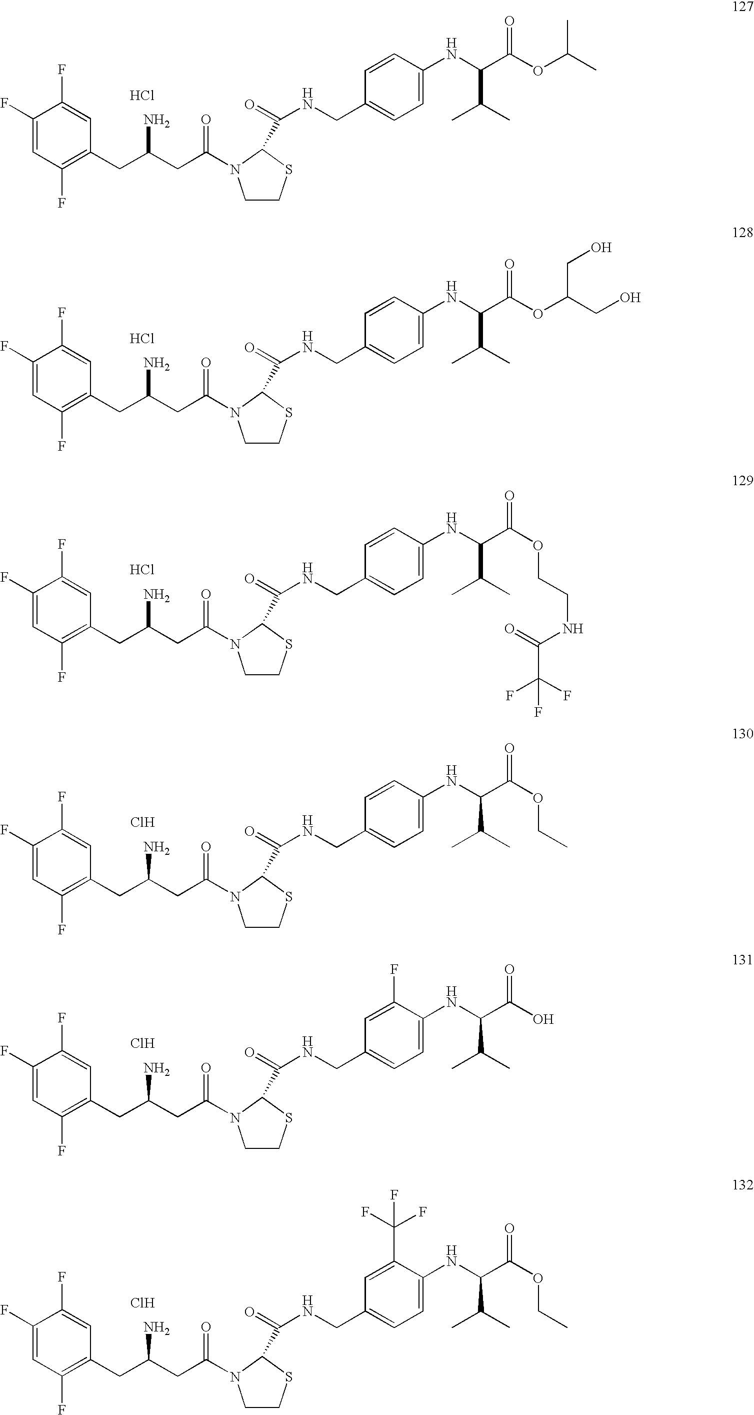 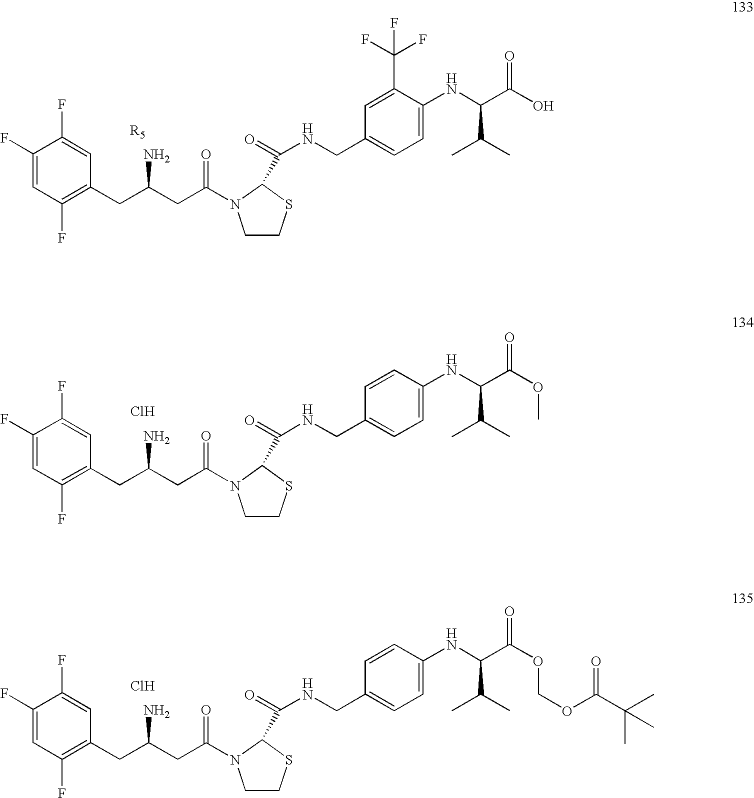
in free, salt or prodrug form.

13. The compound according to claim 1, wherein the salt is formed with a hydrochloric acid.

14. The compound according to claim 1, wherein said compound is a Compound of formula 1 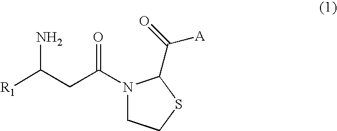
wherein,
A is 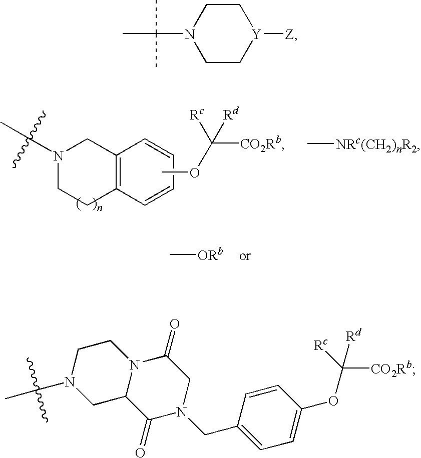
R₁ is 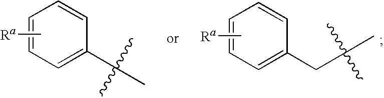
R₂ is 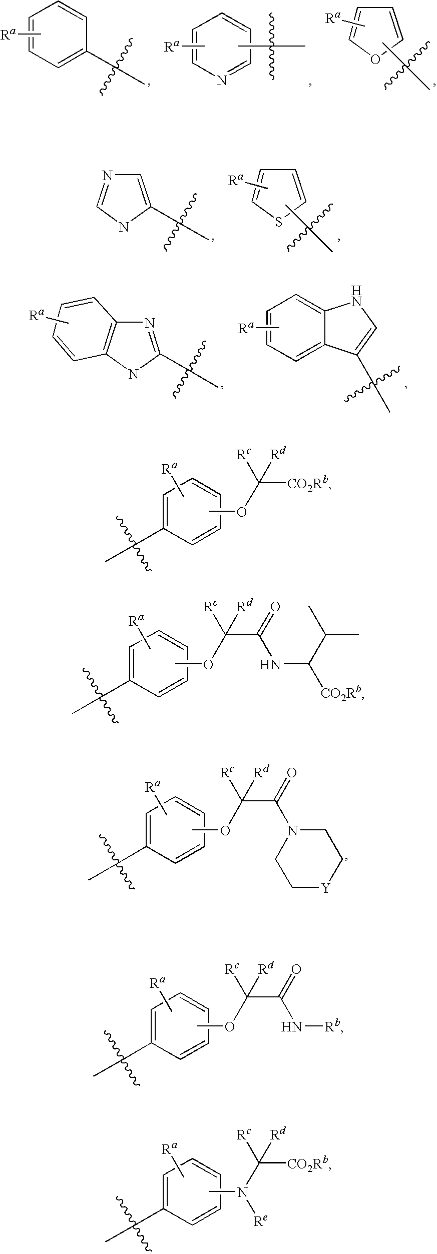 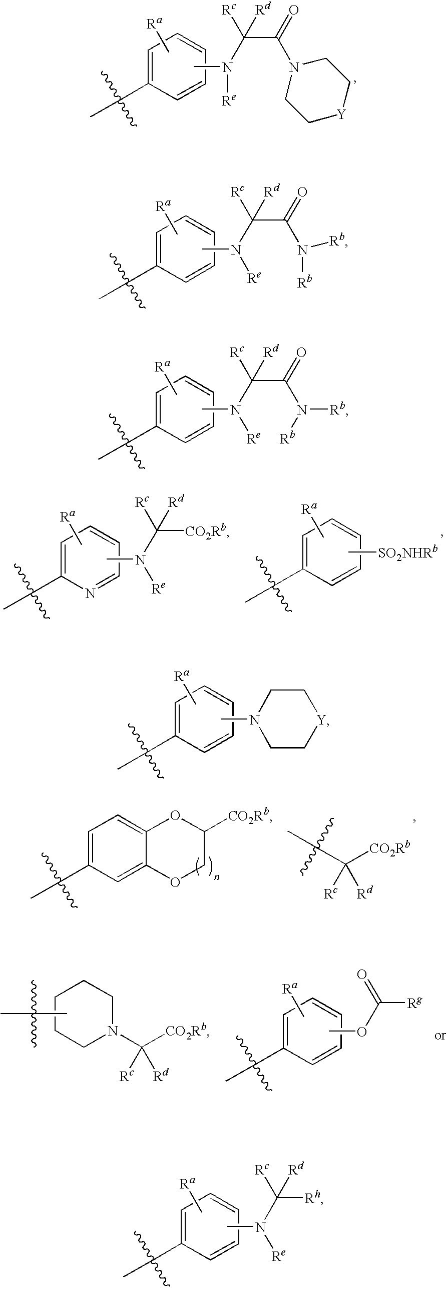
R^{a} is one or more substitutents selected from the group consisting of hydrogen, C₁₋₆ alkyl, C₃₋₆ cycloalkyl, C₁₋₆ alkoxy, —OCF₃, hydroxy, halogen, —CN, —CF₃, —COOR^{b}, —COOR^{b} and —NR^{d}R^{e};
R^{b} is hydrogen, C₁₋₆ alkyl, C₃₋₆ cycloalkyl, isopropyl, t-butyl, —CH₂CH₂OH, —CH₂CH₂NH₂, —CH₂CH₂N(CH₂CH₂)₂O, —CH₂CH₂N(CH₂CH₃)₂ or —CH₂CH₂NHCOCH₃;
R^{c} is hydrogen, C₁₋₆ alkyl, C₃₋₆ cycloalkyl, benzyl, isopropyl or t-butyl;
R^{d} and R^{e} are each independently hydrogen, C₁₋₆ alkyl or C₃₋₆ cycloalkyl;
Y is C, O, S or N;
Z is hydrogen, C₁₋₆ alkyl, C₃₋₆ cycloalkyl or —CO₂R^{b}; and
n is an integer of 0, 1 or 2,
in free or pharmaceutically acceptable salt form.

15. The compound of claim 14, wherein
R₁ is 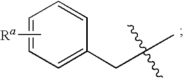
and
R^{a} is one or more substitutents selected from the group consisting of hydrogen, C₁₋₆ alkyl, C₁₋₆ alkoxy, —OCF₃, halogen, —CN and —CF₃
in free or pharmaceutically acceptable salt form.

16. (canceled)

17. The compound of claim 14, wherein A is —NH(CH₂)ₙR₂ and n and R₂ are defined in claim 14.

18. The compound of claim 14, which is selected from the group consisting of:
(1) methyl 3-((R)-3-amino-4-(2,4,5-trifluorophenyl)butanoyl)thiazolidine-2-carboxylate,
(2) 3-((R)-3-amino-4-(2,4,5-trifluorophenyl)butanoyl)thiazolidine-2-carboxylic acid,
(3) 3-((R)-3-amino-4-(2,4,5-trifluorophenyl)butanoyl)-N-benzylthiazolidine-2-carboxamide,
(4) ethyl 2-(4-((3-((R)-3-amino-4-(2,4,5-trifluorophenyl)butanoyl)thiazolidine-2-carboxamido)methyl)phenoxy)acetate,
(5) 2-4-((3-((R)-3-amino-4-(2,4,5-trifluorophenyl)butanoyl)thiazolidine-2-carboxamido)methyl)phenoxy)acetic acid,
(6) ethyl 2-(4-(3-((R)-3-amino-4-(2,4,5-trifluorophenyl)butanoyl)thiazolidine-2-carboxamido)phenoxy)acetate,
(7) 2-(4-(3-((R)-3-amino-4-(2,4,5-trifluorophenyl)butanoyl)thiazolidine-2-carboxamido)phenoxy)acetic acid,
(8) ethyl 2-(4-((3-((R)-3-amino-4-(2,4,5-trifluorophenyl)butanoyl)thiazolidine-2-carboxamido)methyl)phenoxy)-3-methylbutanoate,
(9) 2-(4-((3-((R)-3-amino-4-(2,4,5-trifluorophenyl)butanoyl)thiazolidine-2-carboxamido)methyl)phenoxy)-3-methylbutanoic acid,
(10) pivaloyloxymethyl 2-(4-((3-((R)-3-amino-4-(2,4,5-trifluorophenyl) butanoyl)thiazolidine-2-carboxamido)methyl)phenoxy)-3-methylbutanoate,
(11) ethyl 1-(3-((R)-3-amino-4-(2,4,5-trifluorophenyl)butanoyl)thiazolidine-2-carbonyl)piperidine-4-carboxylate,
(12) 1-(3-((R)-3-amino-4-(2,4,5-trifluorophenyl)butanoyl)thiazolidine-2-carbonyl)piperidine-4-carboxylic acid,
(13) 2-(4-((3-((R)-3-amino-4-(2,4,5-trifluorophenyl)butanoyl)thiazolidine-2-carboxamido)methyl)phenyl)acetic acid,
(14) ethyl 2-(2-(3-((R)-3-amino-4-(2,4,5-trifluorophenyl)butanoyl)thiazolidine-2-carbonyl)-1,2,3,4-tetrahydroisoquinolin-7-yloxy)-3-methylbutanoate,
(15) 2-(2-(3-((R)-3-amino-4-(2,4,5-trifluorophenyl)butanoyl)thiazolidine-2-carbonyl)-1,2,3,4-tetrahydroisoquinolin-7-yloxy)-3-methylbutanoic acid,
(16) ethyl 6-((3-((R)-3-amino-4-(2,4,5-trifluorophenyl)butanoyl)thiazolidine-2-carboxamido)methyl)-2,3-dihydrobenzo[b][1,4]dioxin-2-carboxylate,
(17) 6-((3-((R)-3-amino-4-(2,4,5-trifluorophenyl)butanoyl)thiazolidine-2-carboxamido)methyl)-2,3-dihydrobenzo[b][1,4]dioxin-2-carboxylic acid,
(18) pivaloyloxymethyl 2-(4-((3-((R)-3-amino-4-(2,4,5-trifluorophenyl) butanoyl)thiazolidine-2-carboxamido)methyl)phenoxy)-3-methylbutanoate,
(19) ethyl 2-(4-((3-((R)-3-((1-acetoxyethoxy)carbonylamino)-4-(2,4,5-trifluorophenyl)butanoyl)thiazolidine-2-carboxamido)methyl)phenoxy)-3-methylbutanoate,
(20) (3R)-3-amino-1-(2-(morpolin-4-carbonyl)thiazolidin-3-yl)-4-(2,4,5-trifluorophenyl)butan-1-on,
(21) N-(2-(1H-imidazol-5-yl)ethyl)-3-((R)-3-amino-4-(2,4,5-trifluorophenyl) butanoyl)thiazolidine-2-carboxamide,
(22) (S)-2-(4-(((S)-3-((R)-3-amino-4-(2,4,5-trifluorophenyl)butanoyl) thiazolidine-2-carboxamido)methyl)phenoxy)-3-methylbutanoic acid,
(23) (R)-2-(4-(((S)-3-((R)-3-amino-4-(2,4,5-trifluorophenyl)butanoyl) thiazolidine-2-carboxamido)methyl)phenoxy)-3-methylbutanoic acid,
(24) (S)-2-(4-(((R)-3-((R)-3-amino-4-(2,4,5-trifluorophenyl)butanoyl) thiazolidine-2-carboxamido)methyl)phenoxy)-3-methylbutanoic acid,
(25) (R)-2-(4-(((R)-3-((R)-3-amino-4-(2,4,5-trifluorophenyl)butanoyl) thiazolidine-2-carboxamido)methyl)phenoxy)-3-methylbutanoic acid,
(26) (S)-2-(4-(((S)-3-((R)-3-amino-4-(2,4,5-trifluorophenyl)butanoyl) thiazolidine-2-carboxamido)methyl)phenylamino)-3-methylbutanoic acid,
(27) (R)-2-(4-(((S)-3-((R)-3-amino-4-(2,4,5-trifluorophenyl)butanoyl) thiazolidine-2-carboxamido)methyl)phenylamino)-3-methylbutanoic acid,
(28) (S)-2-(4-(((R)-3-((R)-3-amino-4-(2,4,5-trifluorophenyl)butanoyl) thiazolidine-2-carboxamido)methyl)phenylamino)-3-methylbutanoic acid,
(29) (R)-2-(4-(((R)-3-((R)-3-amino-4-(2,4,5-trifluorophenyl)butanoyl) thiazolidine-2-carboxamido)methyl)phenylamino)-3-methylbutanoic acid,
(30) ethyl 2-(3-((3-((R)-3-amino-4-(2,4,5-trifluorophenyl)butanoyl) thiazolidine-2-carboxamido)methyl)phenoxy)acetate,
(31) 2-(3-((3-((R)-3-amino-4-(2,4,5-trifluorophenyl)butanoyl)thiazolidine-2-carboxamido)methyl)phenoxy)acetic acid,
(32) ethyl 2-(3-(3-((R)-3-amino-4-(2,4,5-trifluorophenyl)butanoyl) thiazolidine-2-carboxamido)phenoxy)acetate,
(33) 2-(3-(3-((R)-3-amino-4-(2,4,5-trifluorophenyl)butanoyl)thiazolidine-2-carboxamido)phenoxy)acetic acid,
(34) 2-(4-(3-((R)-3-amino-4-(2,4,5-trifluorophenyl)butanoyl)thiazolidine-2-carboxamido)piperidine-1-yl)-3-methylbutanoic acid,
(35) (S)-ethyl 2-(4-(((S)-3-((R)-3-amino-4-(2,4,5-trifluorophenyl)butanoyl) thiazolidine-2-carboxamido)methyl)phenylamino)-3-methylbutanoate,
(36) (R)-ethyl 2-(4-(((S)-3-((R)-3-amino-4-(2,4,5-trifluorophenyl)butanoyl) thiazolidine-2-carboxamido)methyl)phenylamino)-3-methylbutanoate,
(37) (S)-ethyl 2-(4-(((R)-3-((R)-3-amino-4-(2,4,5-trifluorophenyl)butanoyl) thiazolidine-2-carboxamido)methyl)phenylamino)-3-methylbutanoate,
(38) (R)-ethyl 2-(4-(((R)-3-((R)-3-amino-4-(2,4,5-trifluorophenyl)butanoyl) thiazolidine-2-carboxamido)methyl)phenylamino)-3-methylbutanoate,
(39) (3R)-3-amino-1-(2-(thiomorpolin-4-carbonyl)thiazolidin-3-yl)-4-(2,4,5-trifluorophenyl)butan-1-on,
(40) (3R)-3-amino-1-(2-(piperazine-1-carbonyl)thiazolidin-3-yl)-4-(2,4,5-trifluorophenyl)butan-1-on,
(41) (3R)-3-amino-1-(2-(4-methylpiperazine-1-carbonyl)thiazolidin-3-yl)-4-(2,4,5-trifluorophenyl)butan-1-one,
(42) 3-((R)-3-amino-4-(2,4,5-trifluorophenyl)butanoyl)-N,N-dimethyl thiazolidine-2-carboxamide,
(43) 3-((R)-3-amino-4-(2,4,5-trifluorophenyl)butanoyl)-N-(furan-3-yl)methyl) thiazolidine-2-carboxamide,
(44) ethyl 2-(3-((R)-3-amino-4-(2,4,5-trifluorophenyl)butanoyl)thiazolidine-2-carboxamido)acetate,
(45) 2-(3-((R)-3-amino-4-(2,4,5-trifluorophenyl)butanoyl)thiazolidine-2-carboxamido)acetic acid,
(46) N-(2-(1H-indol-3-yl)ethyl)-3-((R)-3-amino-4-(2,4,5-trifluorophenyl) butanoyl)thiazolidine-2-carboxamide,
(47) 3-((R)-3-amino-4-(2,4,5-trifluorophenyl)butanoyl)-N-(4-morpholinophenyl) thiazolidine-2-carboxamide,
(48) 3-((R)-3-amino-4-(2,4,5-trifluorophenyl)butanoyl)-N-(4-sulfamoylphenyl) thiazolidine-2-carboxamide,
(49) 3-((R)-3-amino-4-(2,4,5-trifluorophenyl)butanoyl)-N-(4-sulfamoylbenzyl) thiazolidine-2-carboxamide,
(50) N-((1H-benzo[d]imidazol-2-yl)methyl)-3-((R)-3-amino-4-(2,4,5-trifluorophenyl)butanoyl)thiazolidine-2-carboxamide,
(51) ethyl 2-(4-((3-((R)-3-amino-4-(2,4,5-trifluorophenyl)butanoyl) thiazolidine-2-carboxamido)methyl)-3-fluorophenoxy)butanoate,
(52) 2-(4-((3-((R)-3-amino-4-(2,4,5-trifluorophenyl)butanoyl)thiazolidine-2-carboxamido)methyl)-3-fluorophenoxy)butanoic acid,
(53) ethyl 2-(4-((3-((R)-3-amino-4-(2,4,5-trifluorophenyl)butanoyl)thiazolidine-2-carboxamido)methyl)-3-fluorophenoxy)-2-methylpropanoate,
(54) 2-(4-((3-((R)-3-amino-4-(2,4,5-trifluorophenyl)butanoyl)thiazolidine-2-carboxamido)methyl)-3-fluorophenoxy)-2-methylpropanoic acid,
(55) ethyl 2-(4-((3-((R)-3-amino-4-(2,4,5-trifluorophenyl)butanoyl)thiazolidine-2-carboxamido)methyl)-2-fluorophenoxy)-2-methylpropanoate,
(56) 3-((R)-3-amino-4-(2,4,5-trifluorophenyl)butanoyl)-N-(pyridin-4-yl methyl)thiazolidine-2-carboxamide,
(57) (S)-2-(2-(4-((3-((R)-3-amino-4-(2,4,5-trifluorophenyl)butanoyl)thiazolidine-2-carboxamido)methyl)phenoxy)-3-methylbutanamido)-3-methylbutanoic acid,
(58) (R)-ethyl 2-(4-((8-(3-((R)-3-amino-4-(2,4,5-trifluorophenyl)butanoyl) thiazolidine-2-carbonyl)-1,4-dioxo-hexahydro-1H-pyrazino[1,2-a]pyrazin-2(6H)-yl)methyl)phenoxy)-3-methylbutanoate,
(59) (R)-2-(4-((8-(3-((R)-3-amino-4-(2,4,5-trifluorophenyl)butanoyl)thiazolidine-2-carbonyl)-1,4-dioxo-hexahydro-1H-pyrazino[1,2-a]pyrazin-2(6H)-yl)methyl)phenoxy)-3-methylbutanoic acid,
(60) ethyl 2-(2-(3-((R)-3-amino-4-(2,4,5-trifluorophenyl)butanoyl)thiazolidine-2-carbonyl)-1,2,3,4-tetrahydroisoquinoline-6-yloxy)-3-methylbutanoate,
(61) 2-(2-(3-((R)-3-amino-4-(2,4,5-trifluorophenyl)butanoyl)thiazolidine-2-carbonyl)-1,2,3,4-tetrahydroisoquinoline-6-yloxy)-3-methylbutanoic acid,
(62) ethyl 5-((3-((R)-3-amino-4-(2,4,5-trifluorophenyl)butanoyl)thiazolidine-2-carboxamido)methyl)benzo[d][1,3]dioxol-2-carboxylate,
(63) 5-((3-((R)-3-amino-4-(2,4,5-trifluorophenyl)butanoyl)thiazolidine-2-carboxamido)methyl)benzo[d][1,3]dioxol-2-carboxylic acid,
(64) 2-(4-((3-((R)-3-amino-4-(2,4,5-trifluorophenyl)butanoyl)thiazolidine-2-carboxamido)methyl)phenoxy)-2-methylpropanoic acid,
(65) (R)-2-(4-((3-((R)-3-amino-4-(2,4,5-trifluorophenyl)butanoyl)thiazolidine-2-carboxamido)methyl)phenoxy)-3-phenylpropanoic acid,
(66) 2-(4-((3-((R)-3-amino-4-(2,4,5-trifluorophenyl)butanoyl)-N-methyl thiazolidine-2-carboxamido)methyl)phenoxy)-3-methylbutanoic acid,
(67) ethyl 2-(4-((3-((R)-3-amino-4-(2,4,5-trifluorophenyl)butanoyl)thiazolidine-2-carboxamido)methyl)phenylamino)-3-methylbutanoate,
(68) 2-(4-((3-((R)-3-amino-4-(2,4,5-trifluorophenyl)butanoyl)thiazolidine-2-carboxamido)methyl)phenylamino)-3-methylbutanoic acid,
(69) ethyl 2-(4-((3-((R)-3-amino-4-(2,4,5-trifluorophenyl)butanoyl)thiazolidine-2-carboxamido)methyl)-3-fluorophenoxy)-3-methylbutanoate,
(70) 2-(4-((3-((R)-3-amino-4-(2,4,5-trifluorophenyl)butanoyl)thiazolidine-2-carboxamido)methyl)-3-fluorophenoxy)-3-methylbutanoic acid,
(71) 2-(4-((3-((R)-3-amino-4-(2,4,5-trifluorophenyl)butanoyl)thiazolidine-2-carboxamido)methyl)-2-fluorophenoxy)-2-methylpropanoic acid,
(72) ethyl 2-(4-(((S)-3-((R)-3-amino-4-(2,4,5-trifluorophenyl)butanoyl) thiazolidine-2-carboxamido)methyl)phenylamino)-2-methylpropanoate,
(73) 2-(4-(((S)-3-((R)-3-amino-4-(2,4,5-trifluorophenyl)butanoyl)thiazolidine-2-carboxamido)methyl)phenylamino)-2-methylpropanoic acid,
(74) (S)-methyl 2-(4-(((S)-3-((R)-3-amino-4-(2,4,5-trifluorophenyl)butanoyl) thiazolidine-2-carboxamido)methyl)-3-fluorophenylamino)-3-methylbutanoate,
(75) (S)-ethyl 2-(3-((R)-3-amino-4-(2,4,5-trifluorophenyl)butanoyl)thiazolidine-2-carboxamido)-3-methylbutanoate,
(76) (S)-2-(3-((R)-3-amino-4-(2,4,5-trifluorophenyl)butanoyl)thiazolidine-2-carboxamido)-3′-methylbutanoic acid,
(77) (2S,3S)-ethyl 2-(3-((R)-3-amino-4-(2,4,5-trifluorophenyl)butanoyl) thiazolidine-2-carboxamido)-3-methylpentanoate,
(78) (2S,3S)-2-(3-((R)-3-amino-4-(2,4,5-trifluorophenyl)butanoyl)thiazolidine-2-carboxamido)-3-methylpentanoic acid,
(79) ethyl 2-(4-(3-((R)-3-amino-4-(2,4,5-trifluorophenyl)butanoyl) thiazolidine-2-carboxamido)piperidine-1-yl)-3-methylbutanoate,
(80) 4-((3-((R)-3-amino-4-(2,4,5-trifluorophenyl)butanoyl)thiazolidine-2-carboxamido)methyl)phenyl acetate,
(81) 3-((R)-3-amino-4-(2,4,5-trifluorophenyl)butanoyl)-N-(4-hydroxybenzyl) thiazolidine-2-carboxamide,
(82) ethyl 2-((4-((3-((R)-3-amino-4-(2,4,5-trifluorophenyl)butanoyl)thiazolidine-2-carboxamido)methyl)phenyl)(methyl)amino)-3-methylbutanoate,
(83) methyl 4-((3-((R)-3-amino-4-(2,4,5-trifluorophenyl)butanoyl)thiazolidine-2-carboxamido)methyl)-2-hydroxybenzoate,
(84) ethyl 2-(4-((3-((R)-3-amino-4-(2,4,5-trifluorophenyl)butanoyl)thiazolidine-2-carboxamido)methyl)phenoxy)propanoate,
(85) 2-((4-((3-((R)-3-amino-4-(2,4,5-trifluorophenyl)butanoyl)thiazolidine-2-carboxamido)methyl)phenyl)(methyl)amino)-3-methylbutanoic acid,
(86) 4-((3-((R)-3-amino-4-(2,4,5-trifluorophenyl)butanoyl)thiazolidine-2-carboxamido)methyl)-2-hydroxybenzoic acid,
(87) (S)-2-(4-(((S)-3-((R)-3-amino-4-(2,4,5-trifluorophenyl)butanoyl) thiazolidine-2-carboxamido)methyl)-3-fluorophenylamino)-3-methylbutanoic acid,
(88) (S)-ethyl 2-(4-(((S)-3-((R)-3-amino-4-(2,4,5-trifluorophenyl)butanoyl) thiazolidine-2-carboxamido)methyl)phenoxy)-3-methylbutanoate,
(89) (S)-ethyl 2-(4-(((S)-3-((R)-3-amino-4-(2,4,5-trifluorophenyl)butanoyl) thiazolidine-2-carboxamido)methyl)-2-fluorophenylamino)-3-methylbutanoate,
(90) (S)-2-(4-(((S)-3-((R)-3-amino-4-(2,4,5-trifluorophenyl)butanoyl) thiazolidine-2-carboxamido)methyl)-2-fluorophenylamino)-3-methylbutanoic acid,
(91) (S)-ethyl 2-(6-(((S)-3-((R)-3-amino-4-(2,4,5-trifluorophenyl)butanoyl) thiazolidine-2-carboxamido)methyl)pyridin-3-ylamino)-3-methylbutanoate,
(92) (S)-2-(6-(((S)-3-((R)-3-amino-4-(2,4,5-trifluorophenyl)butanoyl) thiazolidine-2-carboxamido)methyl)pyridin-3-ylamino)-3-methylbutanoic acid,
(93) 2-(4-((3-((R)-3-amino-4-(2,4,5-trifluorophenyl)butanoyl)thiazolidine-2-carboxamido)methyl)phenoxy)propanoic acid,
(94) (S)-2-(4-(((S)-3-((R)-3-amino-4-(2,4,5-trifluorophenyl)butanoyl) thiazolidine-2-carboxamido)methyl)phenylamino)-3,3-dimethylbutanoic acid,
(95) (S)-2-(2-(((S)-3-((R)-3-amino-4-(2,4,5-trifluorophenyl)butanoyl) thiazolidine-2-carboxamido)methyl)phenylamino)-3-methylbutanoic acid,
(96) (S)-2-(3-(((S)-3-((R)-3-amino-4-(2,4,5-trifluorophenyl)butanoyl) thiazolidine-2-carboxamido)methyl)phenylamino)-3-methylbutanoic acid,
(97) 4-((3-((R)-3-amino-4-(2,4,5-trifluorophenyl)butanoyl)thiazolidine-2-carboxamido)methyl)benzoic acid,
(98) 3-((R)-3-amino-4-(2,4,5-trifluorophenyl)butanoyl)-N-(4-(2-oxo-2-(piperazine-1-yl)ethoxy)benzyl)thiazolidine-2-carboxamide,
(99) 3-((R)-3-amino-4-(2,4,5-trifluorophenyl)butanoyl)-N-(4-(2-oxo-2-thiomorpholinoethoxy)benzyl)thiazolidine-2-carboxamide, and
(100) 3-((R)-3-amino-4-(2,4,5-trifluorophenyl)butanoyl)-N-(4-(2-morpholino-2-oxoethoxy)benzyl)thiazolidine-2-carboxamide,
in free or a pharmaceutically acceptable salt form.

19. The compound of claim 14, which has the form of R-isomer in the carbon atom having the amino group and R₁ substituent.

20. The compound of claim 14, wherein the salt is formed with an acid selected from hydrochloric, sulfuric, acetic, trifluoroacetic, phosphoric, fumaric, maleic, citric, methanesulfonic and lactic acids.

21. (canceled)

22. A method for preparing a compound of 2-carbonyl-3-acyl-1,3-thiazolidine derivative of formula Q-1b, comprising the steps of:
(i) subjecting an amino acid of formula Q-2 to a condensation reaction with a 2-carbonyl-1,3-thiazolidine-based compound of formula Q-3 to form a compound of formula Q-4;
(ii) forming a compound of formula Q-5 from the compound of formula Q-4; and
(iii) deprotecting the compound of formula Q-5 to obtain the compound of 2-carbonyl-3-acyl-1,3-thiazolidine derivative of formula Q-1b: 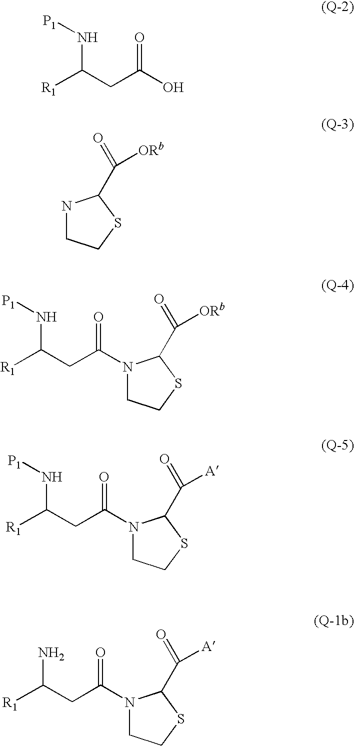
wherein, A′ is 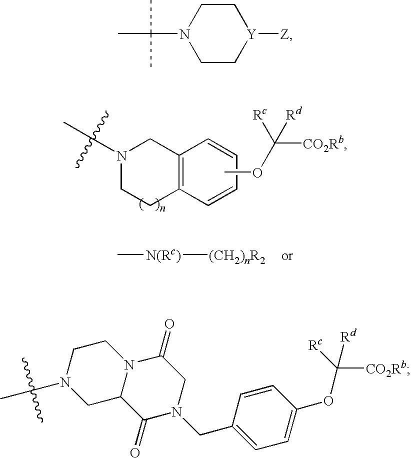
P₁, R₁, R₂, R^{b} to R^{c}, Y, Z and n are the same as defined above in formula (Q).

23. (canceled)

24. (canceled)

25. (canceled)

26. (canceled)

27. A method for preparing a compound of 2-carbonyl-3-acyl-1,3-thiazolidine derivative of formula Q-1b-3, comprising the steps of:
(i) hydrolyzing a compound of formula Q-11 to form a compound of formula Q-12; and
(ii) deprotecting the compound of formula Q-12 to obtain a compound of 2-carbonyl-3-acyl-1,3-thiazolidine derivative of formula Q-1b-3: 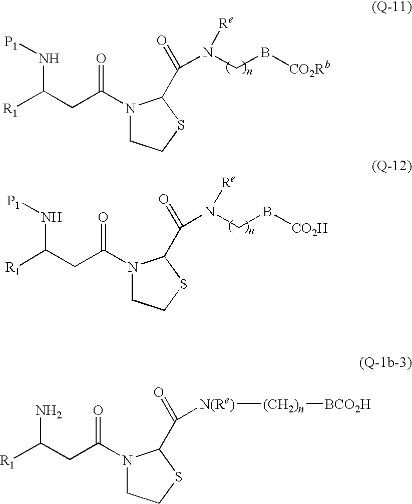
wherein, B is a substitutent selected from the group consisting of, 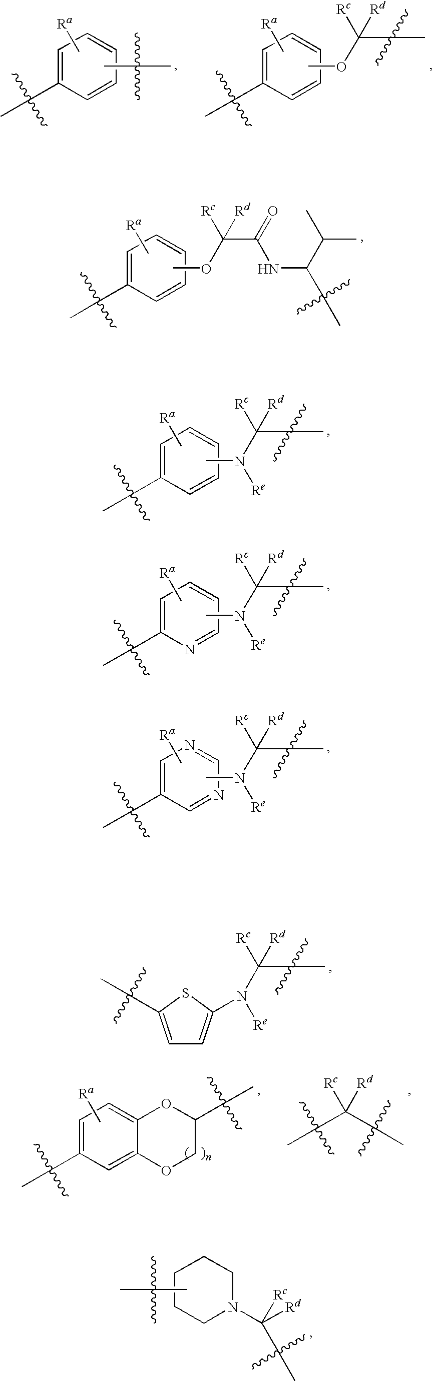 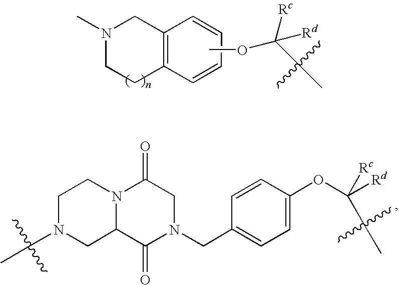
wherein N(R^{e})—(CH₂)ₙ— is attached to the left side of the B and —CO₂R^{b} or CO₂H is attached to the right side of B; and P₁, R₁, R^{a} to R^{g} and n are the same as defined above in formula (Q) of claim 2.

28. (canceled)

29. (canceled)

30. The method of claim 22, further comprising obtaining a stereoisomer of formula 3a or 3b from the compound of formula Q-3 by recrystallization utilizing dynamic kinetic resolution: 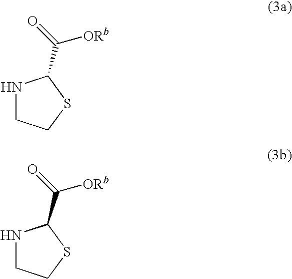
wherein, R^{b} is the same as defined in claim 22.

31. The method of claim 22, wherein step ii) comprises hydrolyzing the compound of formula 4 to form a compound of formula 7 and bringing the compound of formula 7 to react with an A′-containing nucleophilic compound to obtain the compound of formula Q-5: 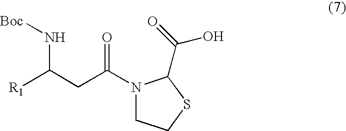
wherein, R₁ and A′ is the same as defined in claim 22.

32. (canceled)

33. (canceled)

34. (canceled)

35. A pharmaceutical composition comprising the compound of claim 1 in free, pharmaceutically acceptable salt or prodrug form in combination or association with a pharmaceutically acceptable diluent or carrier.

36. (canceled)

37. (canceled)

38. A method for inhibiting DPP-IV in a mammal, comprising administering the compound according to claim 1 in free, pharmaceutically acceptable salt or prodrug form to the mammal in an amount effective for the inhibition of DPP-IV.

39. A method for treating DPP-IV-mediated diseases in a mammal, comprising administering the compound according to claim 1 in free, pharmaceutically acceptable salt or prodrug form to the mammal in a therapeutically effective amount.

40-43. (canceled)

44. The method according to claim 38, wherein the compound is a compound according to Formula (Q) of claim 2, in free, pharmaceutically acceptable salt or prodrug form.

45. The method according to claim 44, wherein the compound is selected from a group consisting of: 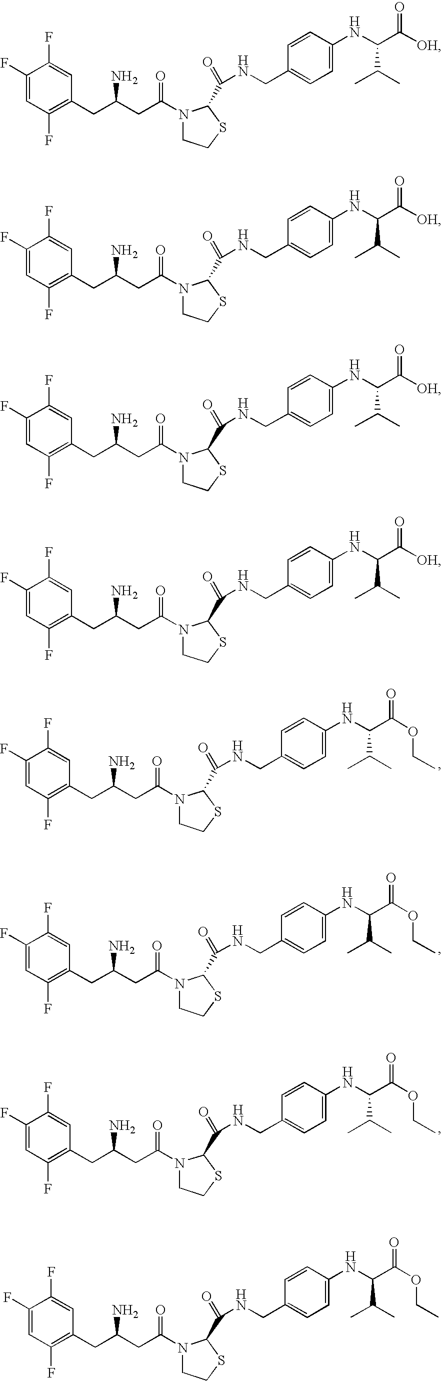
in free, pharmaceutically acceptable salt or prodrug form.

46. The method according to claim 39, wherein the compound is a compound according to Formula (Q) of claim 2, in free, pharmaceutically acceptable salt or prodrug form.

47. The method according to claim 46, wherein the compound is selected from a group consisting of: 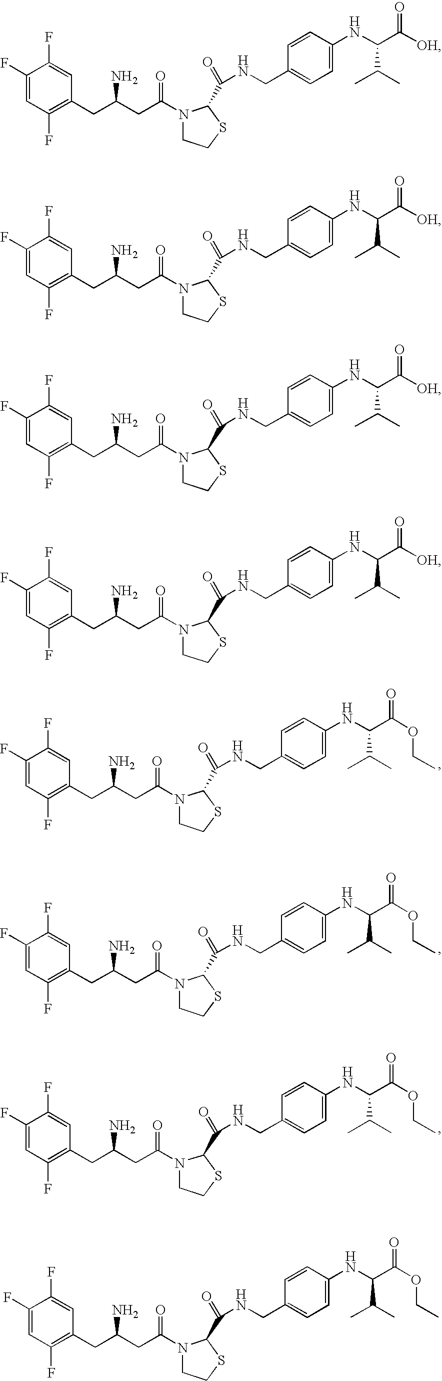
in free, pharmaceutically acceptable salt or prodrug form.
